(19) United States
(12) Patent Application Publication
(10) Pub. No.: US 2026/0317339 A1
(43) Pub. Date: Oct. 15, 2026

(54) DELIVERY OF THERAPEUTIC ALKALOID COMPOUNDS

(71) Applicant: Sensorium Therapeutics, Inc., Boston, MA (US)

(72) Inventors: Jacob M. Hooker, Winchester, MA (US); Michael S. Placzek, Tewksbury, MA (US)

(21) Appl. No.: 19/480,876

(22) PCT Filed: May 2, 2024
(86) PCT No.: PCT/US2024/027330

§ 371 Date: Nov. 3, 2025

Related U.S. Application Data
(60) Provisional application No. 63/463,752, filed on May 3, 2023.

(51) Int. Cl. C07D 491/20; C07D 209/12; C07D 401/12; C07D 403/12; C07D 405/12; C07D 409/12; C07D 487/10; C07D 491/113; C07D 495/10; C07D 497/10; C07D 498/10; C07D 513/10; C07F 7/08

(52) U.S. Cl. CPC C07D 491/20; C07D 209/12; C07D 401/12; C07D 403/12; C07D 405/12; C07D 409/12; C07D 487/10; C07D 491/113; C07D 495/10; C07D 497/10; C07D 498/10; C07D 513/10; C07F 7/0892

(57) ABSTRACT

Certain pharmaceutical compounds are disclosed herein, including compounds of formula (I):

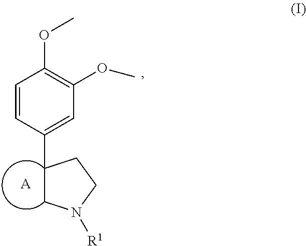

and pharmaceutically acceptable salts thereof, and compounds that can be obtained from Δ⁷-mesembrenone.

### RELATED APPLICATIONS

This application claims the benefit of priority to U.S. Provisional Patent Application No. 63/463,752, filed May 3, 2023; incorporated herein by reference in its entirety.

### TECHNICAL FIELD

The present disclosure relates to the field of medicine, including the discovery of novel alkaloid compounds useful for inhibiting the serotonin transporter protein (5-HTT).

### BACKGROUND

Plants of the genus Sceletium contain indole alkaloids having biological activity useful in treating mental health conditions such as mild to moderate depression. Natural extracts of Sceletium tortuosum, an indigenous herb of South Africa also referred to as “kougoed”, “channa” or “kanna,” can contain the pharmacologically active alkaloids. Mesembrine and mesembrenol are pharmacologically active alkaloids present in Sceletium tortuosum extracts used for treatment of anxiety, stress and mental health conditions.

Natural products obtained from plants of the genus Sceletium contain varying amounts of (−) mesembrine, (+)/(−) mesembrenone, and Δ⁷-mesembrenone (a/k/a D⁷-mesembrenone). The structure of Δ⁷-mesembrenone, also known as 3a-(3,4-dimethoxyphenyl)-1-methyl-2,3,4,5-tetrahydroindol-6-one, has been reported by Jeffs et al., Chemical Communications, 1971 (21) 1338. Naturally occurring (−) mesembrine from Sceletium tortuosum has been reported as having serotonin (5-HT) uptake inhibitory activity useful in treating mental health conditions such as mild to moderate depression.

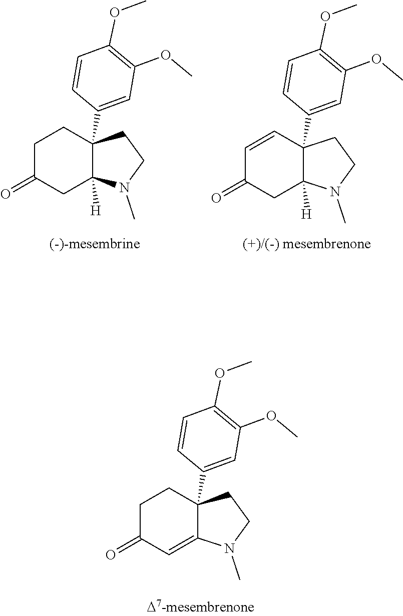 [SMILES: COc1ccc([C@@]23CCC(=O)C[C@@H]2N(C)CC3)cc1OC ; COc1ccc([C@@]23C=CC(=O)C[C@@H]2N(C)CC3)cc1OC ; COc1ccc([C@@]23CCC(=O)C=C2N(C)CC3)cc1OC]

However, bioactive plant extracts for therapeutic consumption can vary widely both seasonally and between different Sceletium tortuosum plants. Extracts fail to provide a sufficiently reproducible and stable phytochemical profile of desired biologically active components. Plants of the genus Sceletium and extracts thereof can vary widely in terms of the total alkaloid content, as well as the chemistry and relative concentrations of individual Sceletium plant derived alkaloids.

The therapeutic use of mesembrine has been limited by the variability and instability of these compounds content in natural extract products and the instability and pharmacokinetic profile of these compounds as obtained from natural products In addition, Δ⁷-mesembrenone is only a minor constituent of plants of the genus Sceletium. The therapeutic use of Δ⁷-mesembrenone has been limited by access to compound as it is only present in trace amounts in plants.

There remains an unmet need for pharmaceutical compositions comprising higher purity, predictable, stable and reproducible forms of therapeutic alkaloid compounds such as Δ⁷-mesembrenone. In addition, there is a need for oral pharmaceutical compositions providing pure therapeutic alkaloid compositions having desired pharmacokinetic properties upon administration. Finally, there is an unmet need for isolated and stabilized forms of Δ⁷-mesembrenone, and pharmaceutical compositions comprising markedly different properties than the naturally occurring compositions obtained from plant extracts.

### SUMMARY

Described herein are compounds that, when administered orally or intravenously to a subject, allow for sustained release of Δ⁷-mesembrenone, thereby extending exposure of Δ⁷-mesembrenone in the brain compared to a subject receiving an equivalent oral or intravenous dose of mesembrine itself. The sustained release and extended brain exposure to mesembrine will address recognized therapeutic shortcomings attributed to the pharmacokinetics of mesembrine. The prodrug compounds provide improved duration of action of Δ⁷-mesembrenone for enhanced therapeutic benefit.

Described herein are compounds of formula (I):

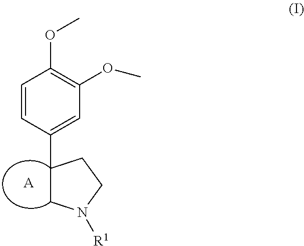

or a pharmaceutically acceptable salt thereof, wherein
R¹ is C₁-C₇ alkyl or H; and
ring A is

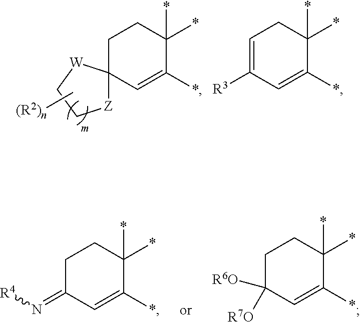

wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, C₃-C₁₀ cycloalkyl, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl or phenyl, wherein each hydrogen atom in 5- to 7-membered heteroaryl or phenyl is optionally substituted by —OH, C₁-C₃ alkyl, or C₁-C₃ alkanol;
m is 1 or 2;
n is 0, 1, 2, or 3;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, phenoxy or —O(CH₂)ₚOCH₃;
p is 2, 3, or 4;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₅ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and
each of R⁶ and R⁷ is independently C₁-C₃ alkyl.

In certain embodiments, the compound is of formula (I-1):

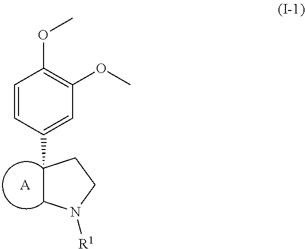

or a pharmaceutically acceptable salt thereof, wherein
R¹ is H or C₁-C₇ alkyl; and
ring A is

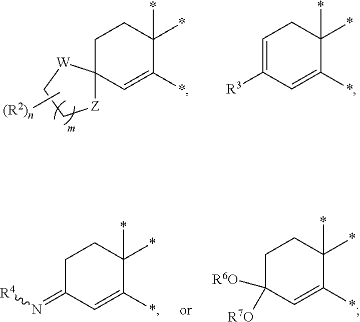

wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, C₃-C₁₀ cycloalkyl, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl or phenyl, wherein each hydrogen atom in 5- to 7-membered heteroaryl or phenyl is optionally substituted by —OH, C₁-C₃ alkyl, or C₁-C₃ alkanol,
m is 1 or 2,
n is 0, 1, or 2,
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and
each of R⁶ and R⁷ is independently C₁-C₃ alkyl.

In certain embodiments, the compound is of formula (I-1):

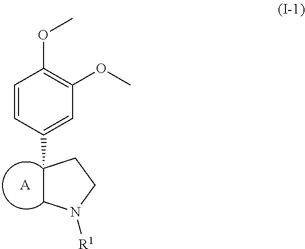

or a pharmaceutically acceptable salt thereof, wherein
R¹ is C₁-C₇ alkyl or H; and
ring A is

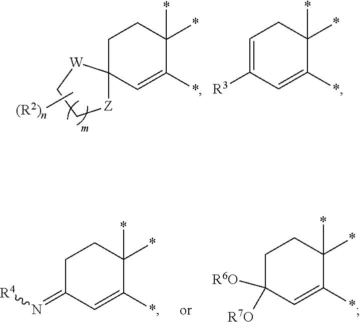

wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, C₃-C₁₀ cycloalkyl, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl or phenyl, wherein each hydrogen atom in 5- to 7-membered heteroaryl or phenyl is optionally substituted by —OH, C₁-C₃ alkyl, or C₁-C₃ alkanol;
m is 1 or 2;
n is 0, 1, or 2;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy or —O(CH₂)ₚOCH₃;
p is 2, 3, or 4;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, —C(O)-5- to 7-membered heteroaryl, —C(O)—C₁-C₈ alkyl, —C(O)—C₃-C₁₀ cycloalkyl, —C(O)— phenyl, —C(O)N(H)-phenyl, C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, C(O)N(H)-5- to 7-membered heteroaryl, and —C(O)N(H)—C₁-C₆ alkyl is optionally substituted by halogen, OH, C₁-C₃ alkyl, C₃—C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl;
each of R⁶ and R⁷ is independently C₁-C₃ alkyl; and
the compound of formula (I-1) has the absolute stereochemistry shown.

In certain embodiments, the compound of formula (I) is a compound of formula (IIa):

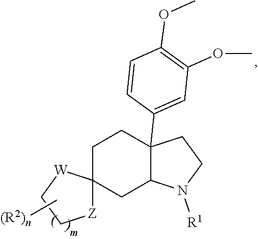

or a pharmaceutically acceptable salt thereof, wherein R¹, R², m, n, W, and Z are as defined herein.

In certain embodiments, the compound is of formula (IIa-1):

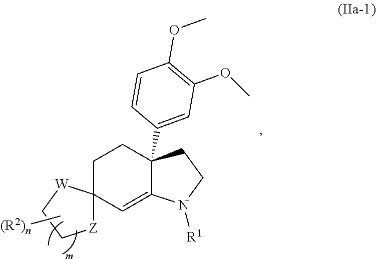

or a pharmaceutically acceptable salt thereof, wherein R¹, R², m, n, W, and Z are as defined herein; and the compound of formula (IIa-1) has the absolute stereochemistry shown.

In certain embodiments, the compound of formula (I) is a compound of formula (IIIa):

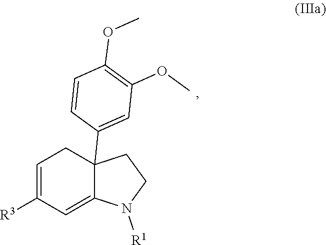

or a pharmaceutically acceptable salt thereof, wherein R¹ and R³ are as defined herein.

In certain embodiments, the compound is of formula (IIIa-1):

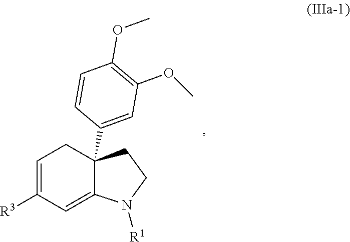

or a pharmaceutically acceptable salt thereof, wherein R¹ and R³ are as defined herein or a pharmaceutically acceptable salt thereof; and the compound of formula (IIIa-1) has the absolute stereochemistry shown.

In certain embodiments, the compound of formula (I) is a compound of formula (IVa):

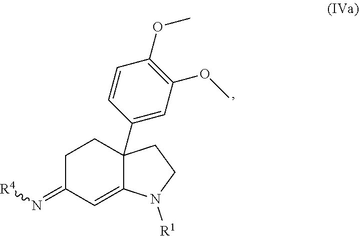

or a pharmaceutically acceptable salt thereof, wherein R¹ and R⁴ are as defined herein.

In certain embodiments, the compound is of formula (IVa-1):

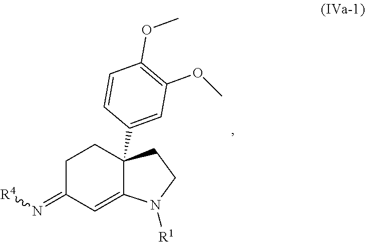

or a pharmaceutically acceptable salt thereof, wherein R¹ and R⁴ are as defined herein; and the compound of formula (IVa-1) has the absolute stereochemistry shown.

In certain embodiments, the compound is of formula (IA):

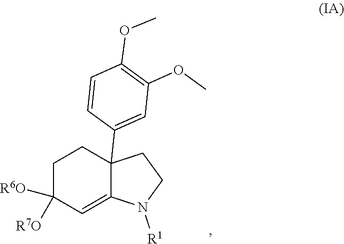

or a pharmaceutically acceptable salt thereof, and R¹, R⁶ and R⁷ are as defined herein.

In some embodiments, the compound is selected from the group consisting of:

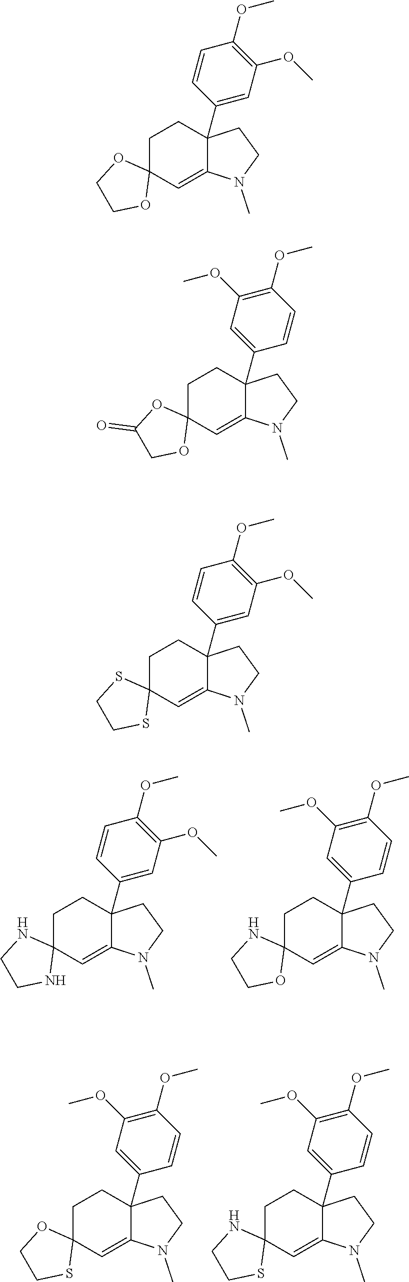

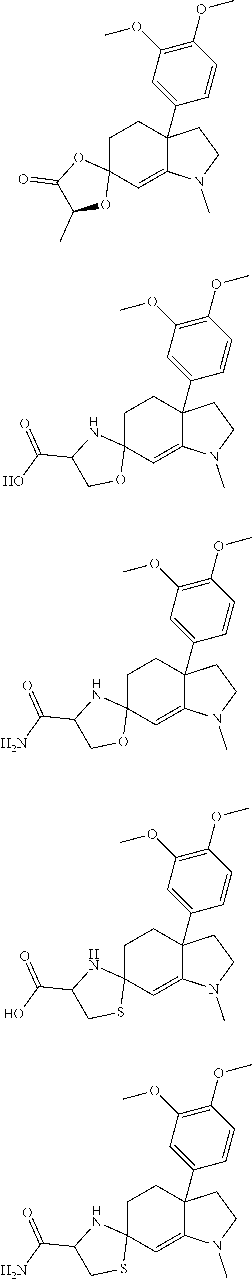

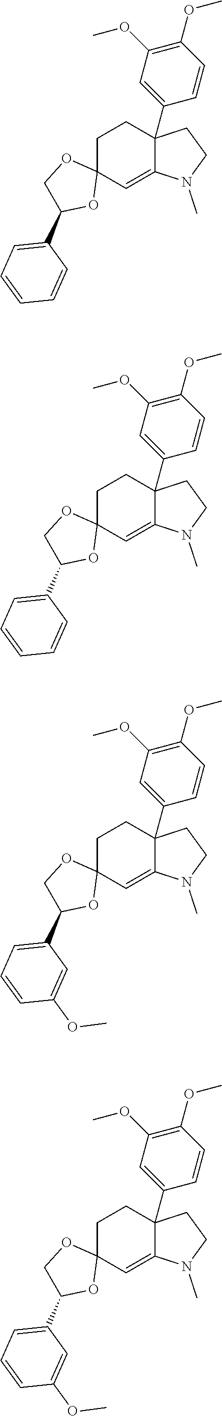 [SMILES: COc1ccc(C23CCN(C)C2=CC2(CC3)OC[C@H](c3ccccc3)O2)cc1OC ; COc1ccc(C23CCN(C)C2=CC2(CC3)OC[C@@H](c3ccccc3)O2)cc1OC ; COc1cccc([C@H]2COC3(C=C4N(C)CCC4(c4ccc(OC)c(OC)c4)CC3)O2)c1 ; COc1cccc([C@@H]2COC3(C=C4N(C)CCC4(c4ccc(OC)c(OC)c4)CC3)O2)c1]

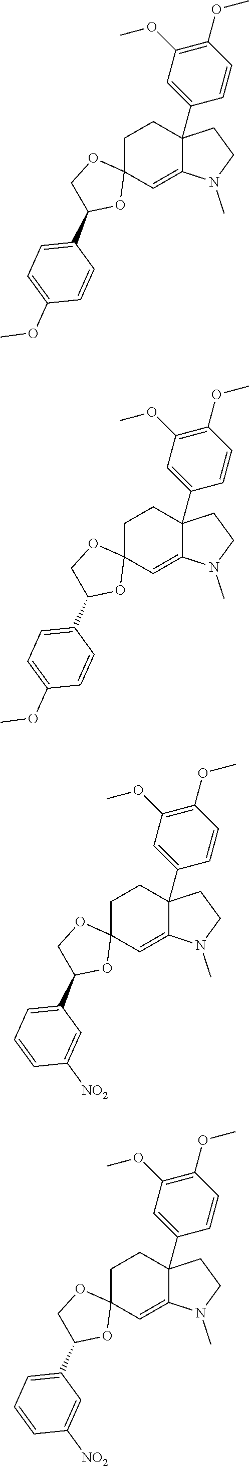

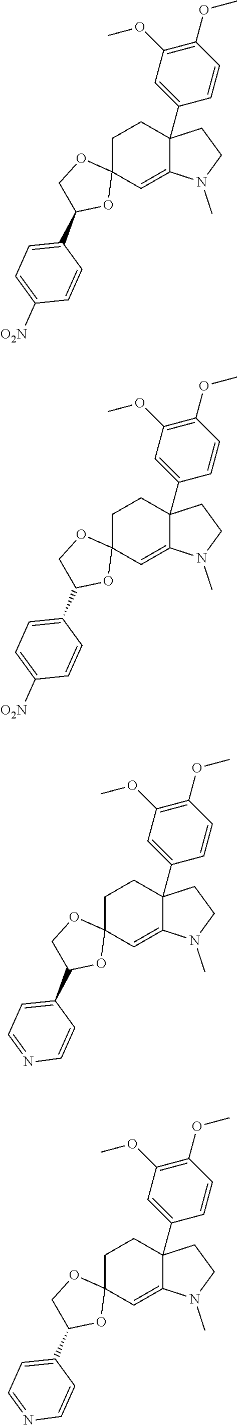

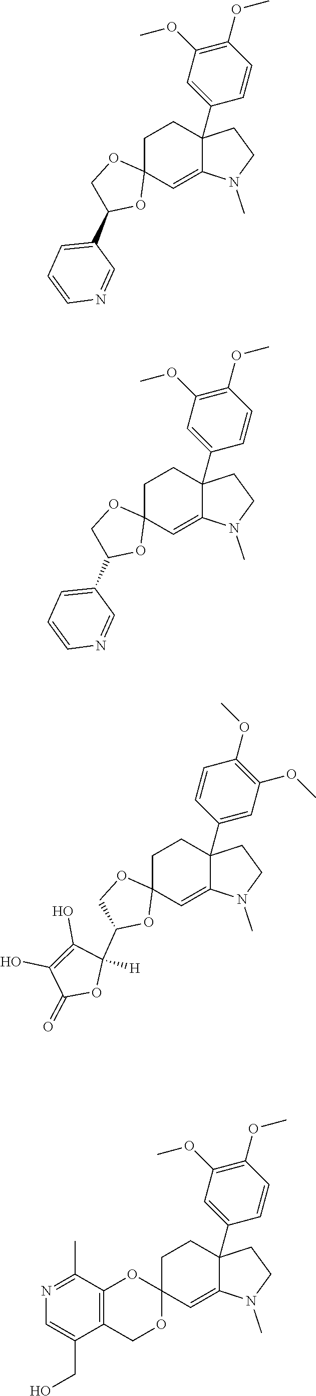

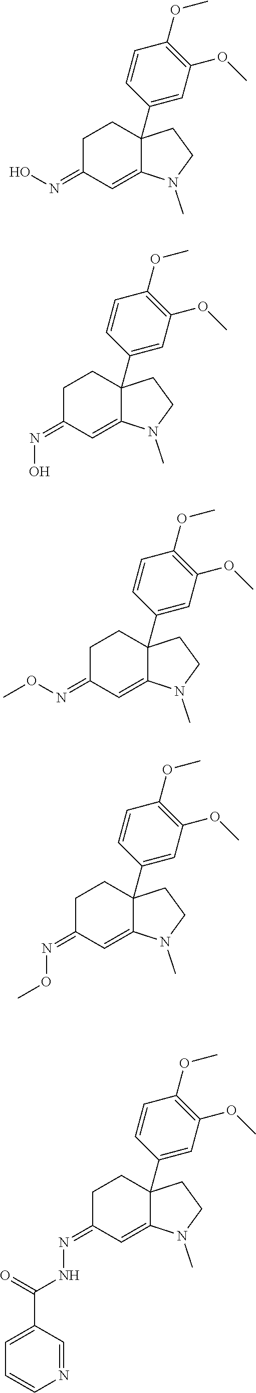

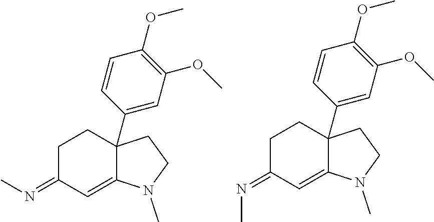

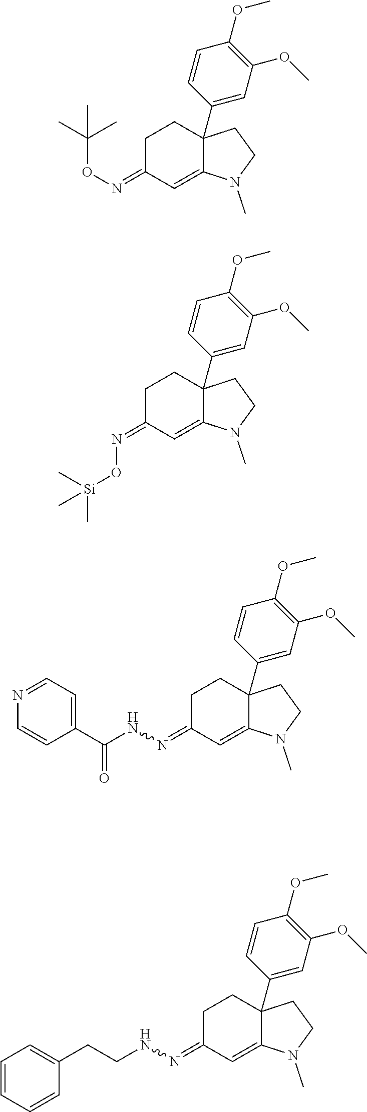

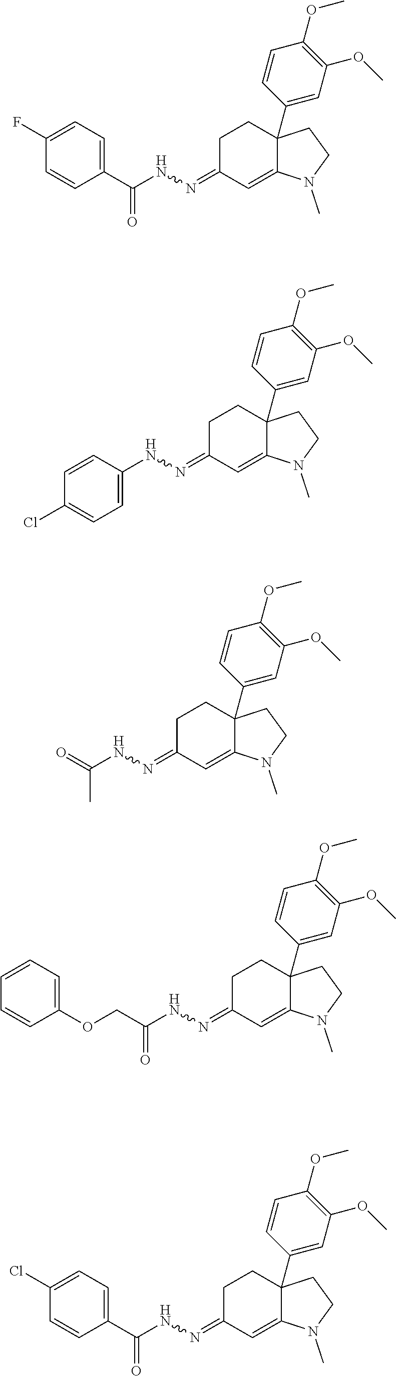 [SMILES: COc1ccc(C23CCC(=NNC(=O)c4ccc(F)cc4)C=C2N(C)CC3)cc1OC ; COc1ccc(C23CCC(=NNc4ccc(Cl)cc4)C=C2N(C)CC3)cc1OC ; COc1ccc(C23CCC(=NNC(C)=O)C=C2N(C)CC3)cc1OC ; COc1ccc(C23CCC(=NNC(=O)COc4ccccc4)C=C2N(C)CC3)cc1OC ; COc1ccc(C23CCC(=NNC(=O)c4ccc(Cl)cc4)C=C2N(C)CC3)cc1OC]

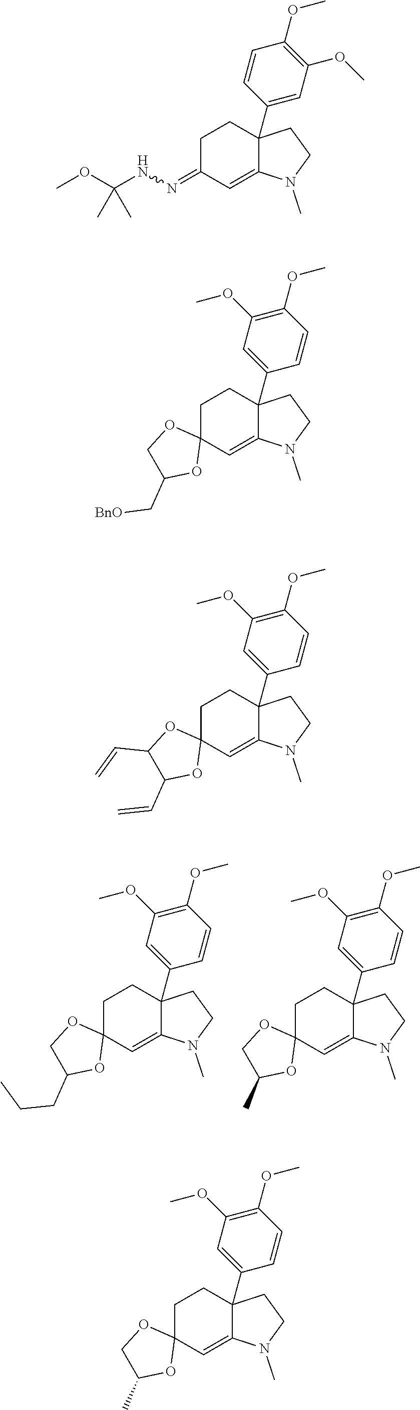

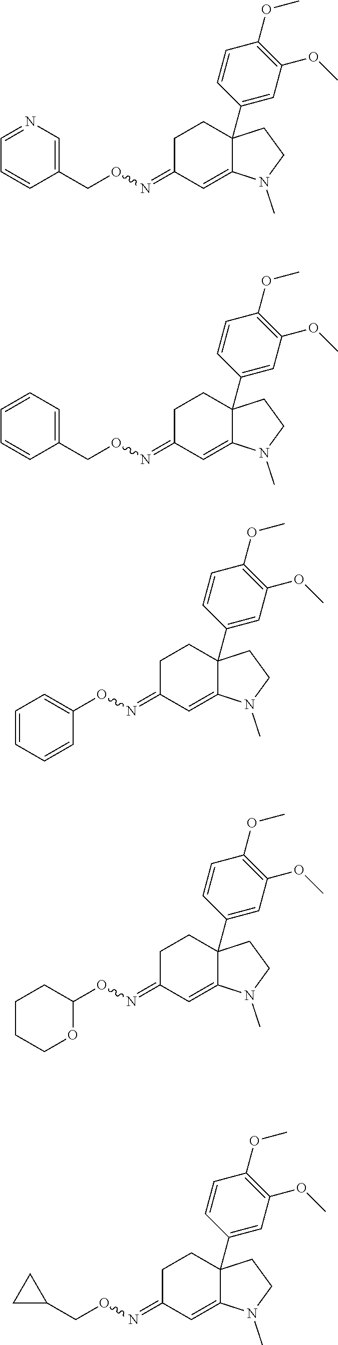

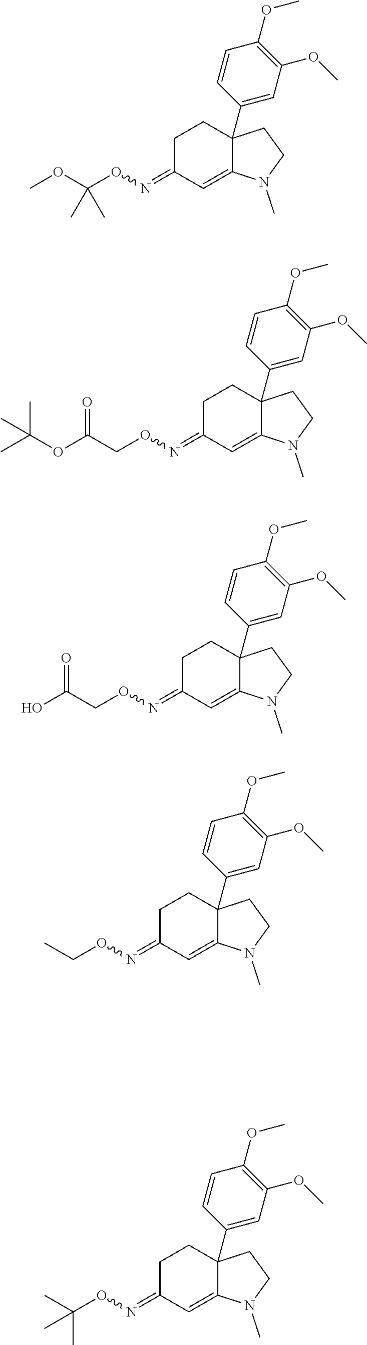

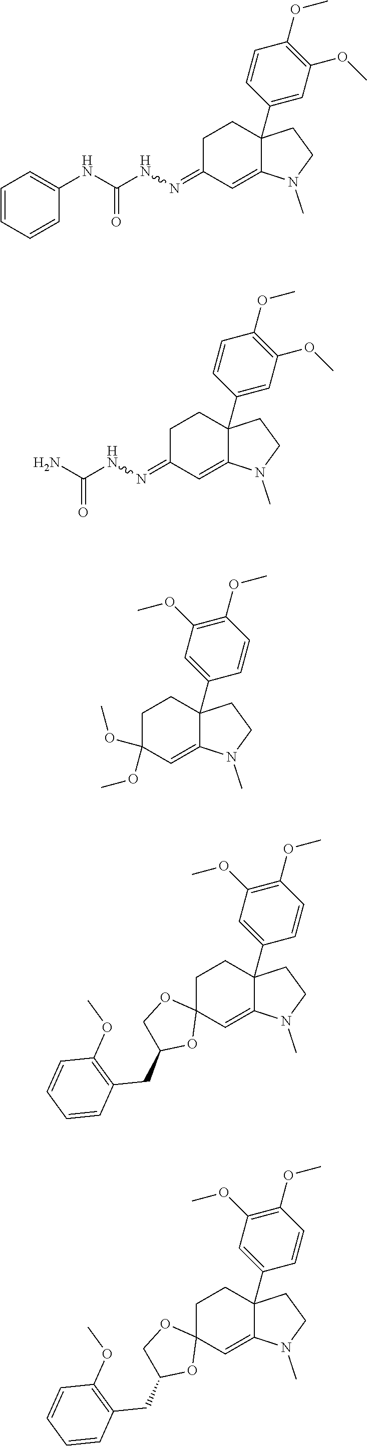

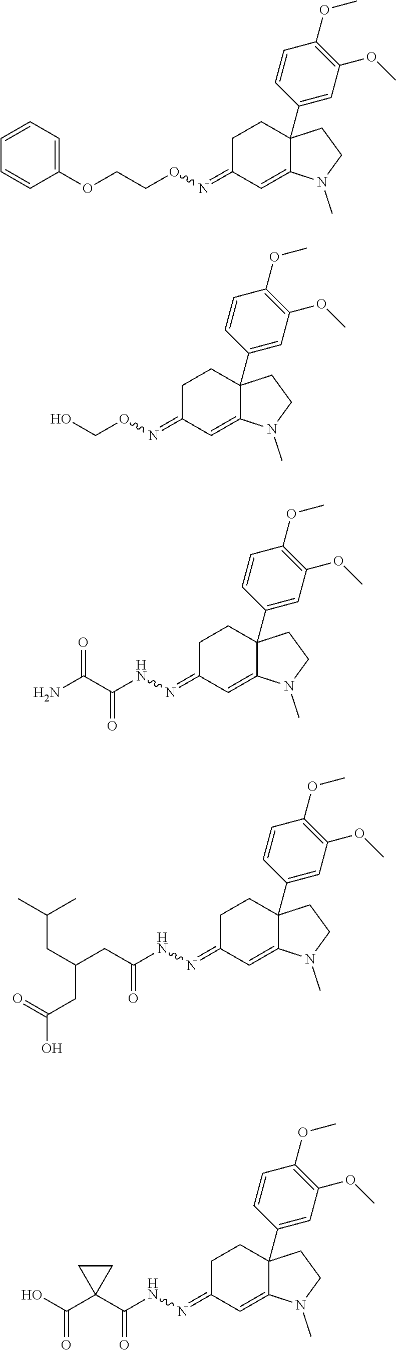

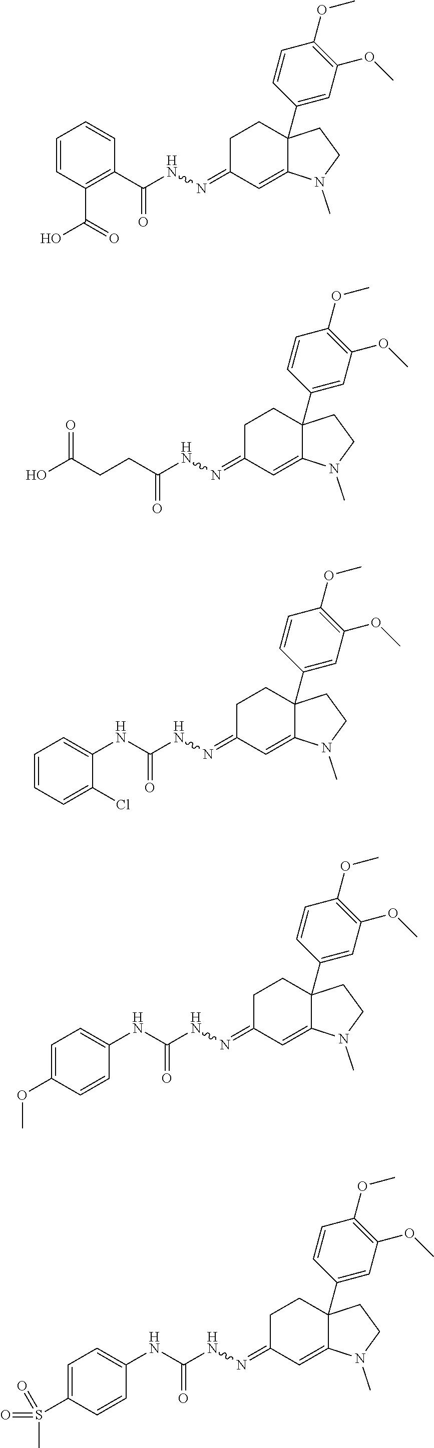 [SMILES: COc1ccc(C23CCC(=NNC(=O)c4ccccc4C(=O)O)C=C2N(C)CC3)cc1OC ; COc1ccc(C23CCC(=NNC(=O)CCC(=O)O)C=C2N(C)CC3)cc1OC ; COc1ccc(C23CCC(=NNC(=O)Nc4ccccc4Cl)C=C2N(C)CC3)cc1OC ; COc1ccc(NC(=O)NN=C2C=C3N(C)CCC3(c3ccc(OC)c(OC)c3)CC2)cc1 ; COc1ccc(C23CCC(=NNC(=O)Nc4ccc(S(C)(=O)=O)cc4)C=C2N(C)CC3)cc1OC]

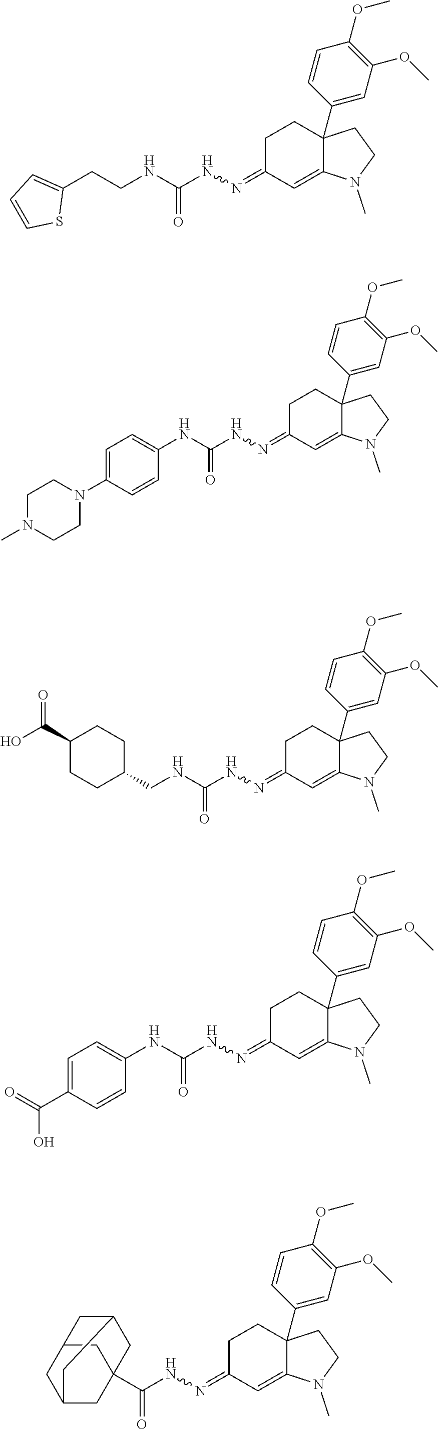

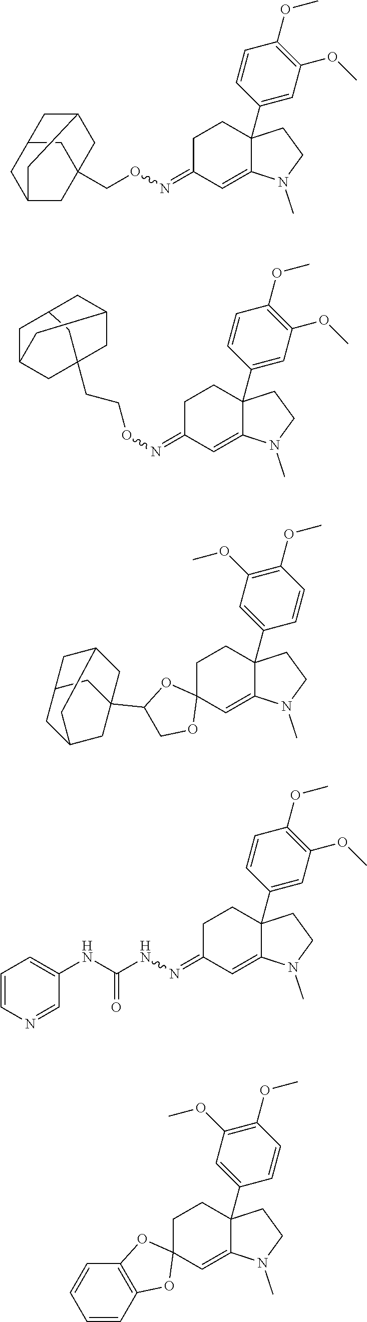 [SMILES: COc1ccc(C23CCC(=NOCC45CC6CC(CC(C6)C4)C5)C=C2N(C)CC3)cc1OC ; COc1ccc(C23CCC(=NOCCC45CC6CC(CC(C6)C4)C5)C=C2N(C)CC3)cc1OC ; COc1ccc(C23CCN(C)C2=CC2(CC3)OCC(C34CC5CC(CC(C5)C3)C4)O2)cc1OC ; COc1ccc(C23CCC(=NNC(=O)Nc4cccnc4)C=C2N(C)CC3)cc1OC ; COc1ccc(C23CCN(C)C2=CC2(CC3)Oc3ccccc3O2)cc1OC]

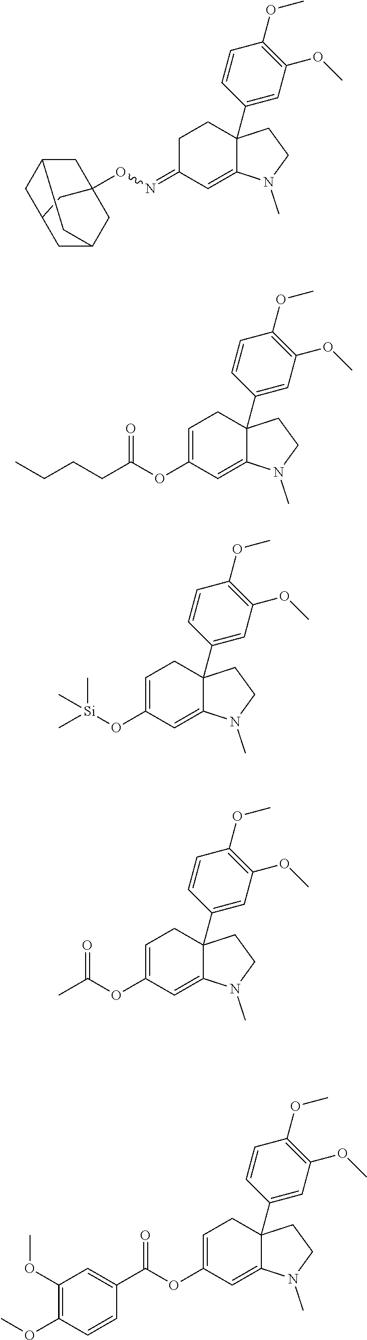

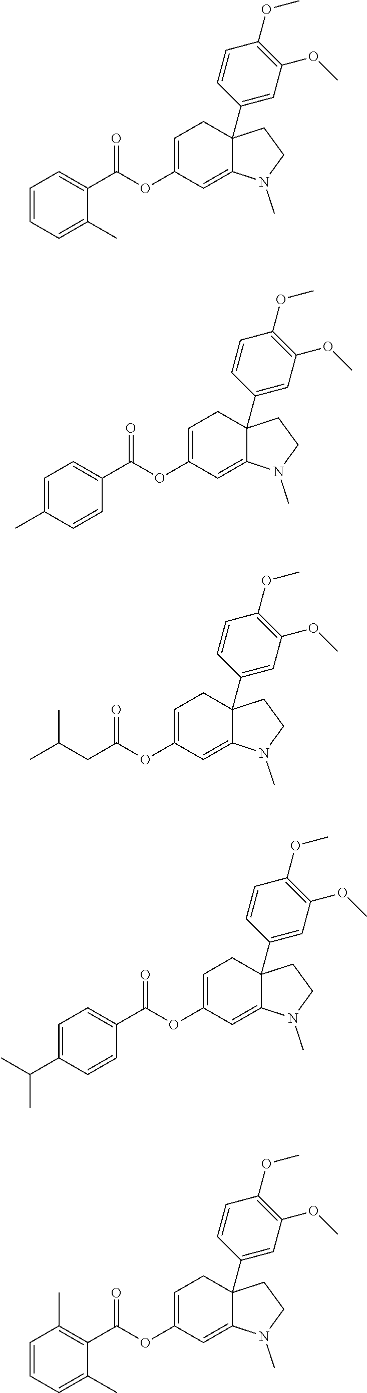

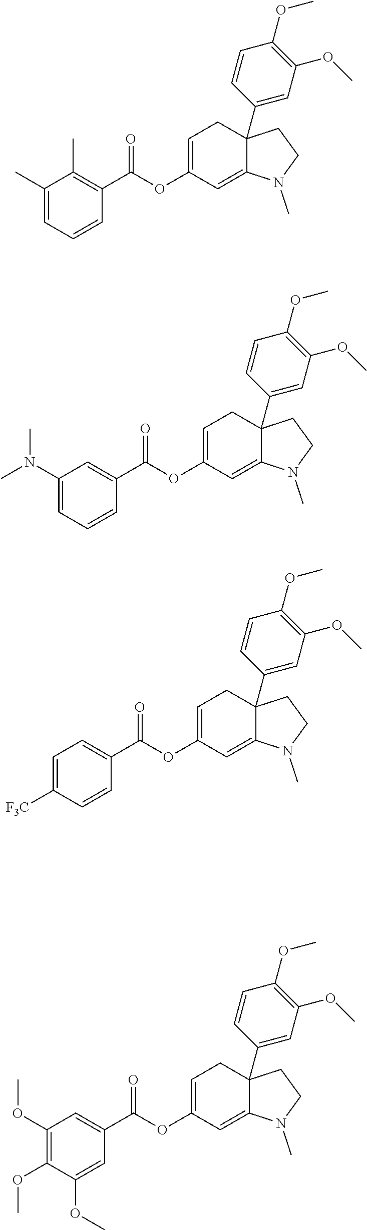

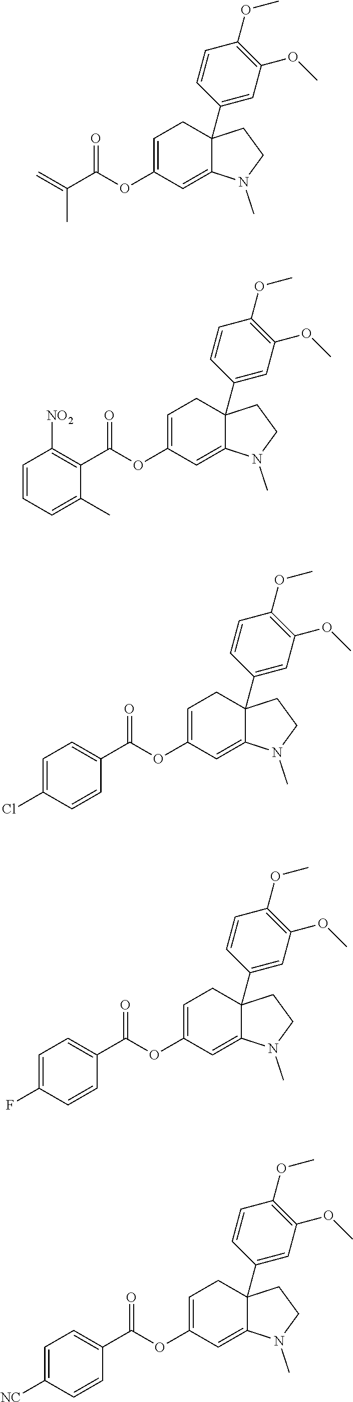 [SMILES: C=C(C)C(=O)OC1=CCC2(c3ccc(OC)c(OC)c3)CCN(C)C2=C1 ; COc1ccc(C23CC=C(OC(=O)c4c(C)cccc4[N+](=O)[O-])C=C2N(C)CC3)cc1OC ; COc1ccc(C23CC=C(OC(=O)c4ccc(Cl)cc4)C=C2N(C)CC3)cc1OC ; COc1ccc(C23CC=C(OC(=O)c4ccc(F)cc4)C=C2N(C)CC3)cc1OC ; COc1ccc(C23CC=C(OC(=O)c4ccc(C#N)cc4)C=C2N(C)CC3)cc1OC]

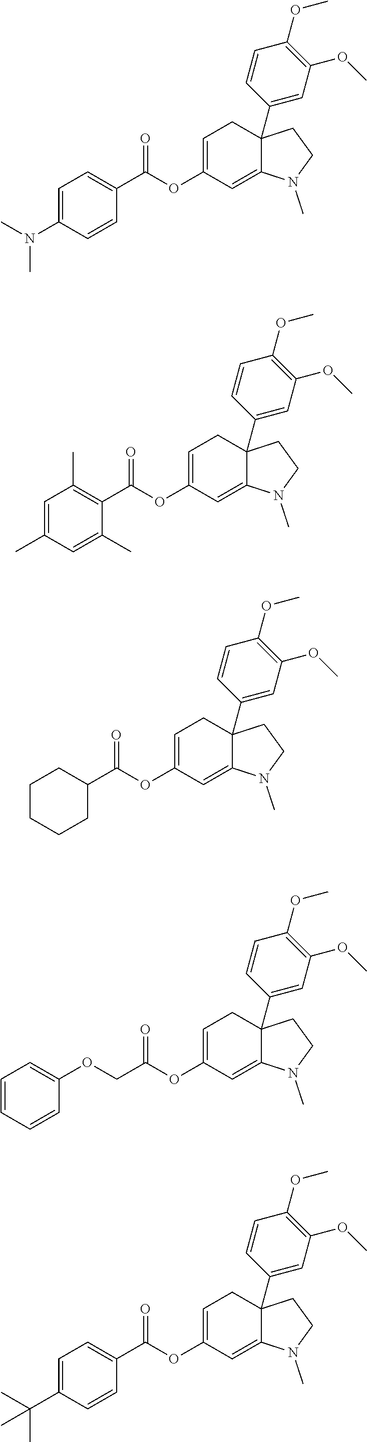

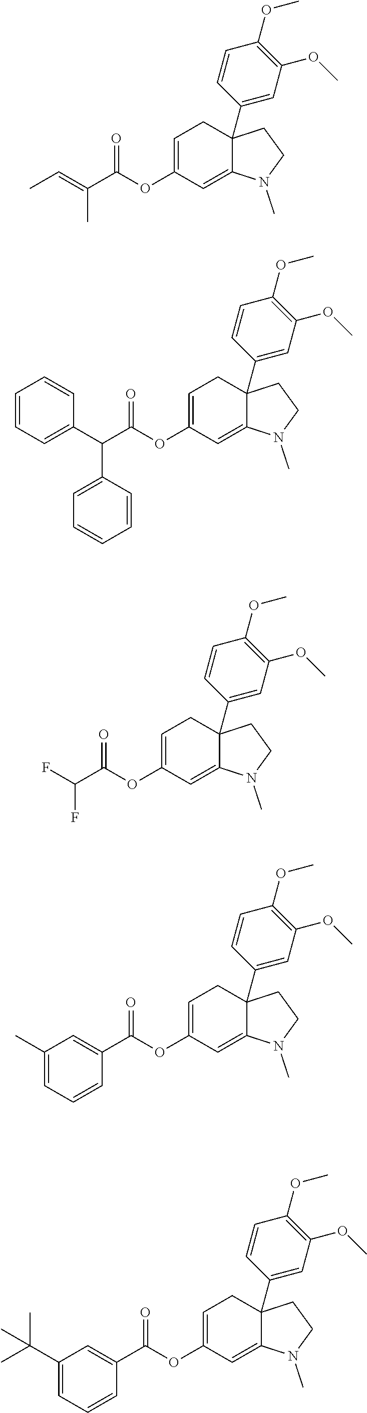

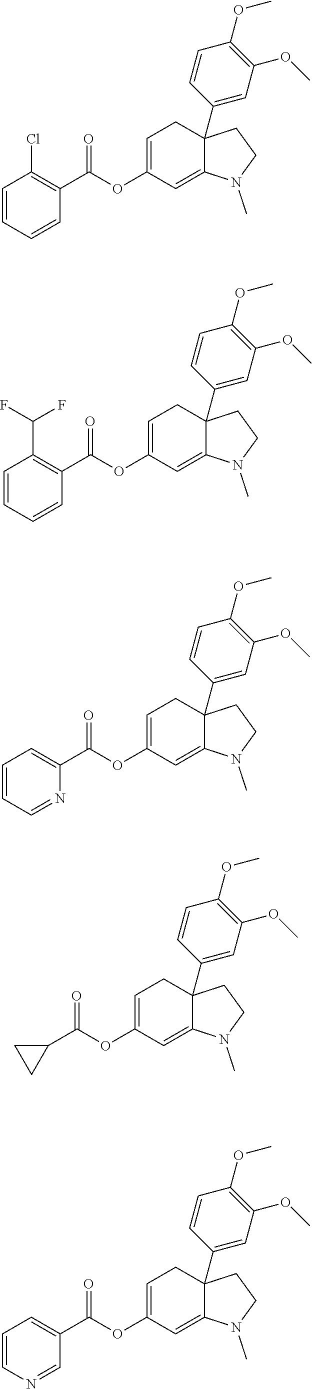 [SMILES: COc1ccc(C23CC=C(OC(=O)c4ccccc4Cl)C=C2N(C)CC3)cc1OC ; COc1ccc(C23CC=C(OC(=O)c4ccccc4C(F)F)C=C2N(C)CC3)cc1OC ; COc1ccc(C23CC=C(OC(=O)c4ccccn4)C=C2N(C)CC3)cc1OC ; COc1ccc(C23CC=C(OC(=O)C4CC4)C=C2N(C)CC3)cc1OC ; COc1ccc(C23CC=C(OC(=O)c4cccnc4)C=C2N(C)CC3)cc1OC]

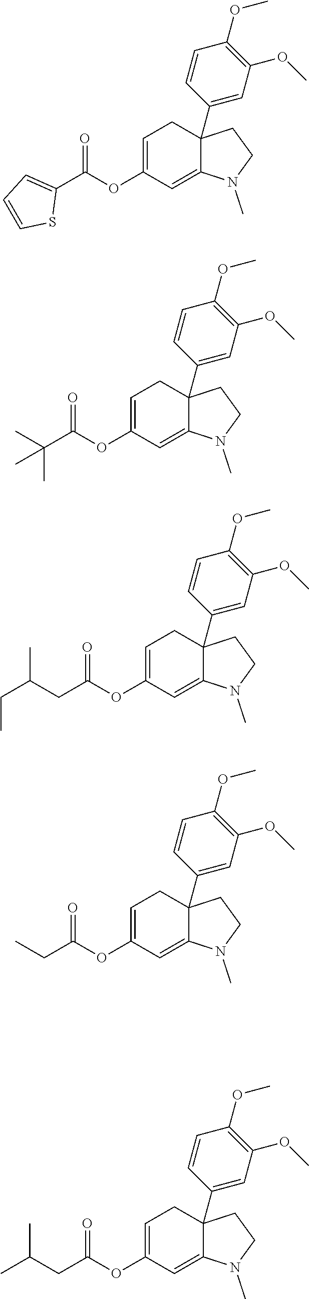

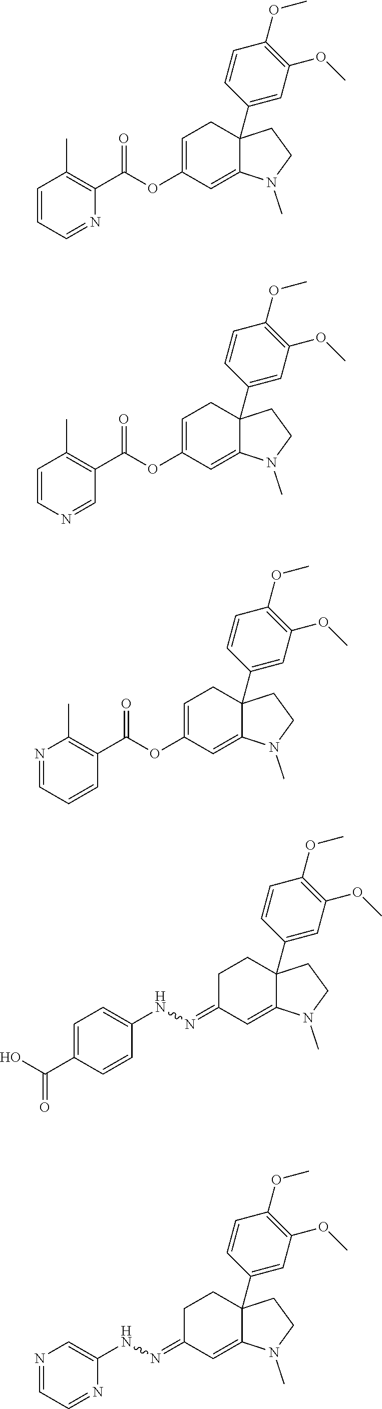 [SMILES: COc1ccc(C23CC=C(OC(=O)c4ncccc4C)C=C2N(C)CC3)cc1OC ; COc1ccc(C23CC=C(OC(=O)c4cnccc4C)C=C2N(C)CC3)cc1OC ; COc1ccc(C23CC=C(OC(=O)c4cccnc4C)C=C2N(C)CC3)cc1OC ; COc1ccc(C23CCC(=NNc4ccc(C(=O)O)cc4)C=C2N(C)CC3)cc1OC ; COc1ccc(C23CCC(=NNc4cnccn4)C=C2N(C)CC3)cc1OC]

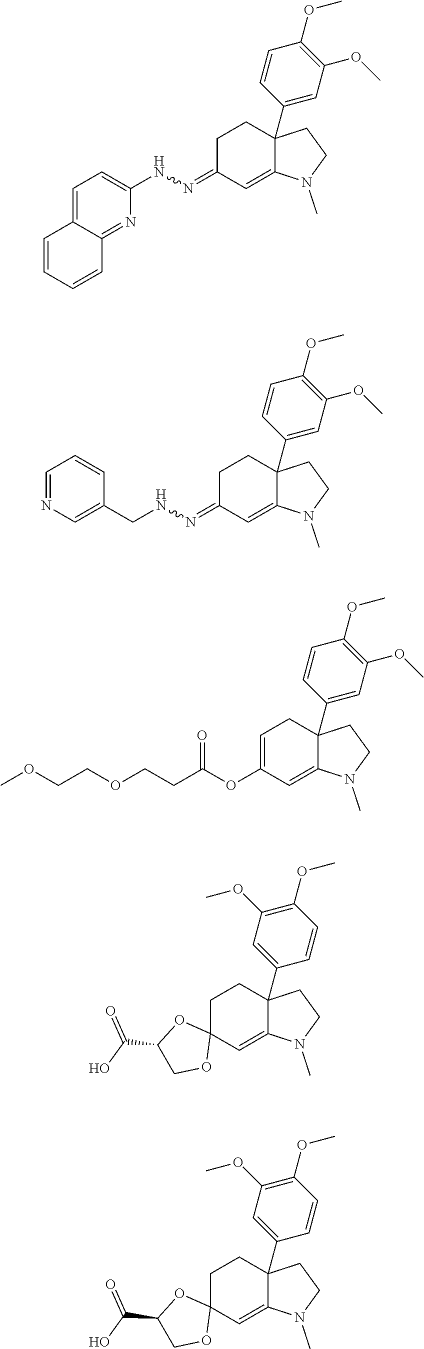

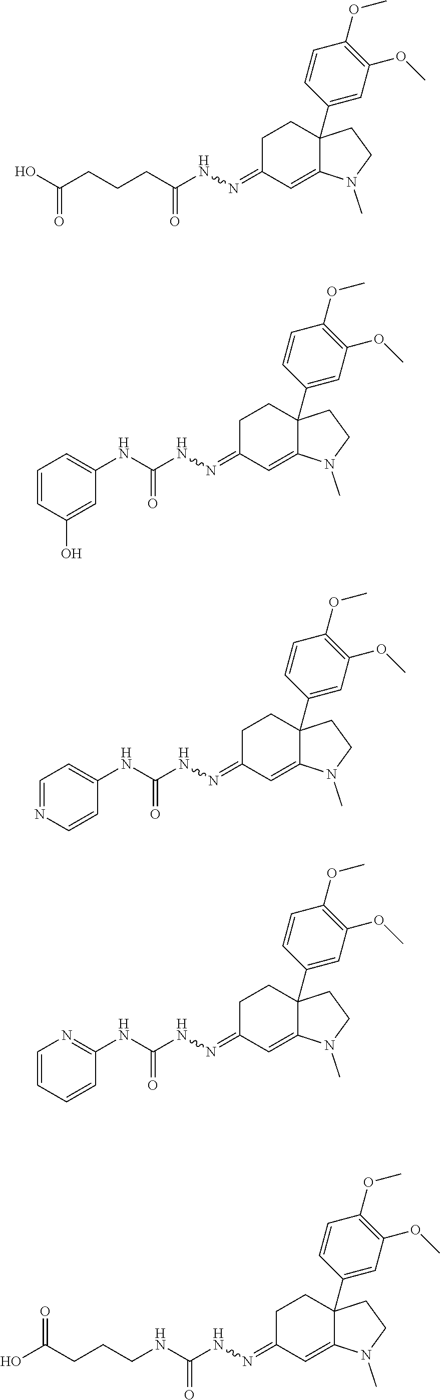 [SMILES: COc1ccc(C23CCC(=NNC(=O)CCCC(=O)O)C=C2N(C)CC3)cc1OC ; COc1ccc(C23CCC(=NNC(=O)Nc4cccc(O)c4)C=C2N(C)CC3)cc1OC ; COc1ccc(C23CCC(=NNC(=O)Nc4ccncc4)C=C2N(C)CC3)cc1OC ; COc1ccc(C23CCC(=NNC(=O)Nc4ccccn4)C=C2N(C)CC3)cc1OC ; COc1ccc(C23CCC(=NNC(=O)NCCCC(=O)O)C=C2N(C)CC3)cc1OC]

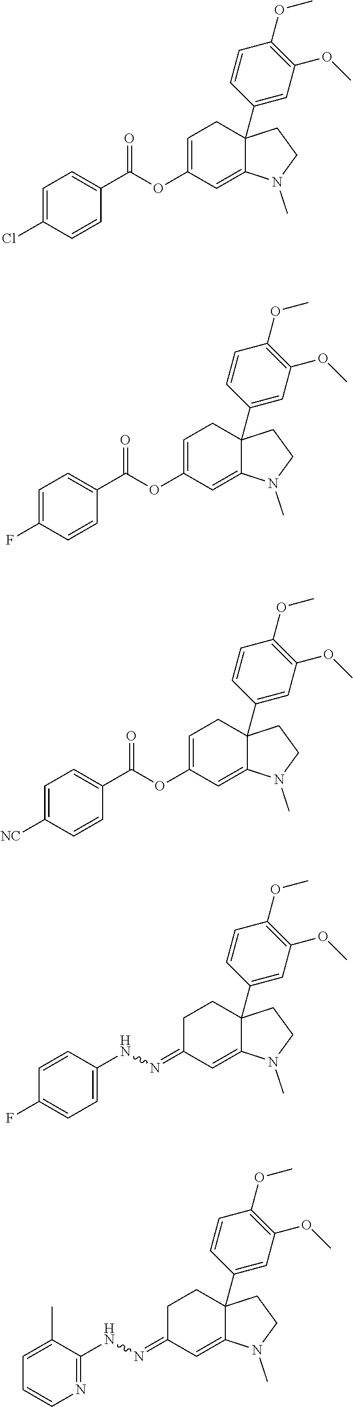 [SMILES: COc1ccc(C23CC=C(OC(=O)c4ccc(Cl)cc4)C=C2N(C)CC3)cc1OC ; COc1ccc(C23CC=C(OC(=O)c4ccc(F)cc4)C=C2N(C)CC3)cc1OC ; COc1ccc(C23CC=C(OC(=O)c4ccc(C#N)cc4)C=C2N(C)CC3)cc1OC ; COc1ccc(C23CCC(=NNc4ccc(F)cc4)C=C2N(C)CC3)cc1OC ; COc1ccc(C23CCC(=NNc4ncccc4C)C=C2N(C)CC3)cc1OC]

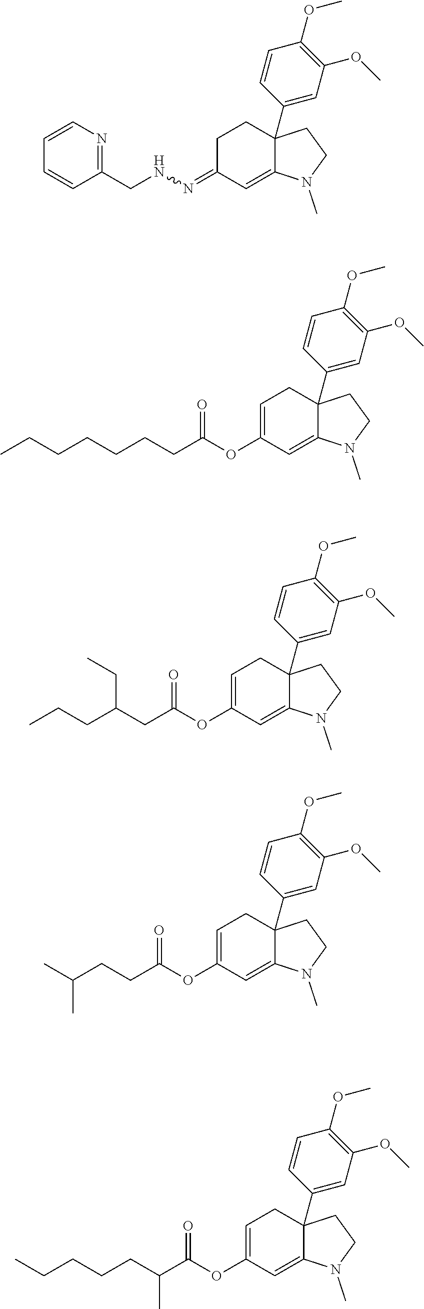

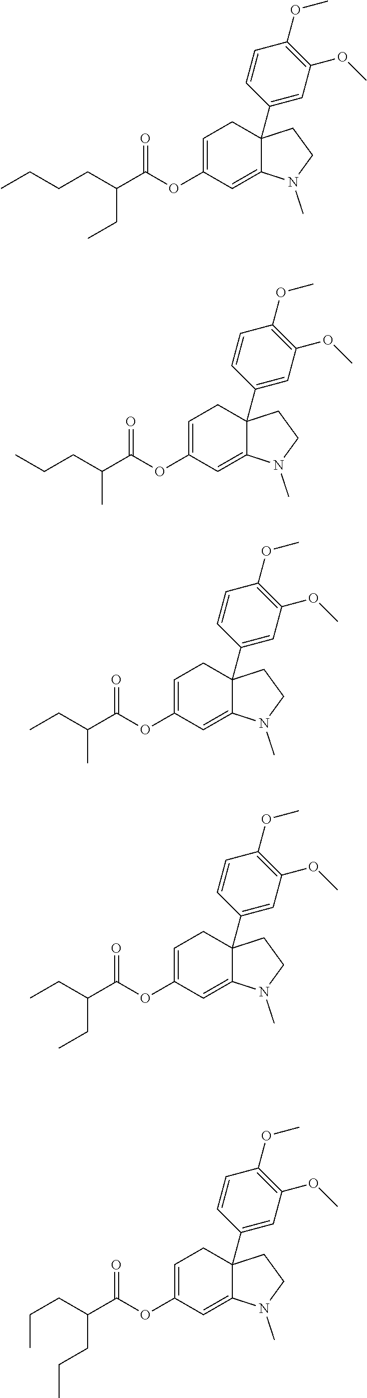

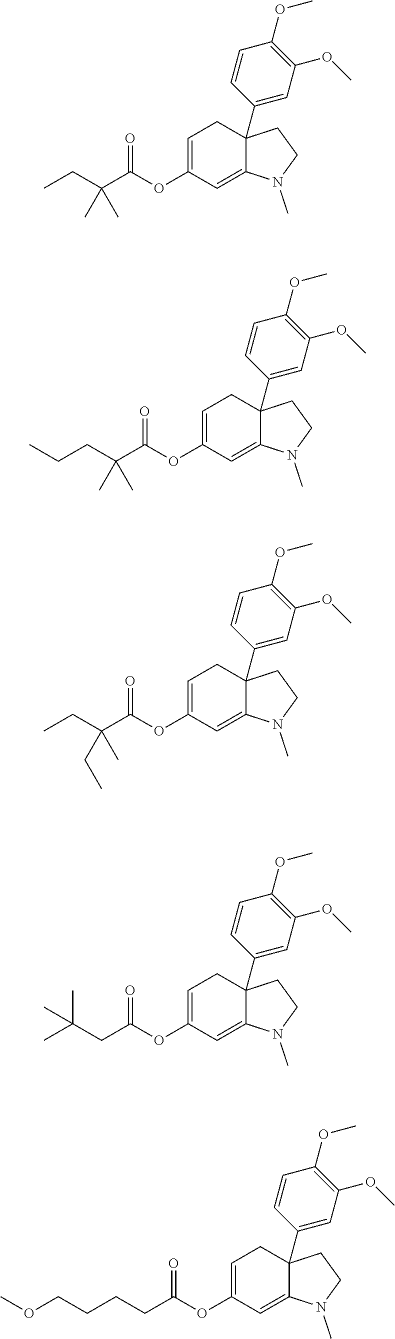

or a pharmaceutically acceptable salt thereof.

In some embodiments, the compound is selected from the group consisting of:

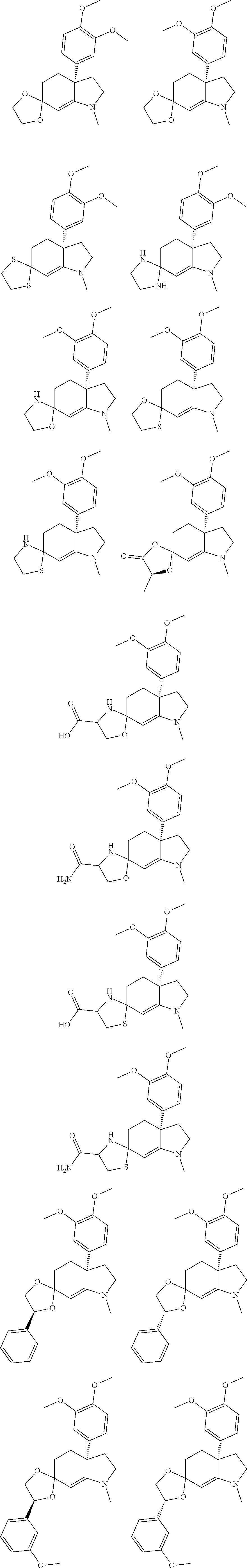

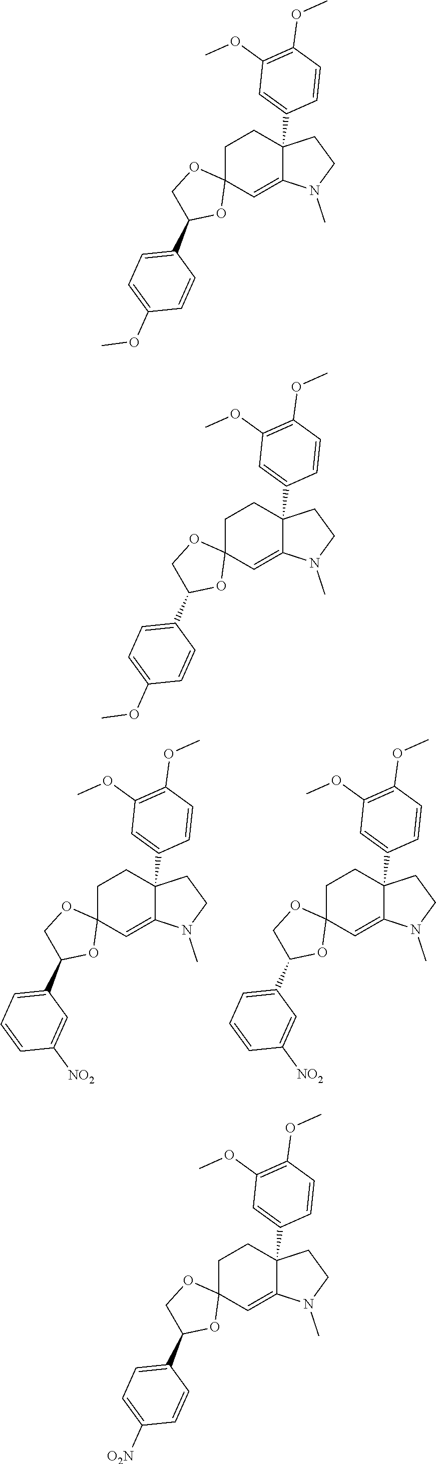

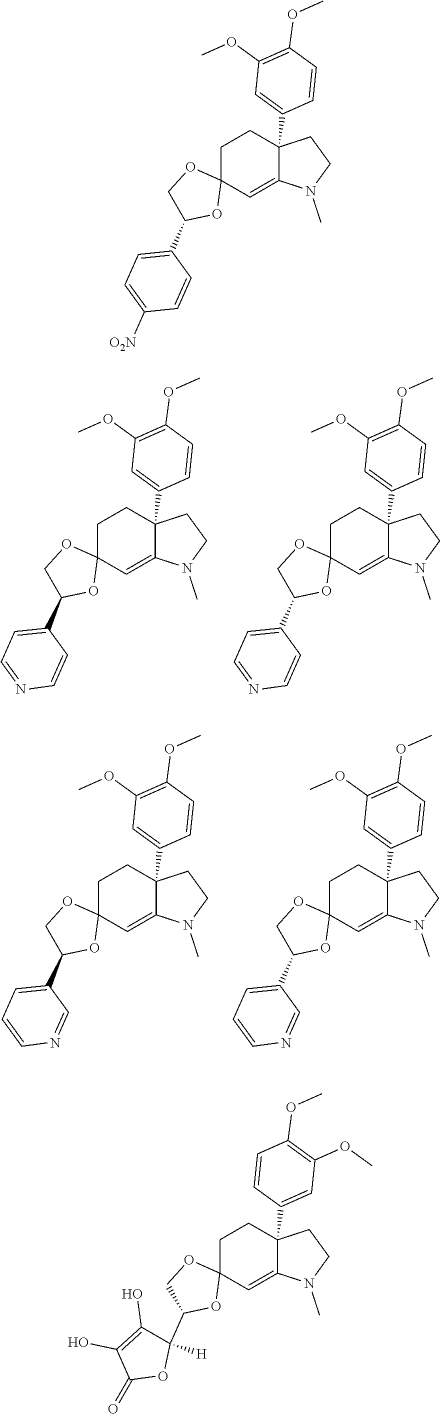

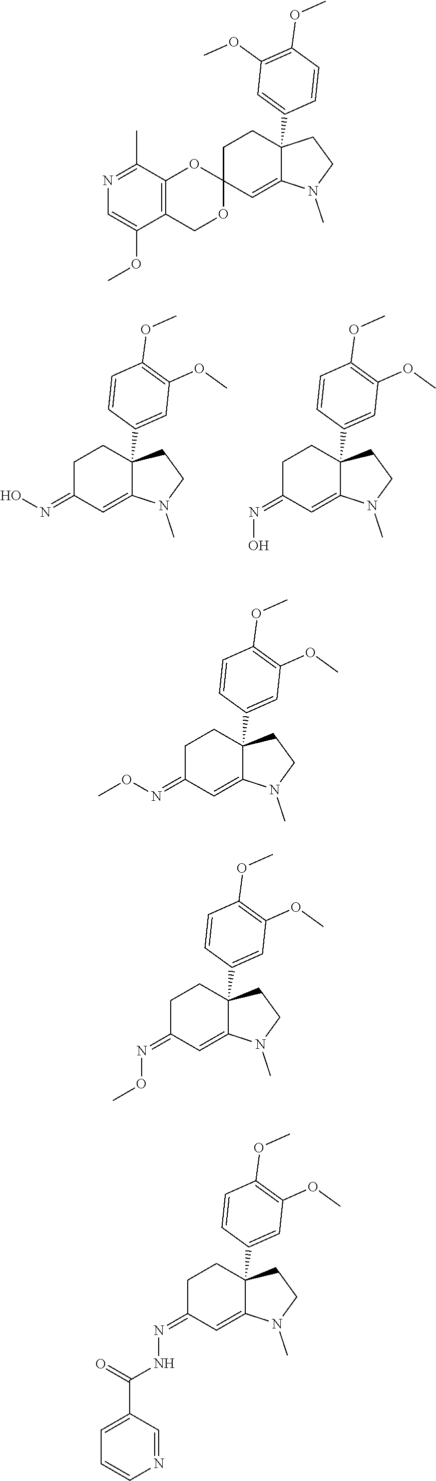

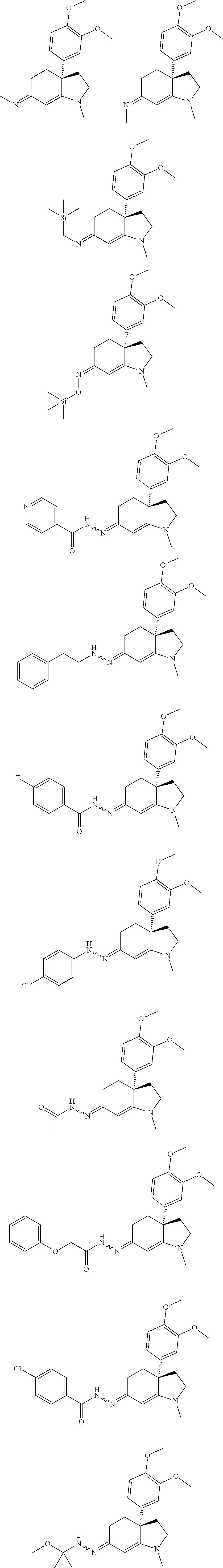

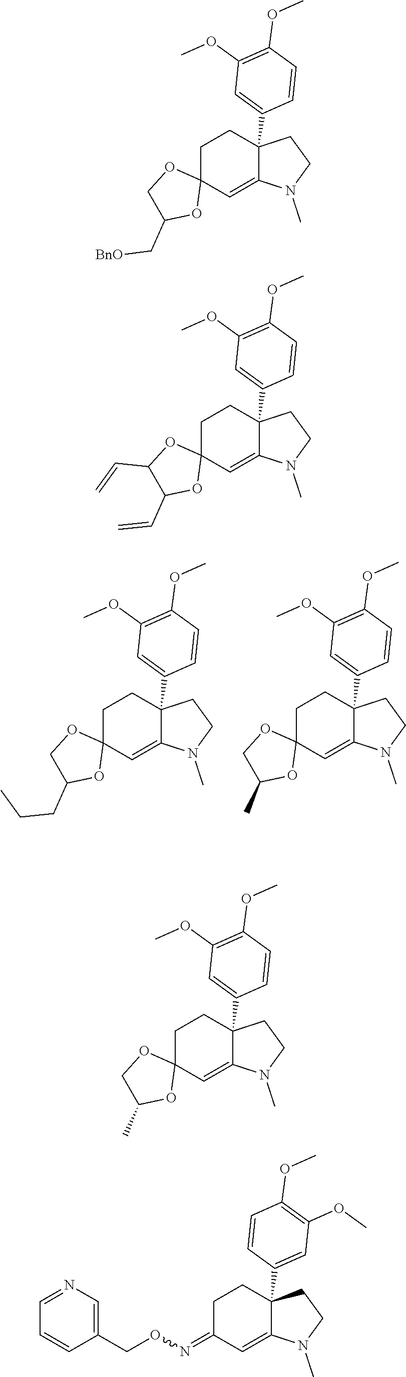

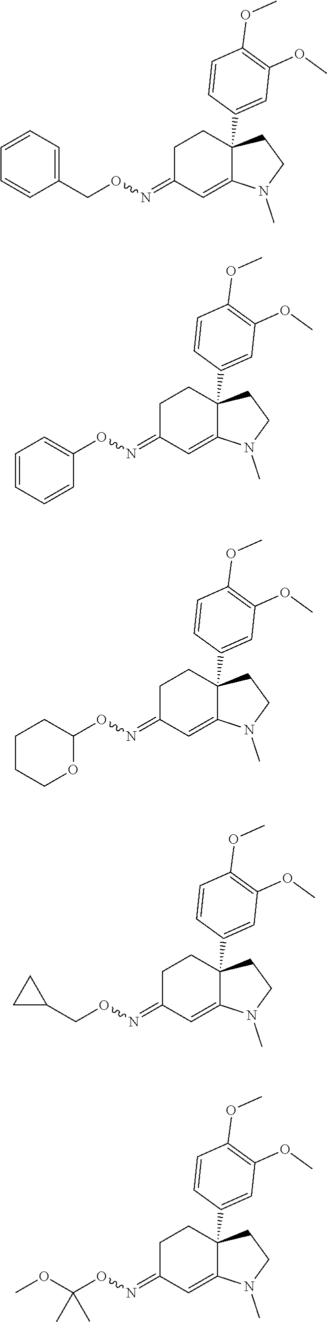

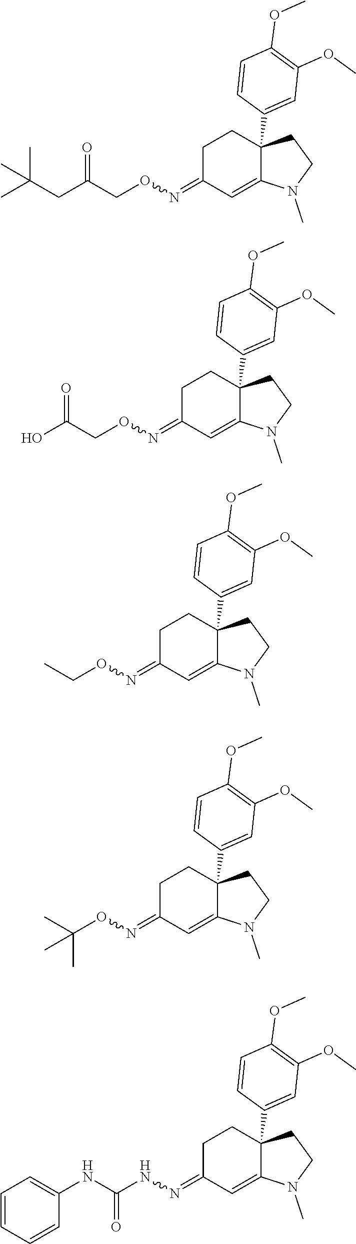

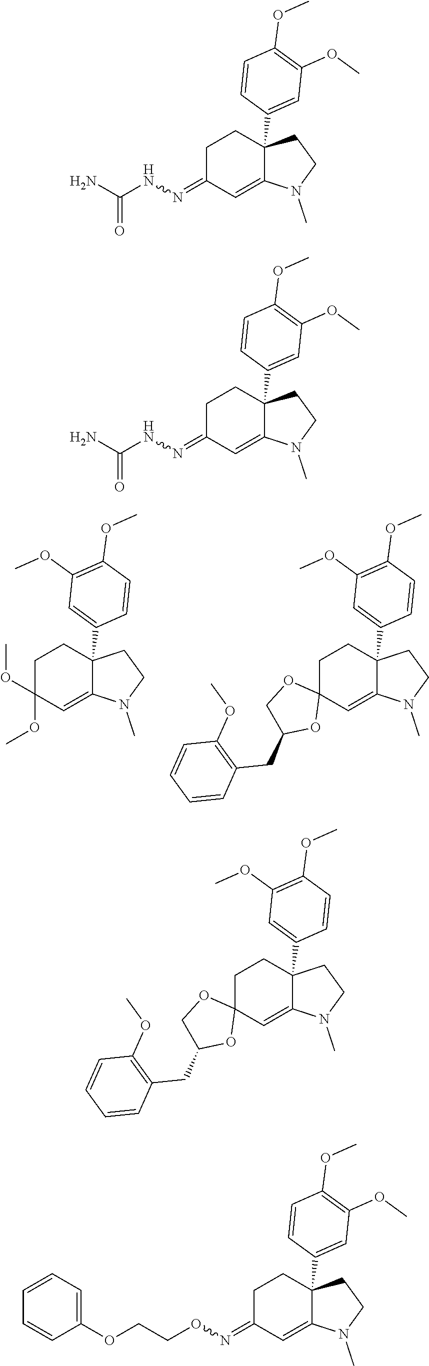

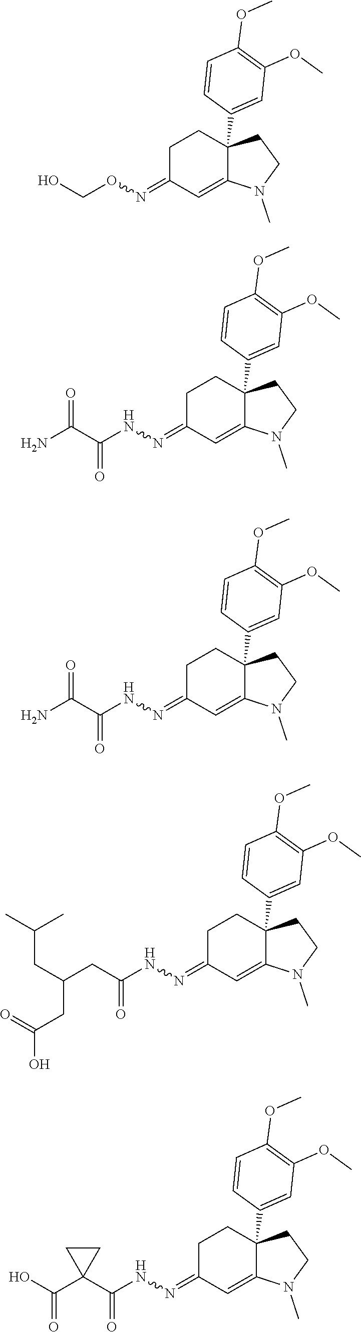

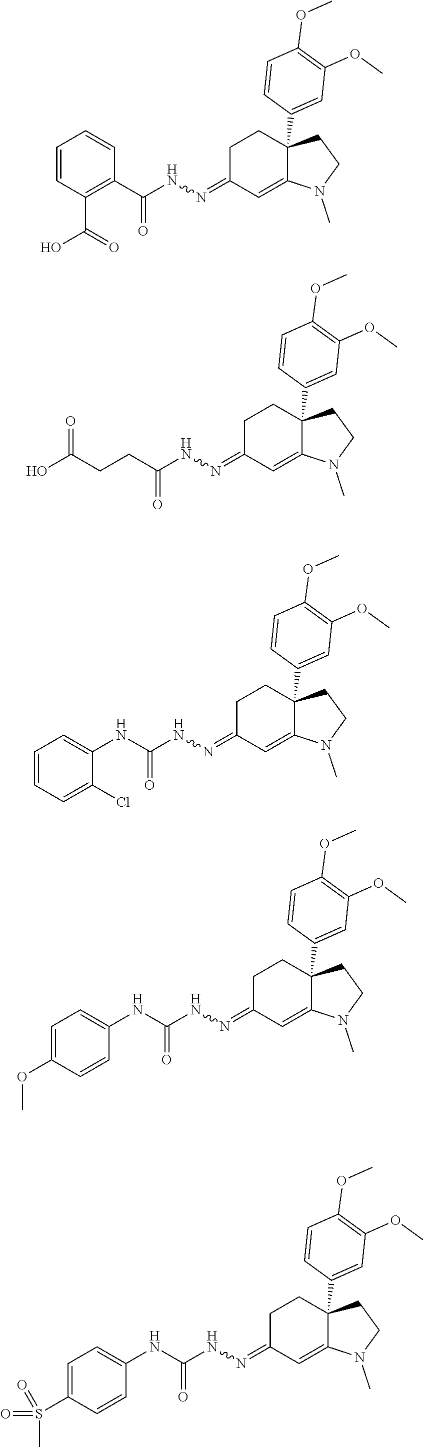 [SMILES: COc1ccc([C@@]23CCC(=NNC(=O)c4ccccc4C(=O)O)C=C2N(C)CC3)cc1OC ; COc1ccc([C@@]23CCC(=NNC(=O)CCC(=O)O)C=C2N(C)CC3)cc1OC ; COc1ccc([C@@]23CCC(=NNC(=O)Nc4ccccc4Cl)C=C2N(C)CC3)cc1OC ; COc1ccc(NC(=O)NN=C2C=C3N(C)CC[C@]3(c3ccc(OC)c(OC)c3)CC2)cc1 ; COc1ccc([C@@]23CCC(=NNC(=O)Nc4ccc(S(C)(=O)=O)cc4)C=C2N(C)CC3)cc1OC]

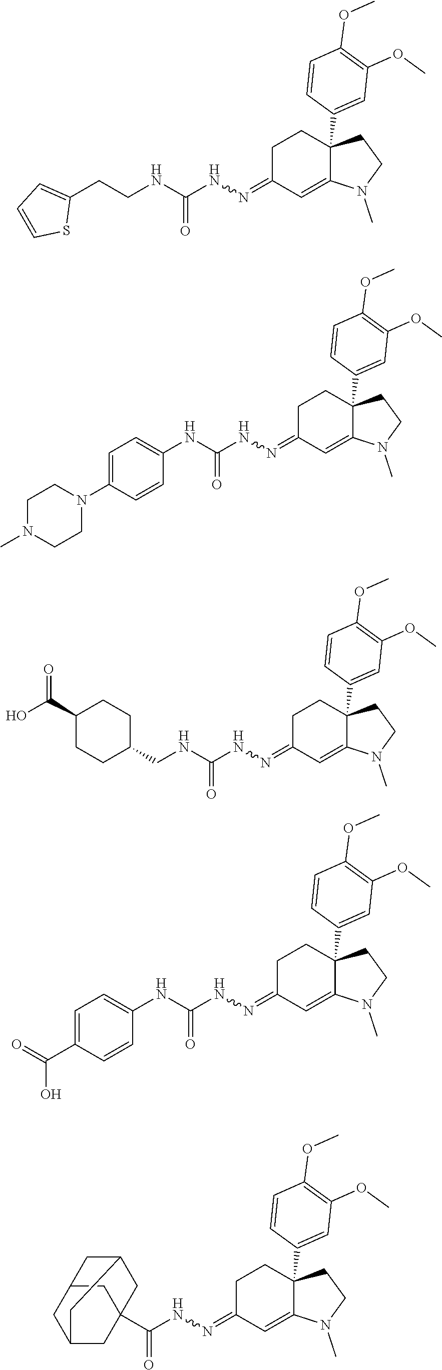

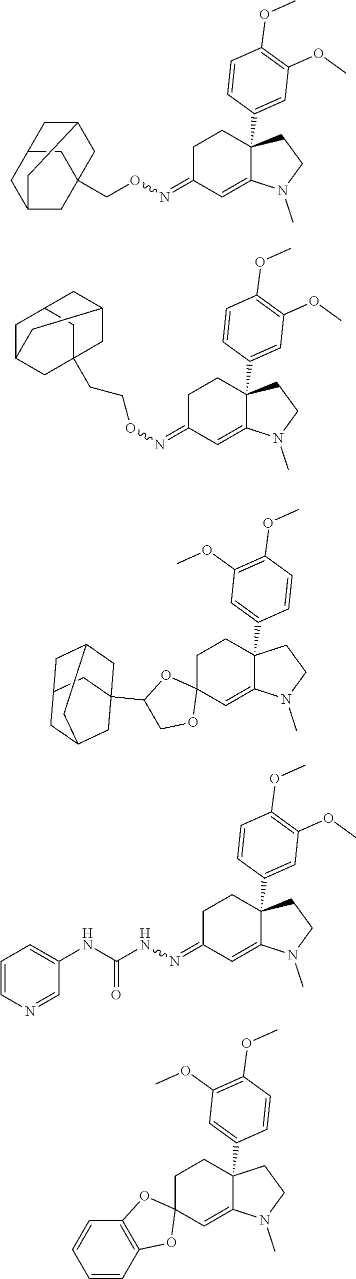

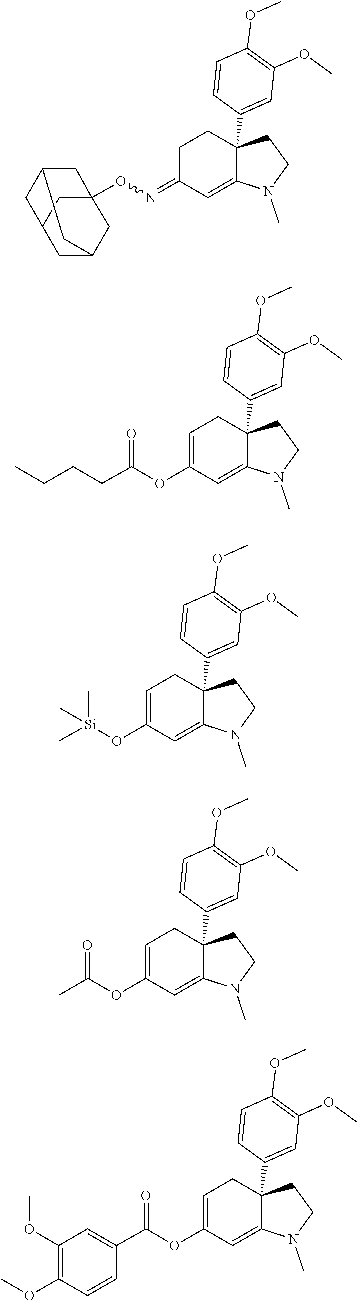

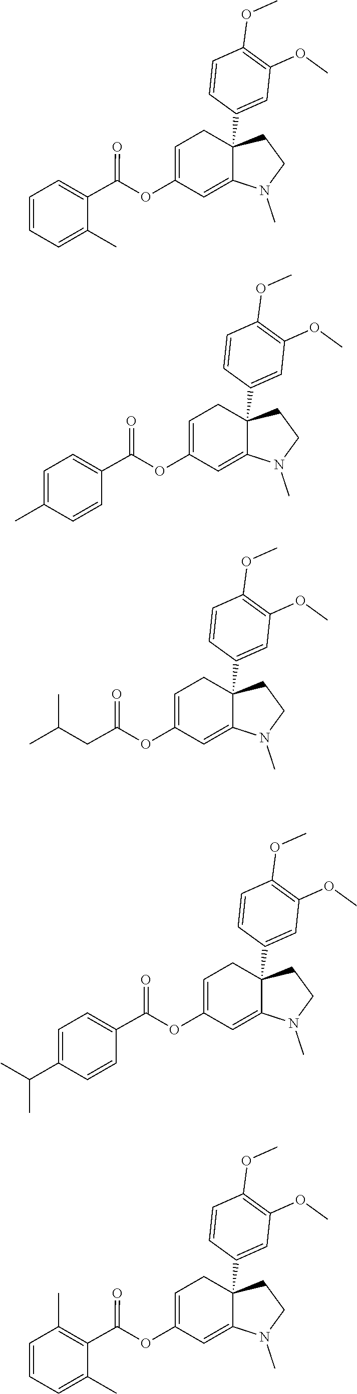

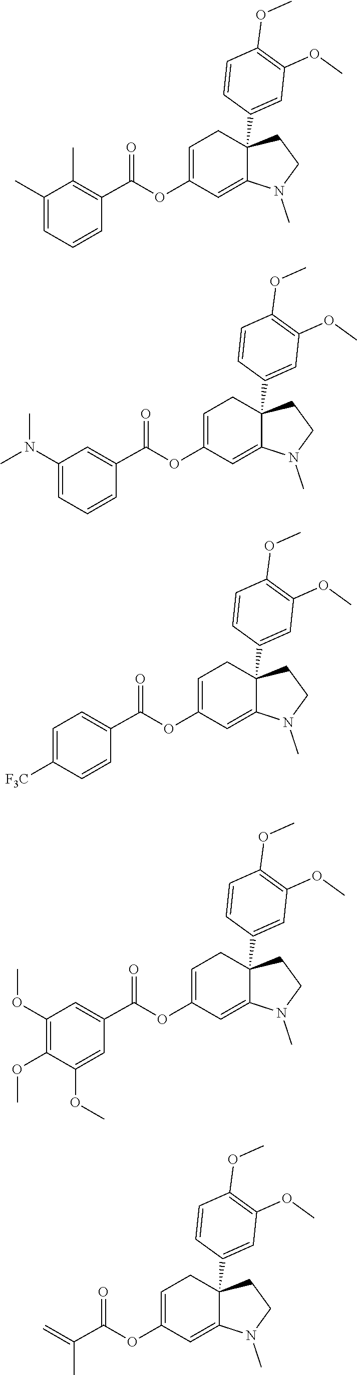

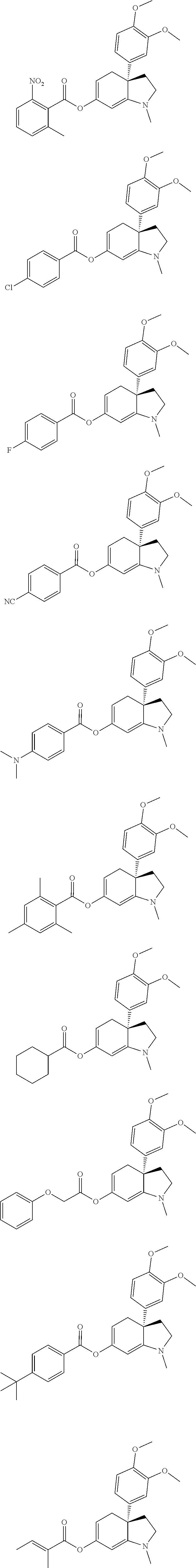

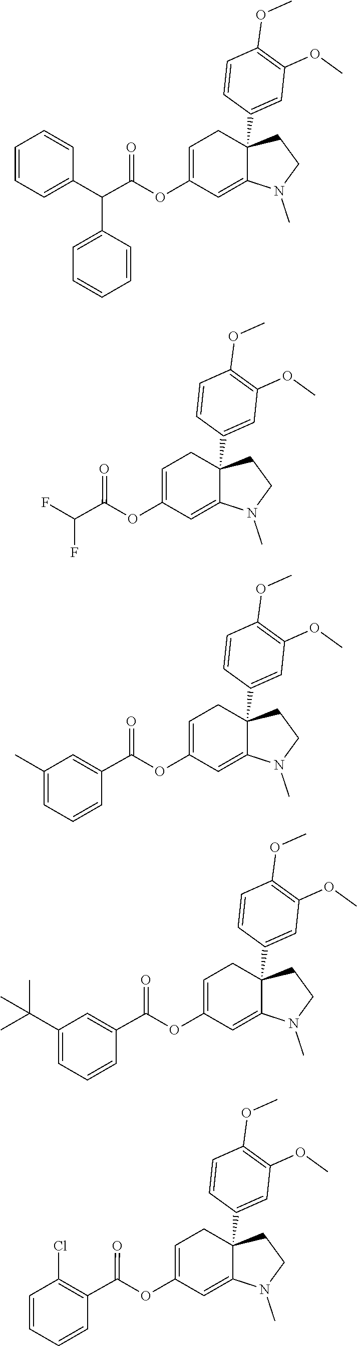

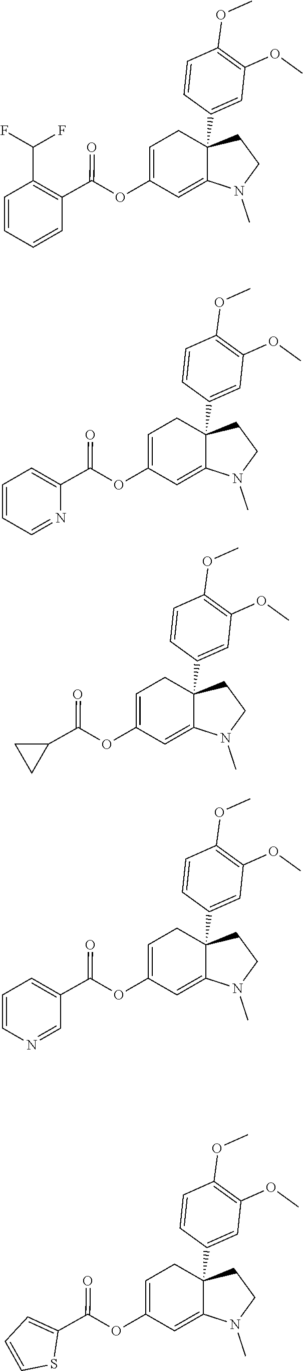

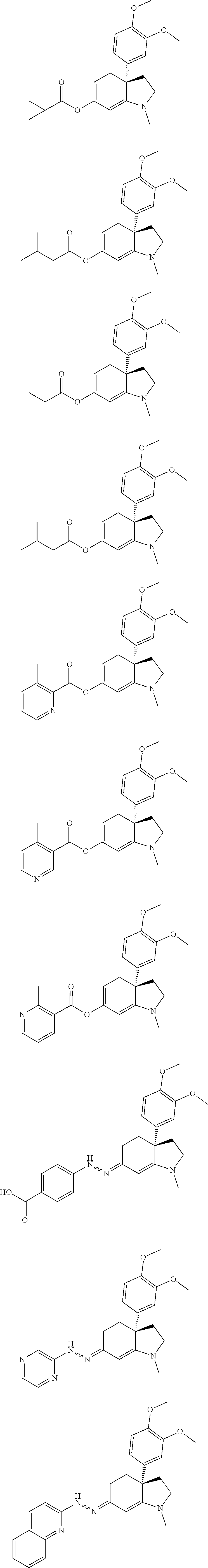

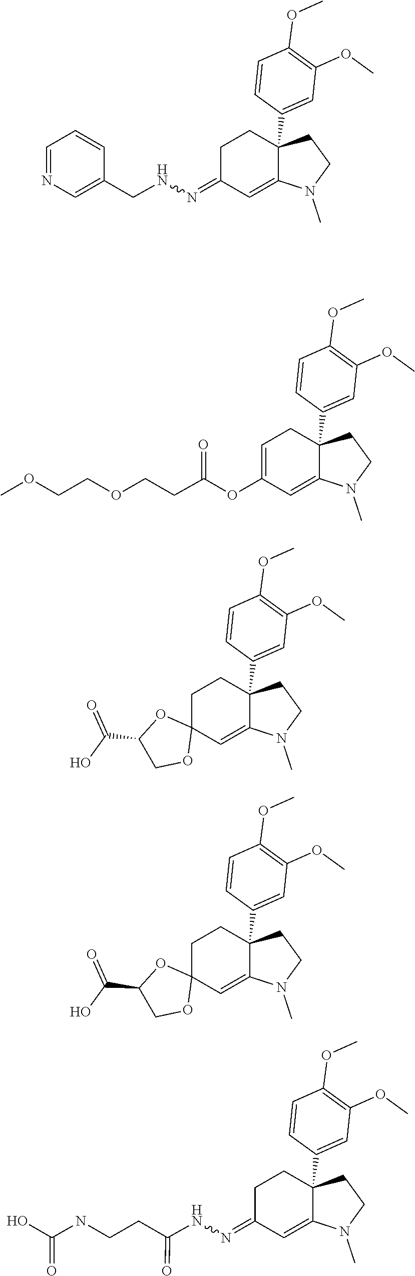

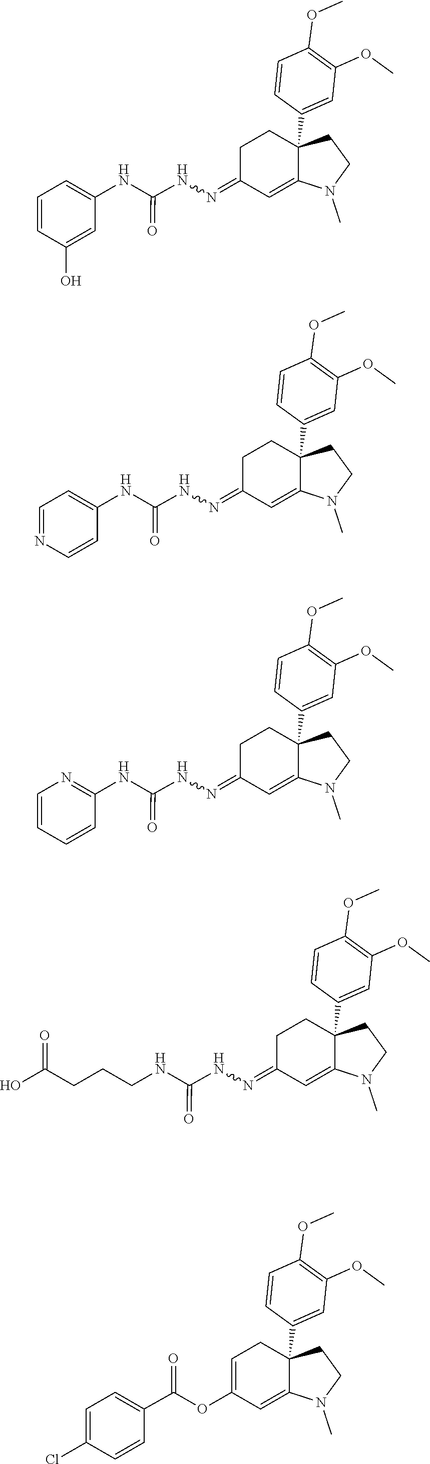 [SMILES: COc1ccc([C@@]23CCC(=NNC(=O)Nc4cccc(O)c4)C=C2N(C)CC3)cc1OC ; COc1ccc([C@@]23CCC(=NNC(=O)Nc4ccncc4)C=C2N(C)CC3)cc1OC ; COc1ccc([C@@]23CCC(=NNC(=O)Nc4ccccn4)C=C2N(C)CC3)cc1OC ; COc1ccc([C@@]23CCC(=NNC(=O)NCCCC(=O)O)C=C2N(C)CC3)cc1OC ; COc1ccc([C@@]23CC=C(OC(=O)c4ccc(Cl)cc4)C=C2N(C)CC3)cc1OC]

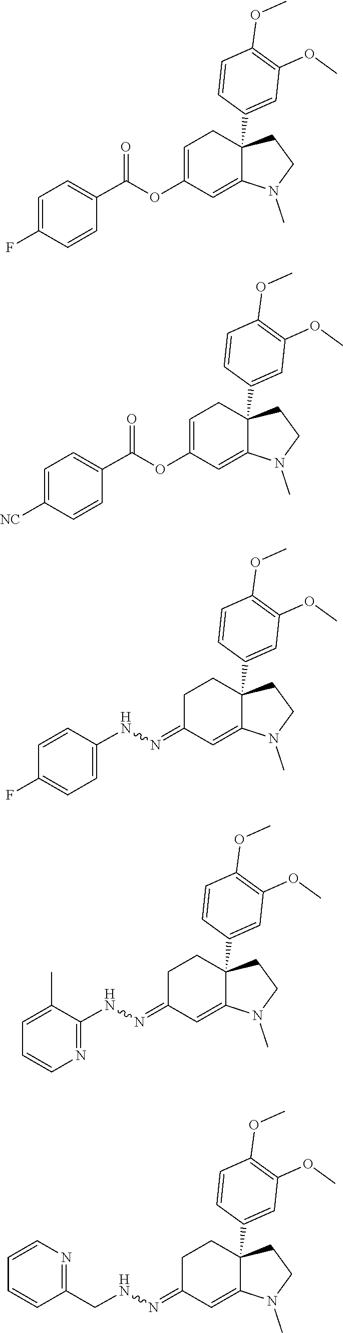 [SMILES: COc1ccc([C@@]23CC=C(OC(=O)c4ccc(F)cc4)C=C2N(C)CC3)cc1OC ; COc1ccc([C@@]23CC=C(OC(=O)c4ccc(C#N)cc4)C=C2N(C)CC3)cc1OC ; COc1ccc([C@@]23CCC(=NNc4ccc(F)cc4)C=C2N(C)CC3)cc1OC ; COc1ccc([C@@]23CCC(=NNc4ncccc4C)C=C2N(C)CC3)cc1OC ; COc1ccc([C@@]23CCC(=NNCc4ccccn4)C=C2N(C)CC3)cc1OC]

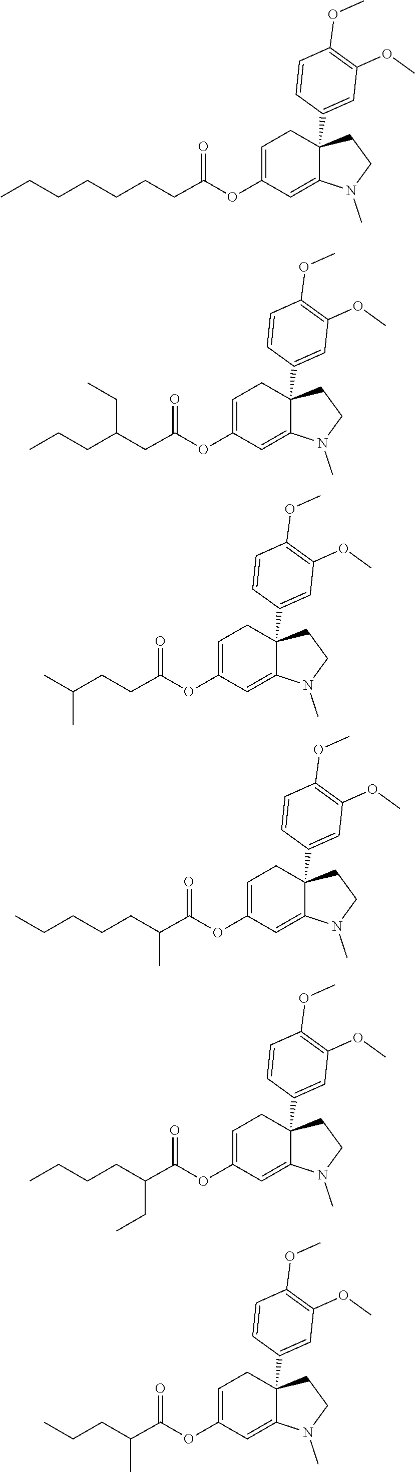

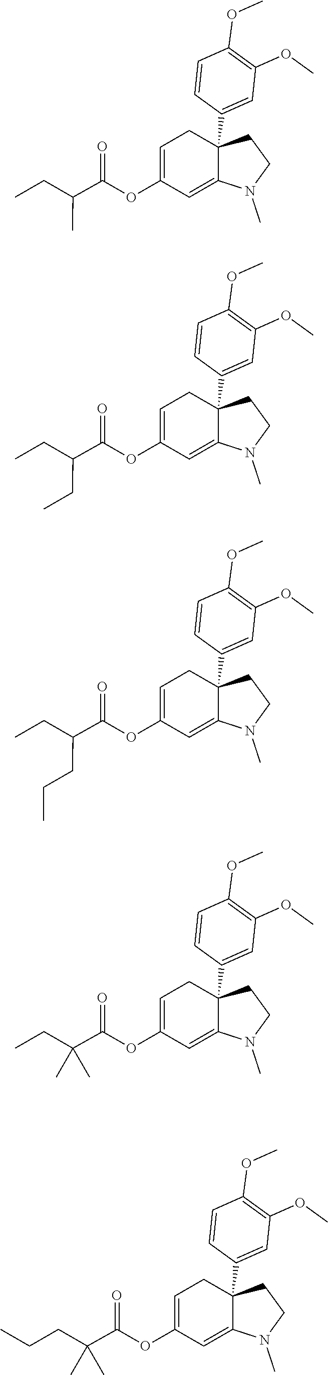

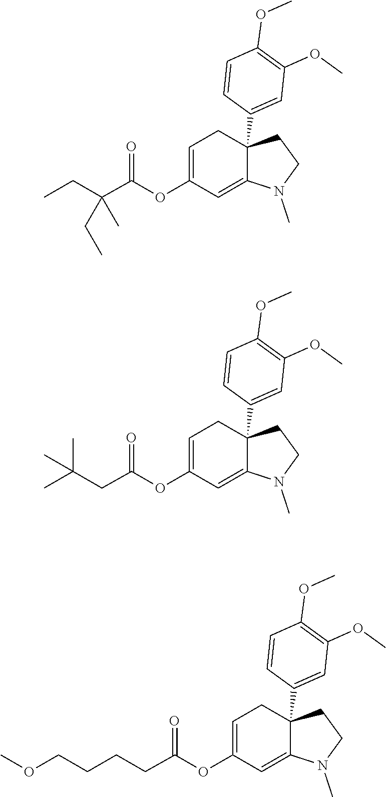

or a pharmaceutically acceptable salt thereof.

In some embodiments, the compound is selected from the group consisting of:

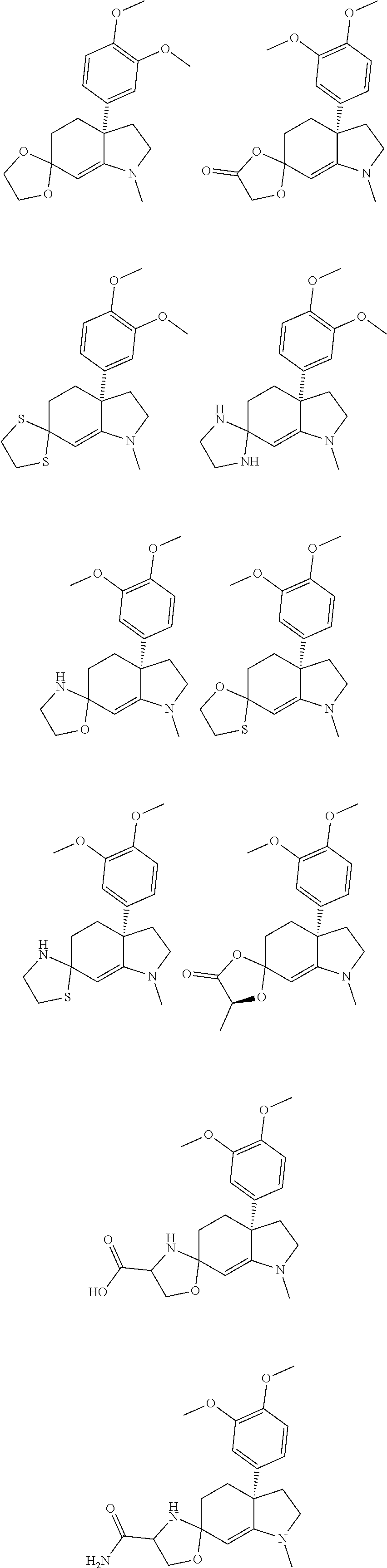

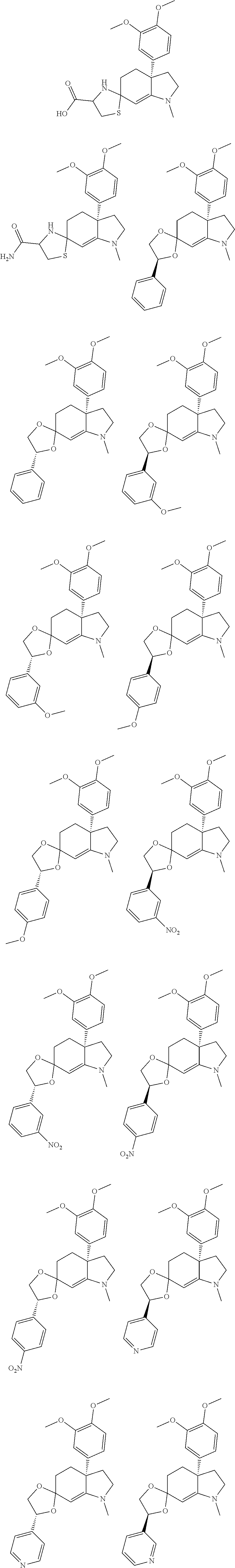

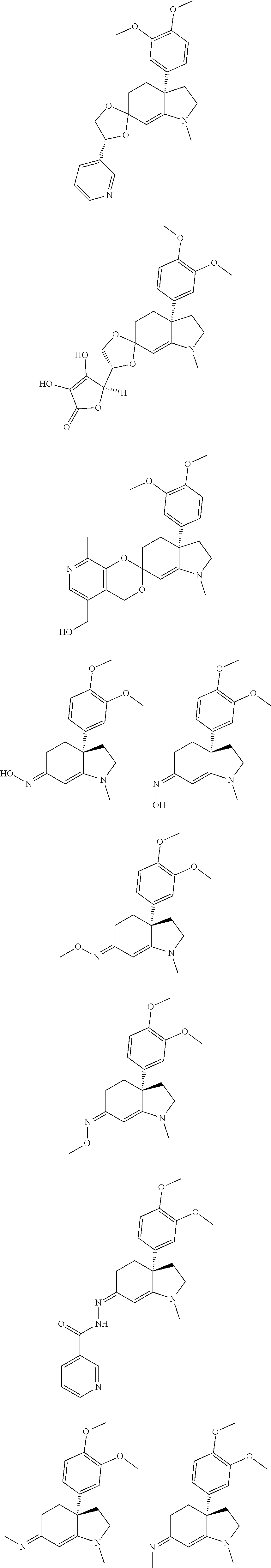

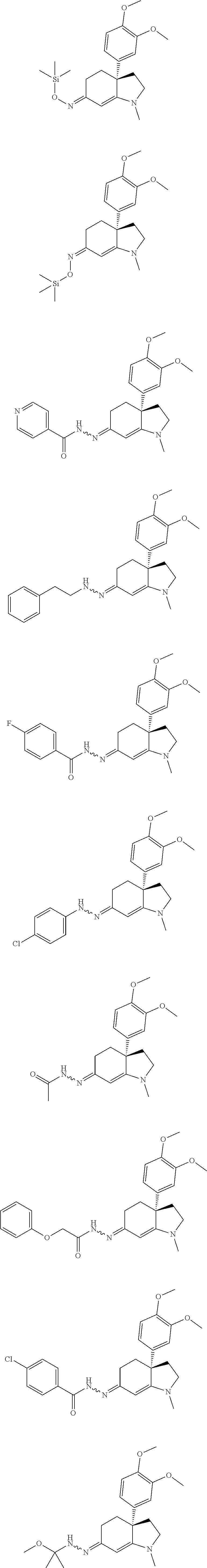

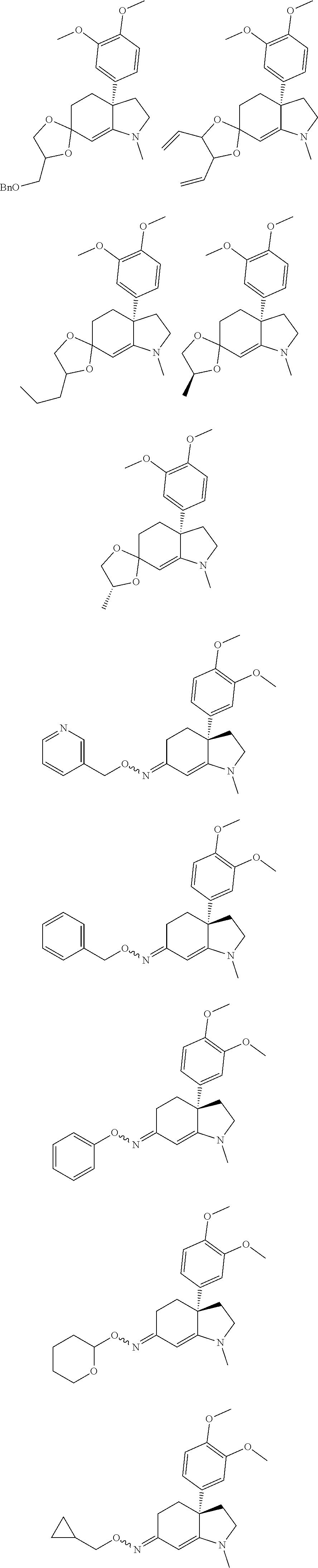

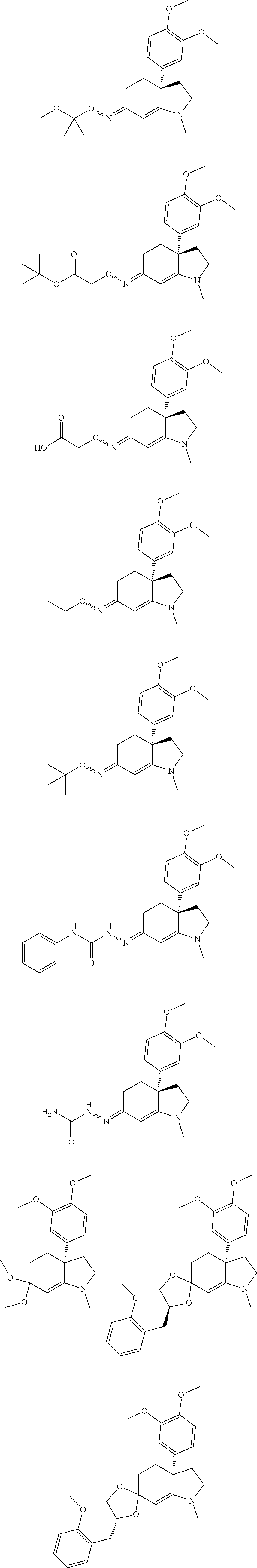

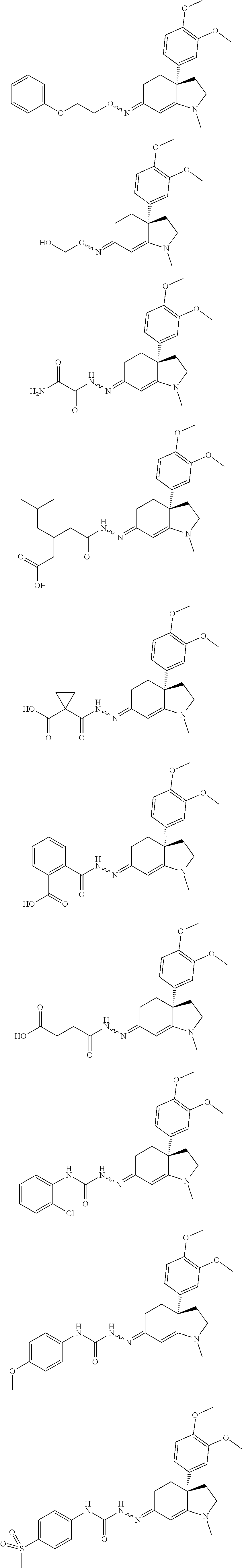

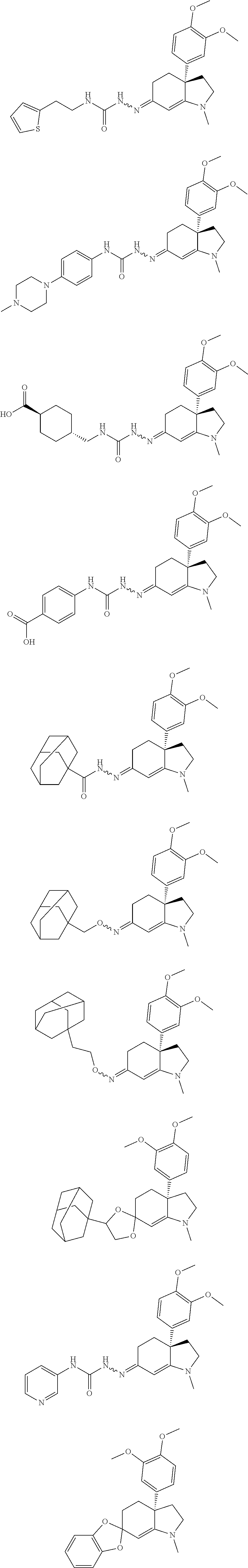

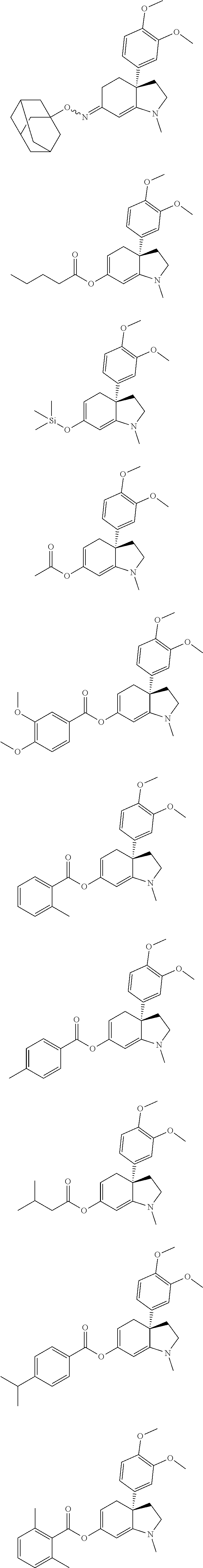

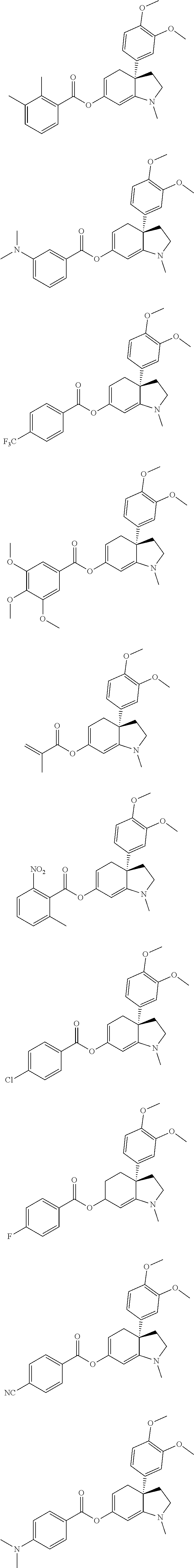

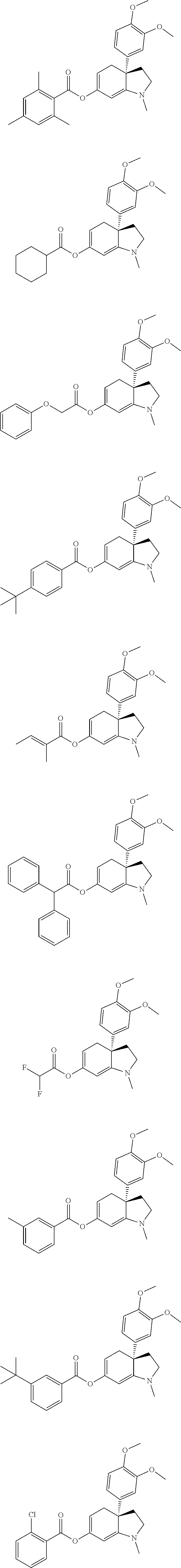

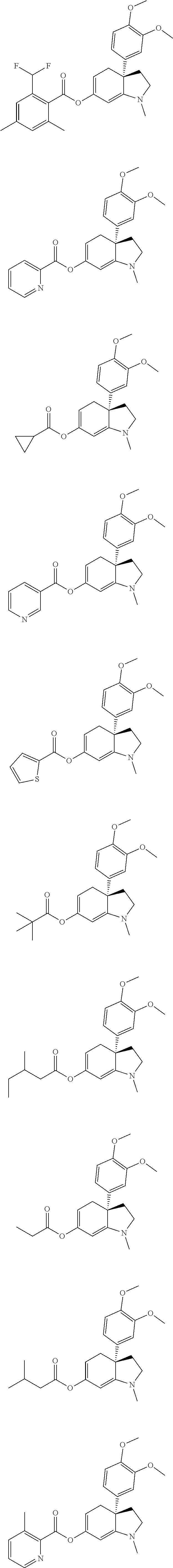

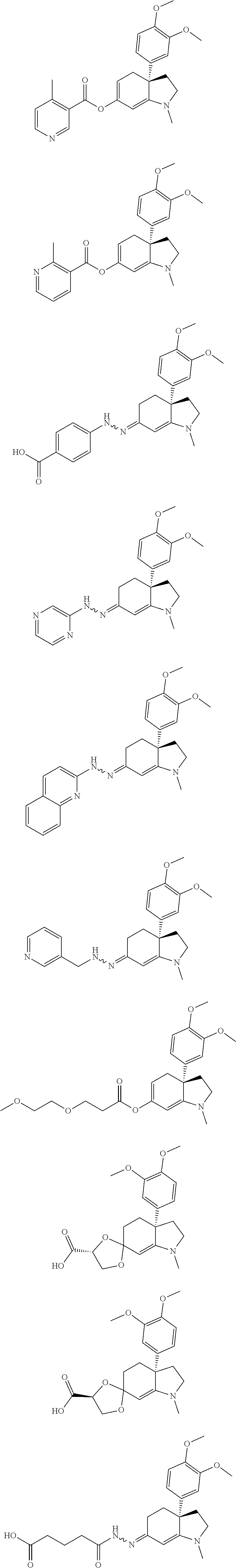

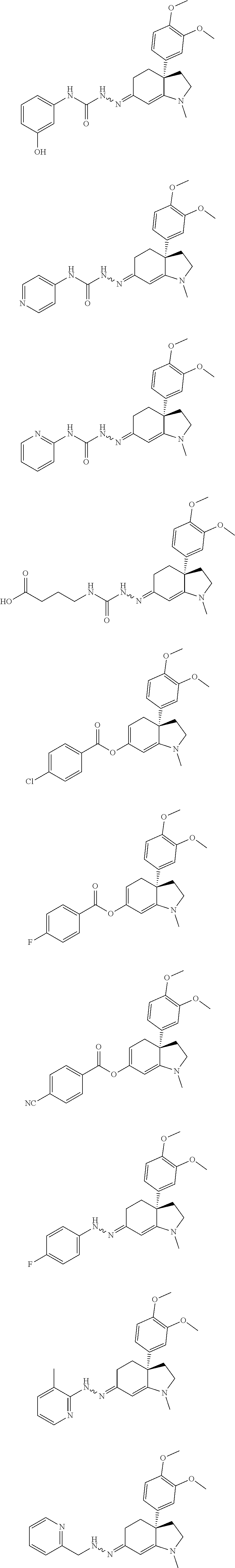

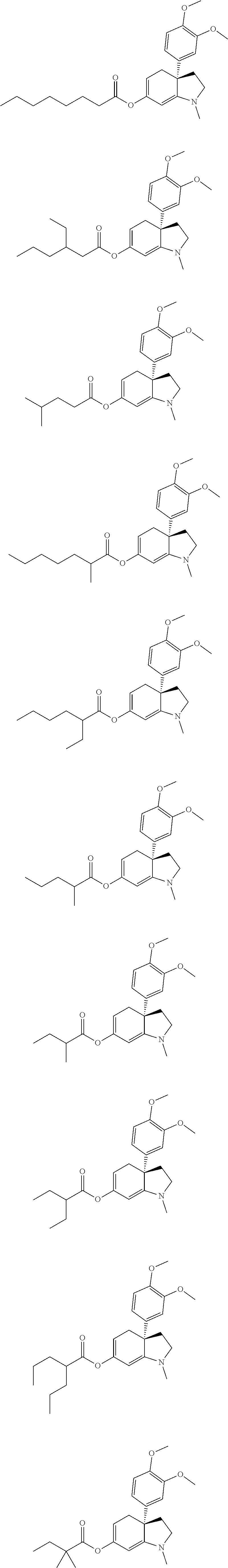

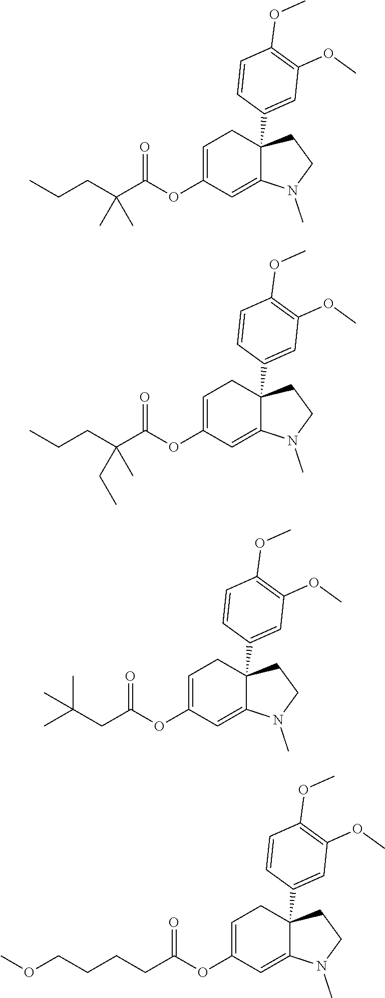

or a pharmaceutically acceptable salt thereof; and the compound has the absolute stereochemistry shown.

The invention is based in part on the discovery of compounds having useful and markedly different properties from naturally occurring 4′-mesembrenone.

In certain embodiments, the present disclosure provides a method of treating a mental disorder, comprising administering to the subject a compound of the present disclosure.

Numerous embodiments are further provided that can be applied to any aspect of the present invention described herein.

### DETAILED DESCRIPTION

The present invention is based, at least in part, on Δ⁷-mesembrenone and analogs thereof.

### EXEMPLARY COMPOUNDS OF THE INVENTION

In some embodiments, a compound according to the present disclosure is a compound of formula (I):

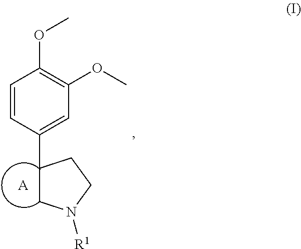

or a pharmaceutically acceptable salt thereof, wherein
R¹ is or C₁-C₇ alkyl or H; and
ring A is

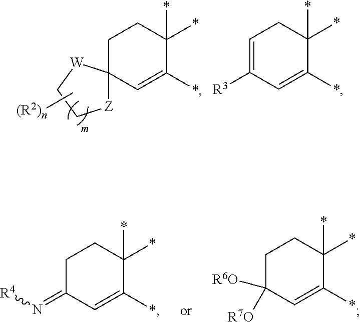

wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, C₃-C₁₀ cycloalkyl, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl or phenyl, wherein each hydrogen atom in 5- to 7-membered heteroaryl or phenyl is optionally substituted by —OH, C₁-C₃ alkyl, or C₁-C₃ alkanol;
m is 1 or 2;
n is 0, 1, 2, or 3;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, phenoxy or —O(CH₂)ₚOCH₃;
p is 2, 3, or 4;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₅ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₅ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and
each of R⁶ and R⁷ is independently C₁-C₃ alkyl.

In some embodiments, a compound according to the present disclosure is of formula (I):

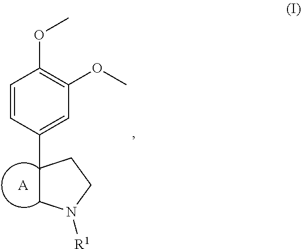

or a pharmaceutically acceptable salt thereof, wherein
R¹ is H or C₁-C₇ alkyl; and
ring A is

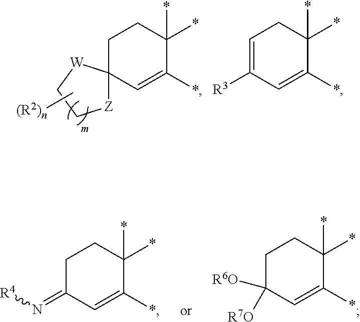

wherein * denotes the attachment points of ring A to the compound of formula (I), and wherein
each of W and Z is independently O, NH, or S;
each R² is independently C₁-C₃ alkyl, —COOH, —CONH₂, phenyl, 5- to 7-membered heterocyclyl, or 5- to 7-membered heteroaryl, wherein each hydrogen atom in phenyl, 5- to 7-membered heterocyclyl and 5- to 7-membered heteroaryl is optionally substituted by —OH, C₁-C₃ alkyl, C₁-C₃ alkoxy, C₁-C₃ alkanol, or —NO₂, or two R²s on a single carbon atom combine to form ═O; or two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol;
m is 1 or 2;
n is 0, 1, or 2;
R³ is —OSi(C₁-C₆ alkyl)₃ or —OC(O)C₁-C₆ alkyl; and
R⁴ is OH, C₁-C₆ alkoxy, or —NHC(O)-5- to 7-membered heteroaryl, wherein each hydrogen atom in 5- to 7-membered heteroaryl is optionally substituted by —OH, C₁-C₃ alkyl, or C₁-C₃ alkanol.

In some embodiments, a compound according to the present disclosure is of formula (I):

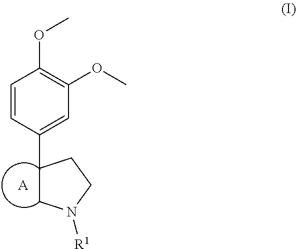

or a pharmaceutically acceptable salt thereof, wherein
R¹ is H or C₁-C₇ alkyl; and
ring A is

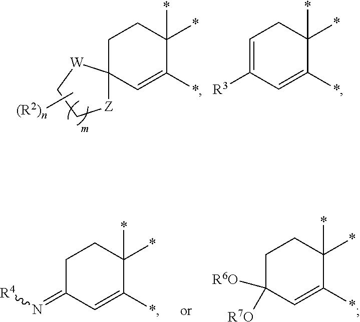

wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, C₃-C₁₀ cycloalkyl, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl or phenyl, wherein each hydrogen atom in 5- to 7-membered heteroaryl or phenyl is optionally substituted by —OH, C₁-C₃ alkyl, or C₁-C₃ alkanol;
m is 1 or 2;
n is 0, 1, or 2;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₅ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and
each of R⁶ and R⁷ is independently C₁-C₃ alkyl.

In some embodiments, the compound is of the compound is of formula (I-1):

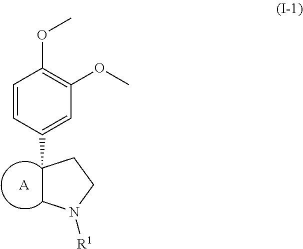

or a pharmaceutically acceptable salt thereof, wherein
R¹ is C₁-C₇ alkyl or H; and
ring A is

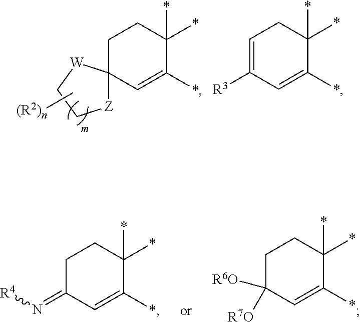

wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, C₃-C₁₀ cycloalkyl, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl or phenyl, wherein each hydrogen atom in 5- to 7-membered heteroaryl or phenyl is optionally substituted by —OH, C₁-C₃ alkyl, or C₁-C₃ alkanol;
m is 1 or 2;
n is 0, 1, 2, or 3;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy or —O(CH₂)ₚOCH₃;
p is 2, 3, or 4;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, —C(O)-5- to 7-membered heteroaryl, —C(O)—C₁-C₈ alkyl, —C(O)—C₃-C₁₀ cycloalkyl, —C(O)— phenyl, —C(O)N(H)-phenyl, C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl;
each of R⁶ and R⁷ is independently C₁-C₃ alkyl; and
the compound of formula (I-1) has the absolute stereochemistry shown.

In certain embodiments, the compound is of formula (I-1):

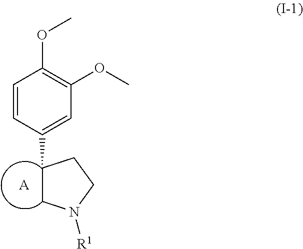

or a pharmaceutically acceptable salt thereof, wherein
R¹ is H or C₁-C₇ alkyl; and
ring A is

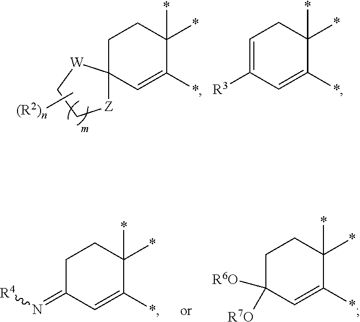

wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, C₃-C₁₀ cycloalkyl, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl or phenyl, wherein each hydrogen atom in 5- to 7-membered heteroaryl or phenyl is optionally substituted by —OH, C₁-C₃ alkyl, or C₁-C₃ alkanol,
m is 1 or 2,
n is 0, 1, or 2,
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₅ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₅ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and
each of R⁶ and R⁷ is independently C₁-C₃ alkyl.

In certain embodiments, the compound is of formula (I-1):

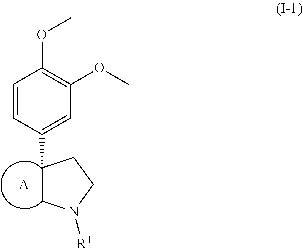

or a pharmaceutically acceptable salt thereof, wherein
R¹ is H or C₁-C₇ alkyl; and
ring A is

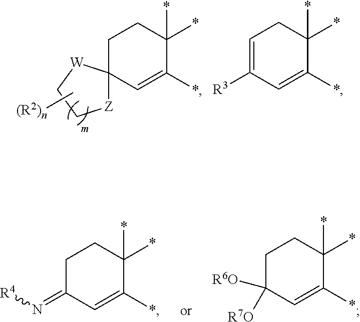

wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, C₃-C₁₀ cycloalkyl, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl or phenyl, wherein each hydrogen atom in 5- to 7-membered heteroaryl or phenyl is optionally substituted by —OH, C₁-C₃ alkyl, or C₁-C₃ alkanol,
m is 1 or 2,
n is 0, 1, or 2,
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₅ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and
each of R⁶ and R⁷ is independently C₁-C₃ alkyl; and
the compound of formula (I-1) has the absolute stereochemistry shown.

In some embodiments, ring A is

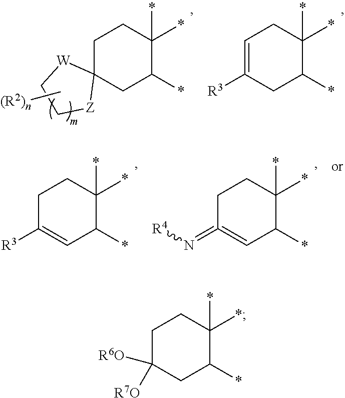

wherein * denotes the attachment points of ring A to the compound of formula (I).

In some embodiments, each of W and Z is independently O, NH, or S. In some embodiments, W is O. In some embodiments, W is NH. In some embodiments, W is S.

In some embodiments, each R² is independently C₁-C₃ alkyl, C₂-C₆ alkenyl, —COOH, —CONH₂, C₃-C₁₀ cycloalkyl, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl. In some embodiments, each R² is independently C₁-C₃ alkyl, C₂-C₆ alkenyl, —COOH, —CONH₂, phenyl, 5- to 7-membered heterocyclyl, or 5- to 7-membered heteroaryl. In some embodiments, each hydrogen atom in C₁—C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, C₁-C₃ alkanol, or —NO₂. In some embodiments, R² is —COOH, —CONH₂, phenyl, 5- to 7-membered heterocyclyl, or 5- to 7-membered heteroaryl, wherein each hydrogen atom in phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl. In some embodiments, R² is phenyl, wherein each hydrogen atom in phenyl is optionally substituted by —OH, C₁-C₃ alkyl, C₁-C₃ alkoxy, or —NO₂. In some embodiments, R² is 5- to 7-membered heterocyclyl or 5- to 7-membered heteroaryl, wherein each hydrogen atom in 5- to 7-membered heterocyclyl and 5- to 7-membered heteroaryl is optionally substituted by —OH, C₁-C₃ alkyl, C₁-C₃ alkoxy, C₁-C₃ alkanol, or —NO₂. In some embodiments, R² is pyridyl. In some embodiments, R² is

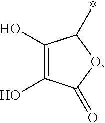 [SMILES: *C1OC(=O)C(O)=C1O]

wherein * denotes the point of attachment of R² the compound. In some embodiments, R² is —COOH or —CONH₂.

In some embodiments, two R²s on a single carbon atom combine to form ═O.

In some embodiments, two instances of R² on a single carbon atom combine to form ═O; or two adjacent R²s together with the carbon atoms to which they are attached combine to form 5- to 7-membered heteroaryl or phenyl, wherein each hydrogen atom in 5- to 7-membered heteroaryl or phenyl is optionally substituted by —OH, C₁-C₃ alkyl, or C₁-C₃ alkanol, In some embodiments, two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol. In some embodiments, the two R²s together with the carbon atoms to which they are attached combine to form pyridyl, wherein each hydrogen atom in pyridyl is optionally substituted by —OH, C₁-C₃ alkyl, or C₁-C₃ alkanol.

In some embodiments, m is 1 or 2.

In some n is 0, 1, or 2. In some embodiments, n is 0. In some embodiments, n is 1. In some embodiments, n is 2.

In some n is 0, 1, 2, or 3. In some embodiments, n is 0. In some embodiments, n is 1. In some embodiments, n is 2. In some embodiments, n is 3.

In some embodiments, R³ is —OSi(C₁-C₆ alkyl)₃ or —OC(O)C₁-C₆ alkyl. In some embodiments, R³ is —OSi(C₁-C₆ alkyl)₃. In some embodiments, R³ is —OC(O)C₁-C₆ alkyl (e.g., methyl).

In some embodiments, R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy. In some embodiments, of R³ is —OC(O)C₁-C₆ alkyl and the C₁-C₆ alkyl is methyl, ethyl, butyl, isobutyl, tertbutyl, or 2-methylbutyl, wherein each hydrogen atom in methyl, ethyl, butyl, isobutyl, tertbutyl, and 2-methylbutyl is optionally substituted by halogen or phenyl. In some embodiments, R³ is —OC(O)C₂-C₆ alkenyl (e.g., isopropenyl or butenyl). In some embodiments, is —OC(O)C₃-C₁₀ cycloalkyl (e.g., cyclopropyl or cyclohexyl). In some embodiments, R³ is —OC(O)phenyl, wherein each hydrogen atom in phenyl is optionally substituted by methyl, isopropyl, tertbutyl, chloro, fluoro, CF₃, —CHF₂, cyano, —N(CH₃)₂, methoxy, or nitro. In some embodiments, —OC(O)-5- to 7-membered heteroaryl, which may be optionally substituted (e.g., pyridyl or thiophenyl, wherein each hydrogen atom in pyridyl or thiophenyl is optionally substituted by methyl).

In some embodiments, R³ is —OC(O)C₁-C₆ alkyl and the C₁-C₆ alkyl is ethyl, propyl, butyl, pentyl, or hexyl, wherein each hydrogen atom in methyl, ethyl, propyl, butyl, pentyl, and hexyl is optionally substituted by methyl, ethyl, methoxy or —O(CH₂)ₚOCH₃; and p is 2, 3, or 4. In some embodiments, R³ is

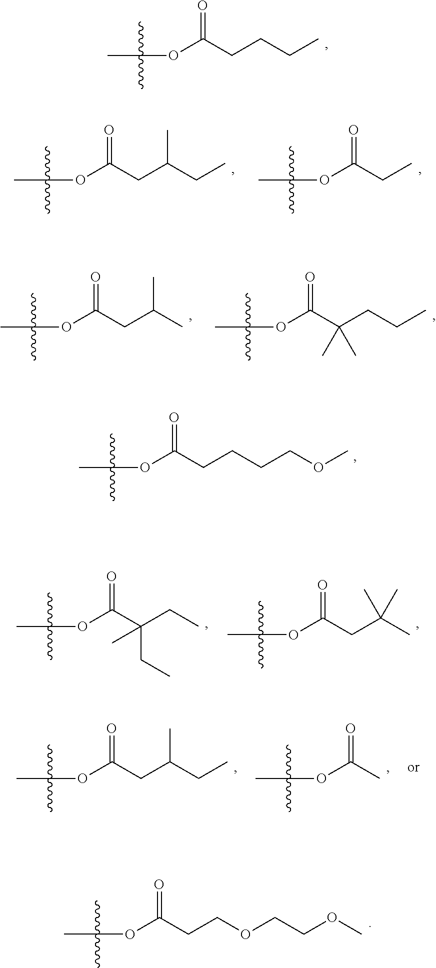

In some embodiments, R³ is —OC(O)C₃-C₁₀ cycloalkyl (e.g., cyclopropyl or cyclohexyl). In some embodiments, the C₃-C₁₀ cycloalkyl is cyclohexyl.

In some embodiments, R³ is —OC(O)phenyl, wherein each hydrogen atom in phenyl is optionally substituted by methyl, isopropyl, tertbutyl, chloro, fluoro, CF₃, —CHF₂, cyano, —N(CH₃)₂, -methoxy, or nitro. In some embodiments, R³ is —OC(O)phenyl, wherein each hydrogen atom in phenyl is optionally substituted by methyl. In some embodiments, R³ is —OC(O)phenyl, wherein one hydrogen atom in phenyl is substituted by methyl.

In some embodiments, R³ is —OC(O)-5- to 7-membered heteroaryl, for example pyridyl or thiophenyl, wherein each hydrogen atom in pyridyl or thiophenyl is optionally substituted by methyl. In some embodiments, the 5- to 7-membered heteroaryl is pyridyl wherein each hydrogen atom in pyridyl is optionally substituted by methyl. In some embodiments, the 5- to 7-membered heteroaryl is unsubstituted pyridyl.

In some embodiments, R³ is —OC(O)C₁-C₆ alkyl, —OC(O)C₃-C₁₀ cycloalkyl, —OC(O)phenyl, —OC(O)-5- to 7-membered heteroaryl, wherein each hydrogen atom in C₁-C₆ alkyl, C₃-C₁₀ cycloalkyl, phenyl, and 5- to 7-membered heteroaryl is optionally substituted by C₁-C₆ alkyl, C₁-C₃ alkoxy, or —O(CH₂)ₚOCH₃; and p is 2, 3, or 4. In some embodiments, the C₁-C₆ alkyl is unsubstituted or is substituted by methoxy. In some embodiments, the C₃-C₁₀ cycloalkyl is unsubstituted cyclohexyl. In some embodiments, phenyl is monosubstituted, for example by methyl. In some embodiments, the 5- to 7-membered heteroaryl is unsubstituted pyridyl.

In some embodiments, R³ is —OC(O)C₁-C₆ alkyl, —OC(O)C₂-C₆ alkenyl, —OC(O)C₃-C₁₀ cycloalkyl, —OC(O)phenyl, or —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or —O(CH₂)ₚOCH₃; and p is 2, 3, or 4.

In some embodiments, R⁴ is OH, C₁-C₆ alkoxy, or —NHC(O)-5- to 7-membered heteroaryl. In some embodiments, each hydrogen atom in 5- to 7-membered heteroaryl is optionally substituted by —OH, C₁-C₃ alkyl, or C₁-C₃ alkanol.

In some embodiments, R⁴ is —OR⁵ or —N(R⁵)₂.

In some embodiments, R⁴ is OR⁵ wherein R⁵ is H, C₁-C₆ alkyl, C₃-C₁₀ cycloalkyl, C₃-C₆ heterocycloalkyl or phenyl, wherein each hydrogen atom in C₁-C₆ alkyl, C₃-C₆ heterocycloalkyl and phenyl is optionally substituted with —OH, C₁-C₆ alkoxy, 5- to 7-membered heteroaryl, phenyl, phenoxy, C₃-C₆ cycloalkyl, —C(O)OC₁-C₆ alkyl, C₃-C₁₀ cycloalkyl, or —COOH.

In some embodiments, R⁴ is —N(R⁵)₂ wherein each R⁵ is independently H (preferably one is H), —C₁-C₆ alkyl, —C(O)-5- to 7-membered heteroaryl, —C(O)-phenyl, phenyl, —C(O)—C₁-C₃ alkyl, —C(O)—C₃-C₁₀ cycloalkyl, —C(O)N(H)-phenyl, —C(O)N(H)-pyridyl, —C(O)COOH, or —CONH₂, wherein each hydrogen atom in —C₁-C₆ alkyl, —C(O)-5- to 7-membered heteroaryl, —C(O)-phenyl, phenyl, —C(O)—C₁-C₃ alkyl, and —C(O)N(H)-phenyl, is optionally substituted by halogen, aryloxy, carboxylate, phenyl, —S(O)₂CH₃, C₁-C₃ alkoxy, C₃-C₆ cycloalkyl optionally substituted by carboxylate, or 5- to 7-membered heterocyclyl optionally substituted by methyl.

In some embodiments, R⁴ is —N(R⁵)₂ and one R⁵ is H and the other R⁵ is-C(O)N(H)-phenyl optionally substituted by —COOH.

In some embodiments, each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl. In some embodiments, each R⁵ is H, C₁-C₆ alkyl, C₃-C₆ heterocycloalkyl, C₁-C₆ alkoxy, phenyl, —C(O)-5- to 7-membered heteroaryl, —C(O)—C₁-C₃ alkyl, —C(O)-phenyl, —C(O)N(H)-phenyl, or —CONH₂. In some embodiments, each hydrogen atom in C₁-C₆ alkyl, C₁-C₆ alkoxy phenyl, and 5- to 7-membered heteroaryl is optionally substituted by halogen, OH, C₁-C₃ alkyl, C₃-C₆ cycloalkyl, phenyl, 5- to 7-membered heteroaryl aryloxy (e.g., phenoxy), C₁-C₆ alkoxy, C₁-C₃ alkanol, —COOH, —C(O)C₁-C₆ alkyl, or —C(O)OC₁-C₆ alkyl. In some embodiments, R⁵ is 5- to 10-membered heteroaryl

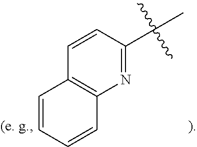 [SMILES: *c1ccc2ccccc2n1]

In some embodiments, each of R⁶ and R⁷ is independently C₁-C₃ alkyl, for example methyl.

In some embodiments, R¹ is H or C₁-C₇ alkyl. In some embodiments, R¹ is C₁-C₃ alkyl. In some embodiments, R¹ is methyl.

Table 2 provides examples of compounds that could be reacted to obtain compounds of Formula (I) by the reaction: Compound M+Compound R 4→Compound C.

TABLE 2

| Reactant M | Reactant R | Product Compound C |
|---|---|---|
| 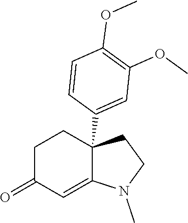 [SMILES: COc1ccc([C@@]23CCC(=O)C=C2N(C)CC3)cc1OC] | 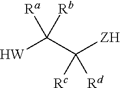 | 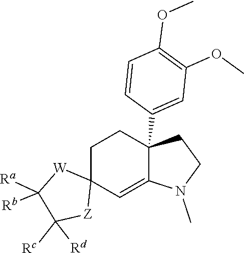 |
| 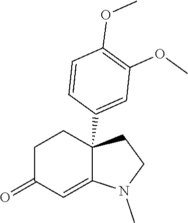 [SMILES: COc1ccc([C@@]23CCC(=O)C=C2N(C)CC3)cc1OC] | 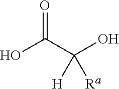 | 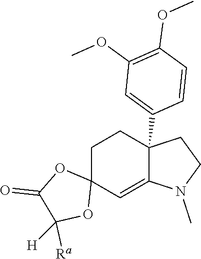 |
| 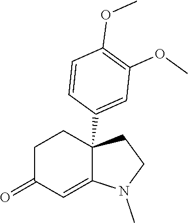 [SMILES: COc1ccc([C@@]23CCC(=O)C=C2N(C)CC3)cc1OC] | 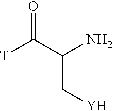 | 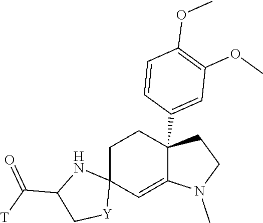 |
| 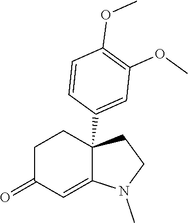 [SMILES: COc1ccc([C@@]23CCC(=O)C=C2N(C)CC3)cc1OC] | 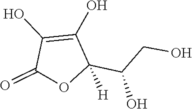 [SMILES: O=C1O[C@H]([C@@H](O)CO)C(O)=C1O] | 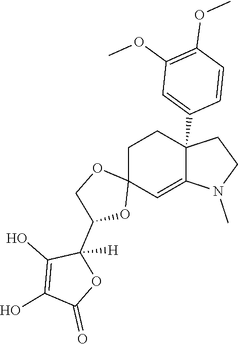 |
| 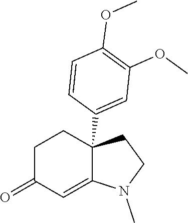 [SMILES: COc1ccc([C@@]23CCC(=O)C=C2N(C)CC3)cc1OC] | 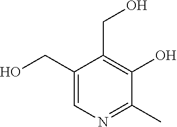 | 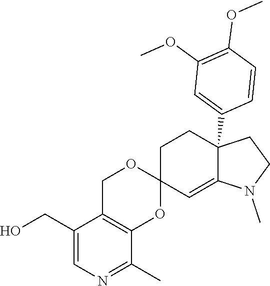 [SMILES: COc1ccc([C@]23CCN(C)C2=CC2(CC3)OCc3c(CO)cnc(C)c3O2)cc1OC] |
| 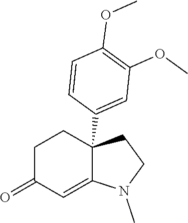 [SMILES: COc1ccc([C@@]23CCC(=O)C=C2N(C)CC3)cc1OC] | (R⁸)₂O or R⁸—X | 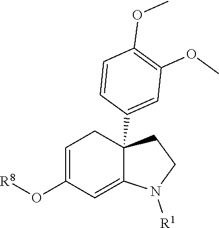 |
| 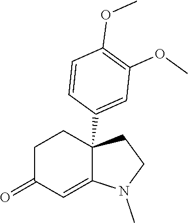 [SMILES: COc1ccc([C@@]23CCC(=O)C=C2N(C)CC3)cc1OC] | 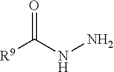 | 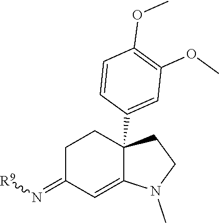 |

Unless otherwise indicated in the tables of compounds herein, the abbreviation RAC or rac indicates a racemic mixture, and DIAST indicates a specific diastereomer. In illustrative embodiments, although a compound may be depicted with [symbol] or [symbol] bonds, such a depiction may be denoting relative stereochemistry based on elution peaks from a chiral separation.

Compounds of Formula (I) including compounds of Formula (IIa) could be prepared by treatment of a ketone, such as Δ⁷-mesembrenone, with an appropriate diol or dithiane, and an acid catalyst, such as p-toluenesulfonic acid or methanesulfonic acid, in a solvent, such as toluene, at an elevated temperature.

TABLE 3
Exemplary Compounds of Formula IIa

| | |
|---|---|
| 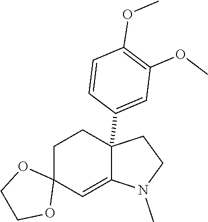 [SMILES: COc1ccc([C@]23CCN(C)C2=CC2(CC3)OCCO2)cc1OC] | 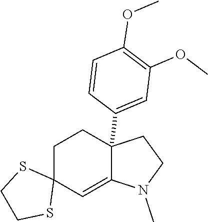 [SMILES: COc1ccc([C@]23CCN(C)C2=CC2(CC3)SCCS2)cc1OC] |
| 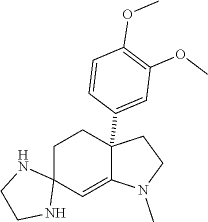 [SMILES: COc1ccc([C@]23CCN(C)C2=CC2(CC3)NCCN2)cc1OC] | 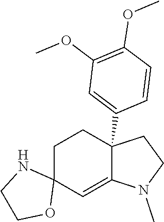 [SMILES: COc1ccc([C@]23CCN(C)C2=CC2(CC3)NCCO2)cc1OC] |
| 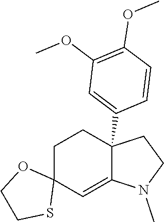 [SMILES: COc1ccc([C@]23CCN(C)C2=CC2(CC3)OCCS2)cc1OC] | 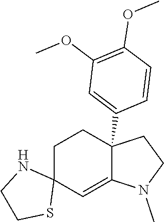 [SMILES: COc1ccc([C@]23CCN(C)C2=CC2(CC3)NCCS2)cc1OC] |
| 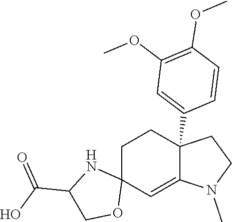 [SMILES: COc1ccc([C@]23CCN(C)C2=CC2(CC3)NC(C(=O)O)CO2)cc1OC] | 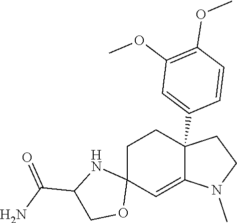 [SMILES: COc1ccc([C@]23CCN(C)C2=CC2(CC3)NC(C(N)=O)CO2)cc1OC] |
| 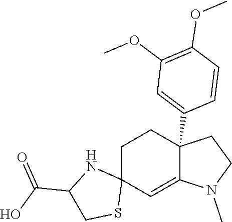 [SMILES: COc1ccc([C@]23CCN(C)C2=CC2(CC3)NC(C(=O)O)CS2)cc1OC] | 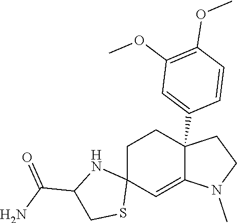 [SMILES: COc1ccc([C@]23CCN(C)C2=CC2(CC3)NC(C(N)=O)CS2)cc1OC] |
| 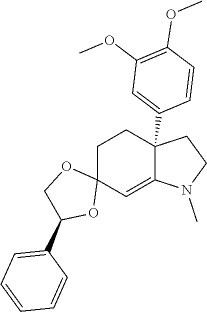 [SMILES: COc1ccc([C@]23CCN(C)C2=CC2(CC3)OC[C@H](c3ccccc3)O2)cc1OC] | 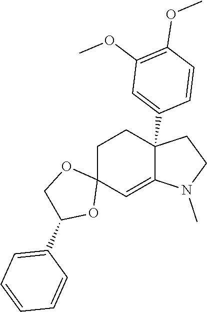 [SMILES: COc1ccc([C@]23CCN(C)C2=CC2(CC3)OC[C@@H](c3ccccc3)O2)cc1OC] |
| 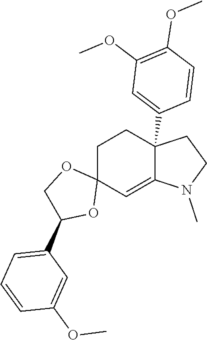 [SMILES: COc1cccc([C@H]2COC3(C=C4N(C)CC[C@]4(c4ccc(OC)c(OC)c4)CC3)O2)c1] | 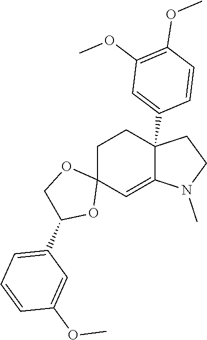 [SMILES: COc1cccc([C@@H]2COC3(C=C4N(C)CC[C@]4(c4ccc(OC)c(OC)c4)CC3)O2)c1] |
| 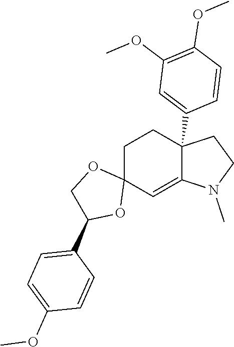 [SMILES: COc1ccc([C@H]2COC3(C=C4N(C)CC[C@]4(c4ccc(OC)c(OC)c4)CC3)O2)cc1] | 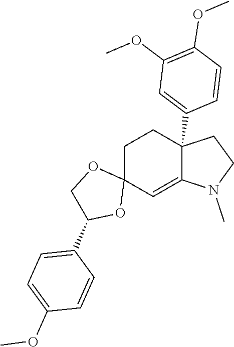 [SMILES: COc1ccc([C@@H]2COC3(C=C4N(C)CC[C@]4(c4ccc(OC)c(OC)c4)CC3)O2)cc1] |
| 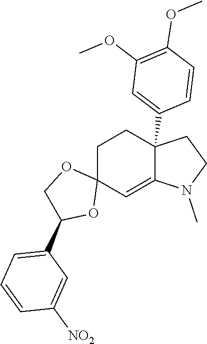 [SMILES: COc1ccc([C@]23CCN(C)C2=CC2(CC3)OC[C@H](c3cccc([N+](=O)[O-])c3)O2)cc1OC] | 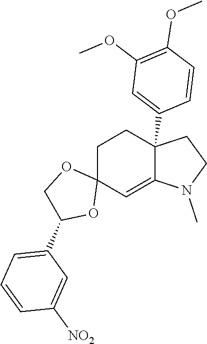 [SMILES: COc1ccc([C@]23CCN(C)C2=CC2(CC3)OC[C@@H](c3cccc([N+](=O)[O-])c3)O2)cc1OC] |
| 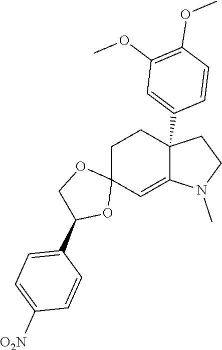 [SMILES: COc1ccc([C@]23CCN(C)C2=CC2(CC3)OC[C@H](c3ccc([N+](=O)[O-])cc3)O2)cc1OC] | 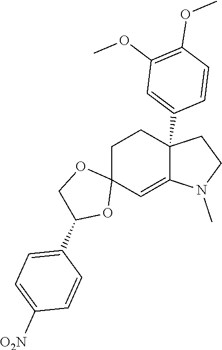 [SMILES: COc1ccc([C@]23CCN(C)C2=CC2(CC3)OC[C@@H](c3ccc([N+](=O)[O-])cc3)O2)cc1OC] |
| 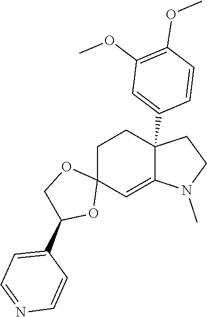 [SMILES: COc1ccc([C@]23CCN(C)C2=CC2(CC3)OC[C@H](c3ccncc3)O2)cc1OC] | 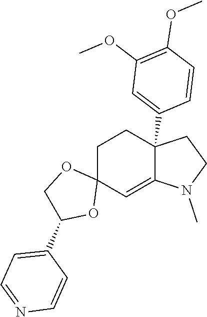 [SMILES: COc1ccc([C@]23CCN(C)C2=CC2(CC3)OC[C@@H](c3ccncc3)O2)cc1OC] |
| 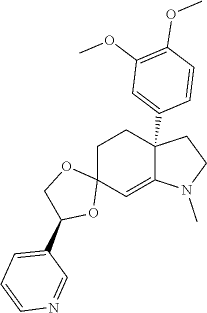 [SMILES: COc1ccc([C@]23CCN(C)C2=CC2(CC3)OC[C@H](c3cccnc3)O2)cc1OC] | 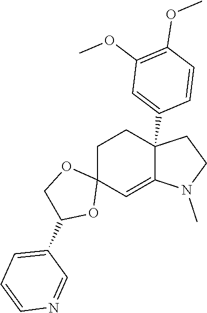 |
| 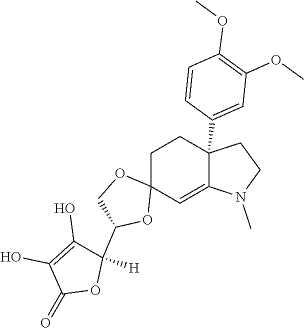 | 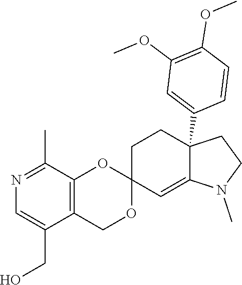 [SMILES: COc1ccc([C@]23CCN(C)C2=CC2(CC3)OCc3c(CO)cnc(C)c3O2)cc1OC] |
| 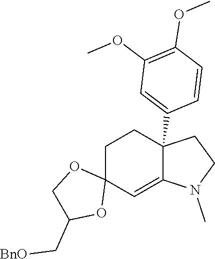 [SMILES: COc1ccc([C@]23CCN(C)C2=CC2(CC3)OCC(COCc3ccccc3)O2)cc1OC] | 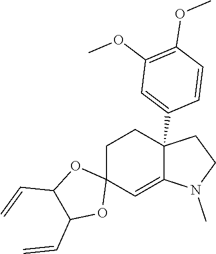 [SMILES: C=CC1OC2(C=C3N(C)CC[C@]3(c3ccc(OC)c(OC)c3)CC2)OC1C=C] |
| 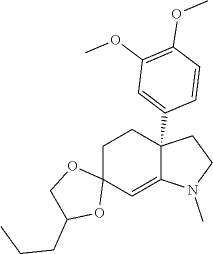 [SMILES: CCCC1COC2(C=C3N(C)CC[C@]3(c3ccc(OC)c(OC)c3)CC2)O1] | 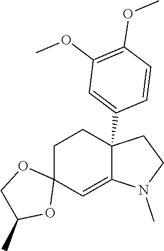 [SMILES: COc1ccc([C@]23CCN(C)C2=CC2(CC3)OC[C@H](C)O2)cc1OC] |
| 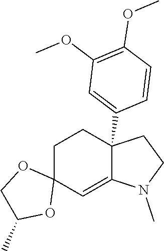 [SMILES: COc1ccc([C@]23CCN(C)C2=CC2(CC3)OC[C@@H](C)O2)cc1OC] | 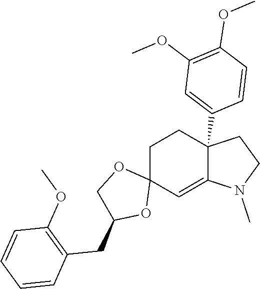 [SMILES: COc1ccccc1C[C@H]1COC2(C=C3N(C)CC[C@]3(c3ccc(OC)c(OC)c3)CC2)O1] |
| 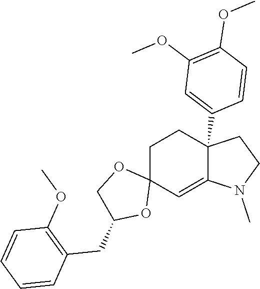 [SMILES: COc1ccccc1C[C@@H]1COC2(C=C3N(C)CC[C@]3(c3ccc(OC)c(OC)c3)CC2)O1] | 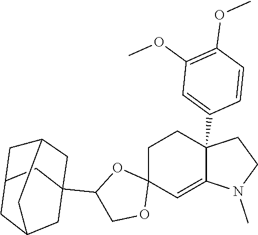 [SMILES: COc1ccc([C@]23CCN(C)C2=CC2(CC3)OCC(C34CC5CC(CC(C5)C3)C4)O2)cc1OC] |
| 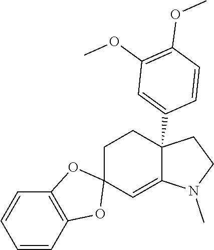 [SMILES: COc1ccc([C@]23CCN(C)C2=CC2(CC3)Oc3ccccc3O2)cc1OC] | 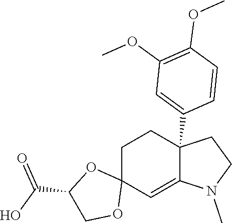 [SMILES: COc1ccc([C@]23CCN(C)C2=CC2(CC3)OC[C@H](C(=O)O)O2)cc1OC] |

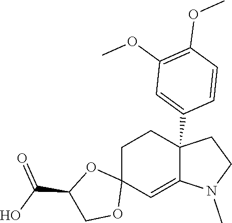 [SMILES: COc1ccc([C@]23CCN(C)C2=CC2(CC3)OC[C@@H](C(=O)O)O2)cc1OC]

In some embodiments, the compound of formula (I) is a compound of formula (IX):

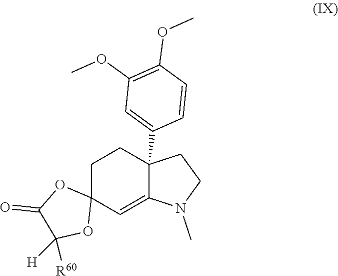

or a pharmaceutically acceptable salt thereof, and R₆₀ is hydrogen or methyl.

Compounds of Formula (IX) could be prepared by treatment of a ketone, such as Δ7-mesembrenone, with an appropriate alpha-hydroxy carboxylic acid, with a Lewis acid, such as boron trifluoride etherate, in a solvent, such as dichloromethane. For example, a compound of formula (IX) where R₆₀ is methyl could be obtained by reacting mesembrine with lactic acid (e.g., with TsOH, toluene). In some embodiments, the compound can be a lactate or glycolate derivative of Δ7-mesembrenone.

Table 4 provides non-limiting examples of certain compounds of Formula (IX).

TABLE 4
Exemplary Compounds of Formula IX

| | |
|---|---|
| 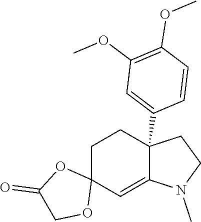 [SMILES: COc1ccc([C@]23CCN(C)C2=CC2(CC3)OCC(=O)O2)cc1OC] | 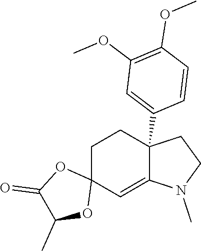 |

In some embodiments, the compound of Formula (I) is a compound of Formula (X) or a pharmaceutically acceptable salt thereof:

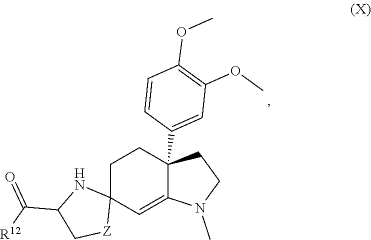

wherein Z is S or O; and R₁₂ is hydroxyl or amino.

Compounds of Formula (X) could be prepared by treatment of a ketone, such as Δ7-mesembrenone, with an appropriate alpha-amino carboxylic acid or alpha-amino amide, in a solvent, such as ethanol, at an elevated temperature.

Table 5 provides non-limiting examples of certain compounds of Formula (X).

TABLE 5
Exemplary Compounds of Formula X

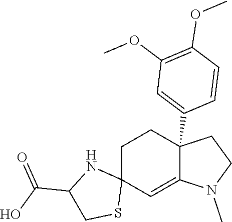 [SMILES: COc1ccc([C@]23CCN(C)C2=CC2(CC3)NC(C(=O)O)CS2)cc1OC]

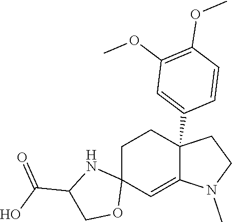 [SMILES: COc1ccc([C@]23CCN(C)C2=CC2(CC3)NC(C(=O)O)CO2)cc1OC]

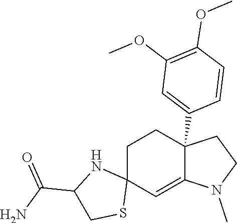 [SMILES: COc1ccc([C@]23CCN(C)C2=CC2(CC3)NC(C(N)=O)CS2)cc1OC]

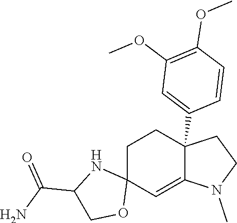 [SMILES: COc1ccc([C@]23CCN(C)C2=CC2(CC3)NC(C(N)=O)CO2)cc1OC]

In some embodiments, the compound of Formula (I) is a compound of Formula (XI) or a pharmaceutically acceptable salt thereof:

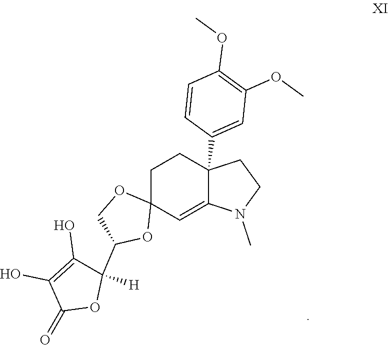

Compounds of Formula (XI) could be prepared by treatment of a ketone such as Δ7-mesembrenone, with a naturally occurring diol, such as ascorbic acid, with an acid catalyst, such as TsOH) in a solvent, such as toluene or acetone.

Table 6 provides non-limiting examples of certain compounds of Formula (XI).

TABLE 6
Exemplary Compound of Formula XI

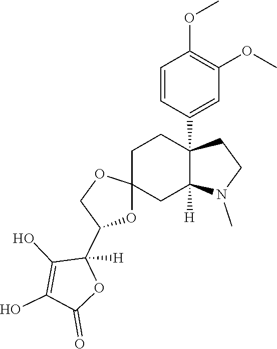

In some embodiments, the compound of Formula (I) is a compound of Formula (XII) or a pharmaceutically acceptable salt thereof:

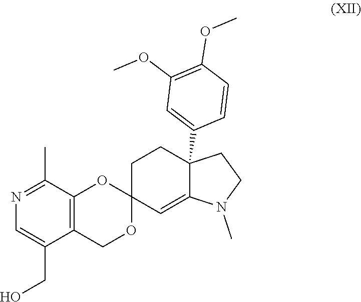 [SMILES: COc1ccc([C@]23CCN(C)C2=CC2(CC3)OCc3c(CO)cnc(C)c3O2)cc1OC]

The compound of formula (XII) could be prepared by treatment of a ketone such as Δ7-mesembrenone, with pyridoxine with an acid catalyst in a solvent.

Table 7 provides non-limiting examples of the compound of Formula (XII).

TABLE 7
Exemplary Compound of Formula XII

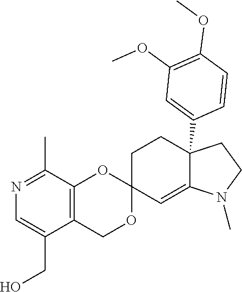 [SMILES: COc1ccc([C@]23CCN(C)C2=CC2(CC3)OCc3c(CO)cnc(C)c3O2)cc1OC]

In some embodiments, a compound of Formula (I) can be a compound of Formula (XIII) or a pharmaceutically acceptable salt thereof:

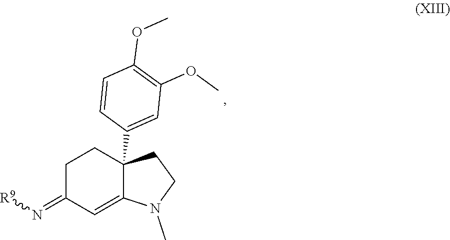

wherein R₉ is hydroxyl, methoxy, or —NH—C(O)-6-member nitrogen-containing heteroaryl. In some embodiments, the 6-member nitrogen-containing heteroaryl is pyridine.

Table 8 provides non-limiting examples of certain compounds of Formula (XIII).

TABLE 8
Exemplary Compounds of Formula (XIII)

| | |
|---|---|
| 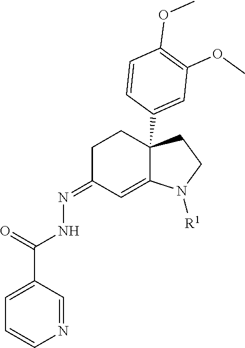 | 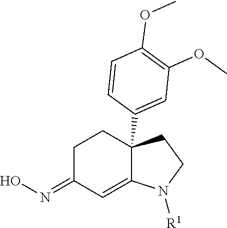 |
| 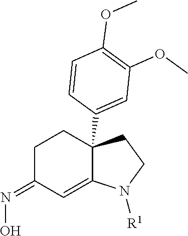 | 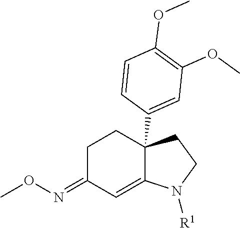 |
| 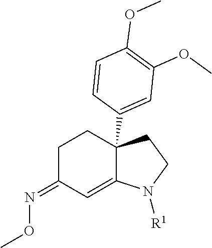 | 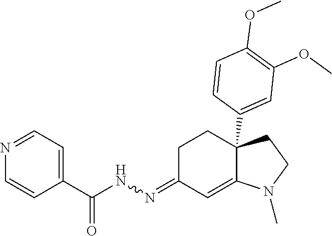 [SMILES: COc1ccc([C@@]23CCC(=NNC(=O)c4ccncc4)C=C2N(C)CC3)cc1OC] |

In some embodiments, the compound is a compound of Formula (XV)

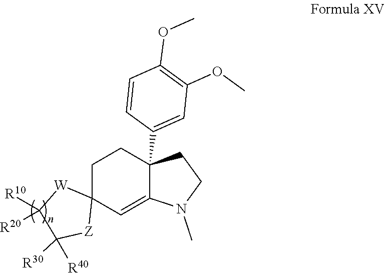

or a pharmaceutically acceptable salt thereof wherein
n is 0 or 1;
W and Z are each independently CH₂, O, S or NH, provided that at least one of W or Z is O, S or NH; and
R₁₀, R₂₀, R₃₀ and R₄₀ are each hydrogen.

In some embodiments, W and Z in Formula (XV) are each O. In some embodiments, W and Z in Formula (XV) are each S. In some embodiments, W and Z in Formula (XV) are each NH. In some embodiments, W is NH and Z is S in Formula (XV). In some embodiments, W is O and Z is S in Formula (XV). In some embodiments, W is CH₂ and Z is O in Formula (XV). In some embodiments, W is CH₂ and Z is NH in Formula (XV).

In some embodiments, a compound of Formula (XV) can be a compound of Formula (XV-1):

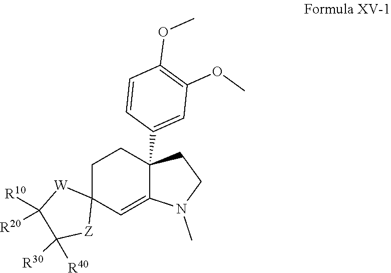

Compounds of Formula (XV) including compounds of Formula (XV-1) could be prepared by treatment of a ketone, such as Δ⁷-mesembrenone, with an appropriate glycol derivative, and an acid catalyst, such as p-toluenesulfonic acid or methanesulfonic acid, in a solvent, such as toluene, at an elevated temperature.

Table 9 provides non-limiting examples of certain compounds of Formula (XV).

TABLE 9
Exemplary Compounds of Formula XV

| | |
|---|---|
| 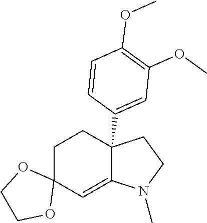 [SMILES: COc1ccc([C@]23CCN(C)C2=CC2(CC3)OCCO2)cc1OC] | 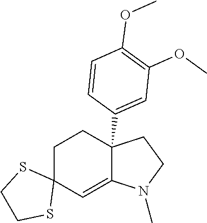 [SMILES: COc1ccc([C@]23CCN(C)C2=CC2(CC3)SCCS2)cc1OC] |
| 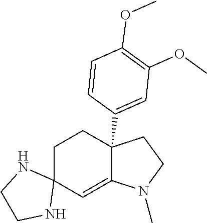 [SMILES: COc1ccc([C@]23CCN(C)C2=CC2(CC3)NCCN2)cc1OC] | 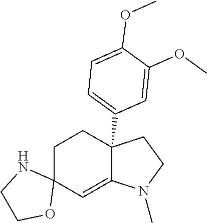 [SMILES: COc1ccc([C@]23CCN(C)C2=CC2(CC3)NCCO2)cc1OC] |
| 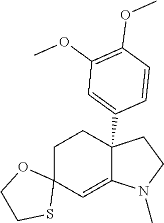 [SMILES: COc1ccc([C@]23CCN(C)C2=CC2(CC3)OCCS2)cc1OC] | 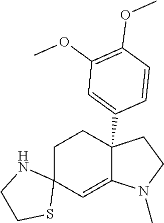 [SMILES: COc1ccc([C@]23CCN(C)C2=CC2(CC3)NCCS2)cc1OC] |

The synthesis of Δ⁷-mesembrenone has been reported (Takano et al. Tet. Lett. 1981, 22, PG 4479-4482). Mesembrine alkaloids have been synthesized in a manner similar to that of Amaryllidaceae alkaloids (e.g., Roe C, Sandoe E J, Stephenson G R, Anson C E. Stereoselectivity in the organoiron-mediated synthesis of (±)-mesembrine. Tetrahedron Lett. 2008; 49(4):650-653; and Shamma M, Rodriguez HR. The synthesis of (+)-mesembrine. Tetrahedron. 1968; 24(22):6583-6589.5714008).

In some embodiments, methods of treating a patient suffering from a disease comprise administering to a patient a composition comprising a compound disclosed herein for the treatment or prevention of a mental health disorder. In some embodiments, methods of treating a patient suffering from a disease comprise administering to a patient a composition comprising a compound disclosed herein for the treatment or prevention of a diagnosed condition selected from anxiety and depression. In some embodiments, a method comprises the administration to a patient in need thereof of a therapeutically effective amount of a compound of Formula (I) for the treatment of depression. In some embodiments, a method comprises the administration to a patient in need thereof of a therapeutically effective amount of a compound of Formula (I) for the treatment of a condition selected from the group consisting of: anxiety associated with depression, anxiety with depression, mixed anxiety and depressive disorder. In some embodiments, a method comprises the administration to a patient in need thereof of a therapeutically effective amount of a compound of Formula (I) for the treatment of anxiety and hysteria or anxiety and depression.

In some embodiments, the compound disclosed herein is administered to the patient in a unit dose. In some embodiments, the compound disclosed herein is prescribed to a patient in an oral unit dose for such as a capsule or tablet once or more times per day. In some embodiments, a compound disclosed herein is administered to a patient for the treatment of a disease or condition for which mesembrine is safe and effective for treatment. In some embodiments, a method comprises the administration to a patient in need thereof of a therapeutically effective amount of a compound of Formula (I) for the treatment of anxiety. In some embodiments, a method comprises the administration to a patient in need thereof of a therapeutically effective amount of a compound of Formula (I) for the treatment of a disease selected from the group consisting of mild to moderate depression and major depressive episodes. In some embodiments, a method comprises the administration to a patient in need thereof of a therapeutically effective amount of a compound of Formula (I) for the treatment of a disease selected from the group consisting of psychological and psychiatric disorders where anxiety is present. In some embodiments, a method comprises the administration to a patient in need thereof of a therapeutically effective amount of a compound of Formula (I) for the treatment of major depressive episodes. In some embodiments, a method comprises the administration to a patient in need thereof of a therapeutically effective amount of a compound of Formula (I) for the treatment of a disease selected from the group consisting of alcohol and drug dependence, bulimia nervosa, and obsessive-compulsive disorders. In some embodiments, an amount of from 20 micrograms to 2 milligrams of a compound of Formula (I) is orally administered to a patient to treat the patient in need thereof with a therapeutic compound. In some embodiments, an amount of from 20 micrograms to 2 milligrams of a compound of Formula (I) is orally administered to a patient to treat the patient in need thereof.

### Pharmaceutical Compositions

In certain embodiments, the present application is directed to a pharmaceutical composition comprising an active pharmaceutical ingredient. In certain embodiments, the pharmaceutical composition comprises a compound as disclosed herein as the active pharmaceutical ingredient (API) and a pharmaceutically acceptable carrier comprising one or more excipients. In some embodiments, the pharmaceutical composition optionally further comprises an additional therapeutic compound (i.e., agent) with the pharmaceutically acceptable carrier. The pharmaceutical composition can be a medicament.

Pharmaceutically acceptable carriers include those known in the art. The choice of a pharmaceutically acceptable carrier can depend, for example, on the desired route of administration of the composition. A pharmaceutical composition (preparation) can be administered to a subject by any of a number of routes of administration including, for example, parenteral administration (e.g. intravenously, subcutaneously, or intramuscularly), oral administration (for example, tablets, and capsules); absorption through the oral mucosa (e.g., sublingually) or transdermally (for example as a patch applied to the skin) or topically (for example, as a cream, ointment or spray applied to the skin).

In some embodiments, pharmaceutical compositions comprising compounds of Formula (I) or pharmaceutically acceptable salts thereof can be formulated for oral administration. For example, a compound provided herein can be combined with suitable compendial excipients to form an oral unit dosage form, such as a capsule or tablet, containing a target dose of a compound of Formula (I). The drug product can be prepared by first manufacturing the compound of Formula (I) as an active pharmaceutical ingredient (API), followed by roller compaction/milling with intragranular excipients and blending with extra granular excipients. A Drug Product can contain the selected compound of Formula (I) as the API and excipient components in a tablet in a desired dosage strength of a compound of Formula (1). The blended material can be compressed to form tablets and then film coated. The excipients can be selected from materials appropriate for inclusion in a pharmaceutical composition for an intended purpose and route of delivery including providing a desired manufacturing and stability properties and/or desired in vivo characteristics or other properties to the pharmaceutical composition. In some embodiments, the pharmaceutical composition can include a compound of Formula (I) as the API in combination with a filler (e.g., a form of microcrystalline cellulose), a dry binder or disintegrant (e.g., a cross-linked polymer), a glidant (e.g., colloidal silicon dioxide) and/or a lubricant (e.g., magnesium stearate). In some embodiments, the pharmaceutical composition can comprise a material such as an extended release or disintegrant involved in carrying or transporting the API pharmaceutical agent from one organ, or portion of the body, to another organ, or portion of the body of a subject, including materials to desirable control the absorption of the API in the intestine.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

To prepare solid dosage forms for oral administration, the active ingredient is mixed with one or more pharmaceutically acceptable carriers, such as sodium citrate or dicalcium phosphate, and/or any of the following: (1) fillers or extenders, (2) binders, (3) humectants, (4) disintegrating agents, (5) solution retarding agents, (6) absorption accelerators, (7) wetting agents, (8) absorbents, (9) lubricants, (10) complexing agents, and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using suitable excipients. The pharmaceutical compositions according to the present invention may contain conventional pharmaceutical carriers and/or auxiliary agents. In some embodiments, he pharmaceutical compositions according to the present invention may contain conventional carrier agents including a binder, a lubricant and/or a glidant selected from those products and materials generally used in pharmaceutical industry for preparation of pharmaceutical compositions for an intended route of administration.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

Liquid dosage forms useful for oral administration include pharmaceutically acceptable carriers and the active ingredient provided as a solid form for reconstitution prior to administration or as a liquid (e.g., solutions, suspensions, or emulsions). In addition to the active ingredient, a liquid dosage forms may contain inert diluents commonly used in the art. For example, formulations of pharmaceutically acceptable compositions for injection can include aqueous solutions such as water or physiologically buffered saline or other solvents or vehicles suitable for the intended route of administration. In some embodiments, the pharmaceutical composition is formulated for parenteral administration.

The therapeutically effective amount of a pharmaceutical composition can be determined by human clinical trials to determine the safe and effective dose for a patient with a relevant diagnosis. It is generally understood that the effective amount of the compound may vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the pharmaceutical composition at a dose and dose interval determined to be safe and effective for the patient.

The present disclosure includes the use of pharmaceutically acceptable salts of compounds of the invention in the compositions and methods of the present invention. Pharmaceutically-acceptable salts include, for example, acid-addition salts and base-addition salts. The acid that is added to a compound to form an acid-addition salt can be an organic acid or an inorganic acid. A base that is added to a compound to form a base-addition salt can be an organic base or an inorganic base. In some embodiments, a pharmaceutically-acceptable salt is a metal salt, in some embodiments, a pharmaceutically-acceptable salt is an ammonium salt. For example, a pharmaceutically acceptable acid addition salt can exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups, C₁-C₁₀ branched-chain alkyl groups, cycloalkyl (alicyclic) groups, alkyl-substituted cycloalkyl groups, and cycloalkyl-substituted alkyl groups. Preferably, the “alkyl” group refers to C₁-C₇ straight-chain alkyl groups or C₁-C₇ branched-chain alkyl groups. Most preferably, the “alkyl” group refers to C₁-C₃ straight-chain alkyl groups or C₁-C₃ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “haloalkyl” refers to an alkyl group substituted with at least one hydrogen atom on a carbon replaced by a halogen. Illustrative halogens include fluoro, chloro, bromo, and iodo. Illustrative haloalkyl groups include trifluoromethyl and 2,2,2-trifluoroethyl, etc.

The term “alkoxyalkyl” refers to an alkyl group substituted with an alkoxy group and may be represented by the general formula alkyl-O-alkyl.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS-.

The term “amide”, as used herein, refers to a group

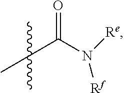

wherein R^{e} and R^{f} each independently represent a hydrogen or hydrocarbyl group, or R^{e} and R^{f} taken together with the N atom to which they are attached complete a heterocycle having from 4 to 8 atoms in the ring structure.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Preferably, the “alkoxy” group refers to C₁-C₇ straight-chain alkoxy groups or C₁-C₇ branched-chain alkoxy groups. Representative alkoxy groups include methoxy, ethoxy, propoxy, tert-butoxy and the like.

The term “aryloxy” refers to an aryl group having an oxygen attached thereto. Preferably, the “aryloxy” group refers to C₆-C₁₀ aryloxy groups or 5-7-membered heteroaryloxy groups. Representative aryloxy groups include phenoxy (C₆H₅—O—) and the like.

The terms “amine” and “amino” are art-recognized and refer to both unsubstituted and substituted amines and salts thereof, e.g., a moiety that can be represented by

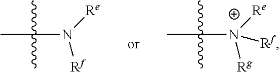

wherein R^{e}, R^{f}, and R^{g}, each independently represent a hydrogen or a hydrocarbyl group, or R^{e} and R^{f} taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring, for example a phenyl. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

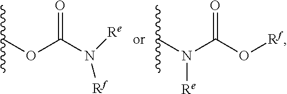

wherein R^{e} and R^{f} independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains six or fewer carbon atoms, preferably four or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

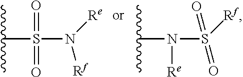

wherein R^{e} and R^{f} independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR^{e} or —SC(O)R^{e}
wherein R^{e} represents a hydrocarbyl.

The term “thioether”, as used herein, is equivalent to an ether, wherein the oxygen is replaced with a sulfur.

The term “urea” is art-recognized and may be represented by the general formula

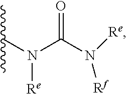

wherein R^{e} and R^{f} independently represent hydrogen or a hydrocarbyl.

The term “modulate” as used herein includes the inhibition or suppression of a function or activity (such as cell proliferation) as well as the enhancement of a function or activity.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. The mono- or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form mesembrine. Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs include using ester or phosphoramidate as biologically labile or cleavable (protecting) groups. The prodrugs of this disclosure are metabolized to produce mesembrine. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

### ALTERNATIVE EMBODIMENTS

In some embodiments, compounds described herein are compounds of formula (I):

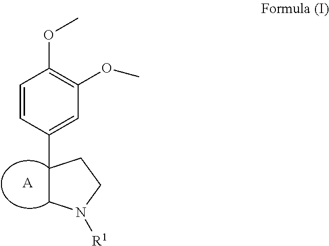

or a pharmaceutically acceptable salt thereof, wherein
R¹ is H or C₁-C₇ alkyl; and
ring A is

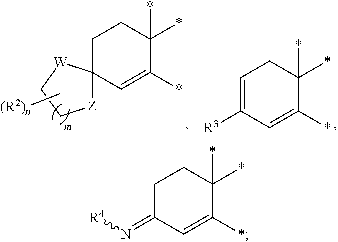

wherein * denotes the attachment points of ring A to the compound of formula (I), and wherein
   each of W and Z is independently O, NH, or S;
   each R² is independently C₁-C₃ alkyl, —COOH, —CONH₂, phenyl, 5- to 7-membered heterocyclyl, or 5- to 7-membered heteroaryl, wherein each hydrogen atom in phenyl, 5- to 7-membered heterocyclyl and 5- to 7-membered heteroaryl is optionally substituted by —OH, C₁-C₃ alkyl, C₁-C₃ alkoxy, C₁-C₃ alkanol, or —NO₂, or two R⁴s on a single carbon atom combine to form ═O; or two adjacent R⁴s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol,
   m is 1 or 2,
   n is 0, 1, or 2,
   R³ is —OSi(C₁-C₆ alkyl)₃ or —OC(O)C₁-C₆ alkyl; and
   R⁴ is OH, C₁-C₆ alkoxy, or —NHC(O)-5- to 7-membered heteroaryl, wherein each hydrogen atom in 5- to 7-membered heteroaryl is optionally substituted by —OH, C₁-C₃ alkyl, or C₁-C₃ alkanol.

In certain embodiments, the compound is of formula (I-1):

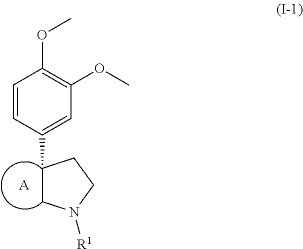

or a pharmaceutically acceptable salt thereof, wherein
R¹ is H or C₁-C₇ alkyl; and
ring A is

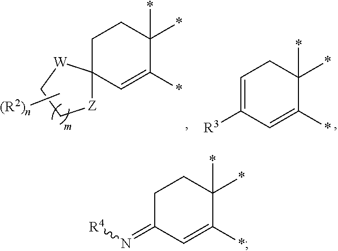

wherein * denotes the attachment points of ring A to the compound of formula (I), and wherein
each of W and Z is independently O, NH, or S;
each R² is independently C₁-C₃ alkyl, —COOH, —CONH₂, phenyl, 5- to 7-membered heterocyclyl, or 5- to 7-membered heteroaryl, wherein each hydrogen atom in phenyl, 5- to 7-membered heterocyclyl and 5- to 7-membered heteroaryl is optionally substituted by —OH, C₁-C₃ alkyl, C₁-C₃ alkoxy, C₁-C₃ alkanol, or —NO₂, or two R⁴s on a single carbon atom combine to form ═O; or two adjacent R⁴s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol,
m is 1 or 2,
n is 0, 1, or 2,
R³ is —OSi(C₁-C₆ alkyl)₃ or —OC(O)C₁-C₆ alkyl; and
R⁴ is OH, C₁-C₆ alkoxy, or —NHC(O)-5- to 7-membered heteroaryl, wherein each hydrogen atom in 5- to 7-membered heteroaryl is optionally substituted by —OH, C₁-C₃ alkyl, or C₁-C₃ alkanol; and
the compound of formula (I-1) has the absolute stereochemistry shown.

In certain embodiments, the compound of formula (I) is a compound of formula (IIa):

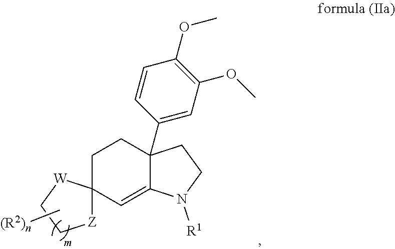

or a pharmaceutically acceptable salt thereof, wherein R¹, R², m, n, W, and Z are as defined herein.

In certain embodiments, the compound is of formula (IIa-1):

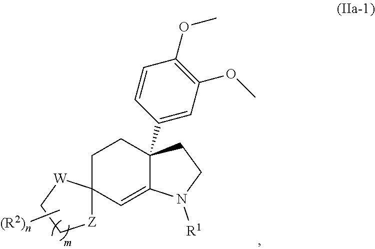

or a pharmaceutically acceptable salt thereof, wherein R¹, R², m, n, W, and Z are as defined herein; and the compound of formula (IIa-1) has the absolute stereochemistry shown.

In certain embodiments, the compound of formula (I) is a compound of formula (IIIa):

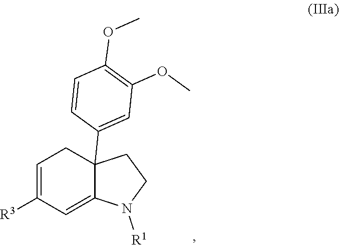

or a pharmaceutically acceptable salt thereof, wherein R¹, and R³ are as defined herein.

In certain embodiments, the compound is of formula (IIIa-1):

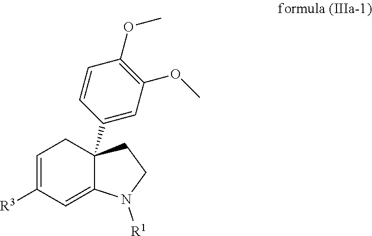

or a pharmaceutically acceptable salt thereof, wherein R¹ and R³ are as defined herein; and the compound of formula (IIIa-1) has the absolute stereochemistry shown.

In certain embodiments, the compound of formula (I) is a compound of formula (IVa):

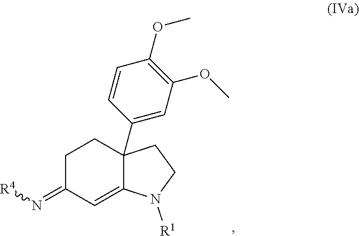

or a pharmaceutically acceptable salt thereof, wherein R¹ and R⁴ are as defined herein.

In certain embodiments, the compound is of formula (IVa-1):

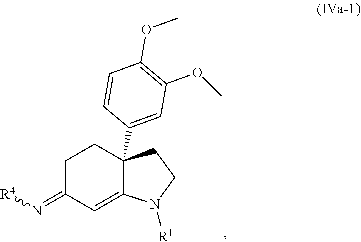

or a pharmaceutically acceptable salt thereof, wherein R¹ and R⁴ are as defined herein; and the compound of formula (IVa-1) has the absolute stereochemistry shown.

In certain embodiments, the compound is of formula (IA):

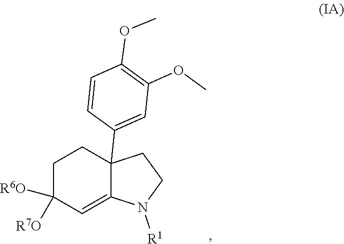

or a pharmaceutically acceptable salt thereof, wherein R¹, R⁶ and R⁷ are as defined herein.

In some embodiments, the compound is selected from the group consisting of:

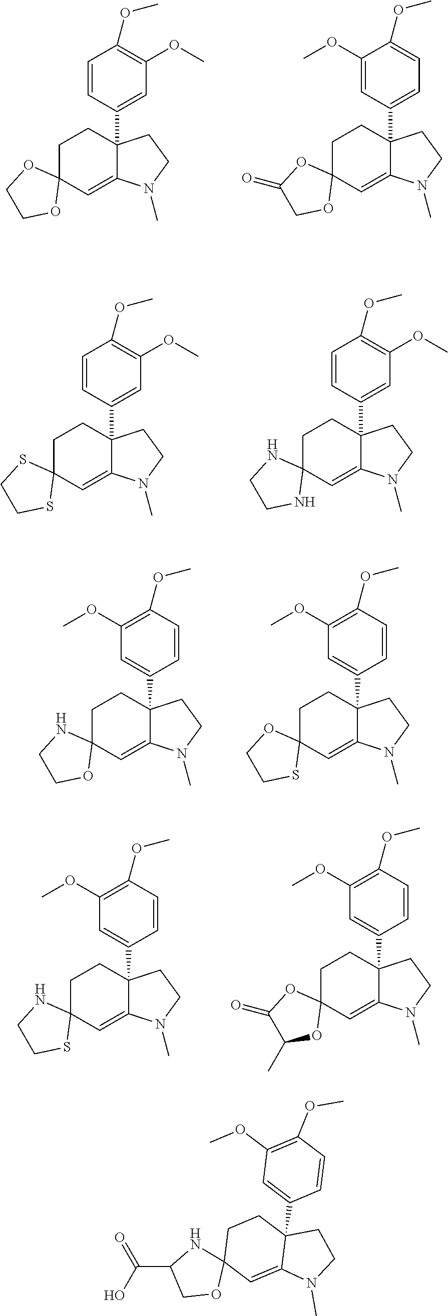

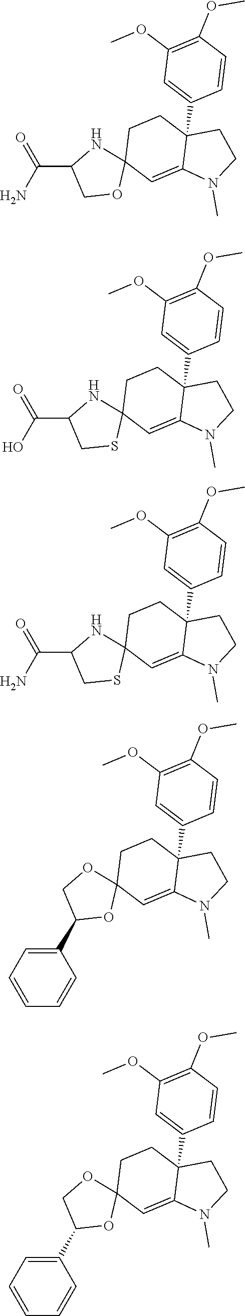

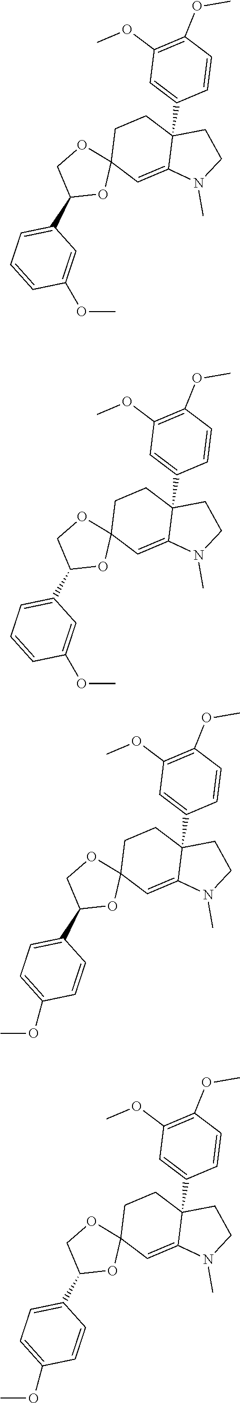

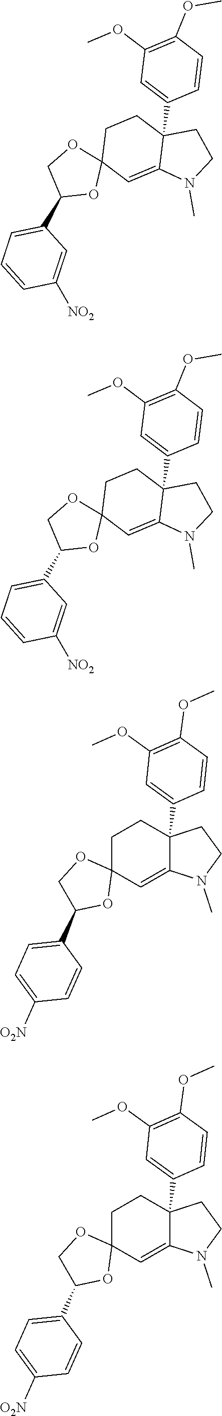 [SMILES: COc1ccc([C@]23CCN(C)C2=CC2(CC3)OC[C@H](c3cccc([N+](=O)[O-])c3)O2)cc1OC ; COc1ccc([C@]23CCN(C)C2=CC2(CC3)OC[C@@H](c3cccc([N+](=O)[O-])c3)O2)cc1OC ; COc1ccc([C@]23CCN(C)C2=CC2(CC3)OC[C@H](c3ccc([N+](=O)[O-])cc3)O2)cc1OC ; COc1ccc([C@]23CCN(C)C2=CC2(CC3)OC[C@@H](c3ccc([N+](=O)[O-])cc3)O2)cc1OC]

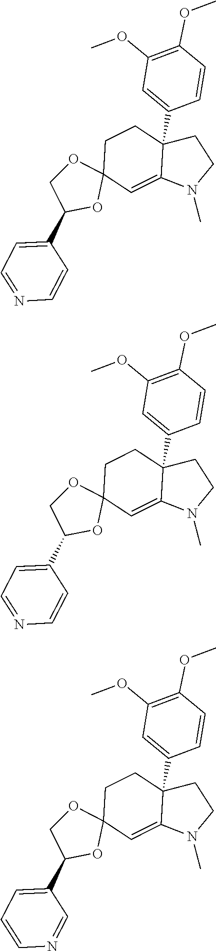 [SMILES: COc1ccc([C@]23CCN(C)C2=CC2(CC3)OC[C@H](c3ccncc3)O2)cc1OC ; COc1ccc([C@]23CCN(C)C2=CC2(CC3)OC[C@@H](c3ccncc3)O2)cc1OC ; COc1ccc([C@]23CCN(C)C2=CC2(CC3)OC[C@H](c3cccnc3)O2)cc1OC]

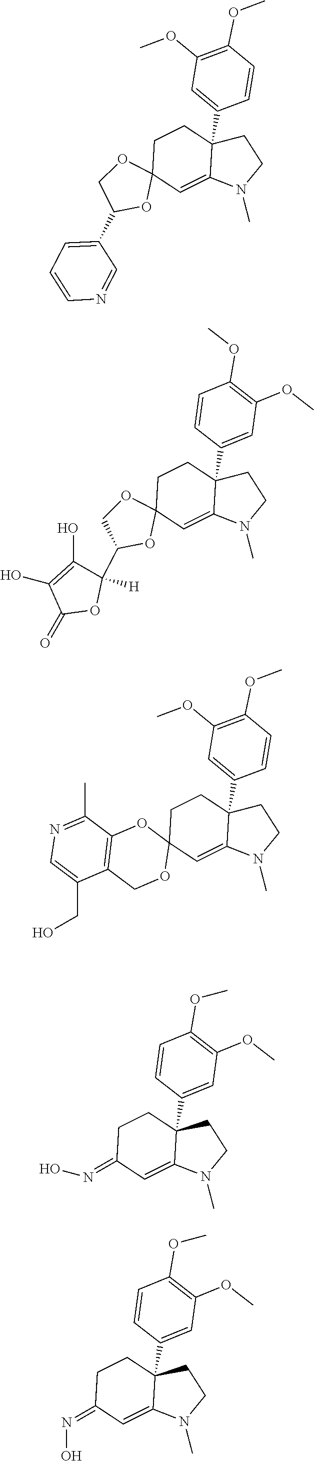

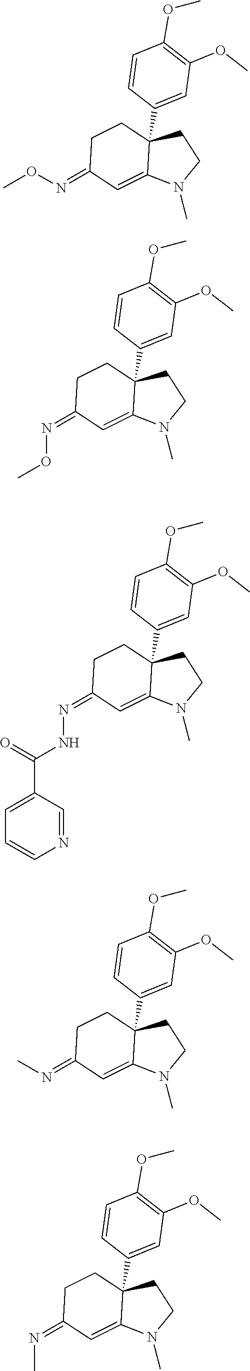

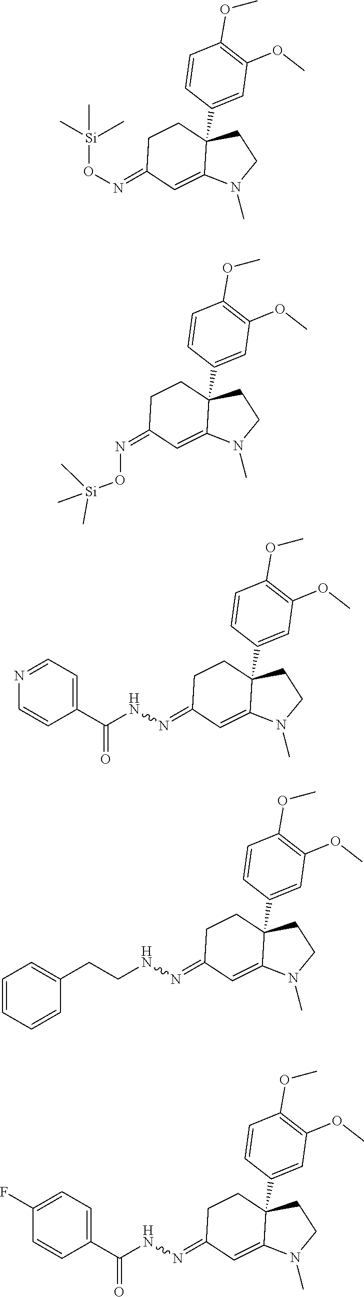

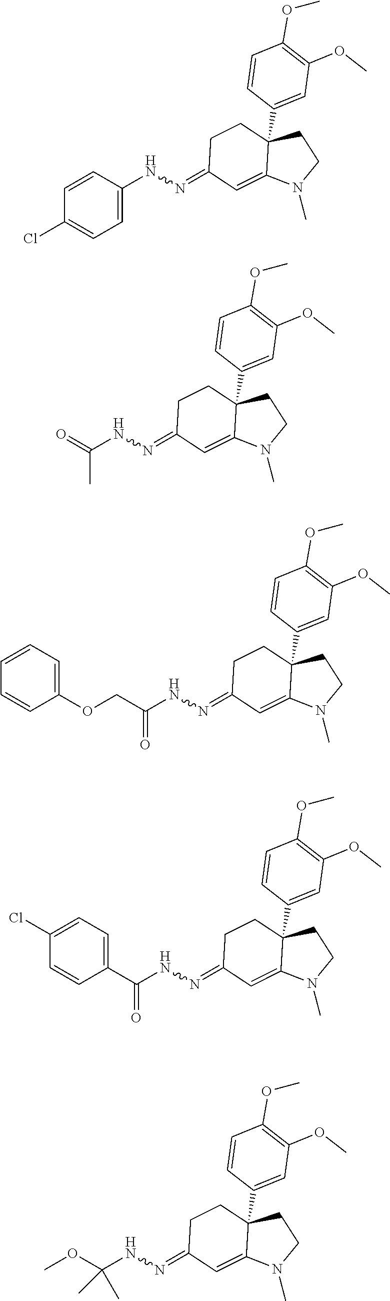

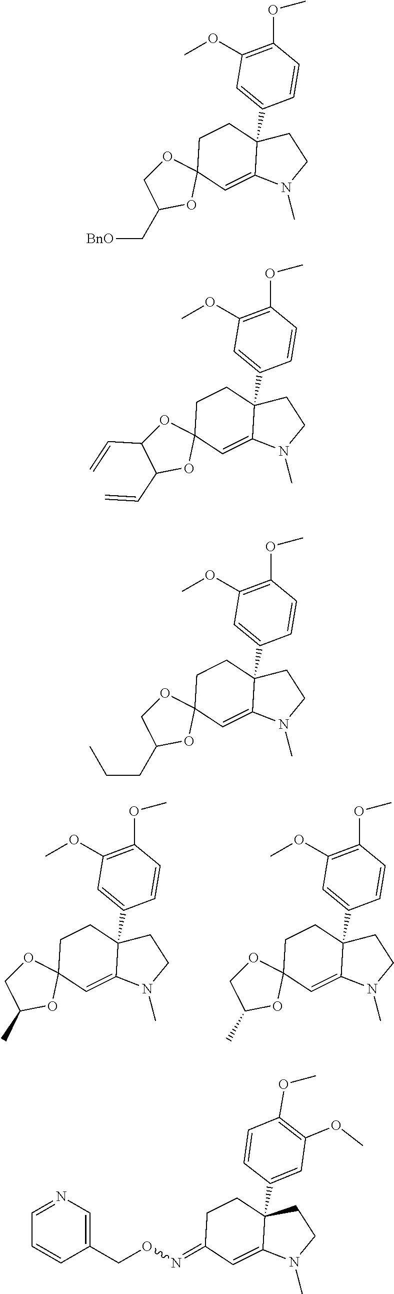

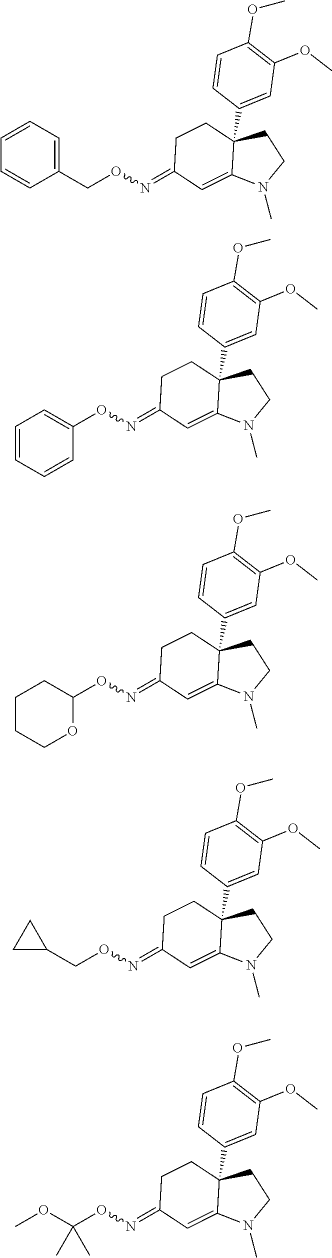

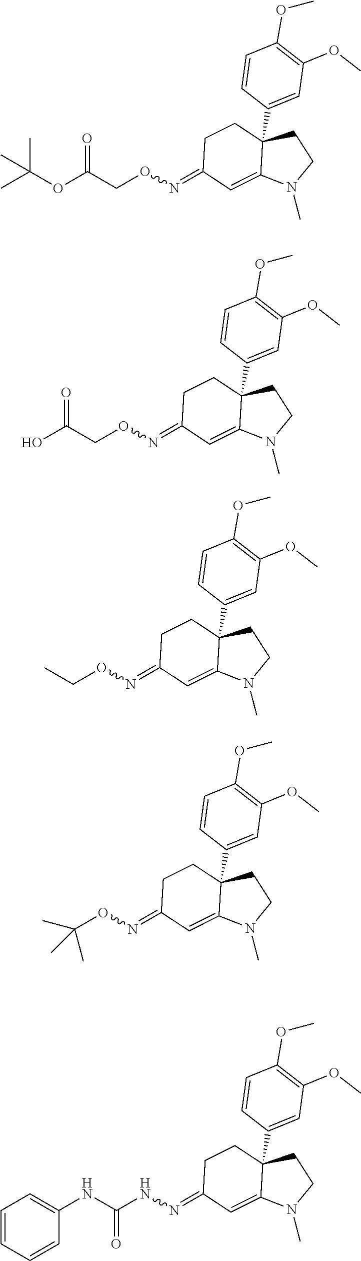

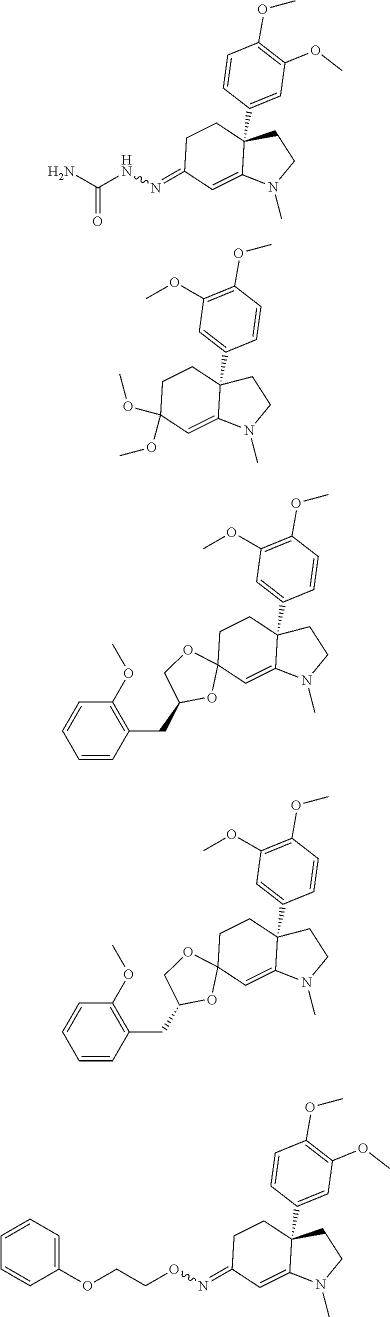

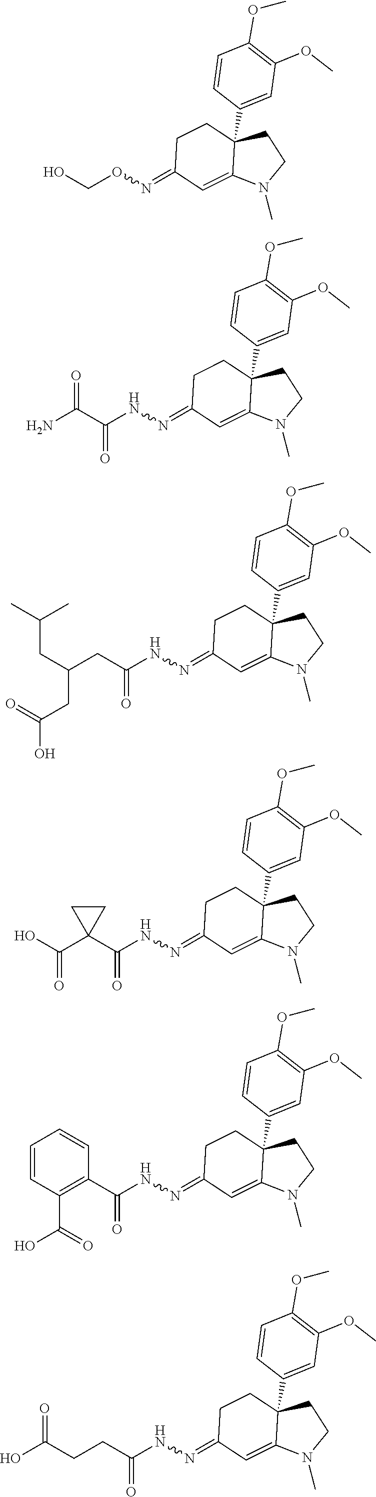

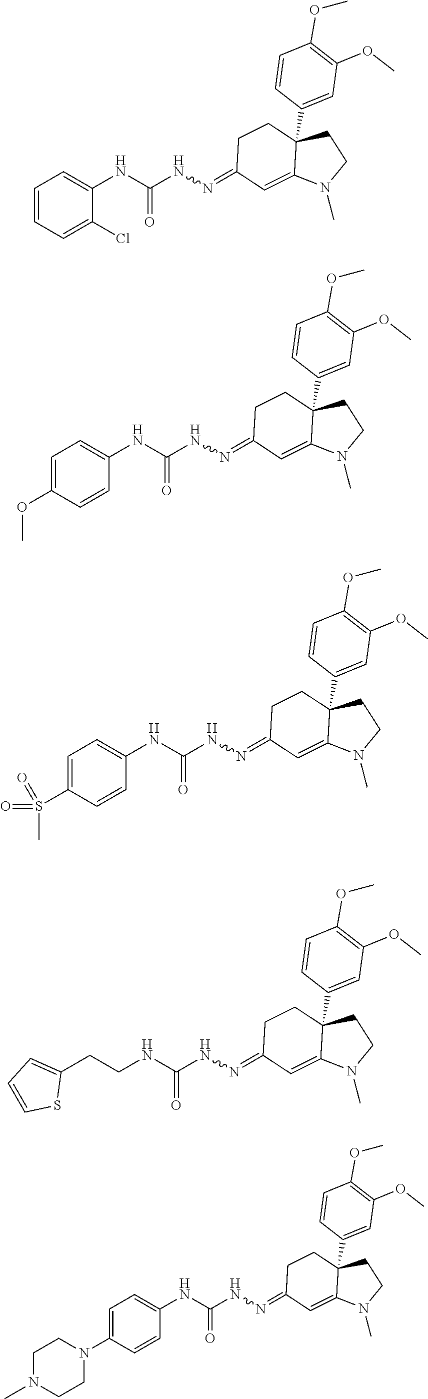

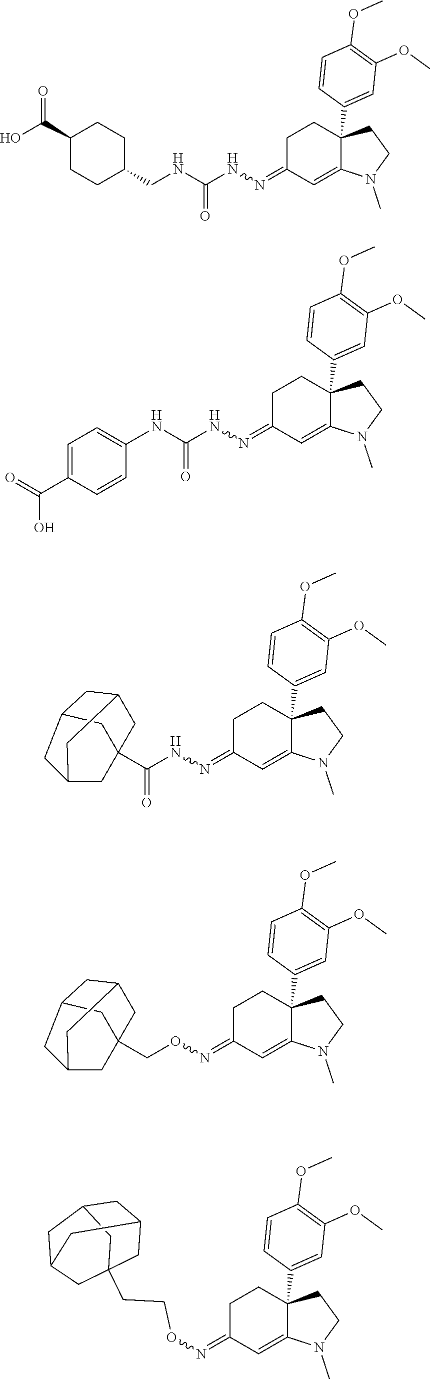

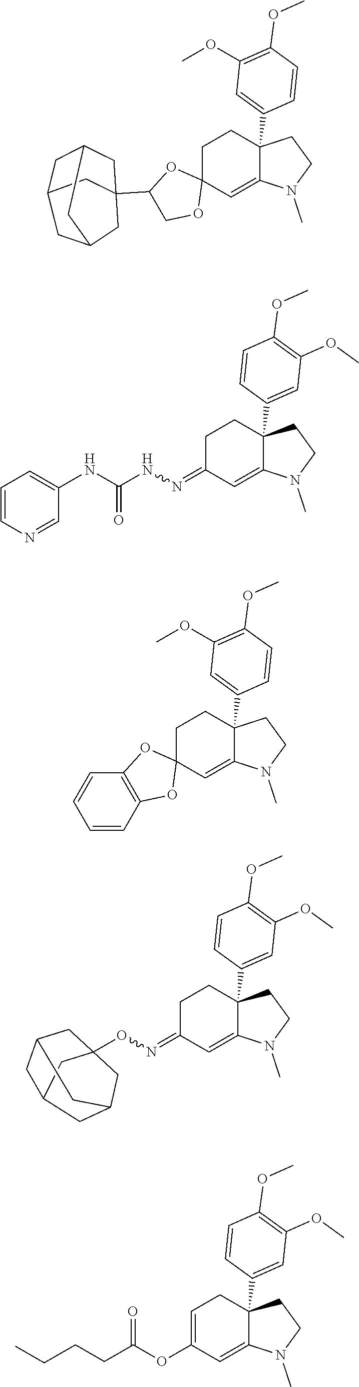

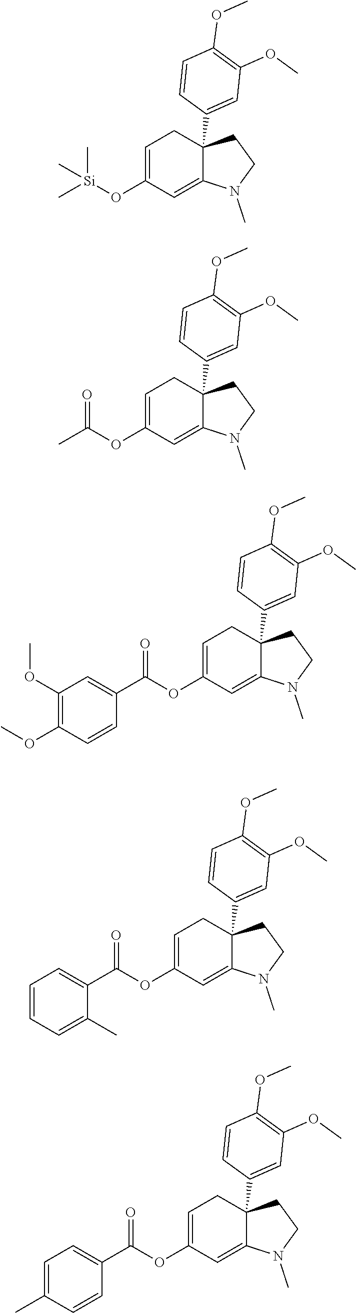

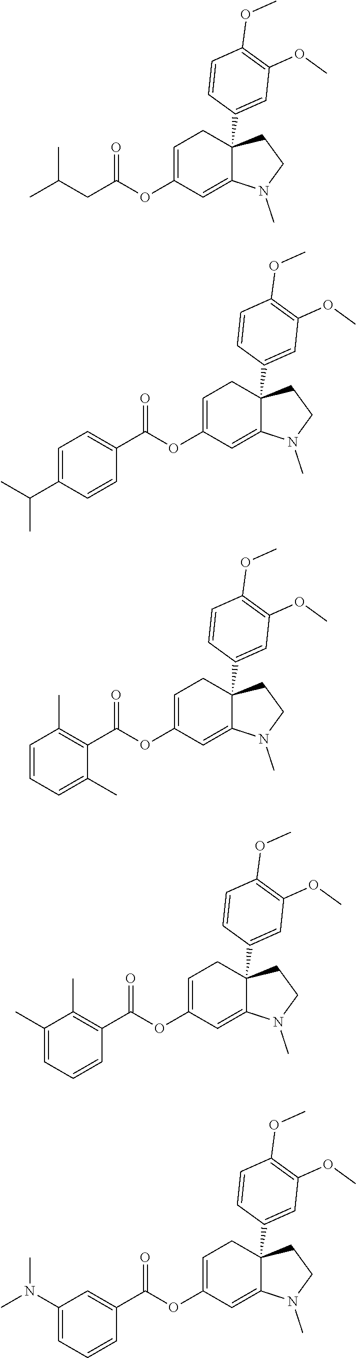

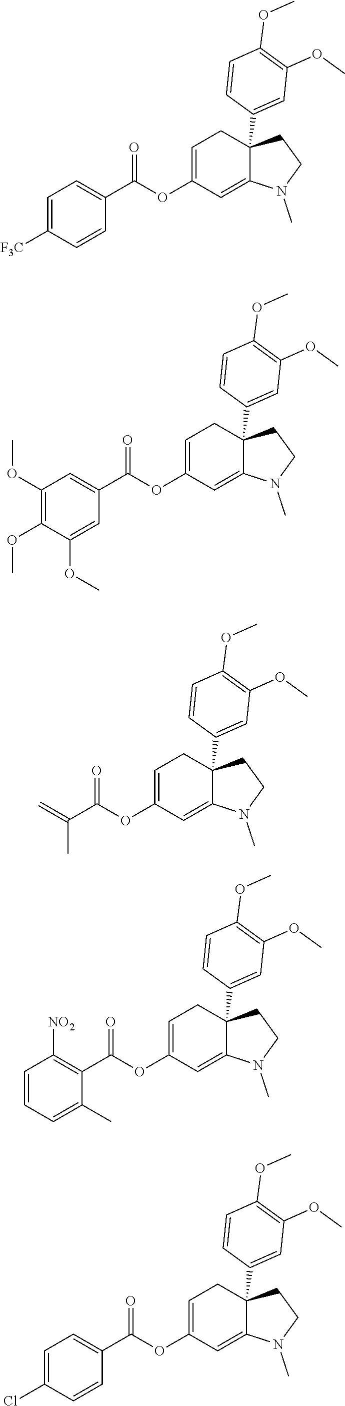

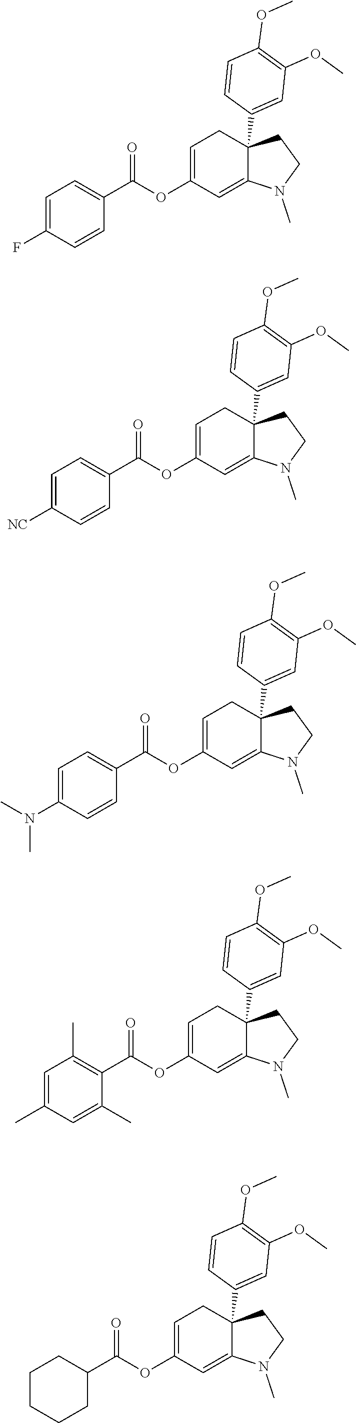

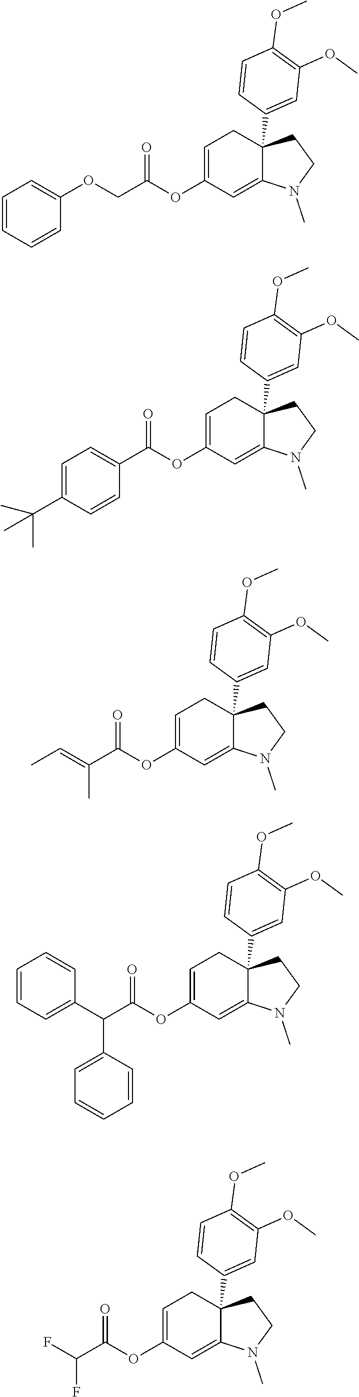

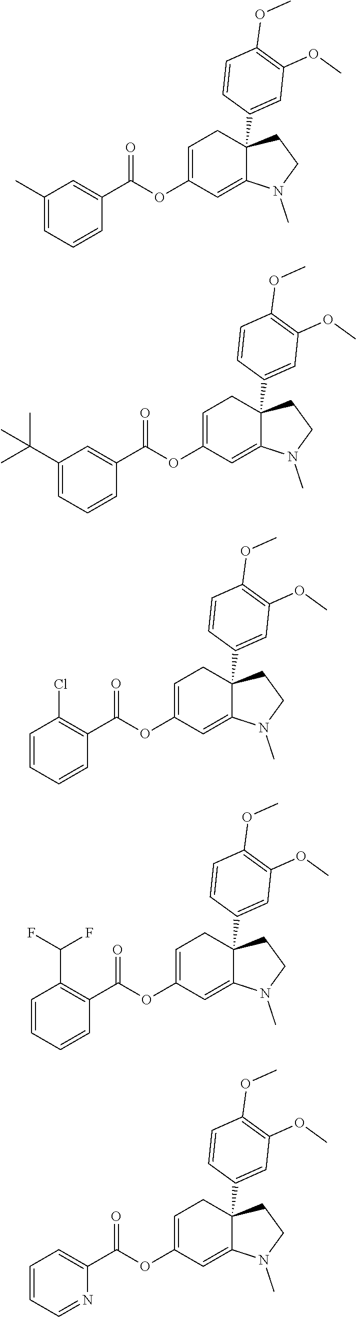

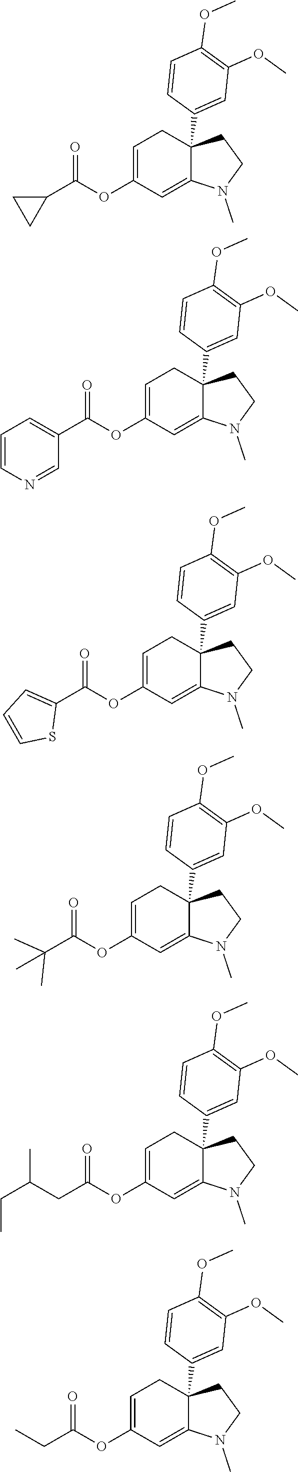

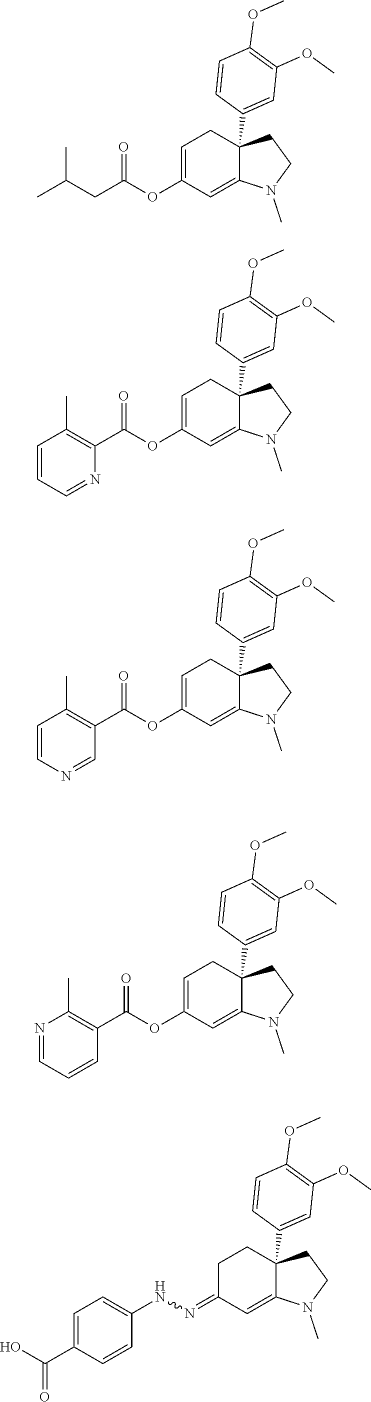

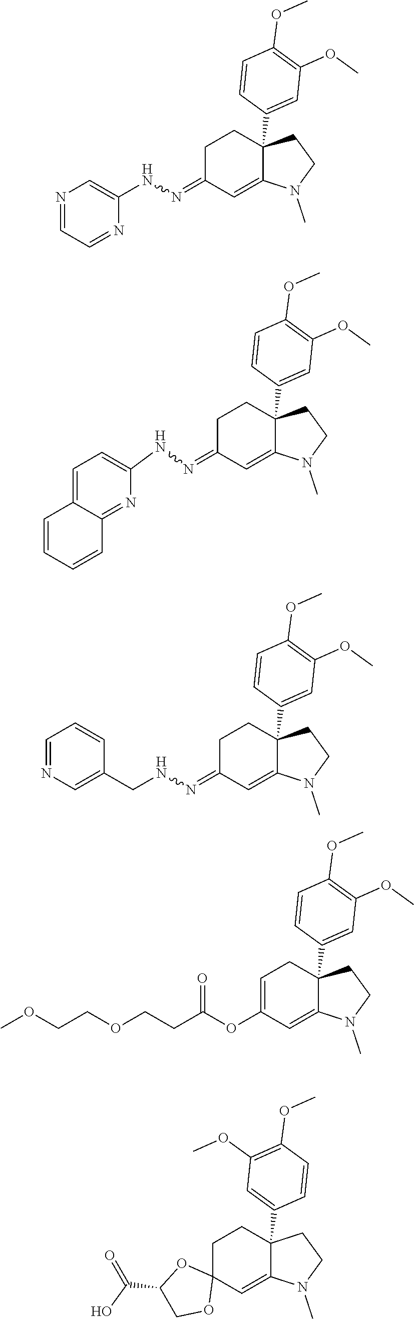

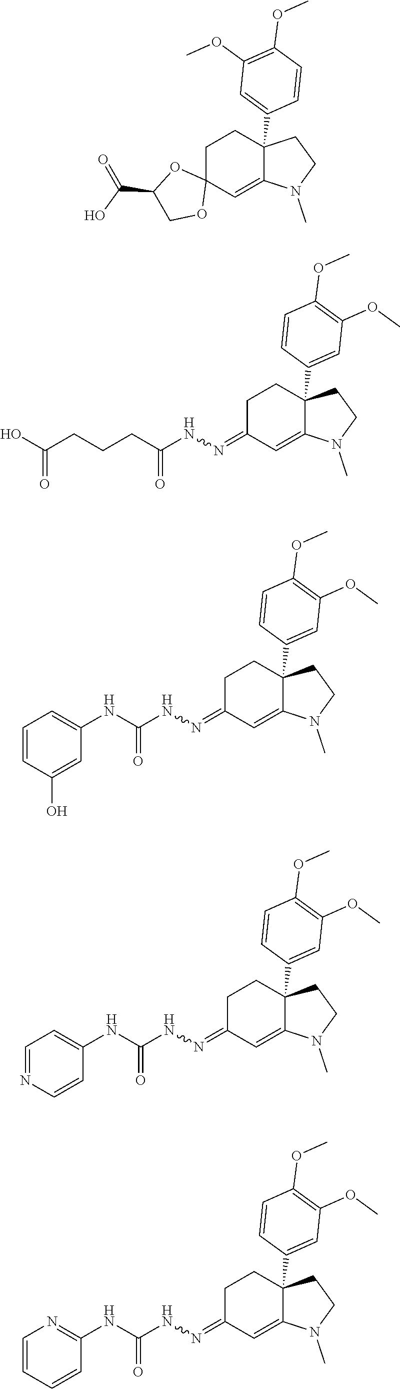 [SMILES: COc1ccc([C@]23CCN(C)C2=CC2(CC3)OC[C@@H](C(=O)O)O2)cc1OC ; COc1ccc([C@@]23CCC(=NNC(=O)CCCC(=O)O)C=C2N(C)CC3)cc1OC ; COc1ccc([C@@]23CCC(=NNC(=O)Nc4cccc(O)c4)C=C2N(C)CC3)cc1OC ; COc1ccc([C@@]23CCC(=NNC(=O)Nc4ccncc4)C=C2N(C)CC3)cc1OC ; COc1ccc([C@@]23CCC(=NNC(=O)Nc4ccccn4)C=C2N(C)CC3)cc1OC]

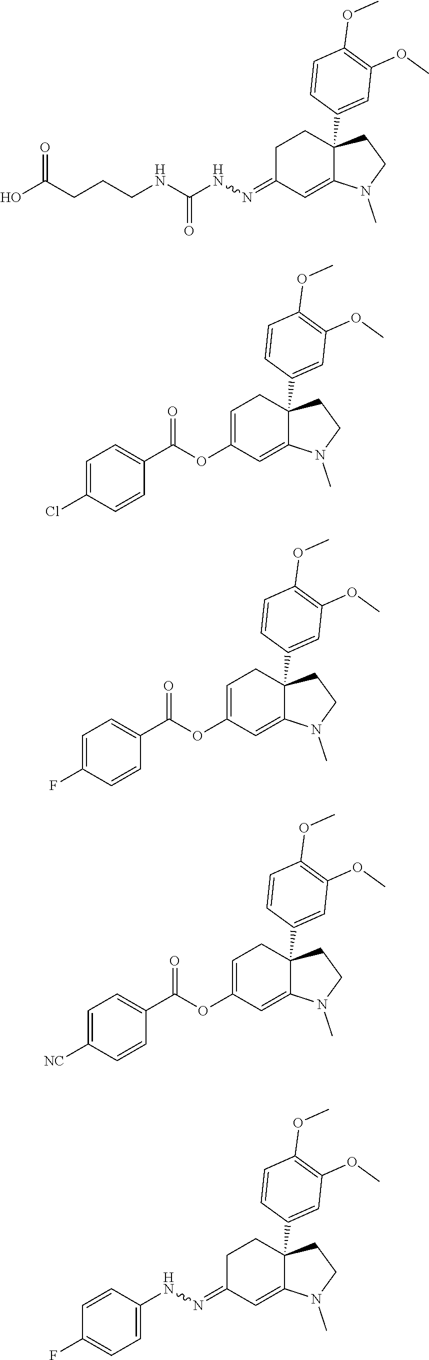 [SMILES: COc1ccc([C@@]23CCC(=NNC(=O)NCCCC(=O)O)C=C2N(C)CC3)cc1OC ; COc1ccc([C@@]23CC=C(OC(=O)c4ccc(Cl)cc4)C=C2N(C)CC3)cc1OC ; COc1ccc([C@@]23CC=C(OC(=O)c4ccc(F)cc4)C=C2N(C)CC3)cc1OC ; COc1ccc([C@@]23CC=C(OC(=O)c4ccc(C#N)cc4)C=C2N(C)CC3)cc1OC ; COc1ccc([C@@]23CCC(=NNc4ccc(F)cc4)C=C2N(C)CC3)cc1OC]

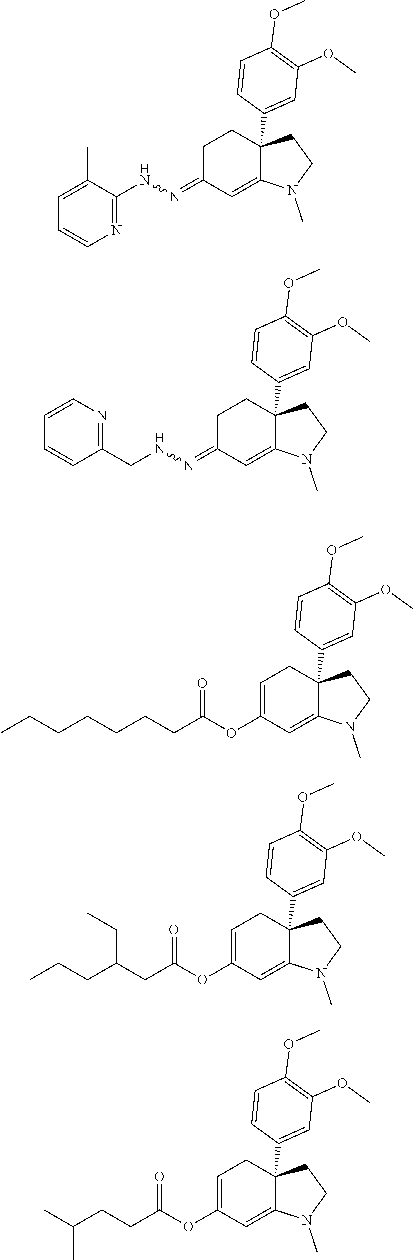

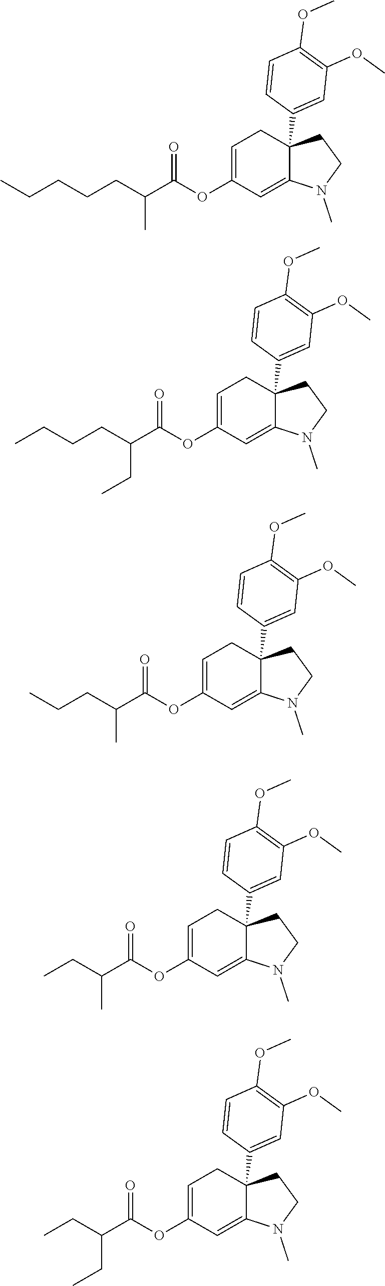

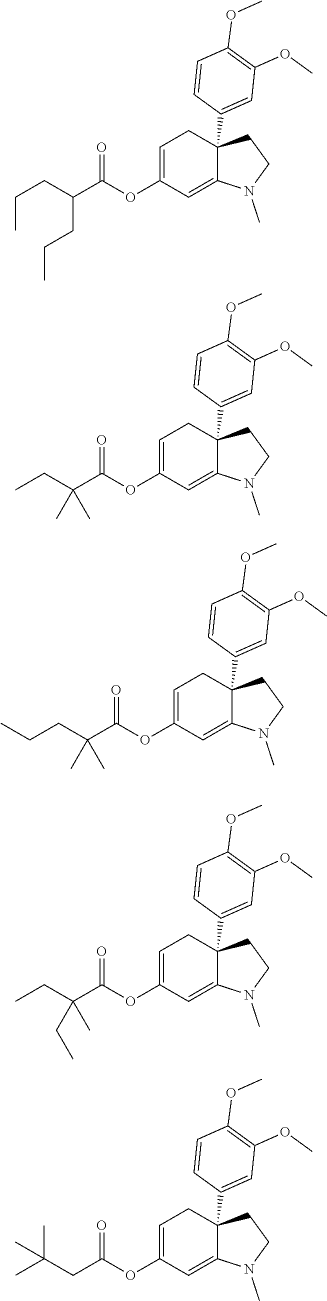

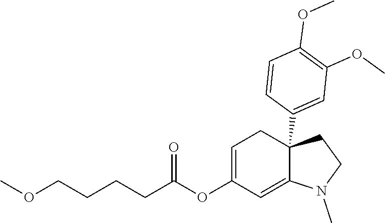

or a pharmaceutically acceptable salt thereof.

In some embodiments, the compound is selected from the group consisting of:

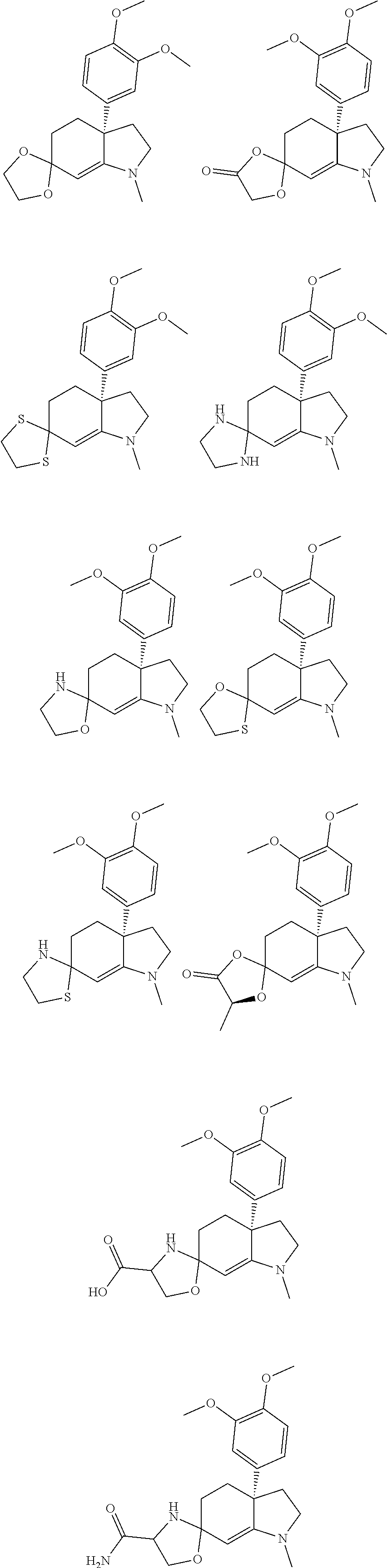

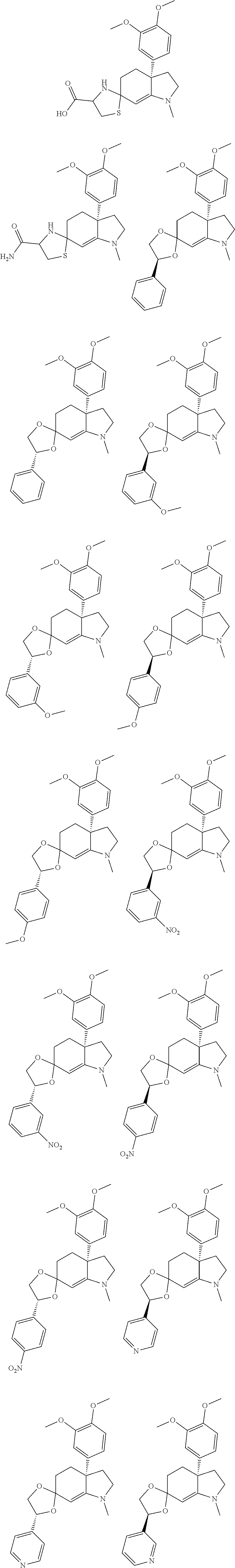

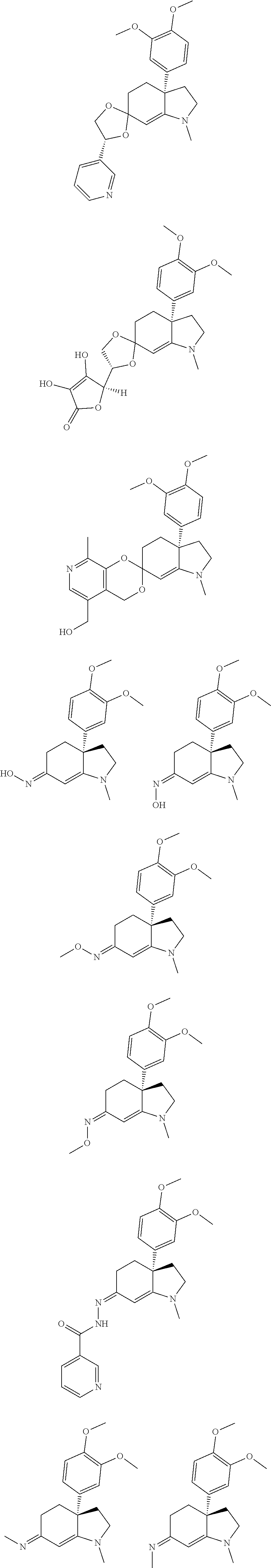

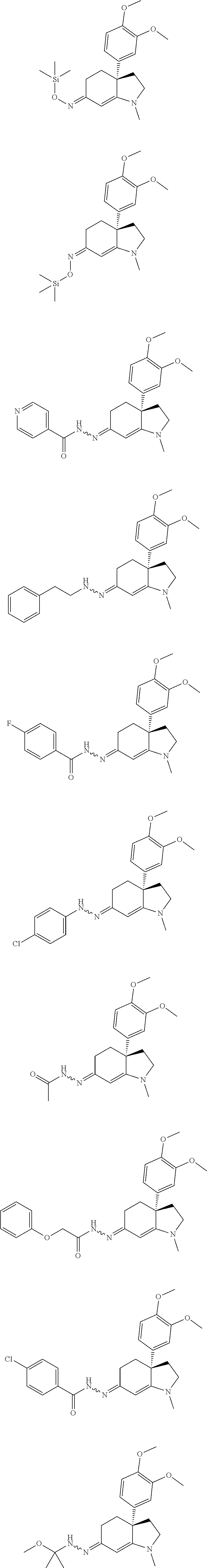

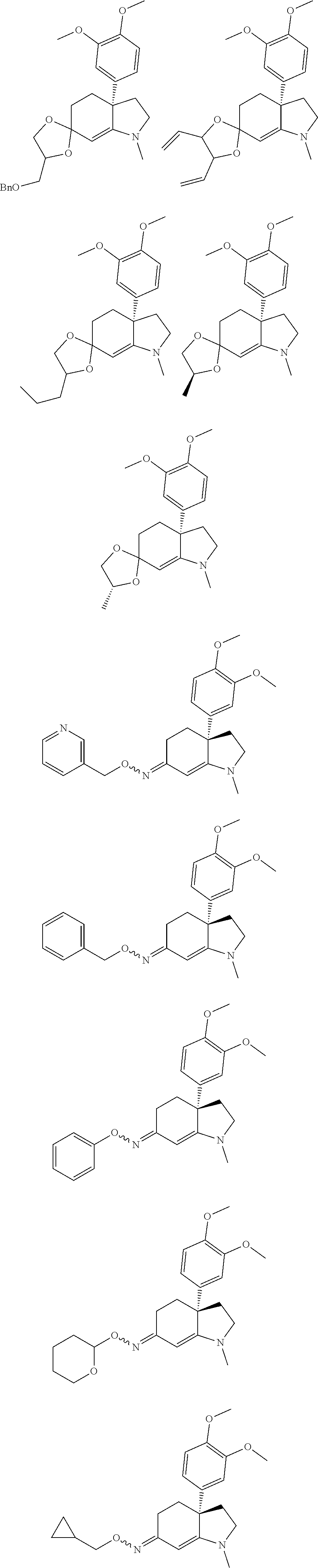

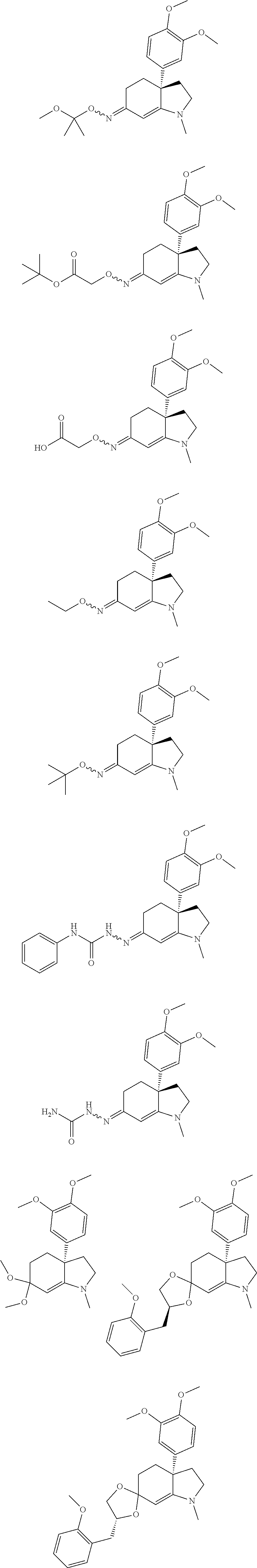

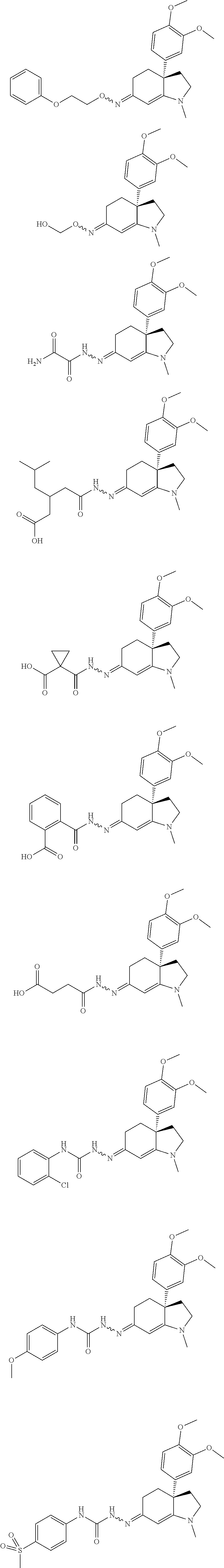

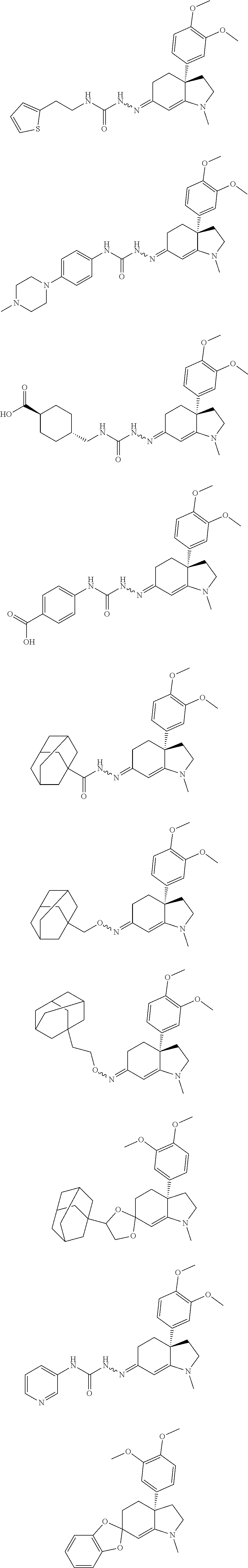

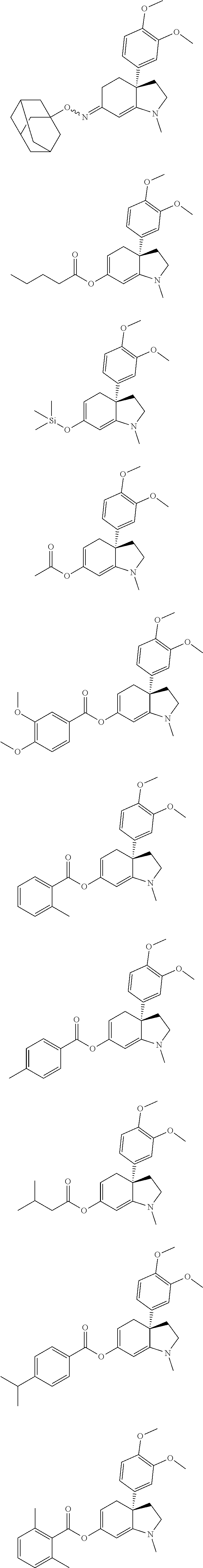

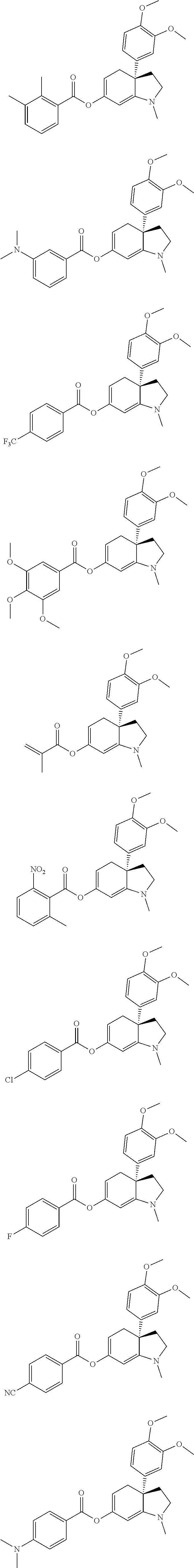

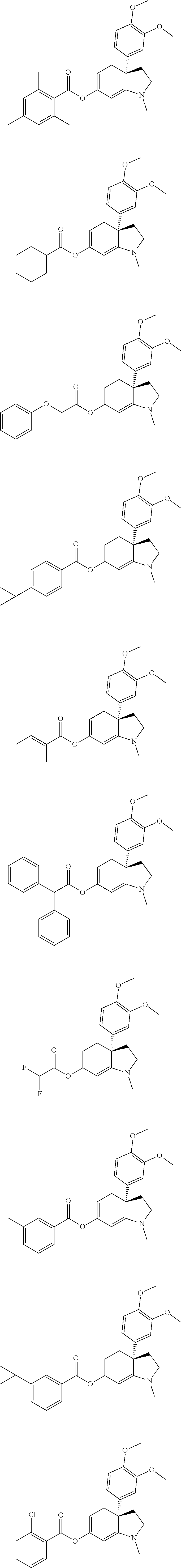

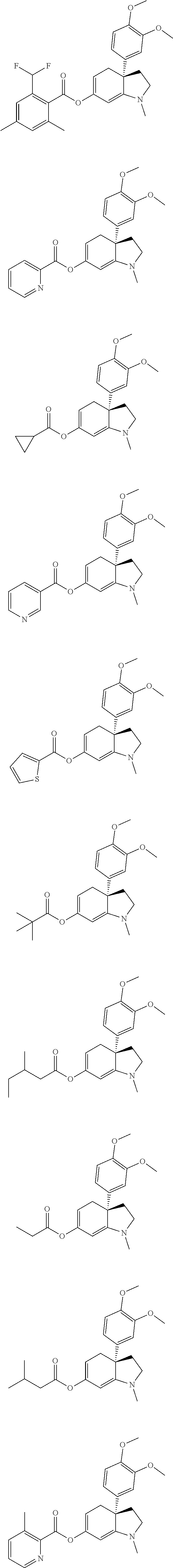

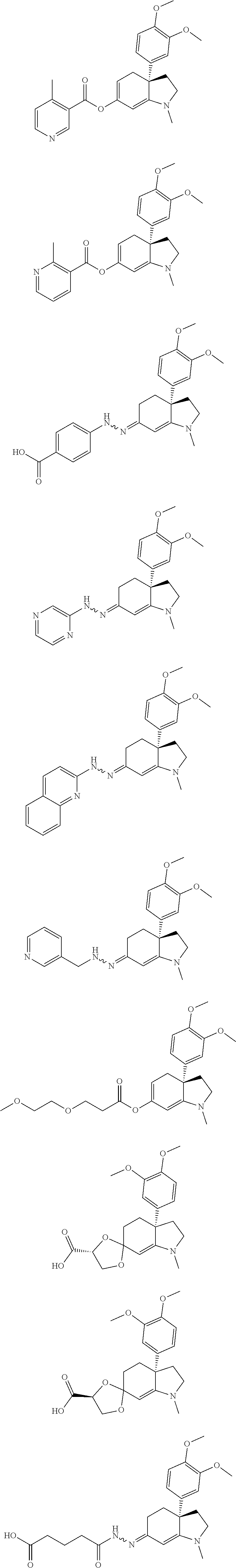

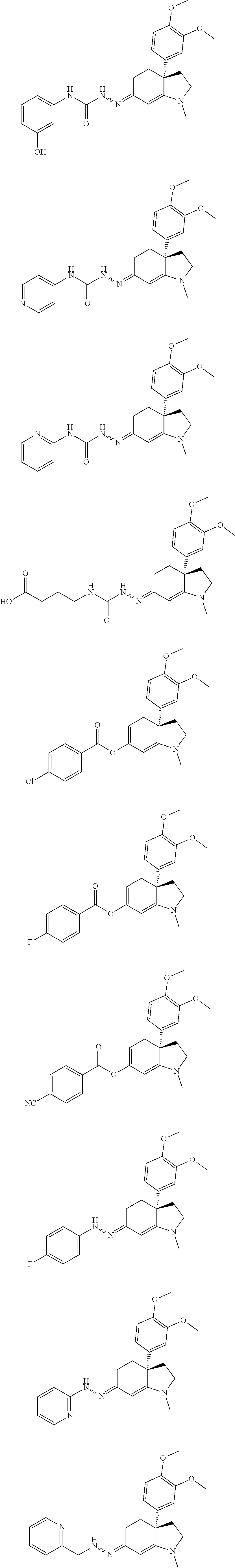

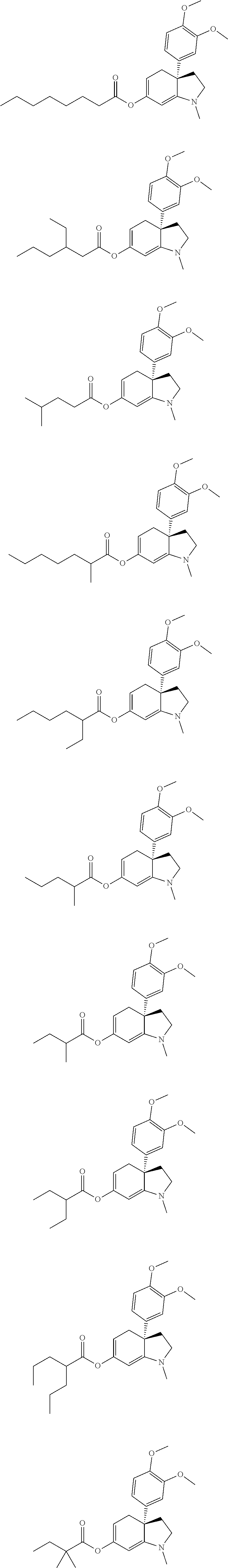

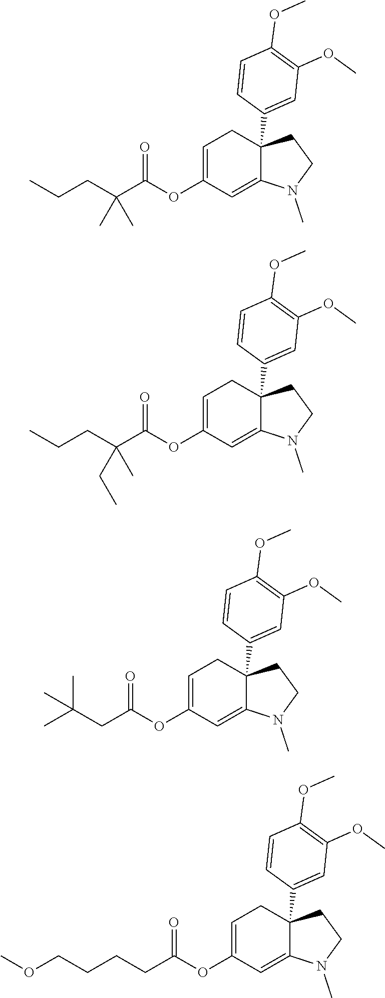

or a pharmaceutically acceptable salt thereof; wherein the compound has the absolute stereochemistry shown.

In some embodiments, a compound according to the present disclosure is of formula (I):

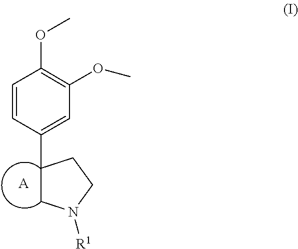

or a pharmaceutically acceptable salt thereof.

In some embodiments, ring A is

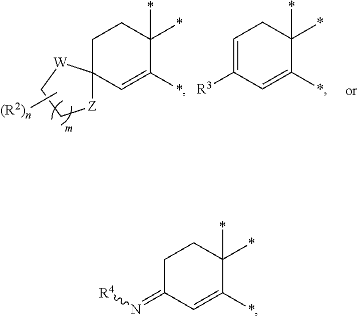

wherein * denotes the attachment points of ring A to the compound of formula (I).

In some embodiments, each of W and Z is independently O, NH, or S. In some embodiments, W is O. In some embodiments, W is NH. In some embodiments, W is S.

In some embodiments, each R² is independently C₁-C₃ alkyl, —COOH, —CONH₂, phenyl, 5- to 7-membered heterocyclyl, or 5- to 7-membered heteroaryl. In some embodiments, each hydrogen atom in phenyl, 5- to 7-membered heterocyclyl and 5- to 7-membered heteroaryl is optionally substituted by —OH, C₁-C₃ alkyl, C₁-C₃ alkoxy, C₁-C₃ alkanol, or —NO₂. In some embodiments, R² is —COOH, —CONH₂, phenyl, 5- to 7-membered heterocyclyl, or 5- to 7-membered heteroaryl, wherein each hydrogen atom in phenyl, 5- to 7-membered heterocyclyl and 5- to 7-membered heteroaryl is optionally substituted by —OH, C₁-C₃ alkyl, C₁-C₃ alkoxy, C₁-C₃ alkanol, or —NO₂. In some embodiments, R² is phenyl, wherein each hydrogen atom in phenyl is optionally substituted by —OH, C₁-C₃ alkyl, C₁-C₃ alkoxy, or —NO₂. In some embodiments, R² is 5- to 7-membered heterocyclyl or 5- to 7-membered heteroaryl, wherein each hydrogen atom in 5- to 7-membered heterocyclyl and 5- to 7-membered heteroaryl is optionally substituted by —OH, C₁-C₃ alkyl, C₁-C₃ alkoxy, C₁-C₃ alkanol, or —NO₂. In some embodiments, R² is pyridyl. In some embodiments, R² is

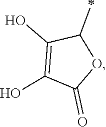 [SMILES: *C1OC(=O)C(O)=C1O]

wherein * denotes the point of attachment of R² the compound. In some embodiments, R² is —COOH or —CONH₂.

In some embodiments, two R²s on a single carbon atom combine to form ═O.

In some embodiments, two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol. In some embodiments, the two R²s together with the carbon atoms to which they are attached combine to form pyridyl, wherein each hydrogen atom in pyridyl is optionally substituted by —OH, C₁-C₃ alkyl, or C₁-C₃ alkanol.

In some embodiments, m is 1 or 2.

In some n is 0, 1, or 2. In some embodiments, n is 0. In some embodiments, n is 1. In some embodiments, n is 2.

In some embodiments, R³ is —OSi(C₁-C₆ alkyl)₃ or —OC(O)C₁-C₆ alkyl. In some embodiments, R³ is —OSi(C₁-C₆ alkyl)₃. In some embodiments, R³ is —OC(O)C₁-C₆ alkyl (e.g., —OC(O)CH₃).

In some embodiments, R⁴ is OH, C₁-C₆ alkoxy, or —NHC(O)-5- to 7-membered heteroaryl. In some embodiments, each hydrogen atom in 5- to 7-membered heteroaryl is optionally substituted by —OH, C₁-C₃ alkyl, or C₁-C₃ alkanol.

In some embodiments, R¹ is H or C₁-C₇ alkyl. In some embodiments, R¹ is C₁-C₃ alkyl. In some embodiments, R¹ is methyl.

Table 2 provides general synthetic methods to obtain of Formula (I) according to the reaction: Compound M+Compound R→Compound C.

TABLE 2

| Reactant M | Reactant R | Product Compound C |
|---|---|---|
| 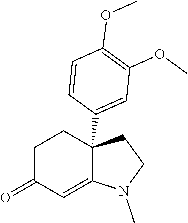 [SMILES: COc1ccc([C@@]23CCC(=O)C=C2N(C)CC3)cc1OC] | 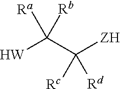 | 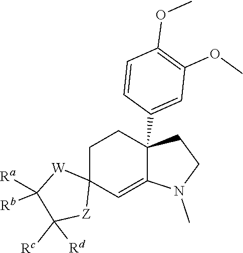 |
| 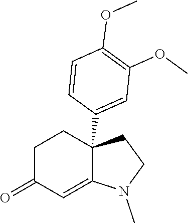 [SMILES: COc1ccc([C@@]23CCC(=O)C=C2N(C)CC3)cc1OC] | 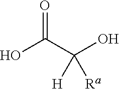 | 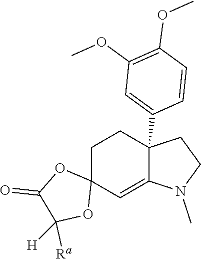 |
| 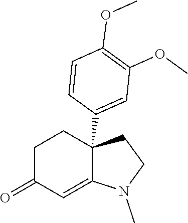 [SMILES: COc1ccc([C@@]23CCC(=O)C=C2N(C)CC3)cc1OC] | 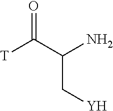 | 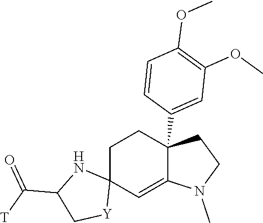 |
| 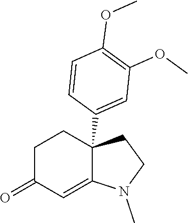 [SMILES: COc1ccc([C@@]23CCC(=O)C=C2N(C)CC3)cc1OC] | 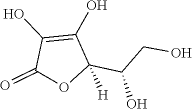 [SMILES: O=C1O[C@H]([C@@H](O)CO)C(O)=C1O] | 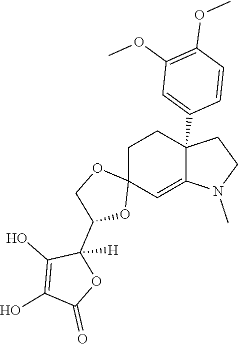 |
| 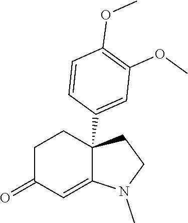 [SMILES: COc1ccc([C@@]23CCC(=O)C=C2N(C)CC3)cc1OC] | 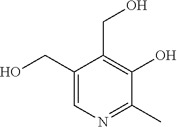 | 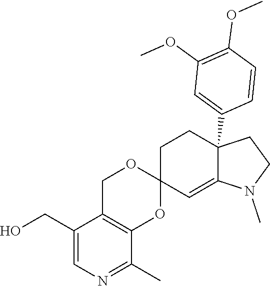 [SMILES: COc1ccc([C@]23CCN(C)C2=CC2(CC3)OCc3c(CO)cnc(C)c3O2)cc1OC] |
| 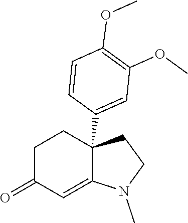 [SMILES: COc1ccc([C@@]23CCC(=O)C=C2N(C)CC3)cc1OC] | (R⁸)₂O or R⁸—X | 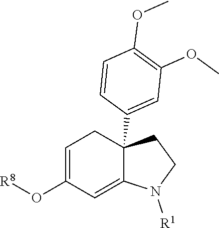 |
| 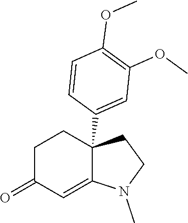 [SMILES: COc1ccc([C@@]23CCC(=O)C=C2N(C)CC3)cc1OC] | 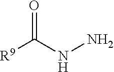 | 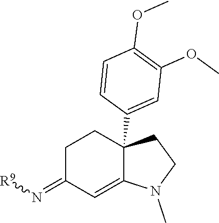 |

Unless otherwise indicated in the tables of compounds herein, the abbreviation RAC or rac indicates a racemic mixture, and DIAST indicates a specific diastereomer. In illustrative embodiments, although a compound may be depicted with [symbol] or [symbol] bonds, such a depiction may be denoting relative stereochemistry based on elution peaks from a chiral separation.

Compounds of Formula (I) including compounds of Formula (IIa) could be prepared by treatment of a ketone, such as Δ7-mesembrenone, with an appropriate glycol derivative, and an acid catalyst, such as p-toluenesulfonic acid or methanesulfonic acid, in a solvent, such as toluene, at an elevated temperature.

TABLE 3
Exemplary Compounds of Formula IIa

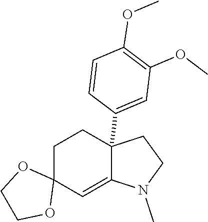 [SMILES: COc1ccc([C@]23CCN(C)C2=CC2(CC3)OCCO2)cc1OC]

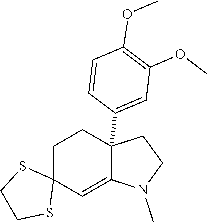 [SMILES: COc1ccc([C@]23CCN(C)C2=CC2(CC3)SCCS2)cc1OC]

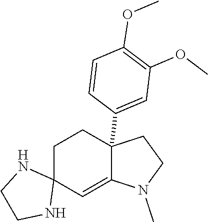 [SMILES: COc1ccc([C@]23CCN(C)C2=CC2(CC3)NCCN2)cc1OC]

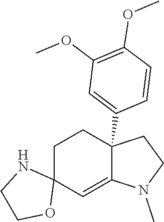 [SMILES: COc1ccc([C@]23CCN(C)C2=CC2(CC3)NCCO2)cc1OC]

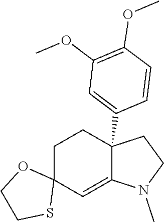 [SMILES: COc1ccc([C@]23CCN(C)C2=CC2(CC3)OCCS2)cc1OC]

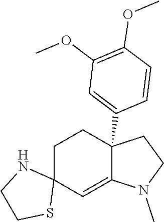 [SMILES: COc1ccc([C@]23CCN(C)C2=CC2(CC3)NCCS2)cc1OC]

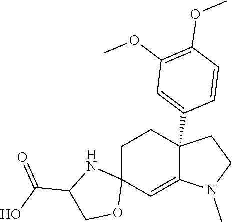 [SMILES: COc1ccc([C@]23CCN(C)C2=CC2(CC3)NC(C(=O)O)CO2)cc1OC]

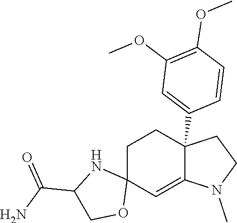 [SMILES: COc1ccc([C@]23CCN(C)C2=CC2(CC3)NC(C(N)=O)CO2)cc1OC]

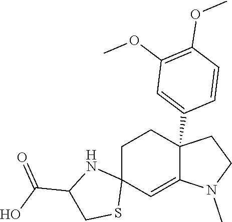 [SMILES: COc1ccc([C@]23CCN(C)C2=CC2(CC3)NC(C(=O)O)CS2)cc1OC]

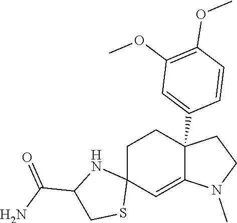 [SMILES: COc1ccc([C@]23CCN(C)C2=CC2(CC3)NC(C(N)=O)CS2)cc1OC]

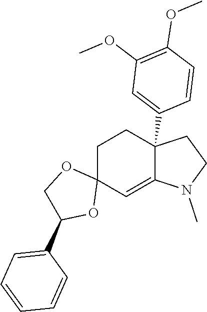 [SMILES: COc1ccc([C@]23CCN(C)C2=CC2(CC3)OC[C@H](c3ccccc3)O2)cc1OC]

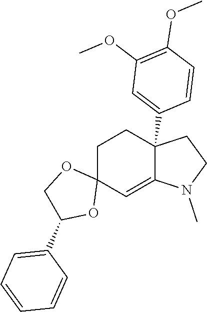 [SMILES: COc1ccc([C@]23CCN(C)C2=CC2(CC3)OC[C@@H](c3ccccc3)O2)cc1OC]

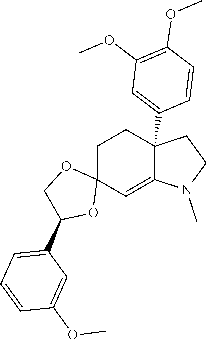 [SMILES: COc1cccc([C@H]2COC3(C=C4N(C)CC[C@]4(c4ccc(OC)c(OC)c4)CC3)O2)c1]

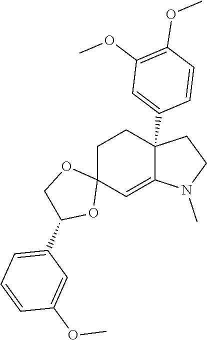 [SMILES: COc1cccc([C@@H]2COC3(C=C4N(C)CC[C@]4(c4ccc(OC)c(OC)c4)CC3)O2)c1]

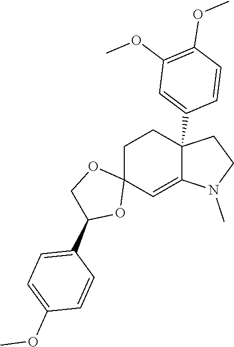 [SMILES: COc1ccc([C@H]2COC3(C=C4N(C)CC[C@]4(c4ccc(OC)c(OC)c4)CC3)O2)cc1]

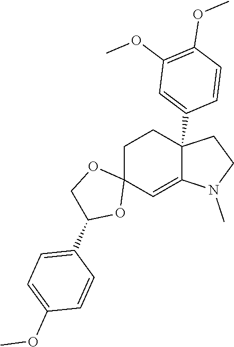 [SMILES: COc1ccc([C@@H]2COC3(C=C4N(C)CC[C@]4(c4ccc(OC)c(OC)c4)CC3)O2)cc1]

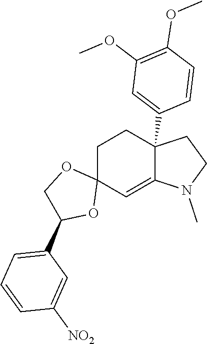 [SMILES: COc1ccc([C@]23CCN(C)C2=CC2(CC3)OC[C@H](c3cccc([N+](=O)[O-])c3)O2)cc1OC]

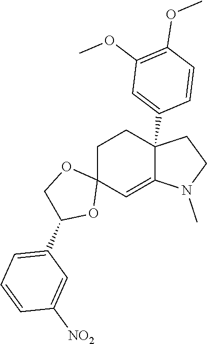 [SMILES: COc1ccc([C@]23CCN(C)C2=CC2(CC3)OC[C@@H](c3cccc([N+](=O)[O-])c3)O2)cc1OC]

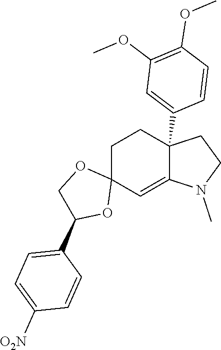 [SMILES: COc1ccc([C@]23CCN(C)C2=CC2(CC3)OC[C@H](c3ccc([N+](=O)[O-])cc3)O2)cc1OC]

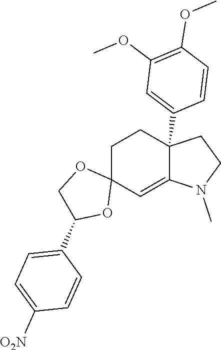 [SMILES: COc1ccc([C@]23CCN(C)C2=CC2(CC3)OC[C@@H](c3ccc([N+](=O)[O-])cc3)O2)cc1OC]

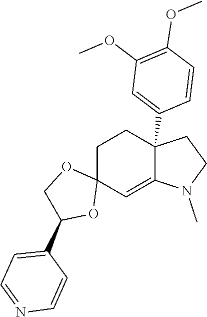 [SMILES: COc1ccc([C@]23CCN(C)C2=CC2(CC3)OC[C@H](c3ccncc3)O2)cc1OC]

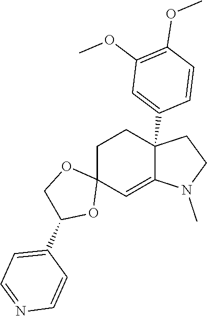 [SMILES: COc1ccc([C@]23CCN(C)C2=CC2(CC3)OC[C@@H](c3ccncc3)O2)cc1OC]

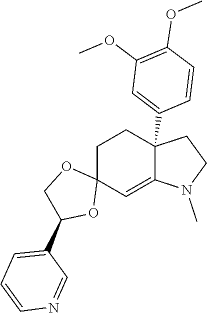 [SMILES: COc1ccc([C@]23CCN(C)C2=CC2(CC3)OC[C@H](c3cccnc3)O2)cc1OC]

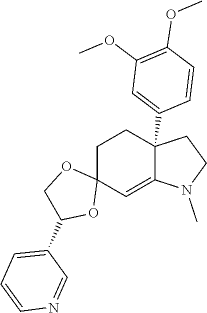

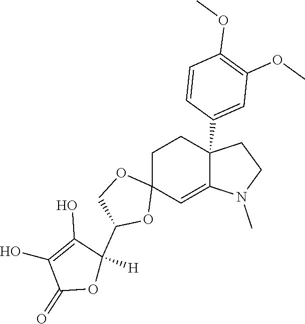

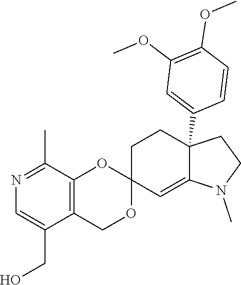 [SMILES: COc1ccc([C@]23CCN(C)C2=CC2(CC3)OCc3c(CO)cnc(C)c3O2)cc1OC]

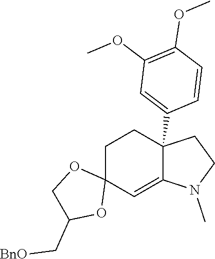 [SMILES: COc1ccc([C@]23CCN(C)C2=CC2(CC3)OCC(COCc3ccccc3)O2)cc1OC]

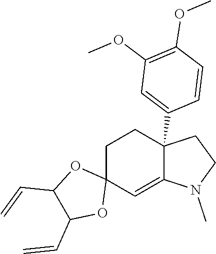 [SMILES: C=CC1OC2(C=C3N(C)CC[C@]3(c3ccc(OC)c(OC)c3)CC2)OC1C=C]

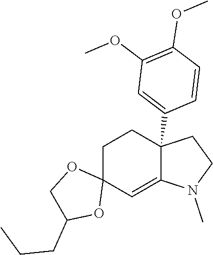 [SMILES: CCCC1COC2(C=C3N(C)CC[C@]3(c3ccc(OC)c(OC)c3)CC2)O1]

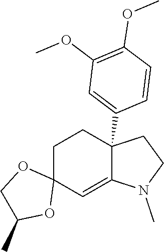 [SMILES: COc1ccc([C@]23CCN(C)C2=CC2(CC3)OC[C@H](C)O2)cc1OC]

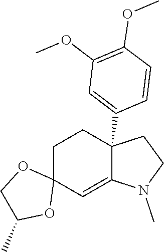 [SMILES: COc1ccc([C@]23CCN(C)C2=CC2(CC3)OC[C@@H](C)O2)cc1OC]

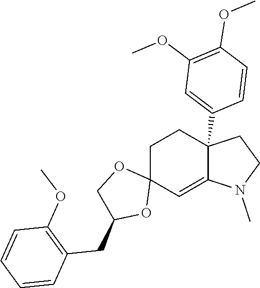 [SMILES: COc1ccccc1C[C@H]1COC2(C=C3N(C)CC[C@]3(c3ccc(OC)c(OC)c3)CC2)O1]

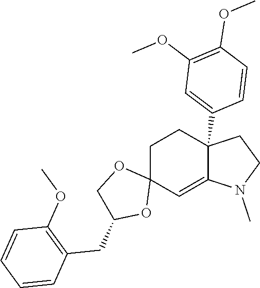 [SMILES: COc1ccccc1C[C@@H]1COC2(C=C3N(C)CC[C@]3(c3ccc(OC)c(OC)c3)CC2)O1]

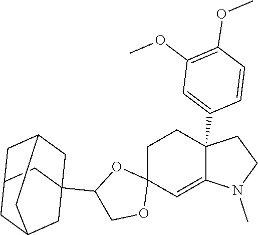 [SMILES: COc1ccc([C@]23CCN(C)C2=CC2(CC3)OCC(C34CC5CC(CC(C5)C3)C4)O2)cc1OC]

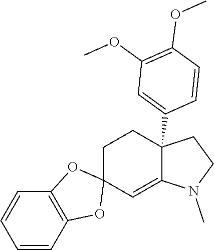 [SMILES: COc1ccc([C@]23CCN(C)C2=CC2(CC3)Oc3ccccc3O2)cc1OC]

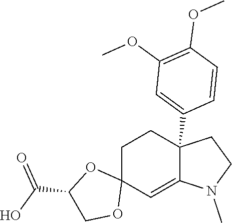 [SMILES: COc1ccc([C@]23CCN(C)C2=CC2(CC3)OC[C@H](C(=O)O)O2)cc1OC]

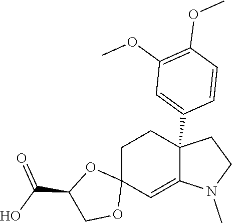 [SMILES: COc1ccc([C@]23CCN(C)C2=CC2(CC3)OC[C@@H](C(=O)O)O2)cc1OC]

In some embodiments, the compound of formula (I) is a compound of formula (IX):

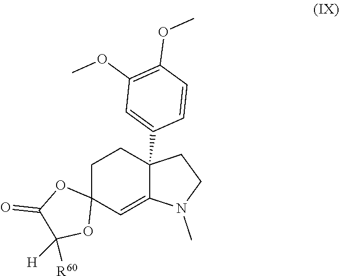

or a pharmaceutically acceptable salt thereof, wherein R₆₀ is hydrogen or methyl.

Compounds of Formula (IX) could be prepared by treatment of a ketone, such as Δ7-mesembrenone, with an appropriate alpha-hydroxy carboxylic acid, with a Lewis acid, such as boron trifluoride etherate, in a solvent, such as dichloromethane. For example, a compound of formula (IX) where R₆₀ is methyl could be obtained by reacting Δ7-mesembrenone with lactic acid (e.g., with TsOH, toluene). In some embodiments, the compound can be a lactate or glycolate derivative of Δ7-mesembrenone. R₆₀ in Scheme 2 is as defined with respect to Formula (IX) above.

Table 4 provides non-limiting examples of certain compounds of Formula (IX).

TABLE 4
Exemplary Compounds of Formula IX

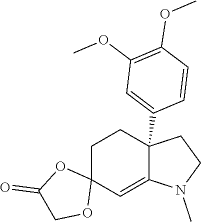 [SMILES: COc1ccc([C@]23CCN(C)C2=CC2(CC3)OCC(=O)O2)cc1OC]

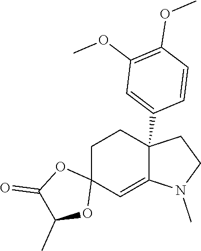

In some embodiments, the compound of Formula (I) is a compound of Formula (X) or a pharmaceutically acceptable salt thereof:

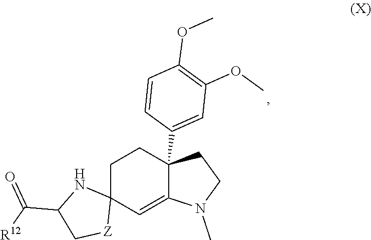

wherein Z is S or O; and R₁₂ is hydroxyl or amino.

Compounds of Formula (X) could be prepared by treatment of a ketone, such as Δ7-mesembrenone, with an appropriate alpha-amino carboxylic acid or alpha-amino amide, in a solvent, such as ethanol, at an elevated temperature.

Table 5 provides non-limiting examples of certain compounds of Formula (X).

TABLE 5
Exemplary Compounds of Formula X

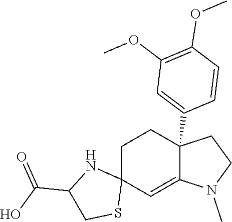 [SMILES: COc1ccc([C@]23CCN(C)C2=CC2(CC3)NC(C(=O)O)CS2)cc1OC]

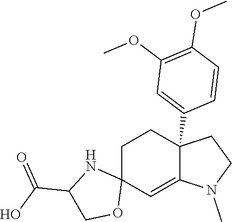 [SMILES: COc1ccc([C@]23CCN(C)C2=CC2(CC3)NC(C(=O)O)CO2)cc1OC]

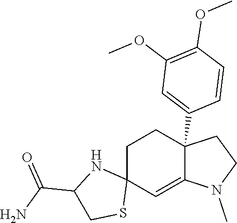 [SMILES: COc1ccc([C@]23CCN(C)C2=CC2(CC3)NC(C(N)=O)CS2)cc1OC]

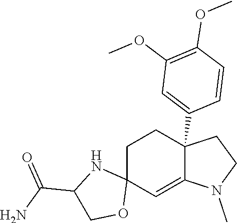 [SMILES: COc1ccc([C@]23CCN(C)C2=CC2(CC3)NC(C(N)=O)CO2)cc1OC]

In some embodiments, the compound of Formula (I) is a compound of Formula (XI) or a pharmaceutically acceptable salt thereof:

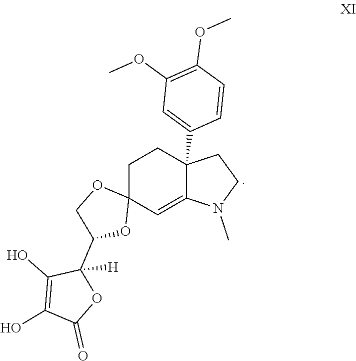

Compounds of Formula (XI) could be prepared by treatment of a ketone such as Δ7-mesembrenone, with a naturally occurring diol, such as ascorbic acid, with an acid catalyst in a solvent, such as toluene. In some embodiments, Δ7-mesembrenone could be reacted with ascorbic acid (e.g., TsOH, acetone) to obtain a compound of Formula (XI).

Table provides non-limiting examples of certain compounds of Formula (XI).

TABLE 6
Exemplary Compound of Formula XI

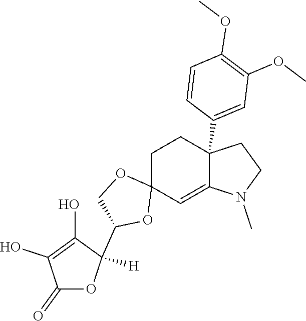

In some embodiments, the compound of Formula (I) is a compound of Formula (XII) or a pharmaceutically acceptable salt thereof:

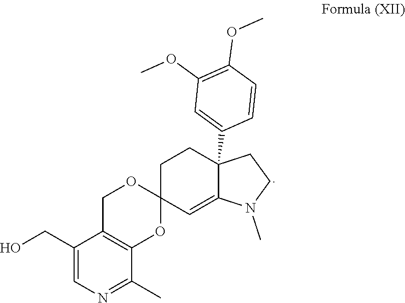 [SMILES: COc1ccc([C@]23CCN(C)C2=CC2(CC3)OCc3c(CO)cnc(C)c3O2)cc1OC]

Compounds of Formula (XII) could be prepared by treatment of a ketone such as Δ7-mesembrenone, with a compound such as pyridoxine or an analog thereof (e.g., a vitamin B₆ analog), with an acid catalyst in a solvent.

Table 7 provides non-limiting examples of certain compounds of Formula (XII).

TABLE 7
Exemplary Compound of Formula XII

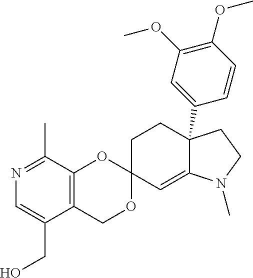 [SMILES: COc1ccc([C@]23CCN(C)C2=CC2(CC3)OCc3c(CO)cnc(C)c3O2)cc1OC]

In some embodiments, a compound of Formula (I) can be a compound of Formula (XIII) or a pharmaceutically acceptable salt thereof:

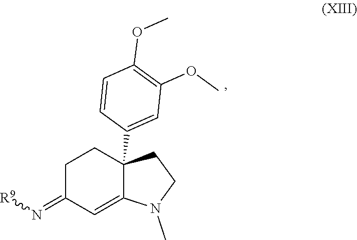

wherein R₉ is hydroxyl, methoxy, or —NH—C(O)-6-member nitrogen-containing heteroaryl. In some embodiments, the 6-member nitrogen-containing heteroaryl is pyridine.

Table 8 provides non-limiting examples of certain compounds of Formula (XIII).

TABLE 8
Exemplary Compounds of Formula (XIII)

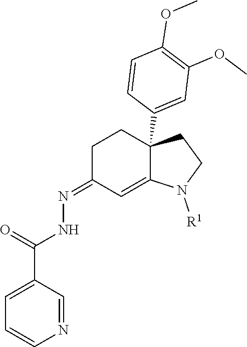

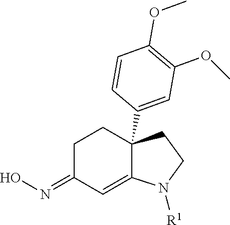

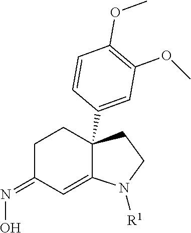

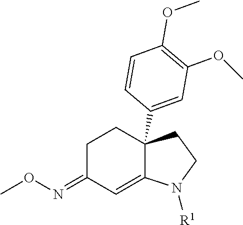

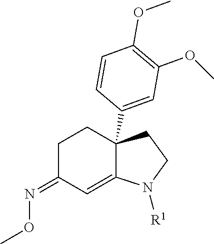

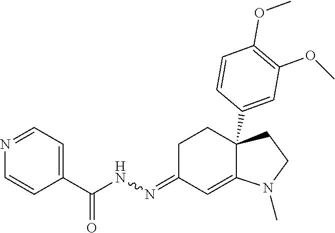 [SMILES: COc1ccc([C@@]23CCC(=NNC(=O)c4ccncc4)C=C2N(C)CC3)cc1OC]

In certain embodiments, the invention relates to a compound of Formula (XIV)

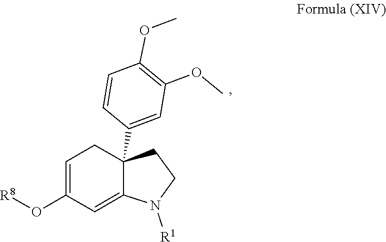

or a pharmaceutically acceptable salt thereof, as defined above.

In some embodiments, a compound of Formula (XIV) can be a compound of Formula (XIV-1) or a pharmaceutically acceptable salt thereof:

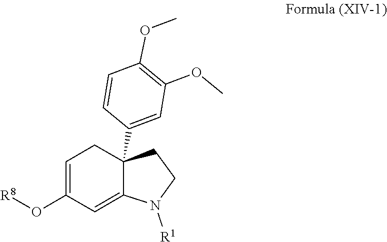

wherein R₈ is —Si(CH₃)₃ or —C(O)—CH₃.

Compounds of Formula (XIV) including compounds of Formula (XIV-1) could be prepared by treating a ketone, such as Δ7-mesembrenone, with an appropriate base, such as potassium tert-butoxide, and an acid anhydride, in a solvent, such as THF.

Table 10 provides non-limiting examples of certain compounds of Formula (XIV).

TABLE 10
Exemplary Compounds of Formula (XIV-1)

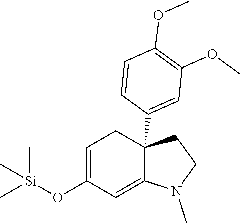

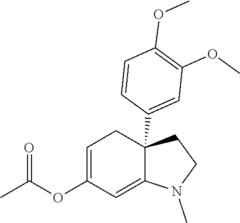 [SMILES: COc1ccc([C@@]23CC=C(OC(C)=O)C=C2N(C)CC3)cc1OC]

In some embodiments, the compound is a compound of Formula (XV) or a pharmaceutically acceptable salt thereof:

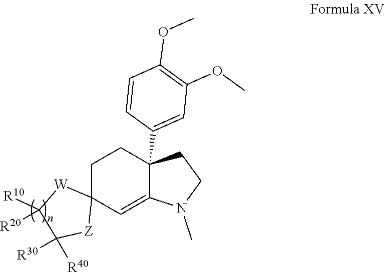

wherein
n is 0 or 1,
W and Z are each independently CH₂, O, S or NH, provided that at least one of W or Z is O, S or NH; and
R₁₀, R₂₀, R₃₀ and R₄₀ are each hydrogen.

In some embodiments, W and Z in Formula (XV) are each O. In some embodiments, W and Z in Formula (XV) are each S. In some embodiments, W and Z in Formula (XV) are each NH. In some embodiments, W is NH and Z is S in Formula (XV). In some embodiments, W is O and Z is S in Formula (XV). In some embodiments, W is CH₂ and Z is O in Formula (XV). In some embodiments, W is CH₂ and Z is NH in Formula (XV).

Compounds of Formula (XV) including compounds of Formula (XV-1) could be prepared by treatment of a ketone, such as Δ7-mesembrenone, with an appropriate glycol derivative, and an acid catalyst, such as p-toluenesulfonic acid or methanesulfonic acid, in a solvent, such as toluene, at an elevated temperature.

Table 9 provides non-limiting examples of certain compounds of Formula (XV).

TABLE 9
Exemplary Compounds of Formula XV

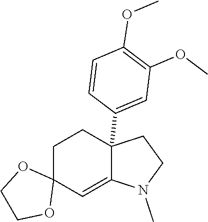 [SMILES: COc1ccc([C@]23CCN(C)C2=CC2(CC3)OCCO2)cc1OC]

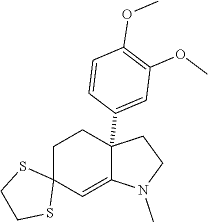 [SMILES: COc1ccc([C@]23CCN(C)C2=CC2(CC3)SCCS2)cc1OC]

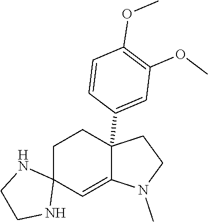 [SMILES: COc1ccc([C@]23CCN(C)C2=CC2(CC3)NCCN2)cc1OC]

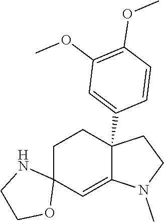 [SMILES: COc1ccc([C@]23CCN(C)C2=CC2(CC3)NCCO2)cc1OC]

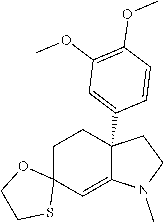 [SMILES: COc1ccc([C@]23CCN(C)C2=CC2(CC3)OCCS2)cc1OC]

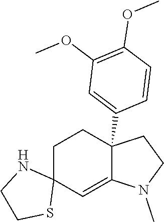 [SMILES: COc1ccc([C@]23CCN(C)C2=CC2(CC3)NCCS2)cc1OC]

In some embodiments, a compound according to the present disclosure is a compound of formula (I):

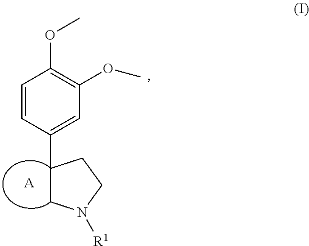

or a pharmaceutically acceptable salt thereof,
wherein
R¹ is C₁-C₇ alkyl; and
ring A is

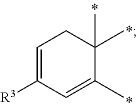

wherein * denotes the attachment points of ring A to the compound of formula (I), and wherein
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, phenoxy or —O(CH₂)ₚOCH₃; and
p is 2, 3, or 4.

In some embodiments, a compound according to the present disclosure is a compound of formula (I):

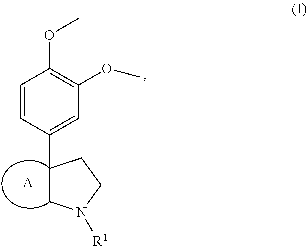

or a pharmaceutically acceptable salt thereof,
wherein
R¹ is C₁-C₇ alkyl; and
ring A is

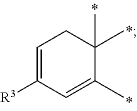

wherein * denotes the attachment points of ring A to the compound of formula (I), and wherein
R³ is —OC(O)C₁-C₆ alkyl, —OC(O)C₃-C₁₀ cycloalkyl, —OC(O)phenyl, wherein each hydrogen atom in C₁-C₆ alkyl, C₃-C₁₀ cycloalkyl, and phenyl, is optionally substituted by halogen, C₁-C₆ alkyl, C₁-C₃ alkoxy, nitro, —N(C₁-C₃ alkyl)₂, C₁-C₃ haloalkyl, cyano, phenyl, phenoxy or —O(CH₂)ₚOCH₃; and
p is 2, 3, or 4.

In some embodiments, a compound according to the present disclosure is a compound of formula (I):

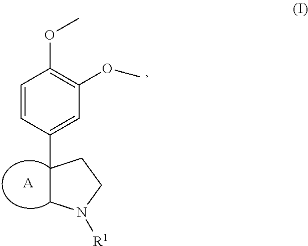

or a pharmaceutically acceptable salt thereof,
wherein
R¹ is methyl; and
ring A is

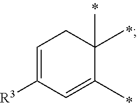

wherein * denotes the attachment points of ring A to the compound of formula (I), and wherein
R³ is —OC(O)C₁-C₆ alkyl, —OC(O)C₃-C₁₀ cycloalkyl, —OC(O)phenyl, wherein each hydrogen atom in C₁-C₆ alkyl, C₃-C₁₀ cycloalkyl, and phenyl, is optionally substituted by halogen, C₁-C₆ alkyl, C₁-C₃ alkoxy, C₁-C₃ haloalkyl, or —O(CH₂)ₚOCH₃; and
p is 2, 3, or 4.

In some embodiments, a compound according to the present disclosure is a compound of formula (I):

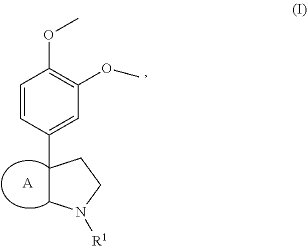

or a pharmaceutically acceptable salt thereof,
wherein
R¹ is methyl; and
ring A is

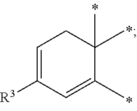

wherein * denotes the attachment points of ring A to the compound of formula (I), and wherein
R³ is —OC(O)C₁-C₆ alkyl.

In some embodiments, a compound according to the present disclosure is a compound of formula (I):

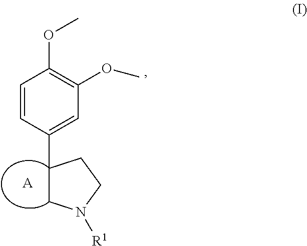

or a pharmaceutically acceptable salt thereof, wherein
R¹ is methyl; and
ring A is

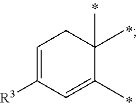

wherein * denotes the attachment points of ring A to the compound of formula (I), and wherein
R³ is —OC(O)phenyl, wherein each hydrogen atom in phenyl is optionally substituted by halogen, C₁-C₆ alkyl, C₁-C₃ alkoxy, nitro, —N(C₁-C₃ alkyl)₂, C₁-C₃ haloalkyl, cyano, phenyl, phenoxy or —O(CH₂)ₚOCH₃; and
p is 2, 3, or 4.

In some embodiments, a compound according to the present disclosure is a compound of formula (I):

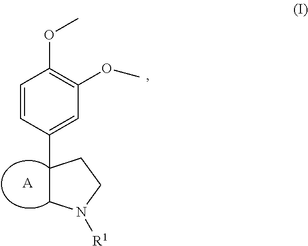

or a pharmaceutically acceptable salt thereof, wherein
R¹ is methyl; and
ring A is

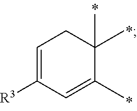

wherein * denotes the attachment points of ring A to the compound of formula (I), and wherein
R³ is —OC(O)phenyl, wherein each hydrogen atom in phenyl is optionally substituted by C₁-C₆ alkyl.

In some embodiments, a compound according to the present disclosure is a compound of formula (I):

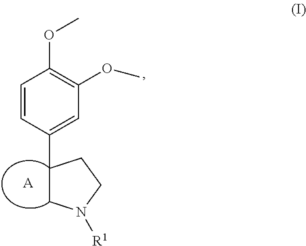

or a pharmaceutically acceptable salt thereof, wherein
R¹ is or C₁-C₇ alkyl; and
ring A is

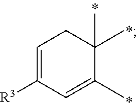

wherein * denotes the attachment points of ring A to the compound of formula (I), and wherein
R³ is —OC(O)phenyl, wherein each hydrogen atom in phenyl is optionally substituted by methyl.

In some embodiments, a compound according to the present disclosure is a compound of formula (I):

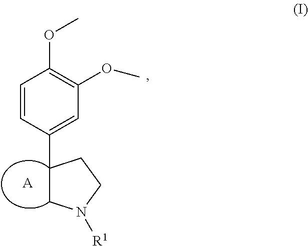

or a pharmaceutically acceptable salt thereof, wherein
R¹ is methyl; and
ring A is

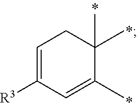

wherein * denotes the attachment points of ring A to the compound of formula (I), and wherein
R³ is —OC(O)C₃-C₁₀ cycloalkyl, wherein C₃-C₁₀ cycloalkyl is optionally substituted by halogen, C₁-C₆ alkyl, C₁-C₃ alkoxy, nitro, —N(C₁-C₃ alkyl)₂, C₁-C₃ haloalkyl, cyano, phenyl, phenoxy or —O(CH₂)ₚOCH₃; and
p is 2, 3, or 4.

In some embodiments, a compound according to the present disclosure is a compound of formula (I):

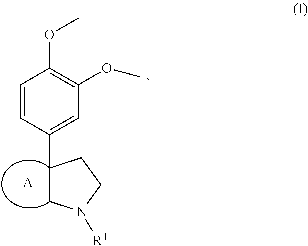

or a pharmaceutically acceptable salt thereof, wherein
R¹ is methyl; and
ring A is

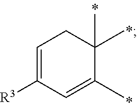

wherein * denotes the attachment points of ring A to the compound of formula (I), and wherein
R³ is —OC(O)C₆ cycloalkyl, wherein C₆ cycloalkyl is optionally substituted by halogen, C₁-C₆ alkyl, C₁-C₃ alkoxy, nitro, —N(C₁-C₃ alkyl)₂, C₁-C₃ haloalkyl, cyano, phenyl, phenoxy or —O(CH₂)ₚOCH₃; and
p is 2, 3, or 4.

In some embodiments, a compound according to the present disclosure is a compound of formula (I):

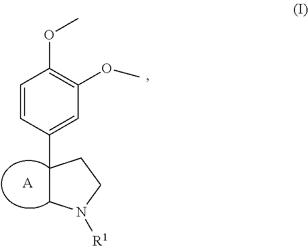

or a pharmaceutically acceptable salt thereof, wherein
R¹ is methyl; and
ring A is

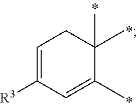

wherein * denotes the attachment points of ring A to the compound of formula (I), and wherein
R³ is —OC(O)C₆ cycloalkyl.

In some embodiments, a compound according to the present disclosure is a compound of formula (IIIb-2):

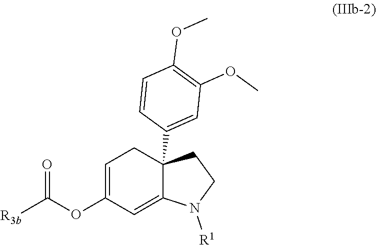

or a pharmaceutically acceptable salt thereof, wherein
R_{3b} is C₁-C₆ alkyl, C₃-C₆ cycloalkyl, phenyl, or 5- to 7-membered heteroaryl, wherein each hydrogen atom in C₁-C₆ alkyl, C₃-C₆ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, phenoxy or —O(CH₂)ₚOCH₃; and
p is 2, 3, or 4.

In some embodiments, a compound according to the present disclosure is a compound of formula (IIIb-2):

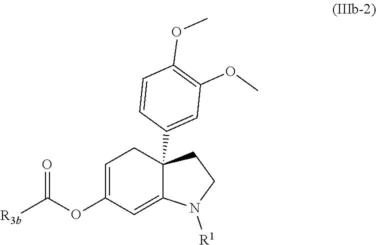

or a pharmaceutically acceptable salt thereof, wherein R_{3b} is C₁-C₆ alkyl optionally substituted with methoxy.

In some embodiments, a compound according to the present disclosure is a compound of formula (IIIb-2):

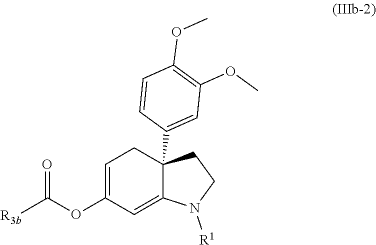

or a pharmaceutically acceptable salt thereof, wherein R_{3b} is C₁-C₆ alkyl.

In some embodiments, a compound according to the present disclosure is a compound of formula (IIIb-2):

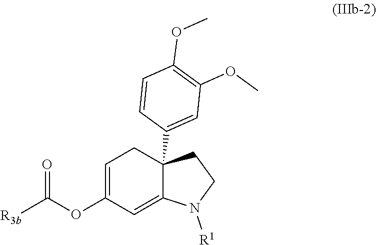

or a pharmaceutically acceptable salt thereof, wherein
R_{3b} is C₃-C₆ cycloalkyl.

In some embodiments, a compound according to the present disclosure is a compound of formula (IIIb-2):

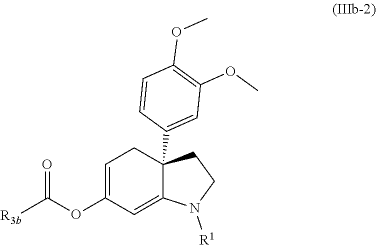

or a pharmaceutically acceptable salt thereof, wherein R_{3b} is phenyl, optionally substituted by C₁-C₆ alkyl.

In some embodiments, a compound according to the present disclosure is a compound of formula (IIIb-2):

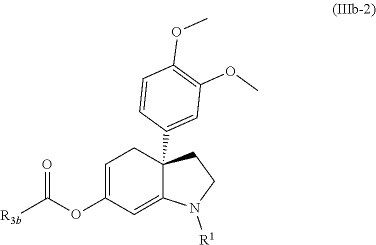

or a pharmaceutically acceptable salt thereof, wherein R_{3b} is phenyl, optionally substituted by methyl.

In certain embodiments, the compound is of formula (I):

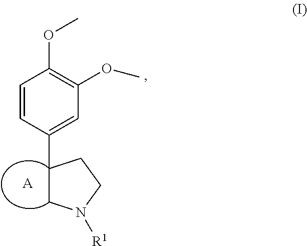

or a pharmaceutically acceptable salt thereof, wherein
R¹ is C₁-C₇ alkyl or H; and
ring A is

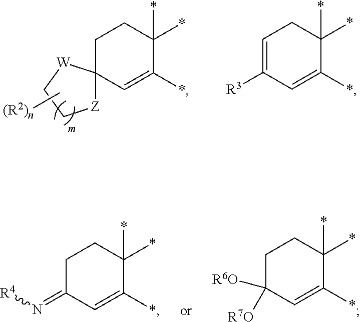

wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol
m is 1 or 2,
n is 0, 1, 2, 3 or 4;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₅ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₅ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and
each of R⁶ and R⁷ is independently C₁-C₃ alkyl.

In certain embodiments, the compound is of formula (I):

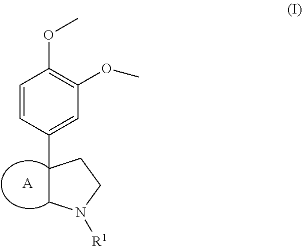

or a pharmaceutically acceptable salt thereof, wherein
R¹ is methyl; and
ring A is

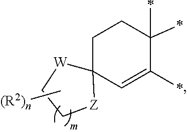

wherein * denotes the attachment points of ring A to the compound of formula (I), and wherein
each of W and Z is independently O, NH, or S;
each R² is independently C₁-C₃ alkyl, —COOH, —CONH₂, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂; or two R²s on a single carbon atom combine to form ═O; or two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol;
m is 1 or 2; and
n is 0, 1, 2, 3 or 4.

In certain embodiments, the compound is of formula (I):

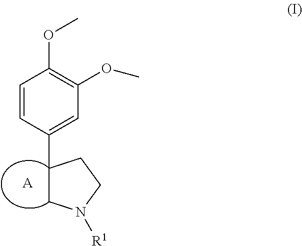

or a pharmaceutically acceptable salt thereof, wherein
R¹ is methyl; and
ring A is

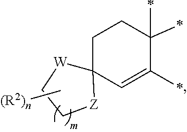

wherein * denotes the attachment points of ring A to the compound of formula (I), and wherein
each of W and Z is independently O, NH, or S;
m is 1 or 2;
n is 0; or n is 1 and R² is C₁-C₃ alkyl, —COOH, —CONH₂, phenyl, or a 6-membered heterocyclyl comprising at least one nitrogen heteroatom; or n is 2, 3 or 4 and two R²s on a single carbon atom combine to form ═O and the remaining R² (if present) is C₁-C₃ alkyl;
wherein each hydrogen atom in phenyl or 6-membered heteroaryl in R² is optionally substituted by —OH, C₁-C₃ alkyl, C₁-C₃ alkoxy, or —NO₂; and
m is 1.

In certain embodiments, the compound is of formula (I):

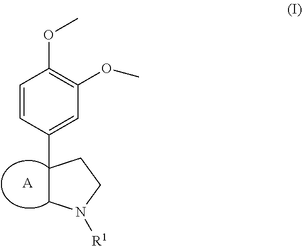

or a pharmaceutically acceptable salt thereof, wherein
R¹ is methyl; and
ring A is

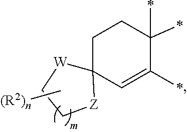

wherein * denotes the attachment points of ring A to the compound of formula (I), and wherein
each of W and Z is independently O, NH, or S;
m is 1; and
n is 0; or n is 1 and R² is methyl, —COOH, —CONH₂, phenyl, or a 6-membered heterocyclyl comprising at least one nitrogen heteroatom; or n is 2, 3 or 4 with two R²s on a single carbon atom combine to form ═O and the remaining R² (if present) is methyl, wherein each hydrogen atom in phenyl or 6-membered heteroaryl in R² is optionally substituted by —OH, methyl, methoxy, or —NO₂.

In certain embodiments, the compound is of formula (I):

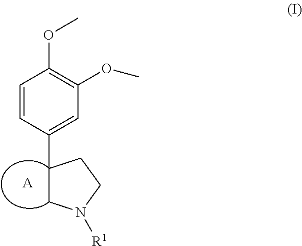

or a pharmaceutically acceptable salt thereof, wherein
R¹ is methyl; and
ring A is

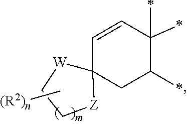

wherein * denotes the attachment points of ring A to the compound of formula (I), and wherein
each of W and Z is independently O, NH, or S;
m is 1; and
n is 1 and R² is

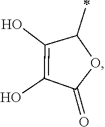 [SMILES: *C1OC(=O)C(O)=C1O]

wherein * denotes the point of attachment of R² the compound.

In certain embodiments, the compound is of formula (I):

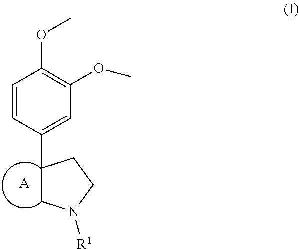

or a pharmaceutically acceptable salt thereof, wherein
R¹ is methyl; and
ring A is

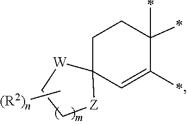

wherein * denotes the attachment points of ring A to the compound of formula (I), and wherein
each of W and Z is independently O, NH, or S;
m is 1; and
n is 2, 3 or 4; and
two adjacent R²s together with the carbon atoms to which they are attached combine to form 5- to 7-membered heteroaryl comprising a nitrogen heteroatom, wherein each hydrogen atom in 5- to 7-membered heteroaryl is optionally substituted by —OH, C₁-C₃ alkyl, or C₁-C₃ alkanol.

In certain embodiments, the compound is of formula (I):

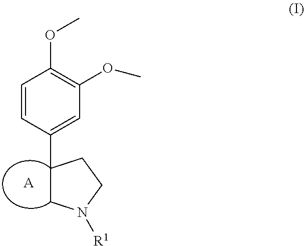

or a pharmaceutically acceptable salt thereof, wherein
R¹ is methyl; and
ring A is

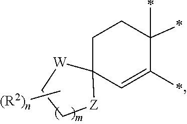

wherein * denotes the attachment points of ring A to the compound of formula (I), and wherein
each of W and Z is independently O, NH, or S;
m is 1; and
n is 2, 3 or 4; and
two adjacent R²s together with the carbon atoms to which they are attached combine to form 6-membered heteroaryl comprising a nitrogen heteroatom, wherein each hydrogen atom in 6-membered heteroaryl is optionally substituted by —OH, C₁-C₃ alkyl, or C₁-C₃ alkanol.

In certain embodiments, the compound is of formula (I):

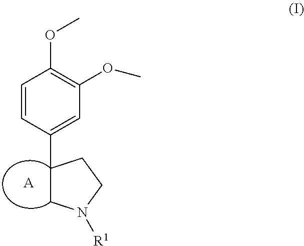

or a pharmaceutically acceptable salt thereof, wherein
R¹ is methyl; and
ring A is

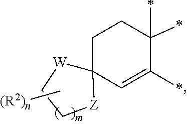

wherein * denotes the attachment points of ring A to the compound of formula (I), and wherein
each of W and Z is independently O, NH, or S;
m is 1; and
n is 0.

In certain embodiments, the compound is of formula (I):

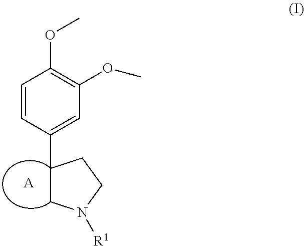

or a pharmaceutically acceptable salt thereof, wherein
R¹ is methyl; and
ring A is

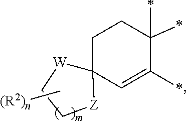

wherein * denotes the attachment points of ring A to the compound of formula (I), and wherein
each of W and Z is independently O, NH, or S;
m is 1; and
n is 1 and R² is methyl, —COOH, or —CONH₂.

In certain embodiments, the compound is of formula (I):

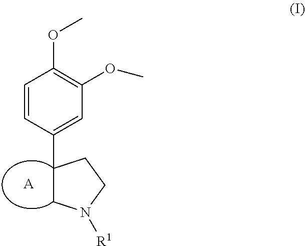

or a pharmaceutically acceptable salt thereof, wherein
R¹ is methyl; and
ring A is

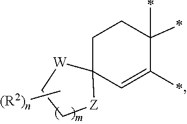

wherein * denotes the attachment points of ring A to the compound of formula (I), and wherein
each of W and Z is independently O, NH, or S;
m is 1; and
n is 1 and R² is phenyl or a 6-membered heterocyclyl comprising at least one nitrogen heteroatom, wherein each hydrogen atom in phenyl or 6-membered heteroaryl in R² is optionally substituted by —OH, methyl, methoxy, or —NO₂.

In certain embodiments, the compound is of formula (I):

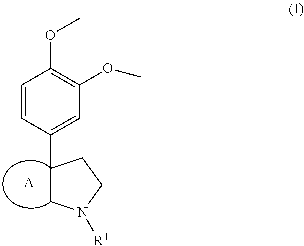

or a pharmaceutically acceptable salt thereof, wherein
R¹ is methyl; and
ring A is

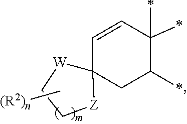

wherein * denotes the attachment points of ring A to the compound of formula (I), and wherein
each of W and Z is independently O, NH, or S;
m is 1; and
n is 2 or 3 with two R²s on a single carbon atom combine to form ═O and the remaining R² (if present) is methyl;
wherein each hydrogen atom in phenyl or 6-membered heteroaryl in R² is optionally substituted by —OH, methyl, methoxy, or —NO₂.

In certain embodiments, the compound is of formula (I):

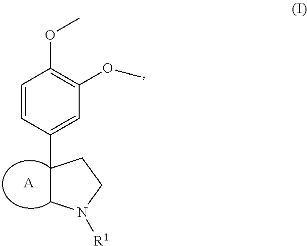

or a pharmaceutically acceptable salt thereof, wherein
R¹ is methyl; and
ring A is

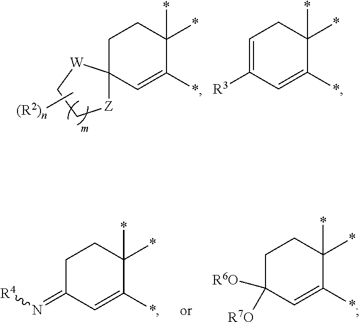

wherein * denotes the attachment points of ring A to the compound of formula (I), and wherein
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)-C₁—C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and
each of R⁶ and R⁷ is independently C₁-C₃ alkyl.

In certain embodiments, the compound is of formula (I):

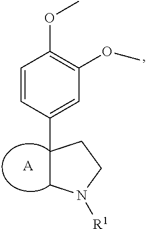

or a pharmaceutically acceptable salt thereof, wherein
R¹ is methyl; and
ring A is

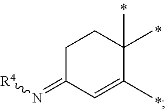

wherein * denotes the attachment points of ring A to the compound of formula (I), and wherein
R⁴ is —OR⁵ or —N(R⁵)₂; and
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₅ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl.

In certain embodiments, the compound is of formula (I):

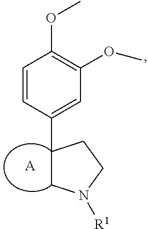

or a pharmaceutically acceptable salt thereof, wherein
R¹ is methyl; and
ring A is

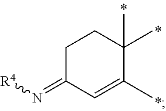

wherein * denotes the attachment points of ring A to the compound of formula (I), and wherein
R⁴ is —OR⁵ or —N(R⁵)₂; and
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₅ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₅ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl.

In certain embodiments, the compound is of formula (I):

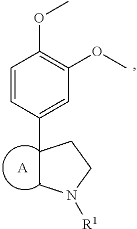

or a pharmaceutically acceptable salt thereof, wherein
R¹ is methyl; and
ring A is

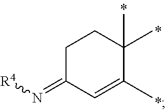

wherein * denotes the attachment points of ring A to the compound of formula (I), and
each of R⁶ and R⁷ is independently C₁-C₃ alkyl.

### ADDITIONAL EMBODIMENTS

In some embodiments, the compound is a compound of one or more of the following embodiments:

1. A compound of formula (I):

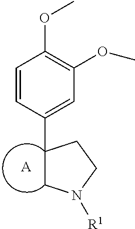

or a pharmaceutically acceptable salt thereof, wherein
R¹ is or C₁-C₇ alkyl or H; and
ring A is

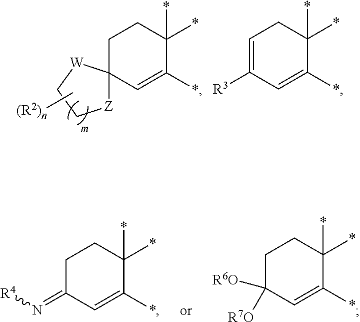

wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, C₃-C₁₀ cycloalkyl, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl or phenyl, wherein each hydrogen atom in 5- to 7-membered heteroaryl or phenyl is optionally substituted by —OH, C₁-C₃ alkyl, or C₁-C₃ alkanol;
m is 1 or 2;
n is 0, 1, 2, or 3;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, phenoxy or —O(CH₂)ₚOCH₃;
p is 2, 3, or 4;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₅ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₅ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and
each of R⁶ and R⁷ is independently C₁-C₃ alkyl.

2. The compound of embodiment 1, wherein the compound is of formula (I):

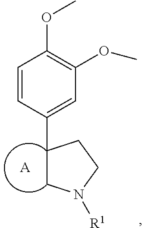

or a pharmaceutically acceptable salt thereof, wherein
R¹ is or C₁-C₇ alkyl or H; and
ring A is

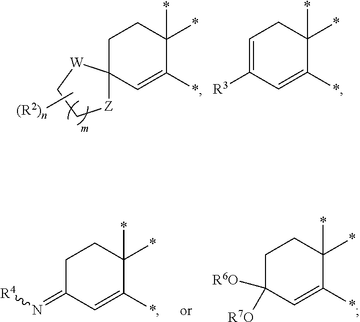

wherein * denotes the attachment points of ring A to the compound of formula (I), and wherein
   each of W and Z is independently O, NH, or S;
   each R² is independently C₁-C₃ alkyl, C₂-C₆ alkenyl, —COOH, —CONH₂, C₃-C₁₀ cycloalkyl, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl or phenyl, wherein each hydrogen atom in 5- to 7-membered heteroaryl or phenyl is optionally substituted by —OH, C₁-C₃ alkyl, or C₁-C₃ alkanol;
m is 1 or 2;
n is 0, 1, or 2;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and each of R⁶ and R⁷ is independently C₁-C₃ alkyl.

3. The compound of embodiment 1 or 2, wherein the compound is of formula (I):

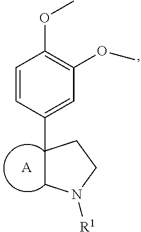

or a pharmaceutically acceptable salt thereof, wherein
R¹ is C₁-C₇ alkyl or H; and
ring A is

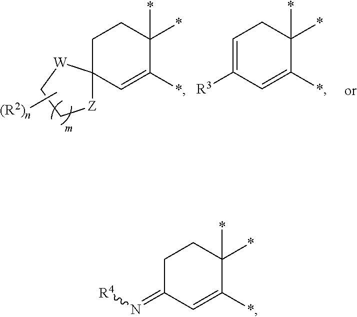

wherein * denotes the attachment points of ring A to the compound of formula (I), and wherein
each of W and Z is independently O, NH, or S;
each R² is independently C₁-C₃ alkyl, —COOH, —CONH₂, phenyl, 5- to 7-membered heterocyclyl, or 5- to 7-membered heteroaryl, wherein each hydrogen atom in phenyl, 5- to 7-membered heterocyclyl and 5- to 7-membered heteroaryl is optionally substituted by —OH, C₁-C₃ alkyl, C₁-C₃ alkoxy, C₁-C₃ alkanol, or —NO₂, or two R²s on a single carbon atom combine to form ═O; or two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol;
m is 1 or 2;
n is 0, 1, or 2;
R³ is —OSi(C₁-C₆ alkyl)₃ or —OC(O)C₁-C₆ alkyl; and
R⁴ is OH, C₁-C₆ alkoxy, or —NHC(O)-5- to 7-membered heteroaryl, wherein each hydrogen atom in 5- to 7-membered heteroaryl is optionally substituted by —OH, C₁-C₃ alkyl, or C₁-C₃ alkanol.

4. The compound of embodiment 1, wherein the compound is of formula (I-1):

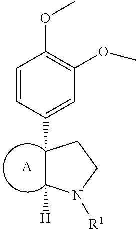

or a pharmaceutically acceptable salt thereof, wherein
R¹ is C₁-C₇ alkyl or H; and
ring A is

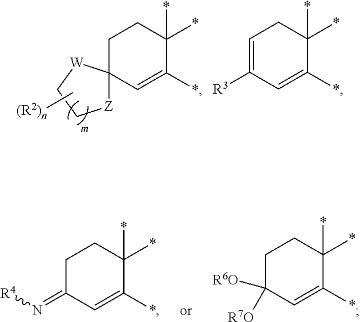

wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, C₃-C₁₀ cycloalkyl, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl or phenyl, wherein each hydrogen atom in 5- to 7-membered heteroaryl or phenyl is optionally substituted by —OH, C₁-C₃ alkyl, or C₁-C₃ alkanol;
m is 1 or 2;
n is 0, 1, 2, or 3;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, or phenoxy or —O(CH₂)ₚOCH₃;
p is 2, 3, or 4;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, —C(O)-5- to 7-membered heteroaryl, —C(O)—C₁-C₈ alkyl, —C(O)—C₃-C₁₀ cycloalkyl, —C(O)— phenyl, —C(O)N(H)-phenyl, C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl;
each of R⁶ and R⁷ is independently C₁-C₃ alkyl; and
the compound of formula (I-1) has the absolute stereochemistry shown.

5. The compound of embodiment 1, 2, or 3, wherein the compound is of formula (IIa):

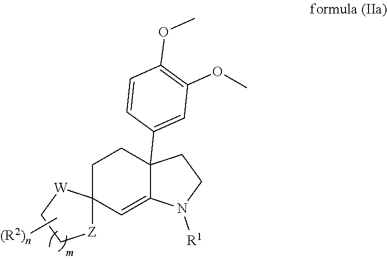

or a pharmaceutically acceptable salt thereof.

6. The compound of any one of embodiments 1-4, wherein the compound is of formula (IIa-1):

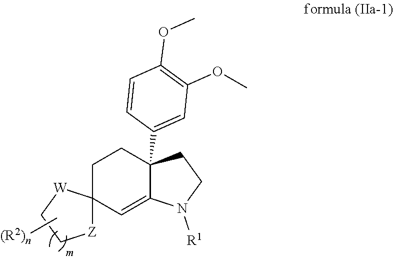

or a pharmaceutically acceptable salt thereof; and the compound of formula (IIa-1) has the absolute stereochemistry shown.

7. The compound of any one of embodiments 1-6, wherein m is 1.

8. The compound of any one of embodiments 1-7, wherein n is 1.

9. The compound of any one of embodiments 1, 2, and 4-8, each R² is C₁-C₃ alkyl, vinyl, COOH, —CONH₂, adamantyl, phenyl,

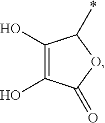 [SMILES: *C1OC(=O)C(O)=C1O]

or pyridyl, wherein each hydrogen atom in C₁-C₃ alkyl, phenyl, and pyridyl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl or phenyl, wherein each hydrogen atom in 5- to 7-membered heteroaryl or phenyl is optionally substituted by —OH, C₁-C₃ alkyl, or C₁-C₃ alkanol.

10. The compound of any one of embodiments 1-8, wherein R² is phenyl, wherein each hydrogen atom in phenyl is optionally substituted by —OH, C₁-C₃ alkyl, C₁-C₃ alkoxy, or —NO₂.

11. The compound of any one of embodiments 1-8, wherein R² is 5- to 7-membered heterocyclyl or 5- to 7-membered heteroaryl, wherein each hydrogen atom in 5- to 7-membered heterocyclyl and 5- to 7-membered heteroaryl is optionally substituted by —OH, C₁-C₃ alkyl, C₁-C₃ alkoxy, C₁-C₃ alkanol, or —NO₂.

12. The compound of any one of embodiments 1, 2, and 4-8, wherein R² is C₁-C₃ alkyl optionally substituted by C₁-C₃ alkoxy or aryloxy, each of which is optionally substituted by C₁-C₃ alkoxy or phenyl.

13. The compound of any one of embodiments 1-8, wherein R² is pyridyl.

14. The compound of any one of embodiments 1-8, wherein R² is

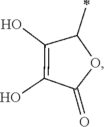 [SMILES: *C1OC(=O)C(O)=C1O]

wherein * denotes the point of attachment of R² to the compound.

15. The compound of any one of embodiments 1-8, wherein R² is —COOH or —CONH₂.

16. The compound of any one of embodiments 1-7, wherein n is 2.

17. The compound of embodiment 16, wherein the two R²s are on adjacent carbon atoms and the two R²s together with the carbon atoms to which they are attached combine to form 5- to 7-membered heteroaryl or phenyl, wherein each hydrogen atom in 5- to 7-membered heteroaryl or phenyl is optionally substituted by —OH, C₁-C₃ alkyl, or C₁-C₃ alkanol.

18. The compound of embodiment 17, the two R²s together with the carbon atoms to which they are attached combine to form pyridyl, wherein each hydrogen atom in pyridyl is optionally substituted by —OH, C₁-C₃ alkyl, or C₁-C₃ alkanol.

19. The compound of any one of embodiments 1-18, wherein W is O.

20. The compound of any one of embodiments 1-19, wherein Z is O, S or NH.

21. The compound of any one of embodiments 1-19, wherein Z is O.

22. The compound of any one of embodiments 1-17, wherein W is NH.

23. The compound of embodiment 22, wherein Z is O, S, or NH.

24. The compound of any one of embodiments 1-18, wherein W is S.

25. The compound of embodiment 24, wherein Z is O, S, or NH.

26. The compound of embodiment 1, 2, or 3, wherein the compound is of formula (IIIa):

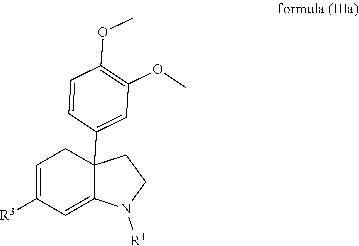

or a pharmaceutically acceptable salt thereof.

27. The compound of embodiment 1, 2, or 3, wherein the compound is of formula (IIIa-1):

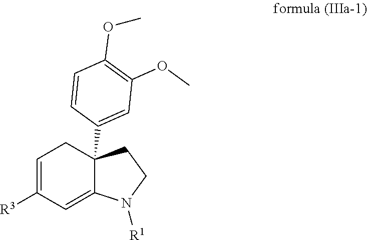

or a pharmaceutically acceptable salt thereof; and the compounds of formula (IIIa-1) have the absolute stereochemistry shown.

28. The compound of embodiment 1, 2, or 3, wherein the compound is of formula (IIIa-1):

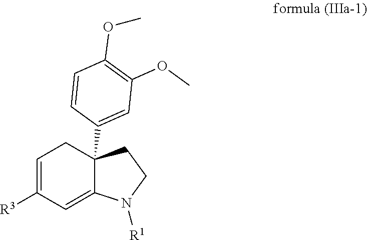

or a pharmaceutically acceptable salt thereof.

29. The compound of any one of embodiments 1, 2, 3, and 26-29, wherein R³ is —OSi(C₁-C₆ alkyl)₃.

30. The compound of embodiment 30, wherein R³ is —OSi(CH₃)₃.

31. The compound of any one of embodiments 1, 2, 3, and 26-29, wherein R³ is —OC(O)C₁-C₆ alkyl.

32. The compound of embodiment 32, wherein R³ is —OC(O)CH₃.

33. The compound of any one of embodiments 1, 2, 3, and 26-29, wherein R³ is —OC(O)C₁-C₆ alkyl and the C₁-C₆ alkyl is methyl, ethyl, butyl, isobutyl, tertbutyl, or 2-methylbutyl, wherein each hydrogen atom in methyl, ethyl, butyl, isobutyl, tertbutyl, and 2-methylbutyl is optionally substituted by halogen or phenyl.

34. The compound of any one of embodiments 1, 2, 3, and 26-29, wherein R³ is —OC(O)C₁-C₆ alkyl and the C₁-C₆ alkyl is ethyl, propyl, butyl, pentyl, or hexyl, wherein each hydrogen atom in methyl, ethyl, propyl, butyl, pentyl, and hexyl is optionally substituted by methyl, ethyl, methoxy or —O(CH₂)ₚOCH₃; and p is 2, 3, or 4.

35. The compound of any one of embodiments 1, 2, 3, and 26-29, wherein R³ is

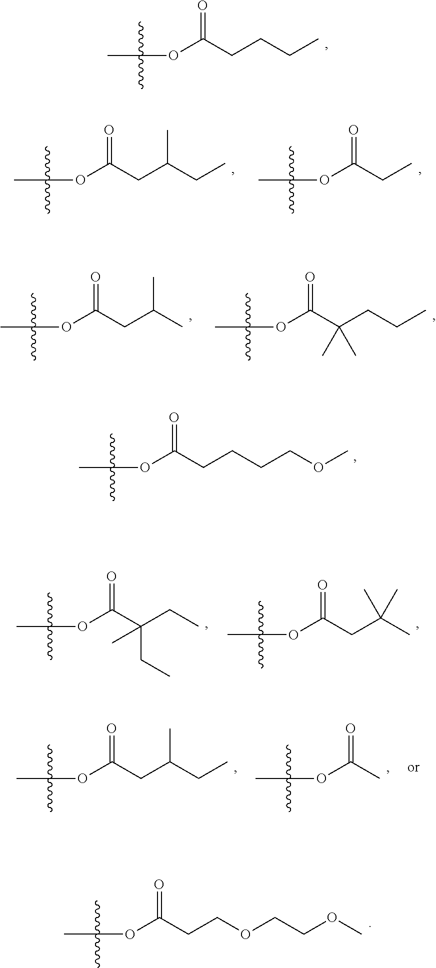

36. The compound of any one of embodiments 1, 2, and 26-29, wherein R³ is —OC(O)C₂-C₆ alkenyl.

37. The compound of embodiment 36, wherein the C₂-C₆ alkenyl is isopropenyl or butenyl.

38. The compound of any one of embodiments 1, 2, and 26-29, wherein R³ is —OC(O)C₃-C₁₀ cycloalkyl.

39. The compound of embodiment 38, wherein the C₃-C₁₀ cycloalkyl is cyclopropyl or cyclohexyl.

40. The compound of embodiment 38, wherein the C₃-C₁₀ cycloalkyl is cyclohexyl.

41. The compound of any one of embodiments 1, 2, and 26-29, wherein R³ is —OC(O)phenyl, wherein each hydrogen atom in phenyl is optionally substituted by methyl, isopropyl, tertbutyl, chloro, fluoro, CF₃, —CHF₂, cyano, —N(CH₃)₂, -methoxy, or nitro.

42. The compound of any one of embodiments 1, 2, and 26-29, wherein R³ is —OC(O)phenyl, wherein each hydrogen atom in phenyl is optionally substituted by methyl.

43. The compound of any one of embodiments 1, 2, and 26-29, wherein R³ is —OC(O)phenyl, wherein one hydrogen atom in phenyl is substituted by methyl.

44. The compound of any one of embodiments 1, 2, and 26-29, wherein R³ is —OC(O)-5- to 7-membered heteroaryl.

45. The compound of embodiment 44, wherein the 5- to 7-membered heteroaryl is pyridyl or thiophenyl, wherein each hydrogen atom in pyridyl or thiophenyl is optionally substituted by methyl.

46. The compound of embodiment 44, wherein the 5- to 7-membered heteroaryl is pyridyl wherein each hydrogen atom in pyridyl is optionally substituted by methyl.

47. The compound of embodiment 44, wherein the 5- to 7-membered heteroaryl is unsubstituted pyridyl.

48. The compound of embodiment 1, 2 or 3, wherein the compound is of formula (IVa):

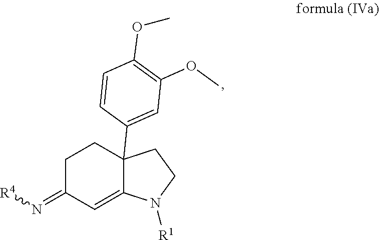

or a pharmaceutically acceptable salt thereof.

49. The compound of embodiment 1, 2, 3, or 4 wherein the compound is of formula (IVa-1):

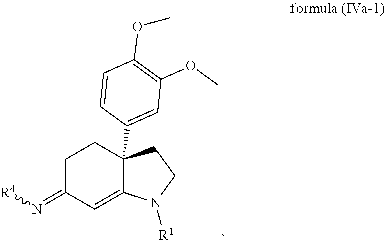

or a pharmaceutically acceptable salt thereof; and the compounds of formula (IVa-1) have the absolute stereochemistry shown.

50. The compound of embodiment 1, 2, 48, or 49, wherein R⁴ is OR⁵.

51. The compound of embodiment 50, wherein R⁵ is H, C₁-C₆ alkyl, C₃-C₁₀ cycloalkyl, C₃-C₆ heterocycloalkyl or phenyl, wherein each hydrogen atom in C₁-C₆ alkyl, C₃-C₆ heterocycloalkyl and phenyl is optionally substituted with —OH, C₁-C₆ alkoxy, 5- to 7-membered heteroaryl, phenyl, phenoxy, C₃-C₆ cycloalkyl, —C(O)OC₁-C₆ alkyl, C₃-C₁₀ cycloalkyl, or —COOH.

52. The compound of embodiment 1, 2, 48, or 49, wherein R⁴ is —N(R⁵)₂.

53. The compound of embodiment 52, wherein each R⁵ is independently H, —C₁-C₆ alkyl, 5- to 10-membered heteroaryl, —C(O)-5- to 7-membered heteroaryl, —C(O)-phenyl, phenyl, —C(O)—C₁-C₃ alkyl, —C(O)—C₃-C₁₀ cycloalkyl, —C(O)N(H)-phenyl, —C(O)N(H)-pyridyl, —C(O)COOH, or —CONH₂, wherein each hydrogen atom in —C₁-C₆ alkyl, 5- to 10-membered heteroaryl, —C(O)-5- to 7-membered heteroaryl, —C(O)-phenyl, phenyl, —C(O)—C₁-C₃ alkyl, and —C(O)N(H)-phenyl is optionally substituted by halogen, aryloxy, carboxylate, phenyl, —S(O)₂CH₃, C₁-C₃ alkoxy, C₃-C₆ cycloalkyl optionally substituted by carboxylate, 5- to 7-membered heteroaryl, or 5- to 7-membered heterocyclyl optionally substituted by methyl.

54. The compound of any one of the preceding embodiments, wherein R¹ is C₁-C₃ alkyl.

55. The compound of any one of the preceding embodiments, wherein R¹ is methyl.

56. The compound of embodiment 1, selected from the group consisting of:

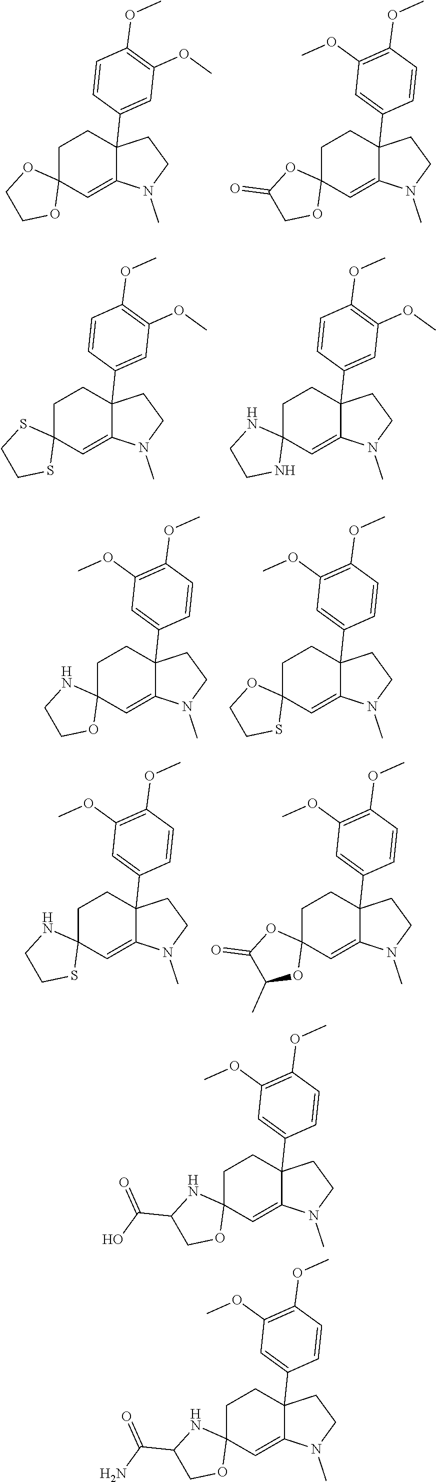

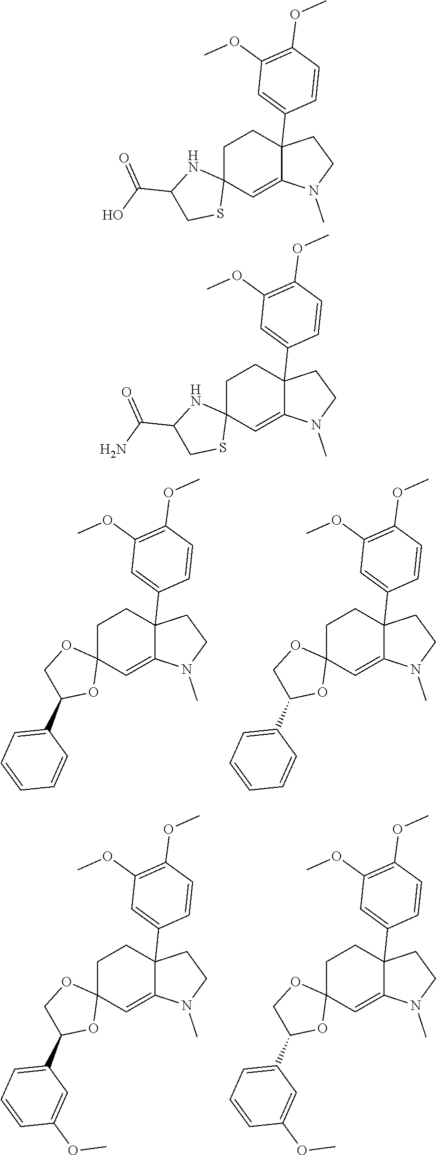

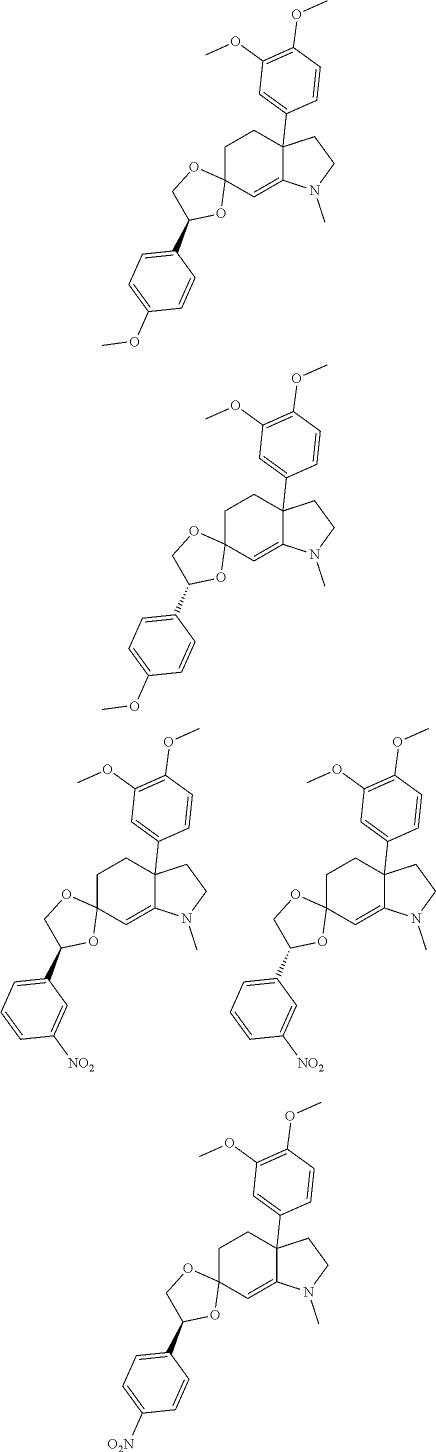

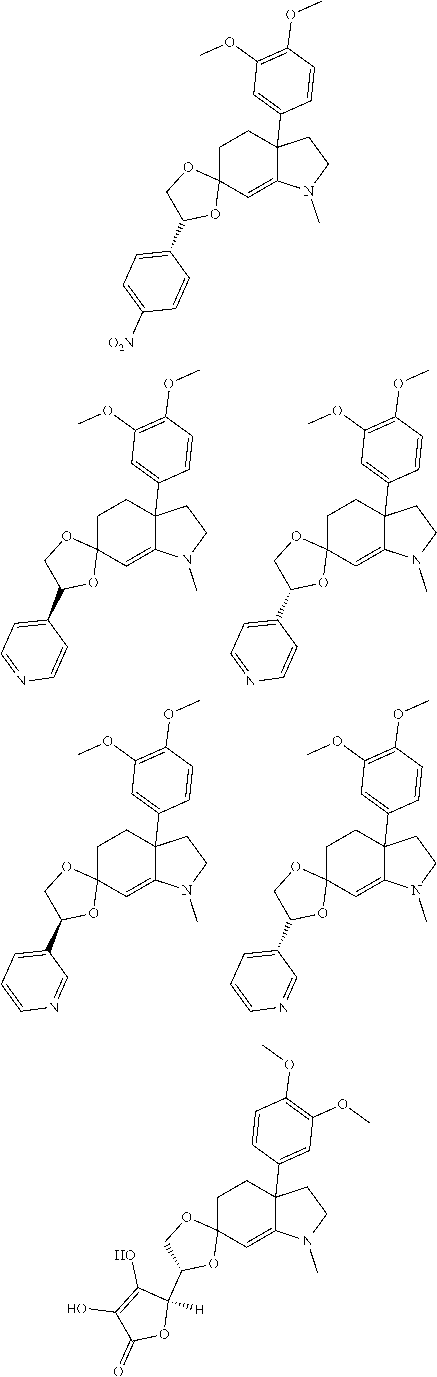

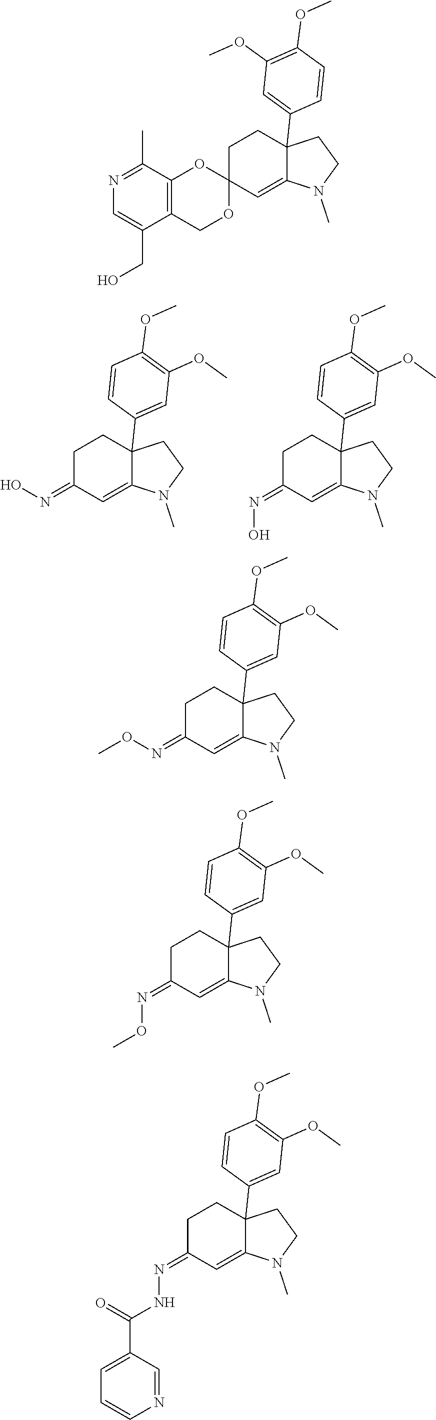

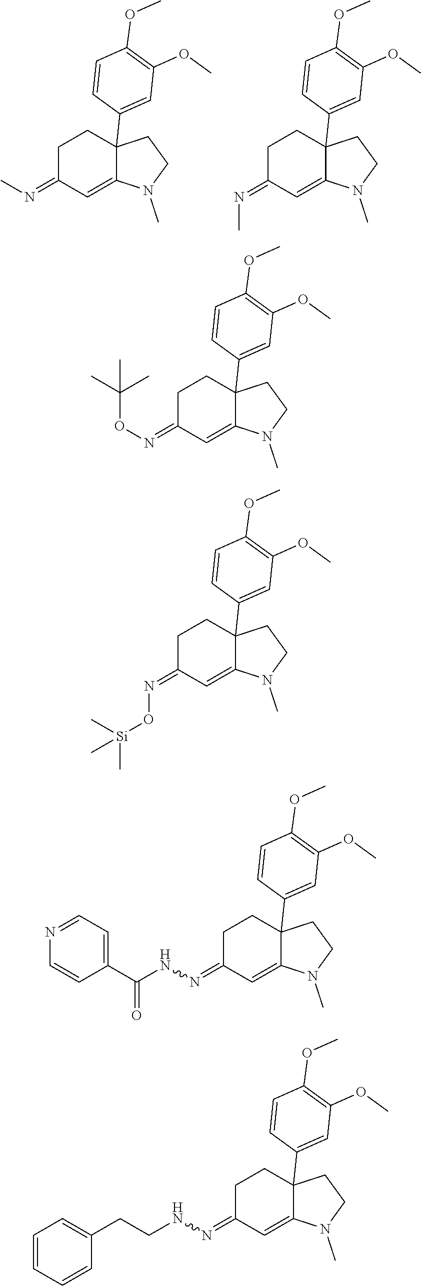

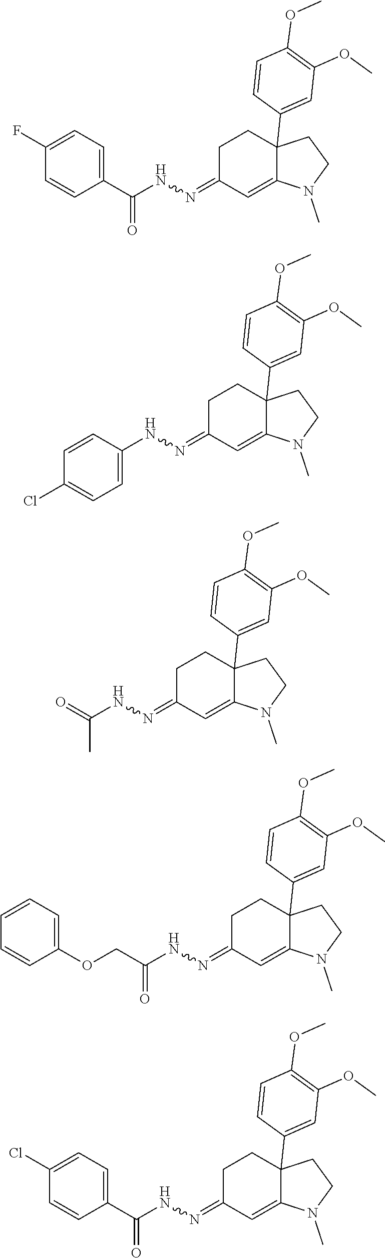

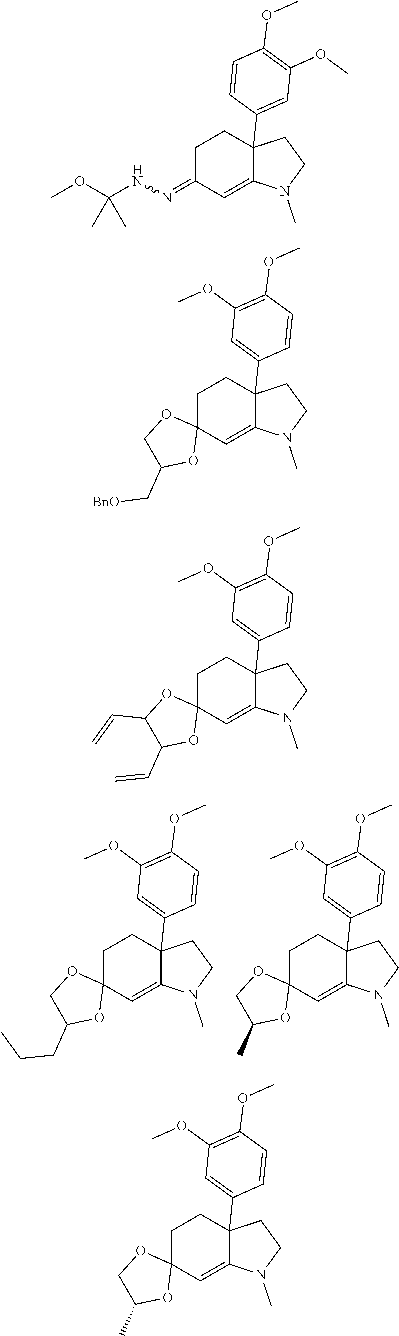

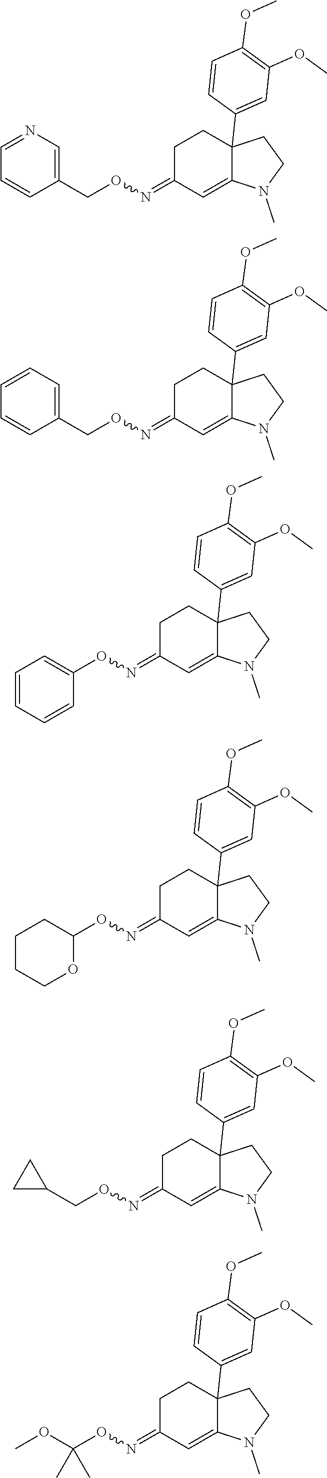

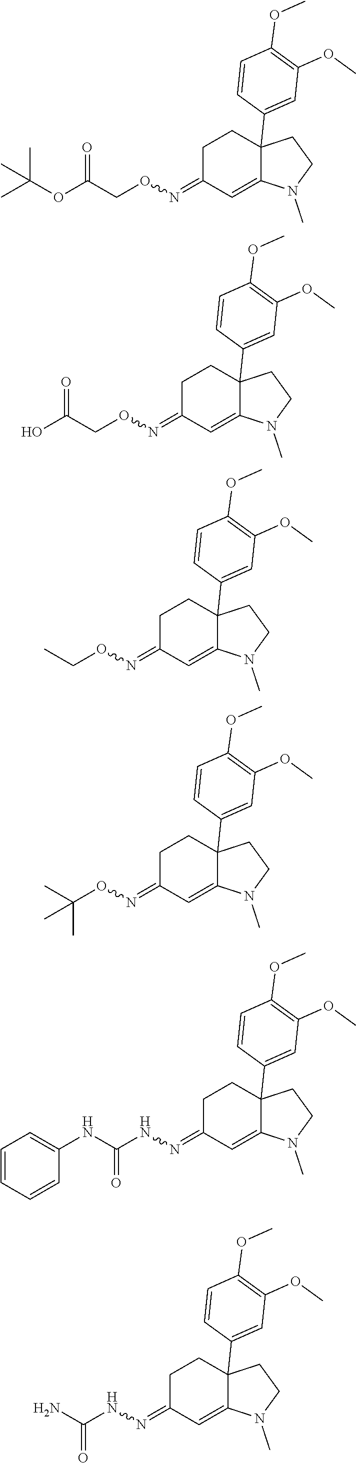

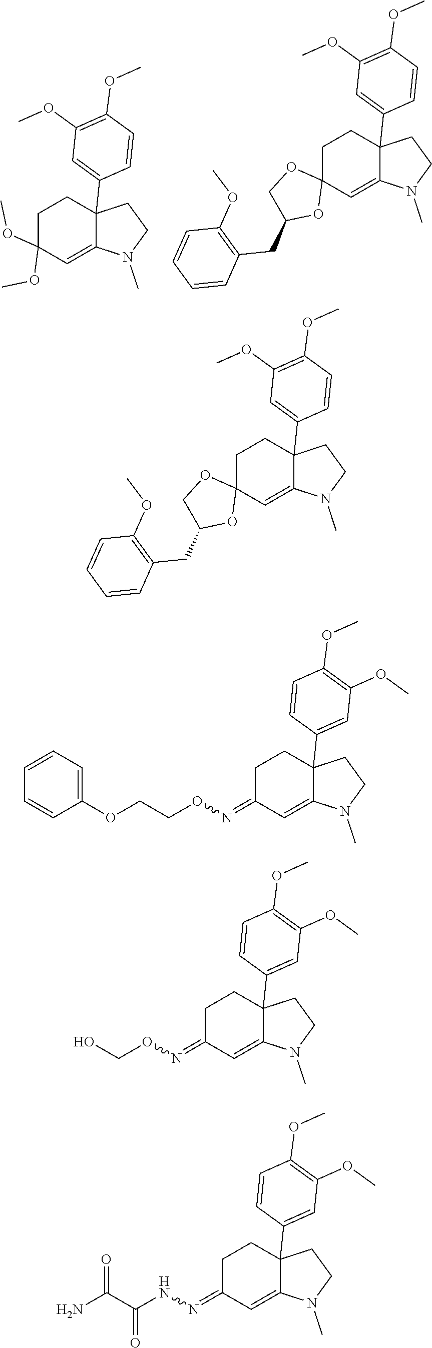

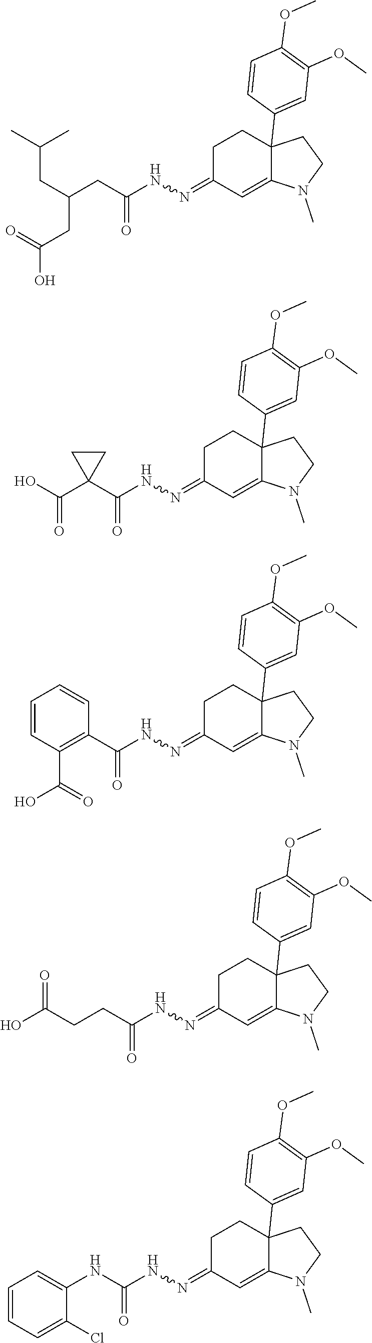

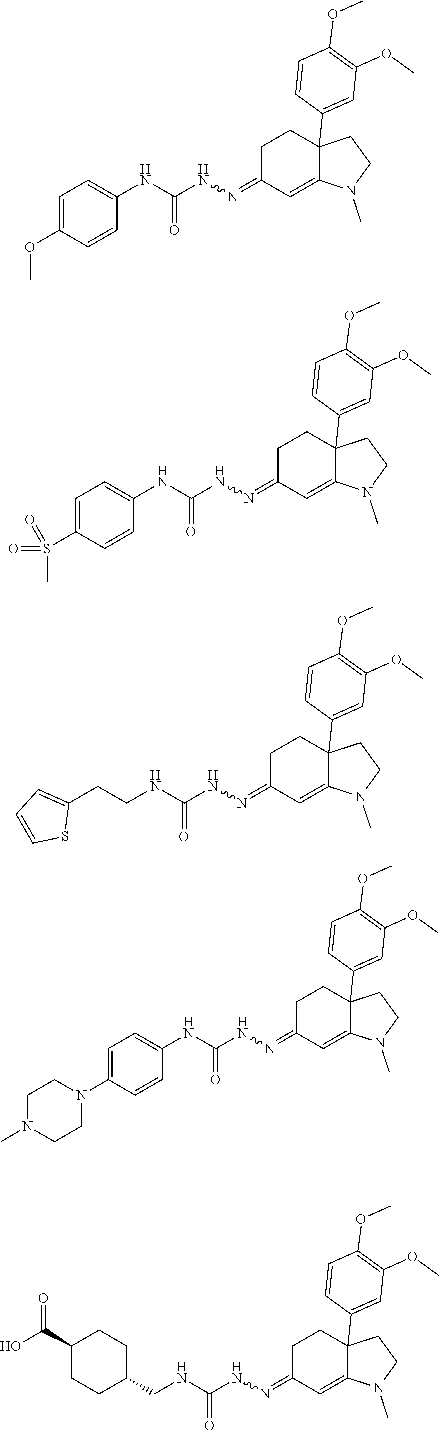

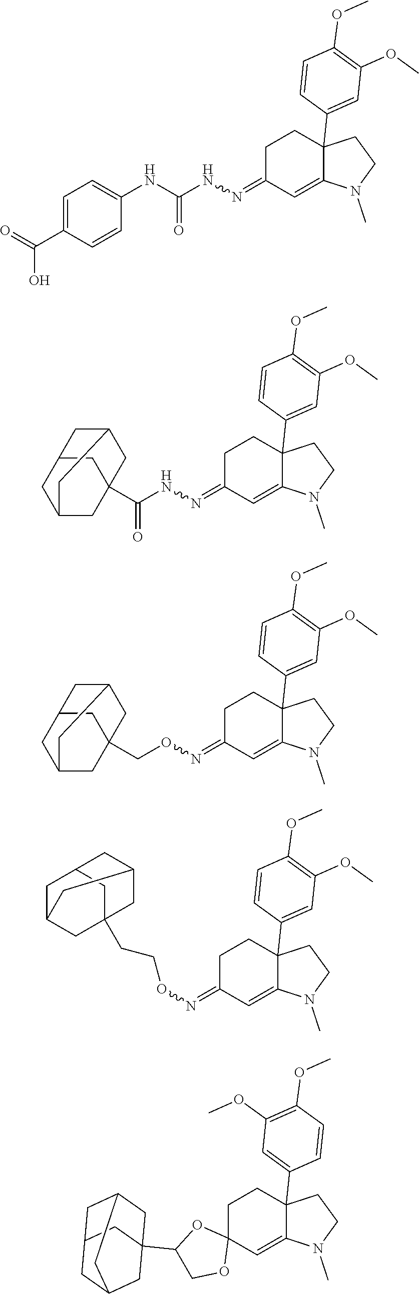

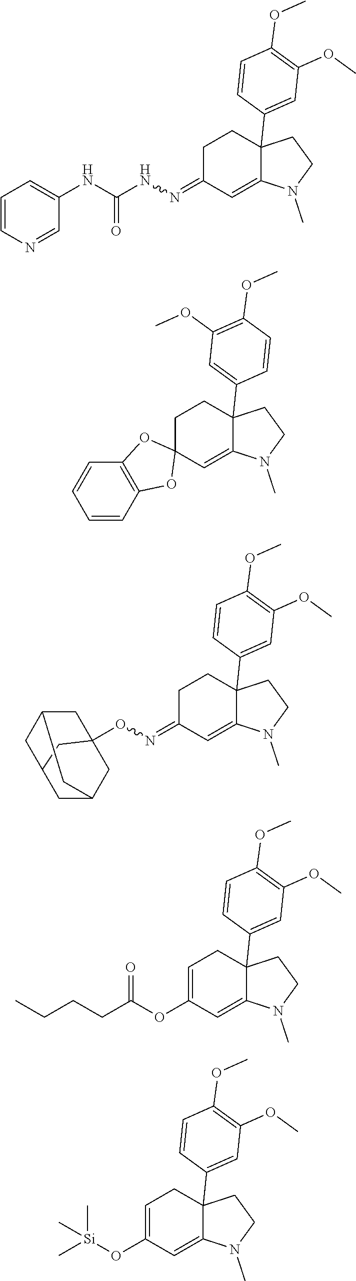

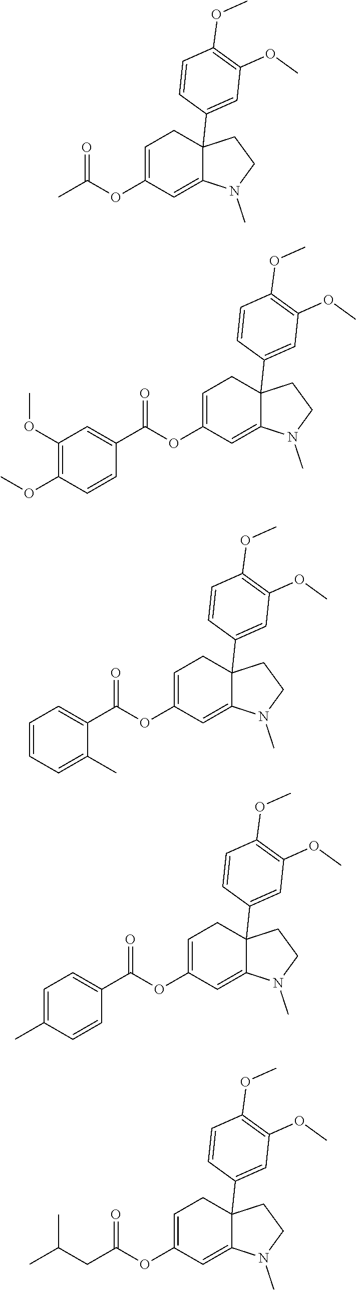

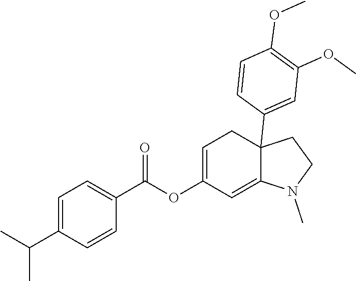 [SMILES: COc1ccc(C23CC=C(OC(=O)c4ccc(C(C)C)cc4)C=C2N(C)CC3)cc1OC]

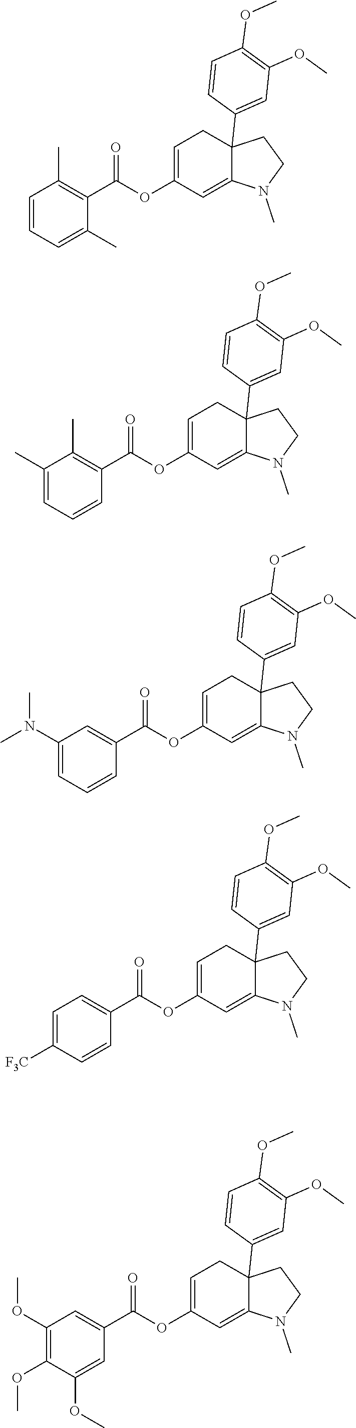

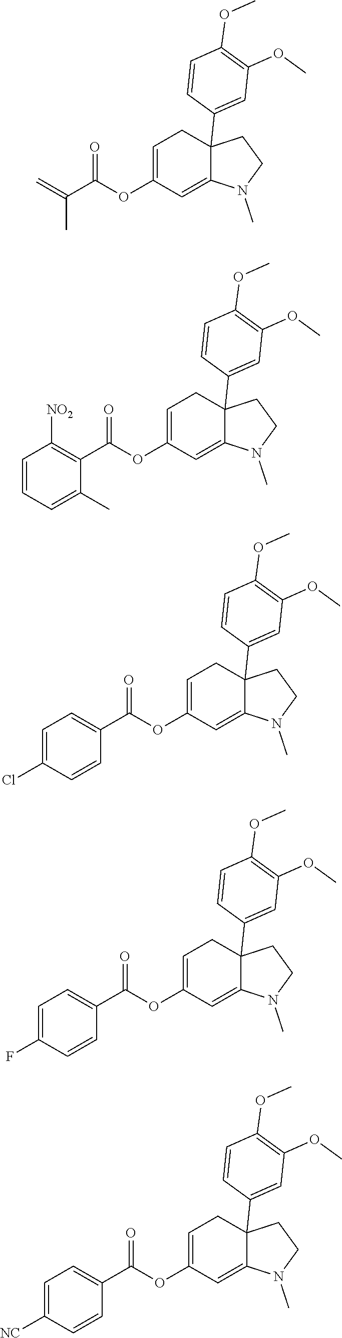

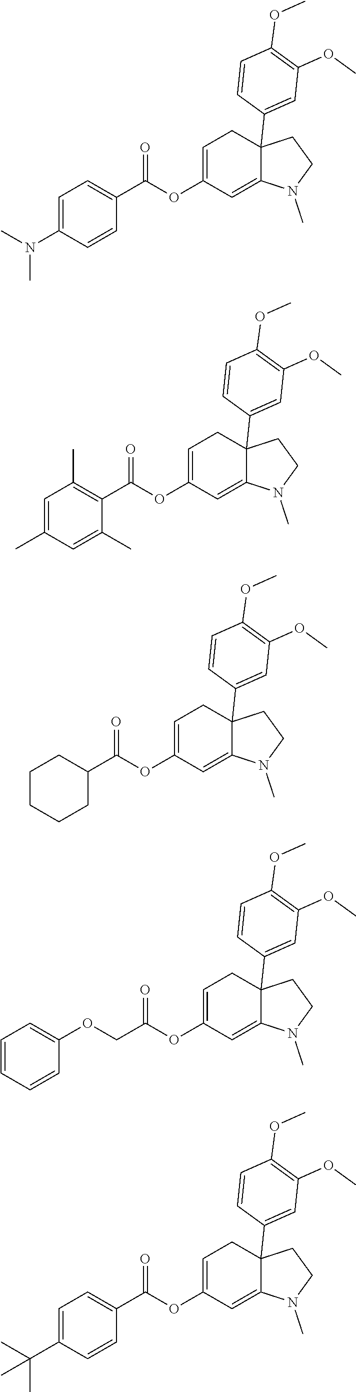

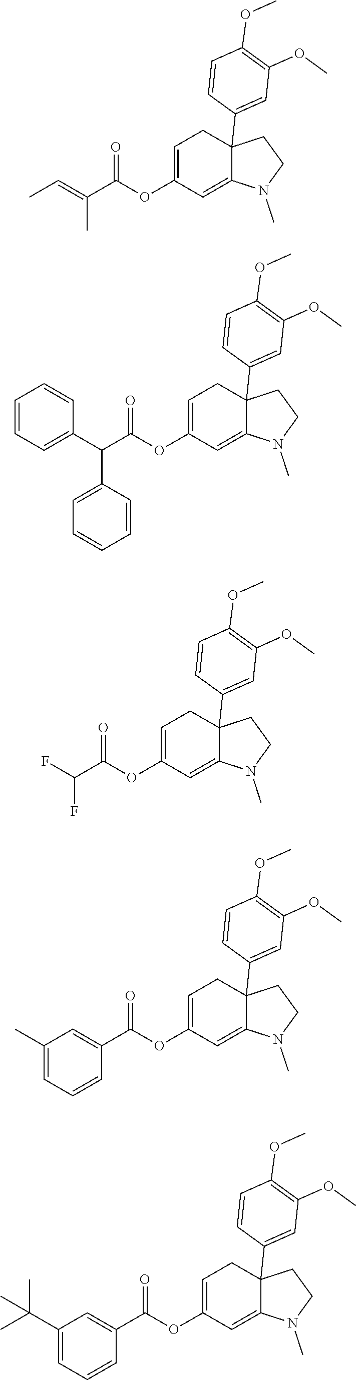

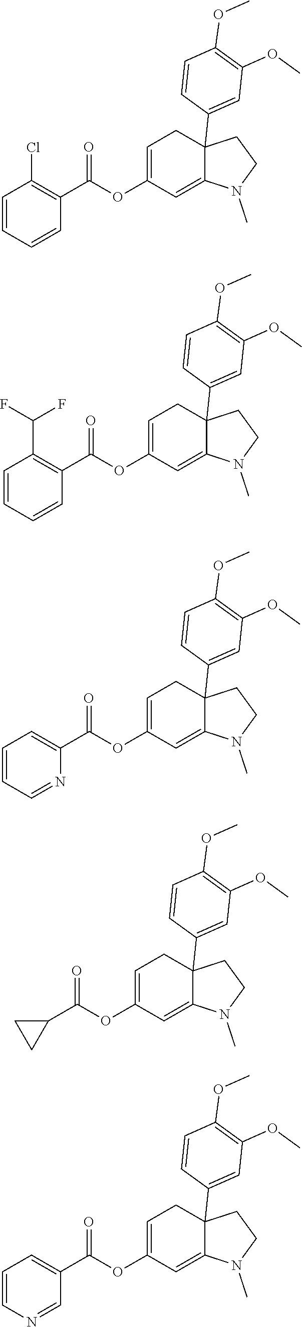

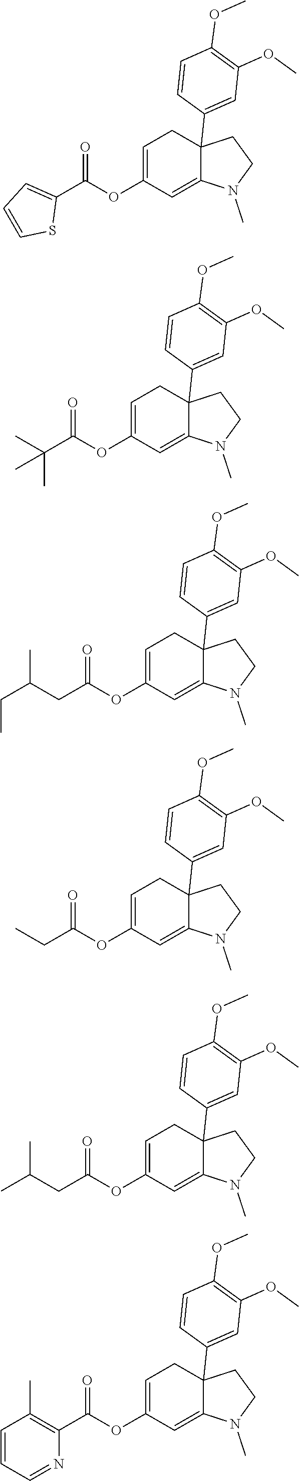

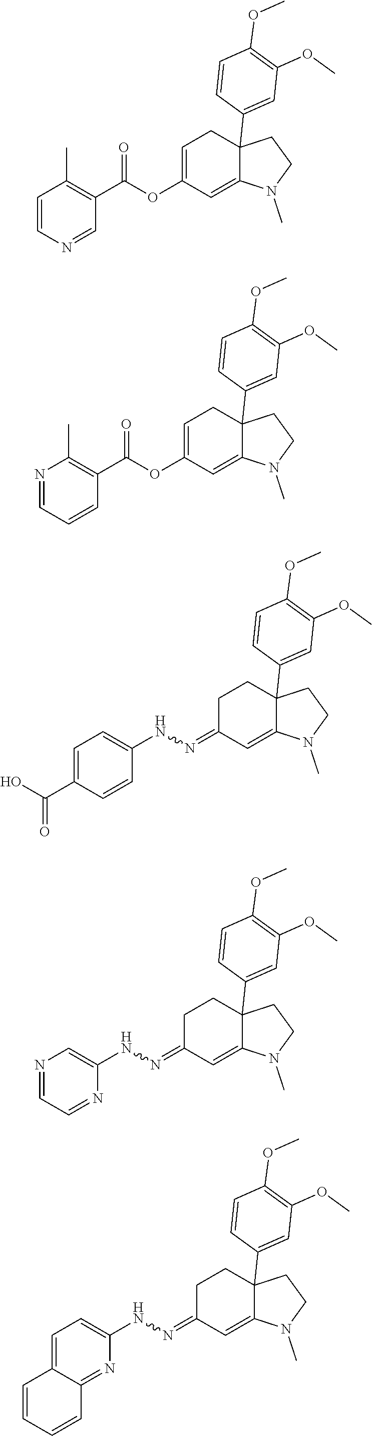 [SMILES: COc1ccc(C23CC=C(OC(=O)c4cnccc4C)C=C2N(C)CC3)cc1OC ; COc1ccc(C23CC=C(OC(=O)c4cccnc4C)C=C2N(C)CC3)cc1OC ; COc1ccc(C23CCC(=NNc4ccc(C(=O)O)cc4)C=C2N(C)CC3)cc1OC ; COc1ccc(C23CCC(=NNc4cnccn4)C=C2N(C)CC3)cc1OC ; COc1ccc(C23CCC(=NNc4ccc5ccccc5n4)C=C2N(C)CC3)cc1OC]

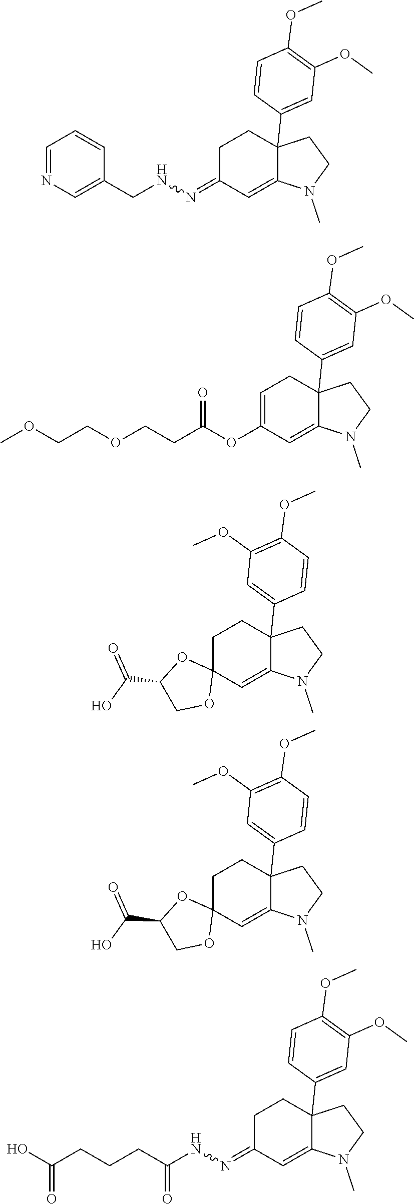

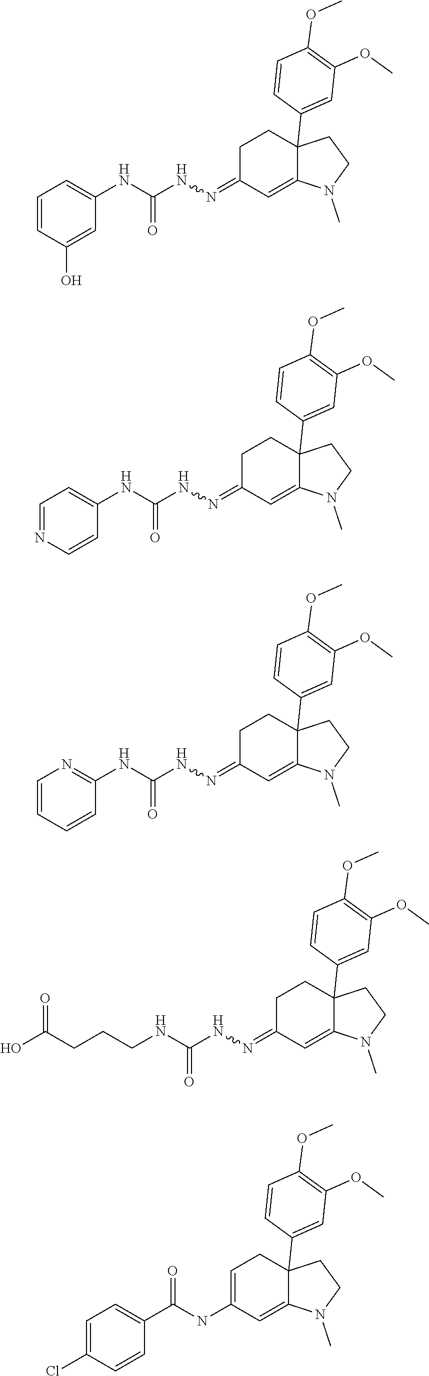 [SMILES: COc1ccc(C23CCC(=NNC(=O)Nc4cccc(O)c4)C=C2N(C)CC3)cc1OC ; COc1ccc(C23CCC(=NNC(=O)Nc4ccncc4)C=C2N(C)CC3)cc1OC ; COc1ccc(C23CCC(=NNC(=O)Nc4ccccn4)C=C2N(C)CC3)cc1OC ; COc1ccc(C23CCC(=NNC(=O)NCCCC(=O)O)C=C2N(C)CC3)cc1OC ; COc1ccc(C23CC=C(NC(=O)c4ccc(Cl)cc4)C=C2N(C)CC3)cc1OC]

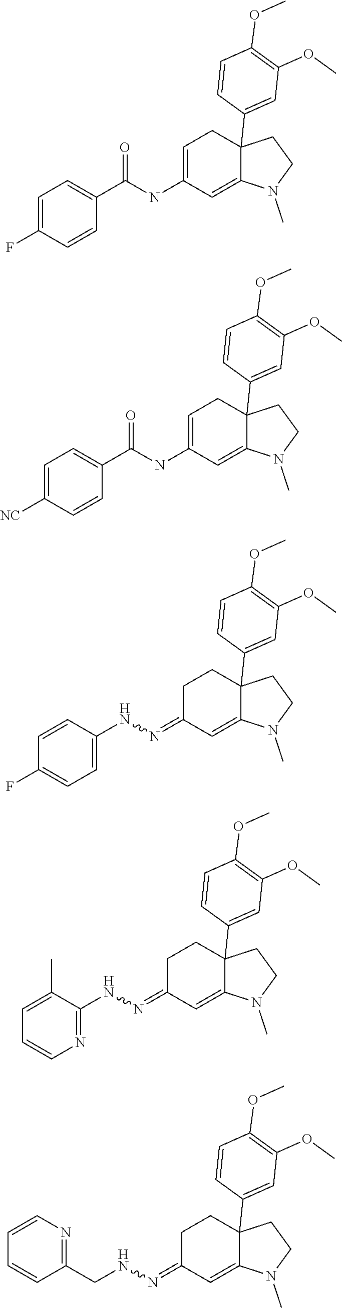 [SMILES: COc1ccc(C23CC=C(NC(=O)c4ccc(F)cc4)C=C2N(C)CC3)cc1OC ; COc1ccc(C23CC=C(NC(=O)c4ccc(C#N)cc4)C=C2N(C)CC3)cc1OC ; COc1ccc(C23CCC(=NNc4ccc(F)cc4)C=C2N(C)CC3)cc1OC ; COc1ccc(C23CCC(=NNc4ncccc4C)C=C2N(C)CC3)cc1OC ; COc1ccc(C23CCC(=NNCc4ccccn4)C=C2N(C)CC3)cc1OC]

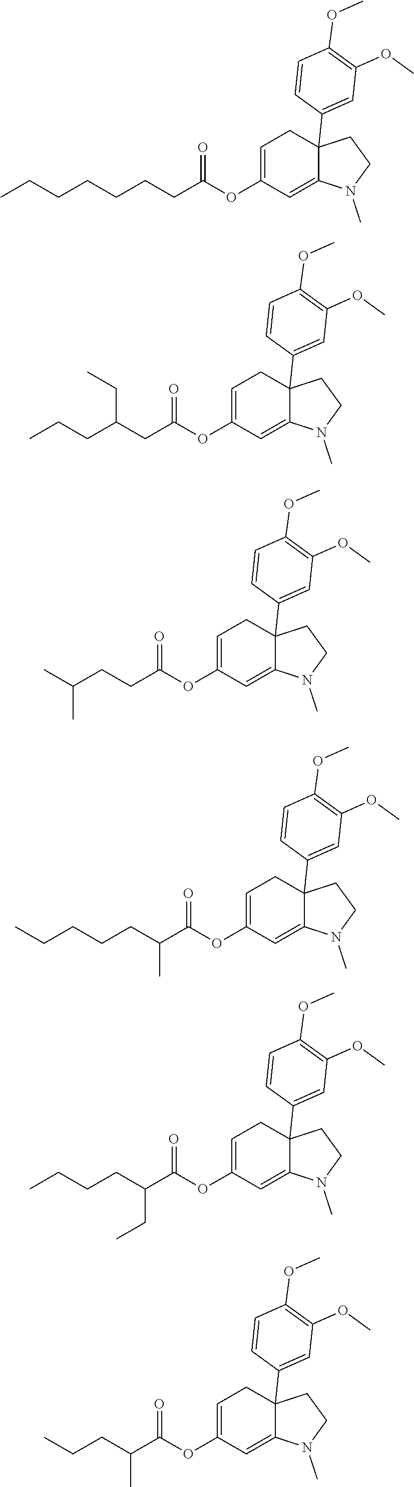

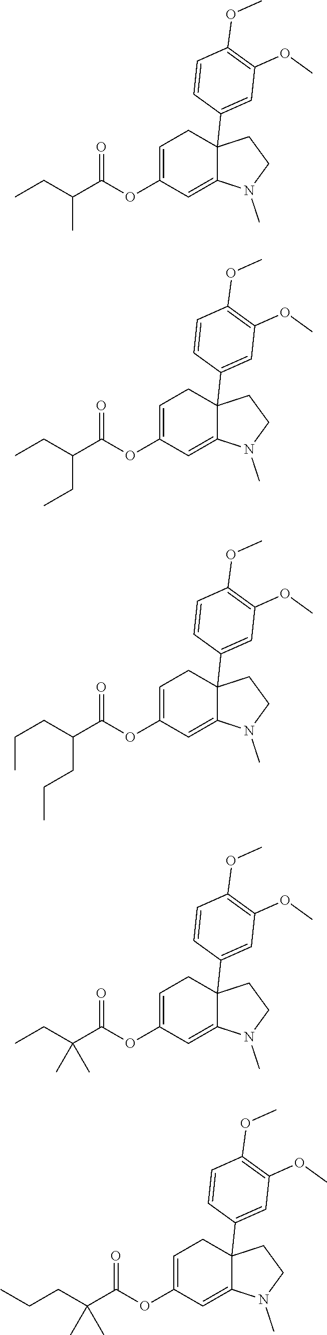

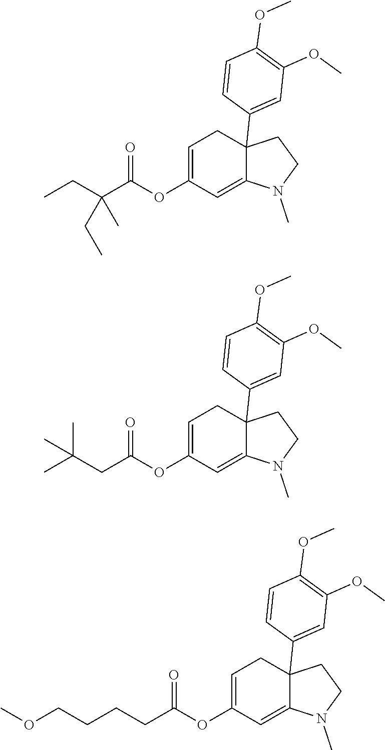

or a pharmaceutically acceptable salt thereof.

57. The compound of embodiment 1, selected from the group consisting of:

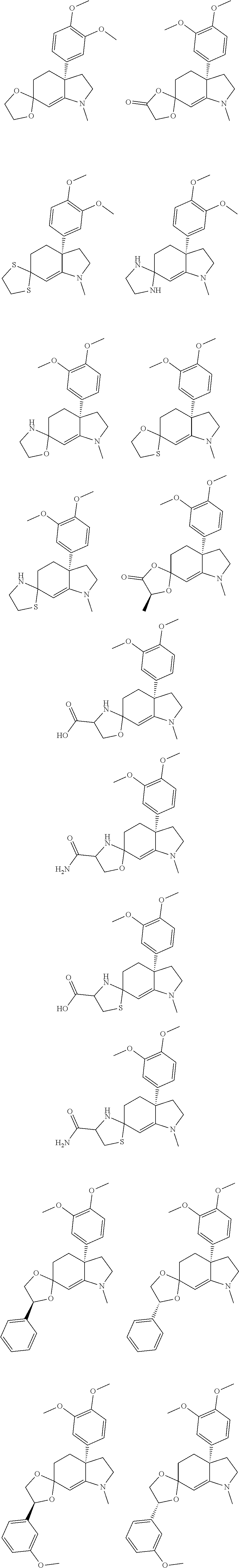

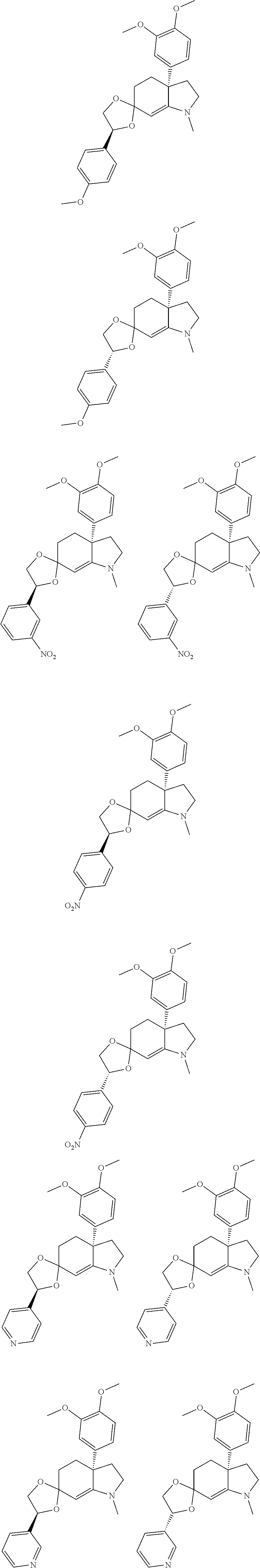

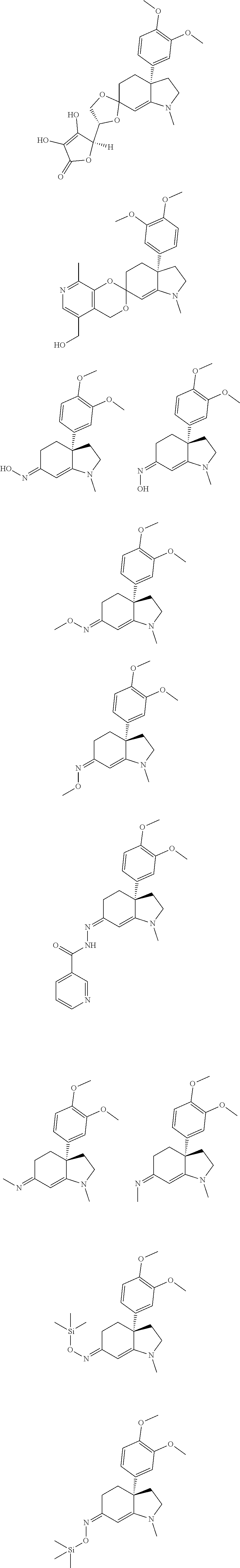

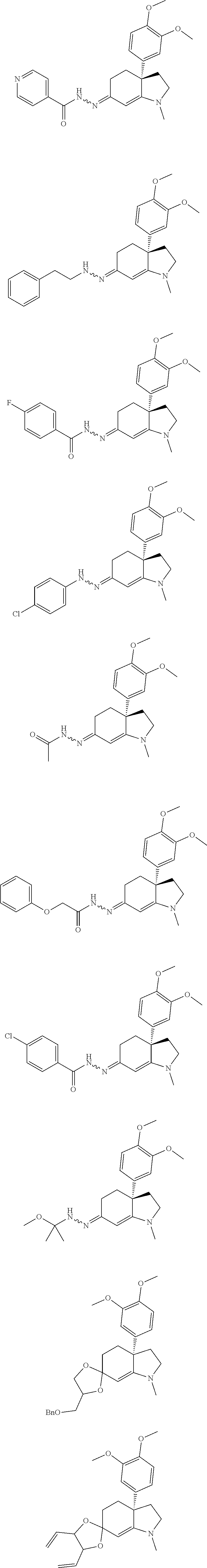

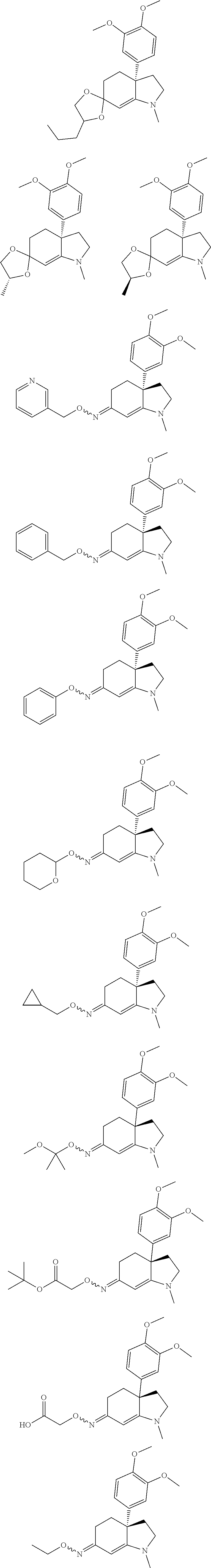

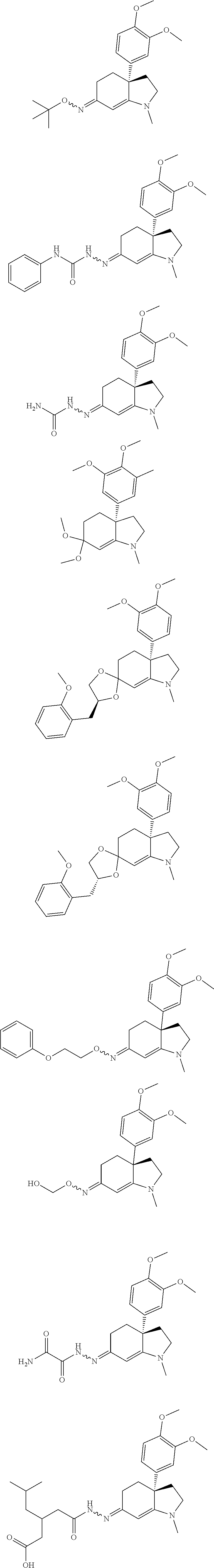

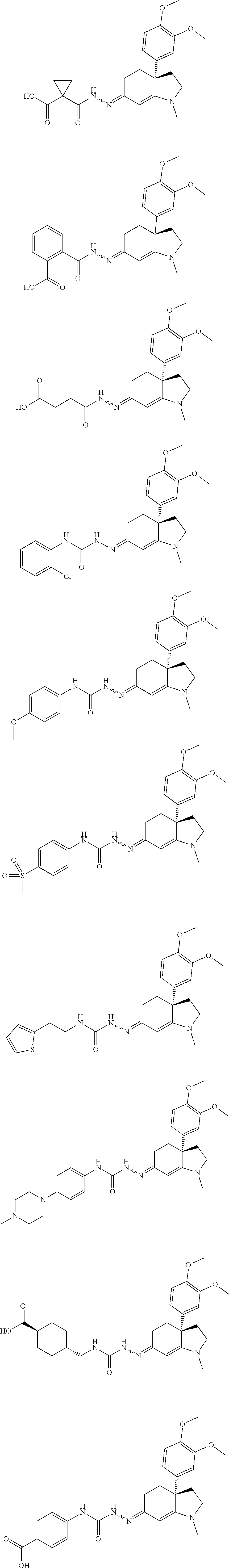

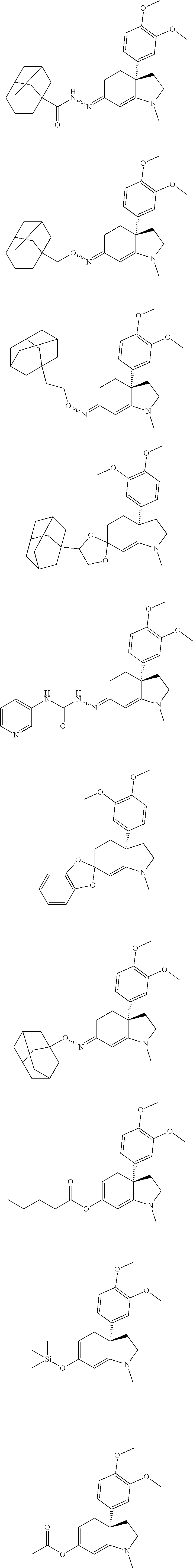

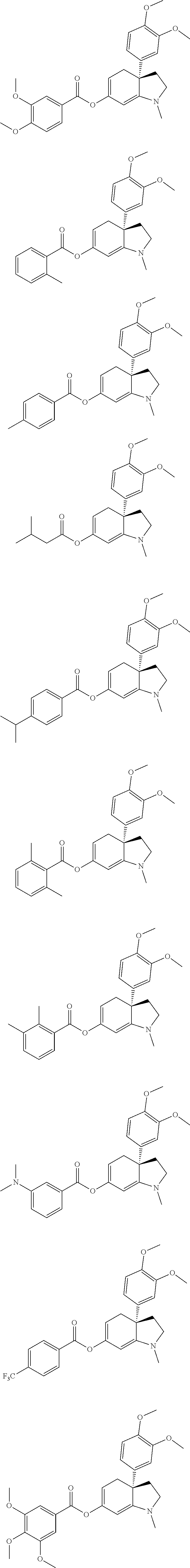

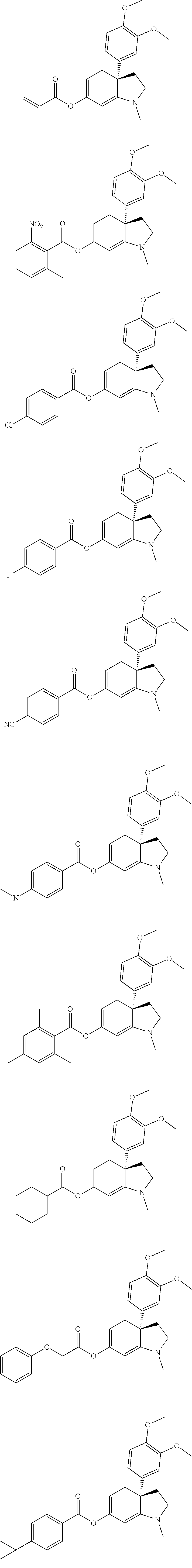

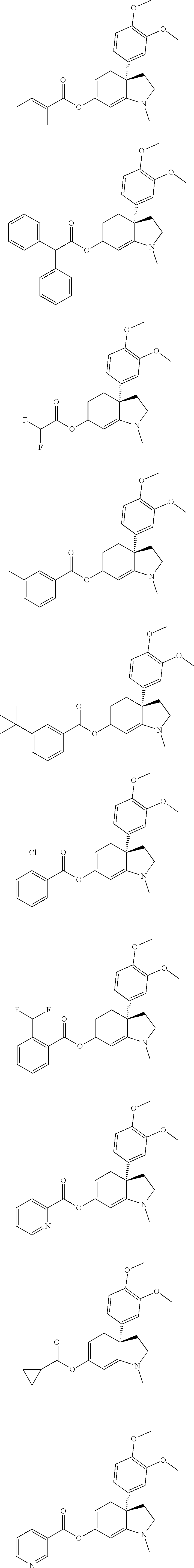

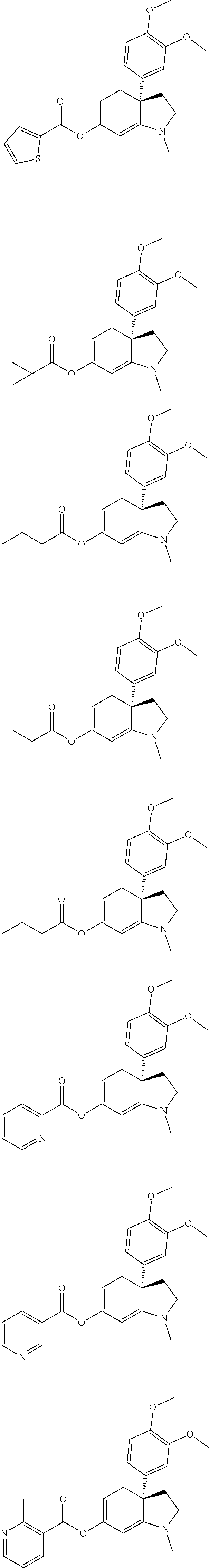

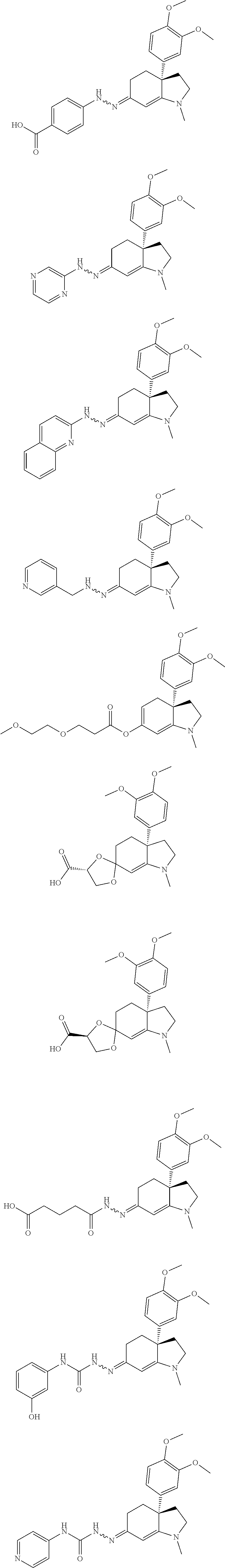

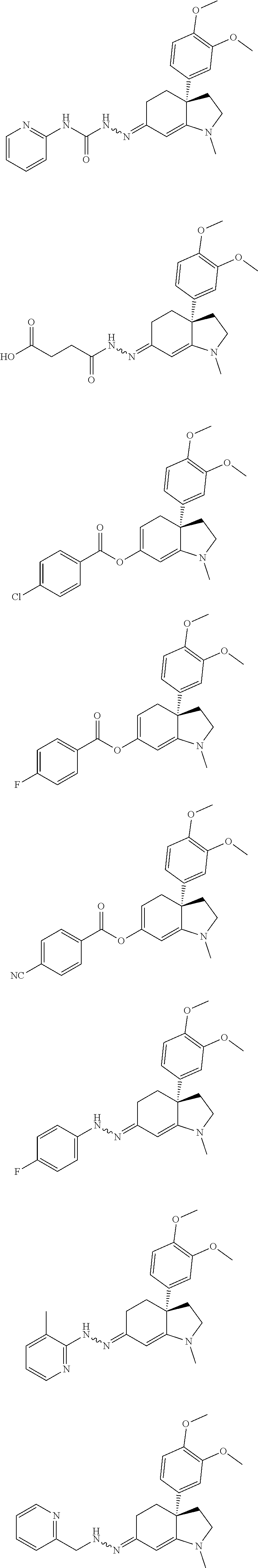

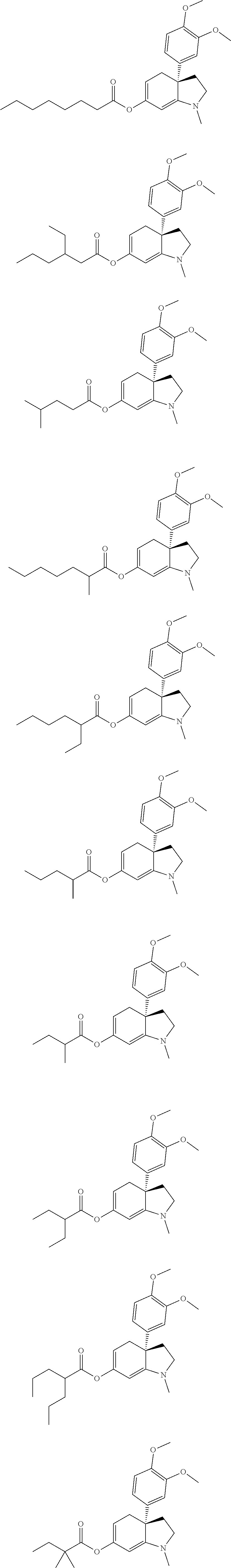

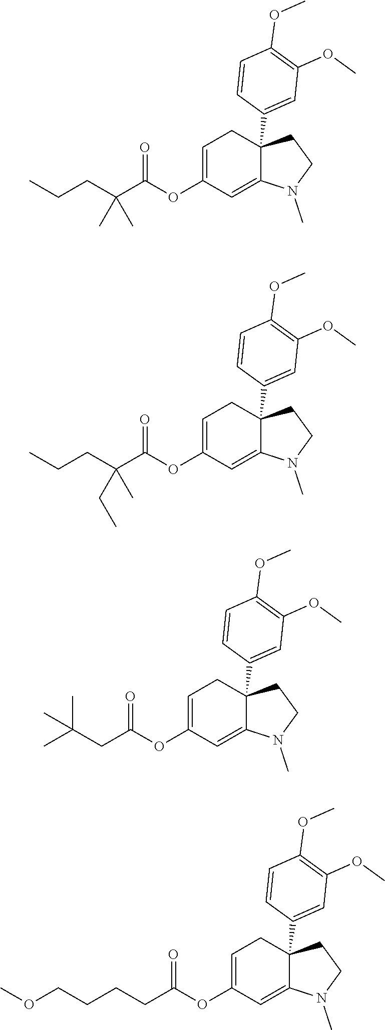

or a pharmaceutically acceptable salt thereof.

58. The compound of embodiment 1, selected from the group consisting of:

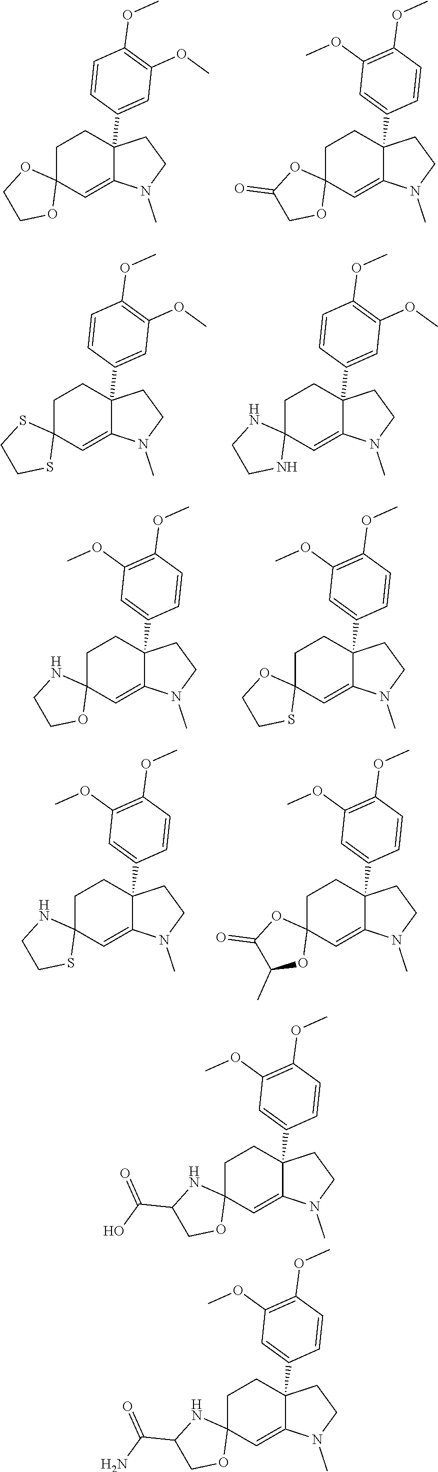

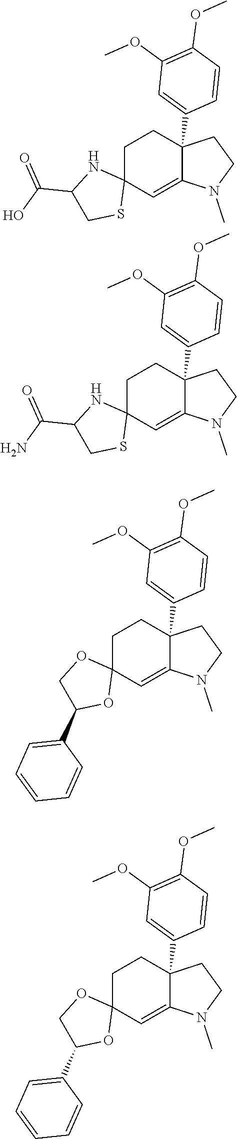

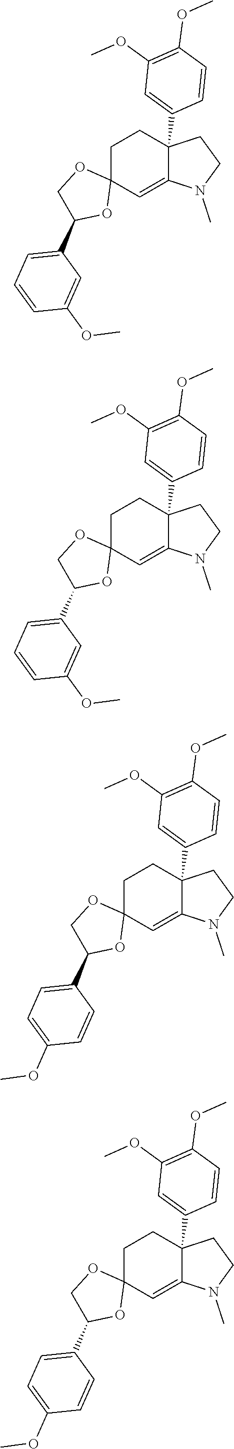 [SMILES: COc1cccc([C@H]2COC3(C=C4N(C)CC[C@]4(c4ccc(OC)c(OC)c4)CC3)O2)c1 ; COc1cccc([C@@H]2COC3(C=C4N(C)CC[C@]4(c4ccc(OC)c(OC)c4)CC3)O2)c1 ; COc1ccc([C@H]2COC3(C=C4N(C)CC[C@]4(c4ccc(OC)c(OC)c4)CC3)O2)cc1 ; COc1ccc([C@@H]2COC3(C=C4N(C)CC[C@]4(c4ccc(OC)c(OC)c4)CC3)O2)cc1]

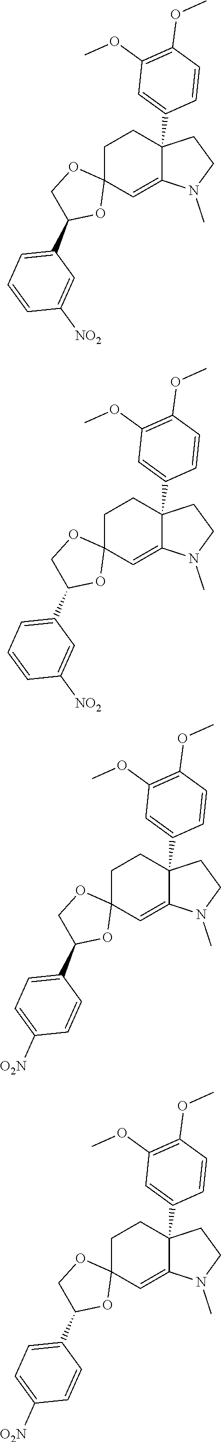 [SMILES: COc1ccc([C@]23CCN(C)C2=CC2(CC3)OC[C@H](c3cccc([N+](=O)[O-])c3)O2)cc1OC ; COc1ccc([C@]23CCN(C)C2=CC2(CC3)OC[C@@H](c3cccc([N+](=O)[O-])c3)O2)cc1OC ; COc1ccc([C@]23CCN(C)C2=CC2(CC3)OC[C@H](c3ccc([N+](=O)[O-])cc3)O2)cc1OC ; COc1ccc([C@]23CCN(C)C2=CC2(CC3)OC[C@@H](c3ccc([N+](=O)[O-])cc3)O2)cc1OC]

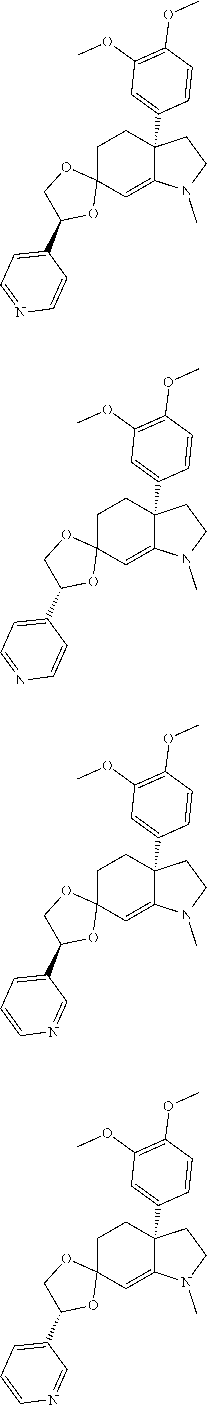 [SMILES: COc1ccc([C@]23CCN(C)C2=CC2(CC3)OC[C@H](c3ccncc3)O2)cc1OC ; COc1ccc([C@]23CCN(C)C2=CC2(CC3)OC[C@@H](c3ccncc3)O2)cc1OC ; COc1ccc([C@]23CCN(C)C2=CC2(CC3)OC[C@H](c3cccnc3)O2)cc1OC ; COc1ccc([C@]23CCN(C)C2=CC2(CC3)OC[C@@H](c3cccnc3)O2)cc1OC]

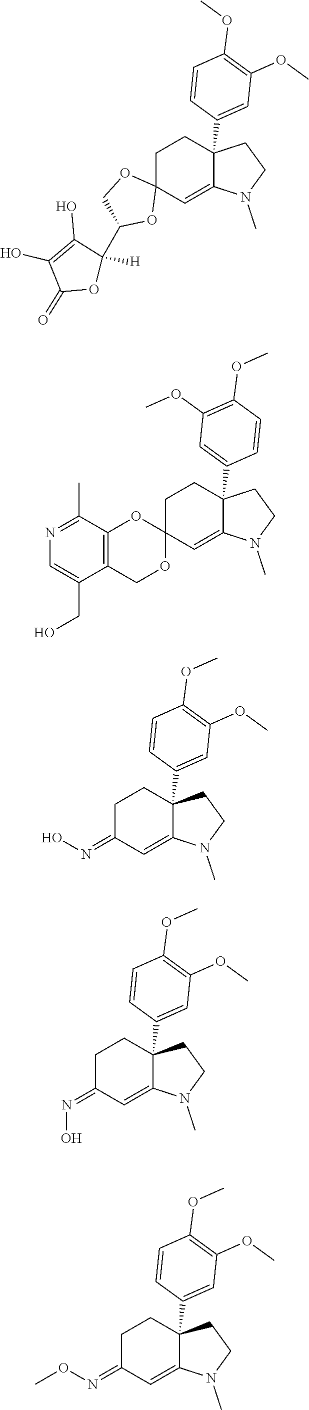

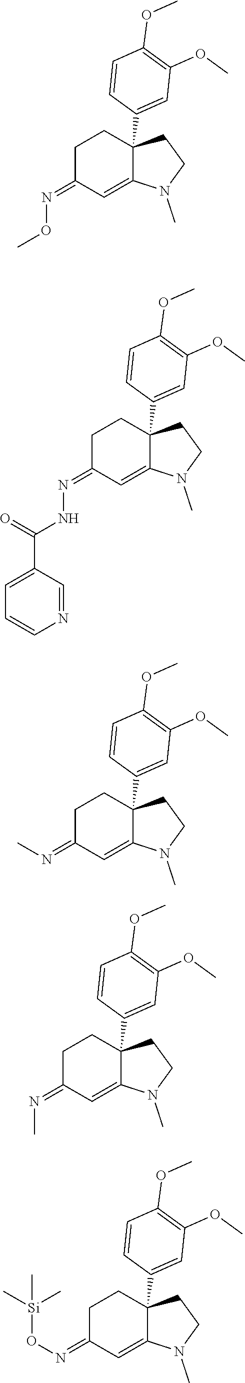

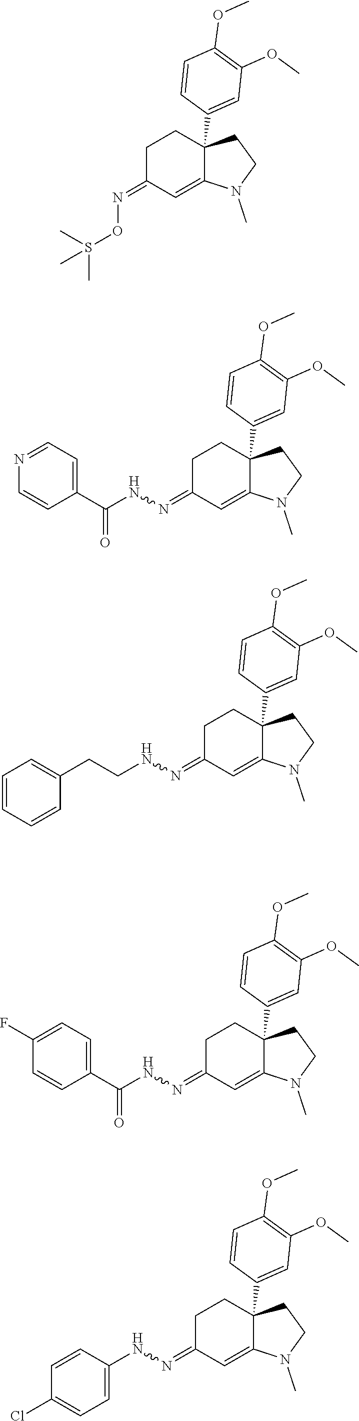

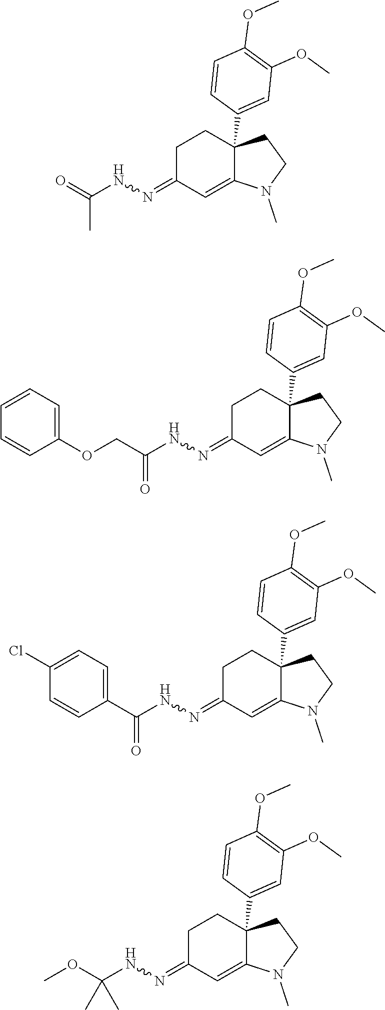

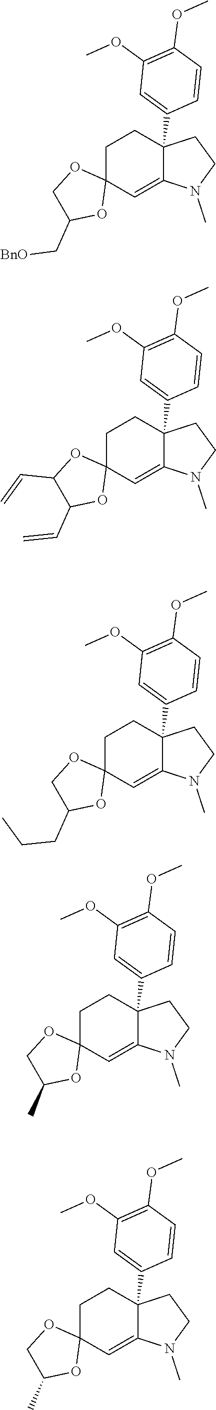 [SMILES: COc1ccc([C@]23CCN(C)C2=CC2(CC3)OCC(COCc3ccccc3)O2)cc1OC ; C=CC1OC2(C=C3N(C)CC[C@]3(c3ccc(OC)c(OC)c3)CC2)OC1C=C ; CCCC1COC2(C=C3N(C)CC[C@]3(c3ccc(OC)c(OC)c3)CC2)O1 ; COc1ccc([C@]23CCN(C)C2=CC2(CC3)OC[C@H](C)O2)cc1OC ; COc1ccc([C@]23CCN(C)C2=CC2(CC3)OC[C@@H](C)O2)cc1OC]

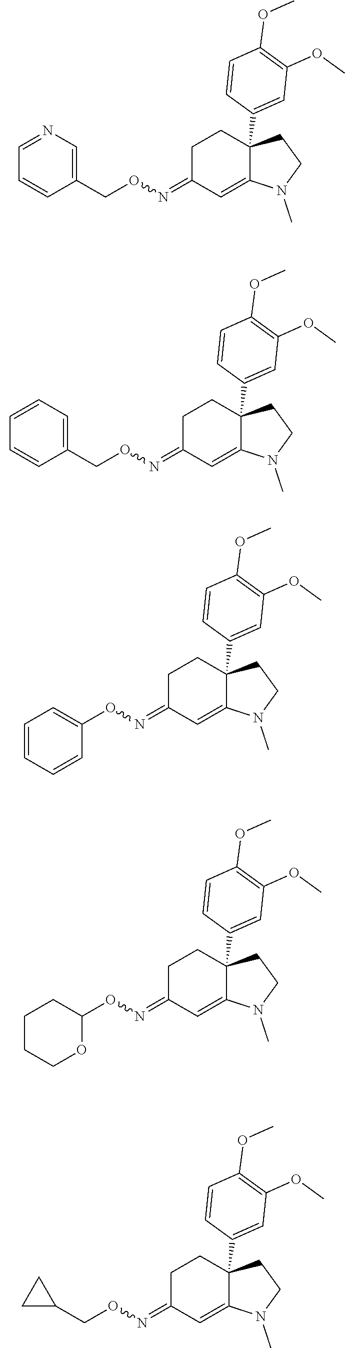

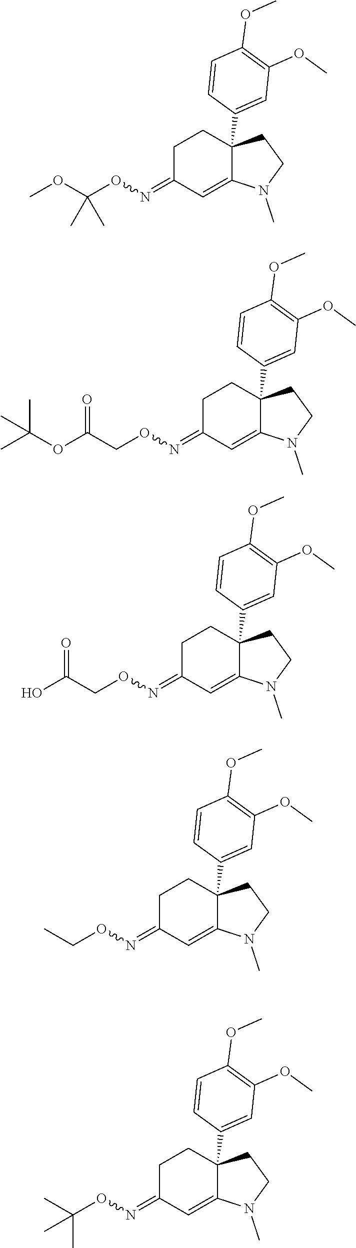

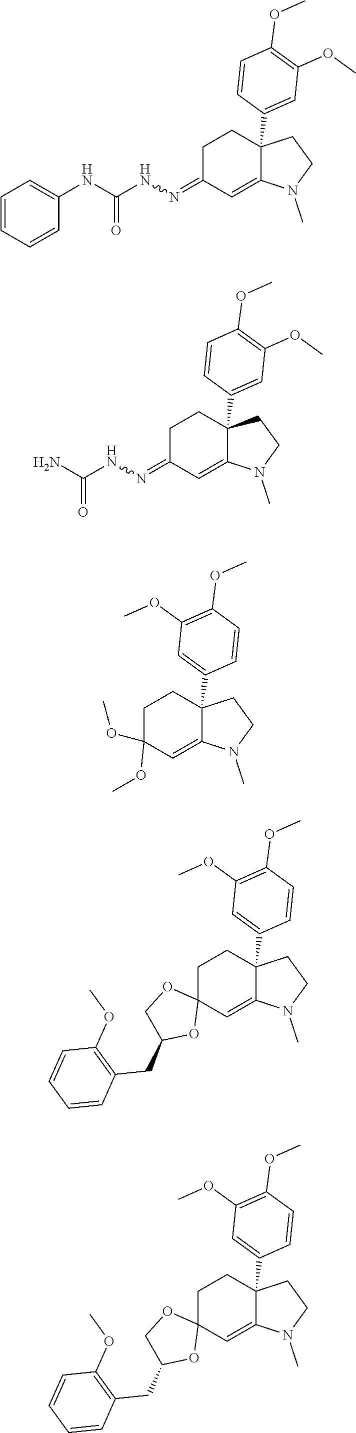

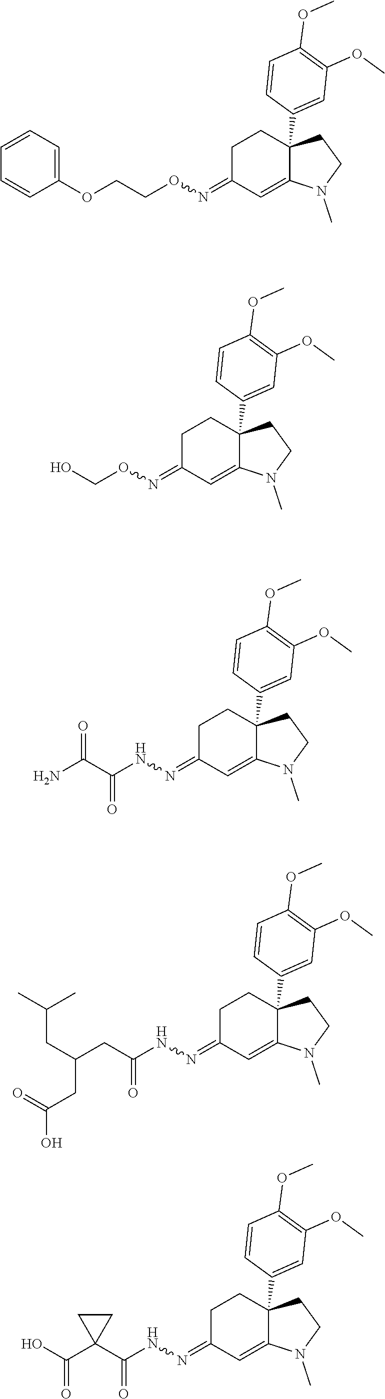 [SMILES: COc1ccc([C@@]23CCC(=NOCCOc4ccccc4)C=C2N(C)CC3)cc1OC ; COc1ccc([C@@]23CCC(=NOCO)C=C2N(C)CC3)cc1OC ; COc1ccc([C@@]23CCC(=NNC(=O)C(N)=O)C=C2N(C)CC3)cc1OC ; COc1ccc([C@@]23CCC(=NNC(=O)CC(CC(=O)O)CC(C)C)C=C2N(C)CC3)cc1OC ; COc1ccc([C@@]23CCC(=NNC(=O)C4(C(=O)O)CC4)C=C2N(C)CC3)cc1OC]

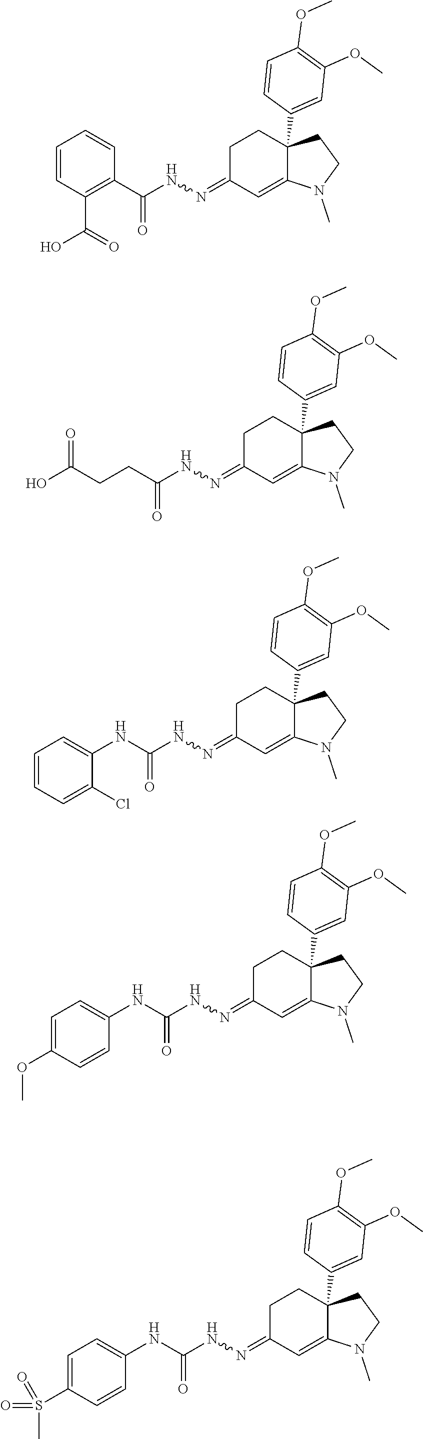

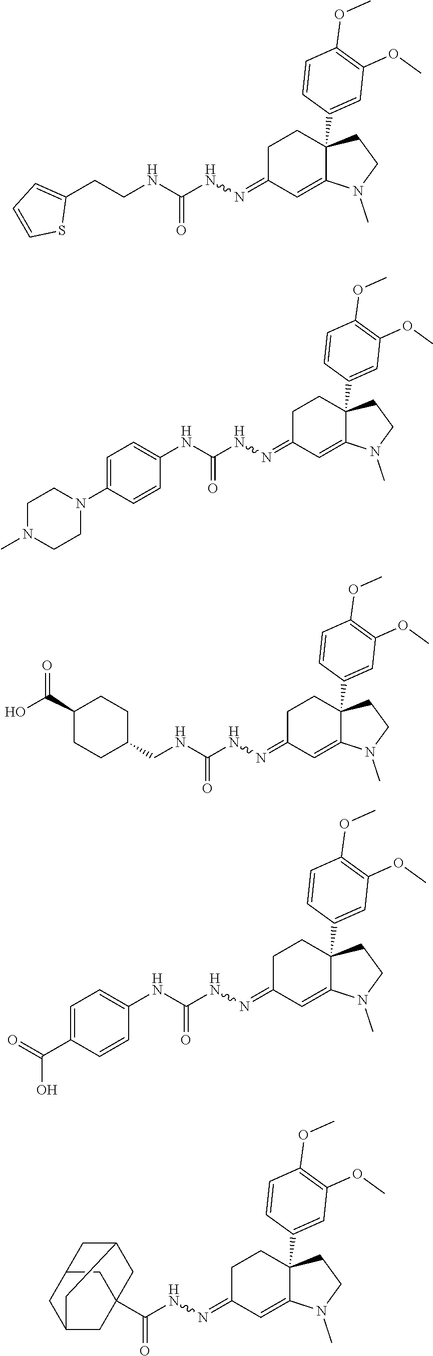

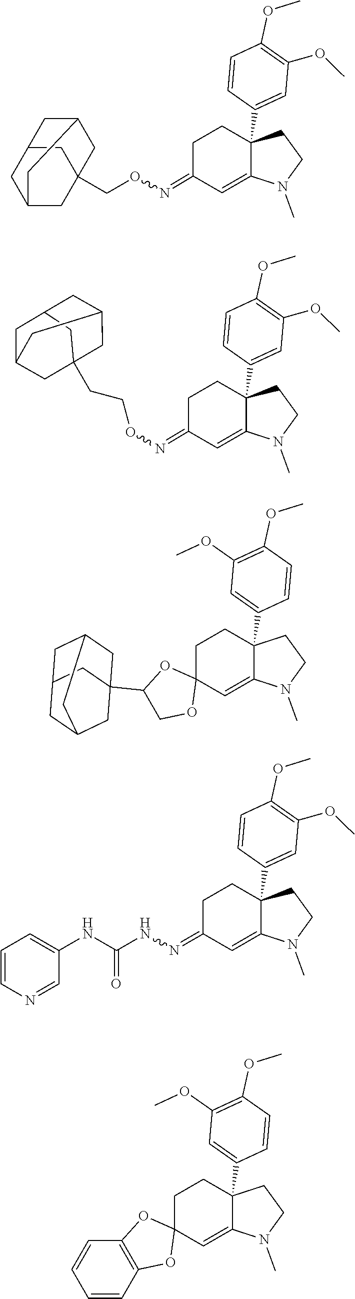

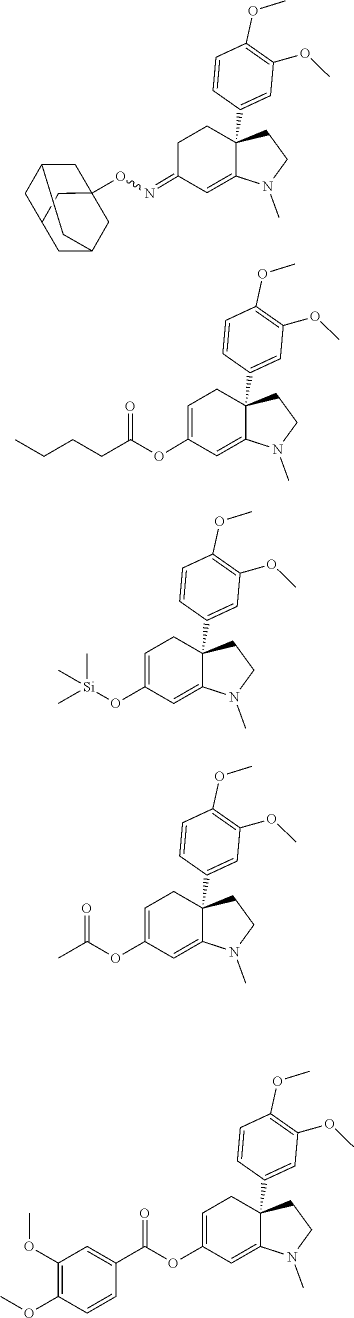

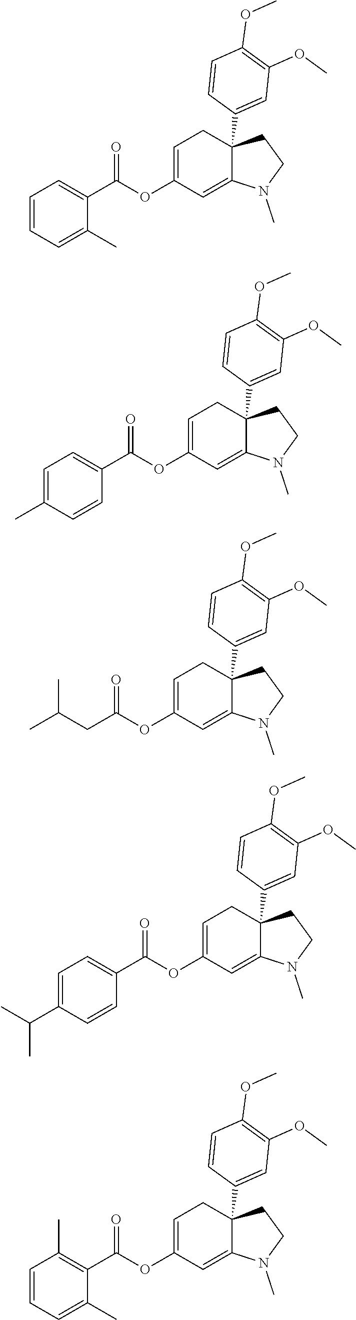

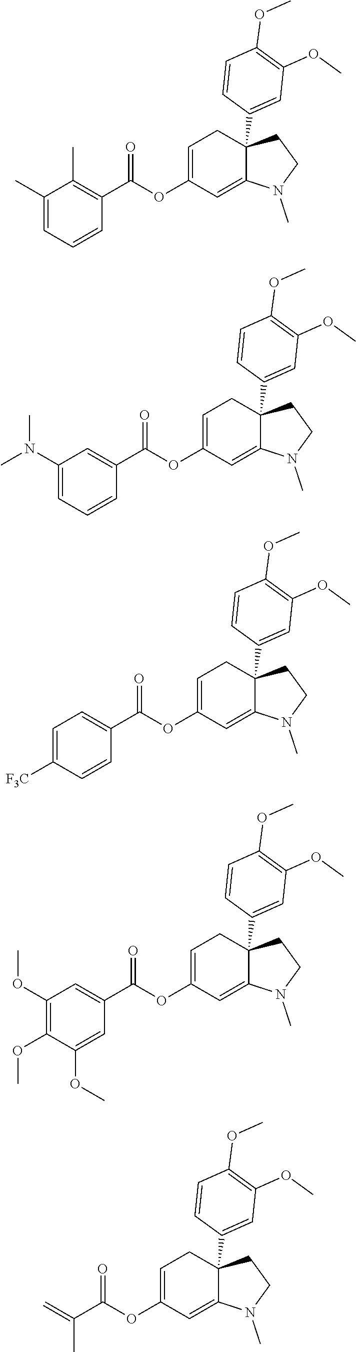

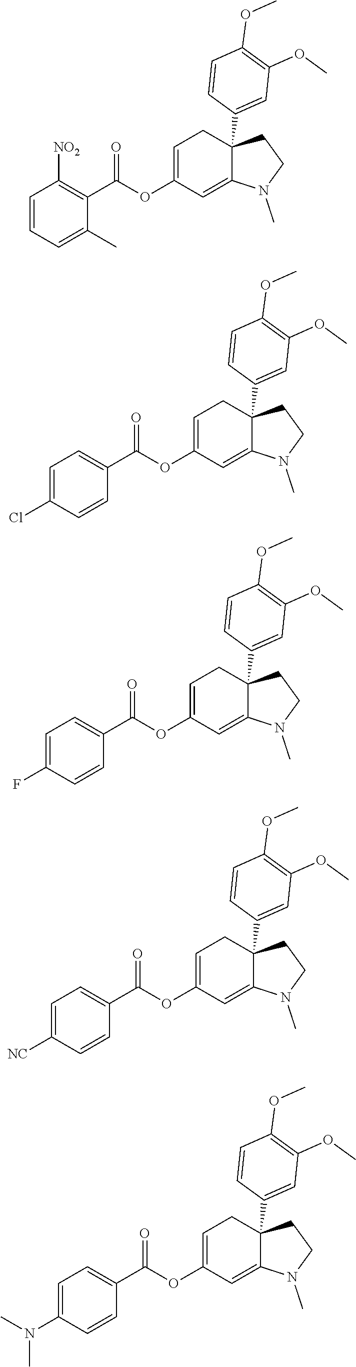

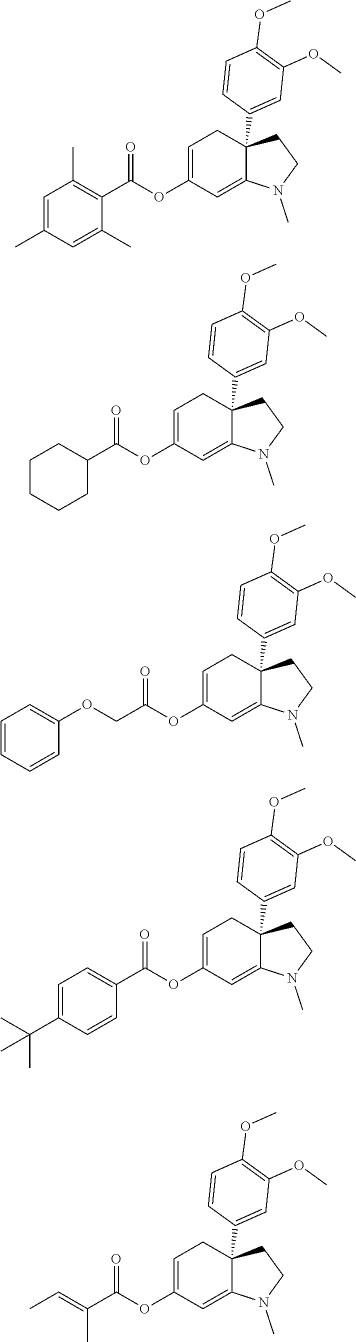

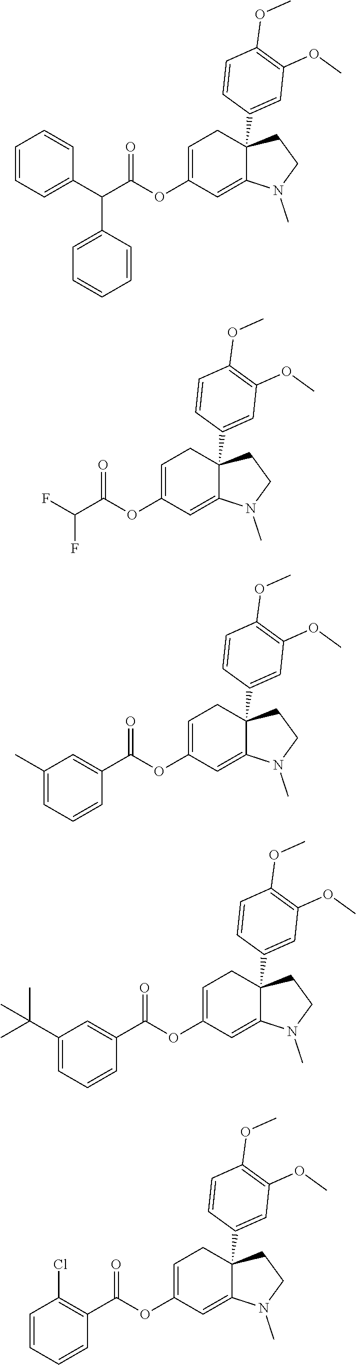

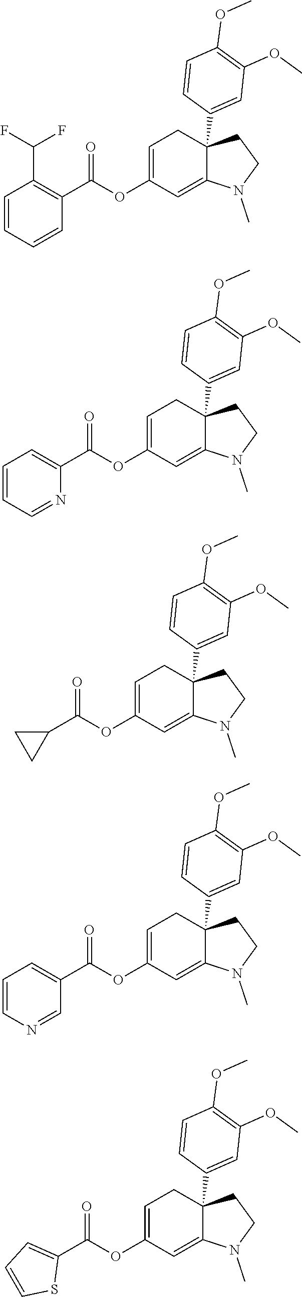

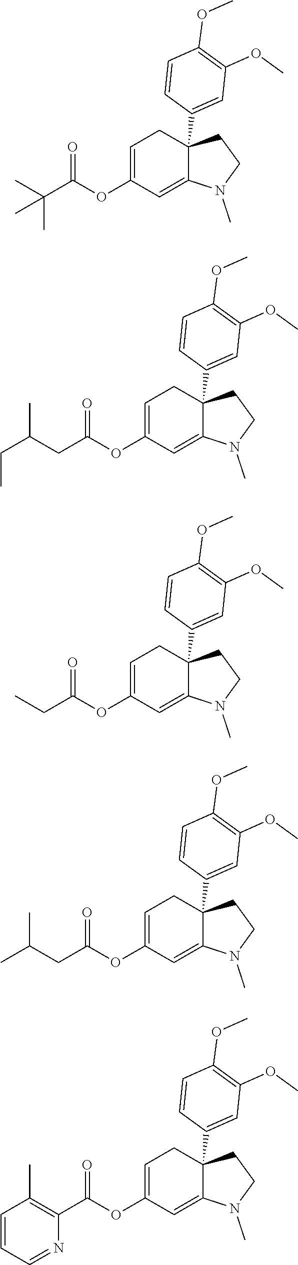

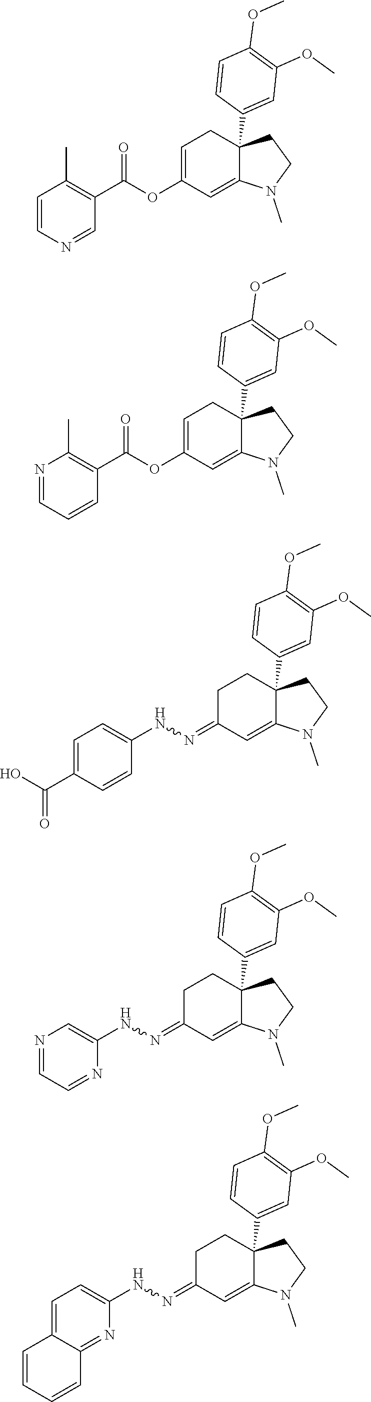

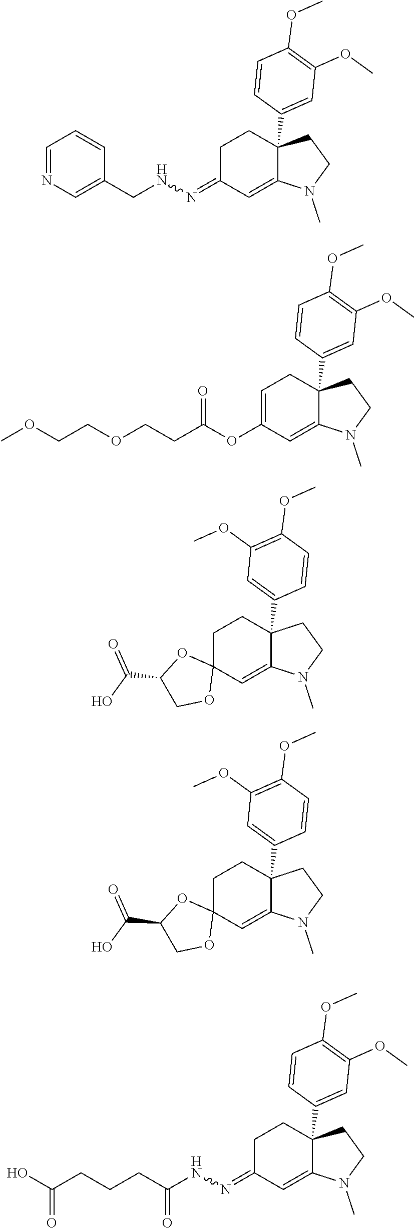

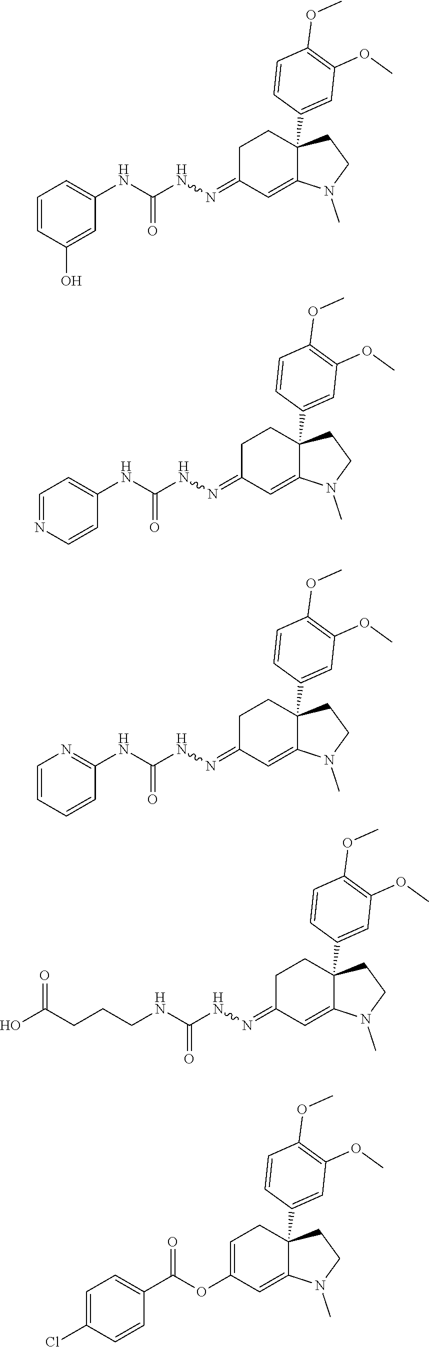 [SMILES: COc1ccc([C@@]23CCC(=NNC(=O)Nc4cccc(O)c4)C=C2N(C)CC3)cc1OC ; COc1ccc([C@@]23CCC(=NNC(=O)Nc4ccncc4)C=C2N(C)CC3)cc1OC ; COc1ccc([C@@]23CCC(=NNC(=O)Nc4ccccn4)C=C2N(C)CC3)cc1OC ; COc1ccc([C@@]23CCC(=NNC(=O)NCCCC(=O)O)C=C2N(C)CC3)cc1OC ; COc1ccc([C@@]23CC=C(OC(=O)c4ccc(Cl)cc4)C=C2N(C)CC3)cc1OC]

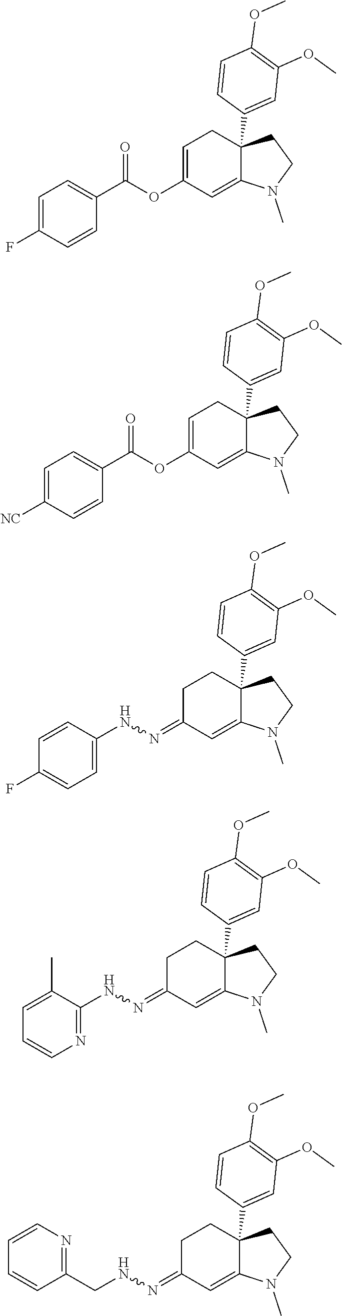 [SMILES: COc1ccc([C@@]23CC=C(OC(=O)c4ccc(F)cc4)C=C2N(C)CC3)cc1OC ; COc1ccc([C@@]23CC=C(OC(=O)c4ccc(C#N)cc4)C=C2N(C)CC3)cc1OC ; COc1ccc([C@@]23CCC(=NNc4ccc(F)cc4)C=C2N(C)CC3)cc1OC ; COc1ccc([C@@]23CCC(=NNc4ncccc4C)C=C2N(C)CC3)cc1OC ; COc1ccc([C@@]23CCC(=NNCc4ccccn4)C=C2N(C)CC3)cc1OC]

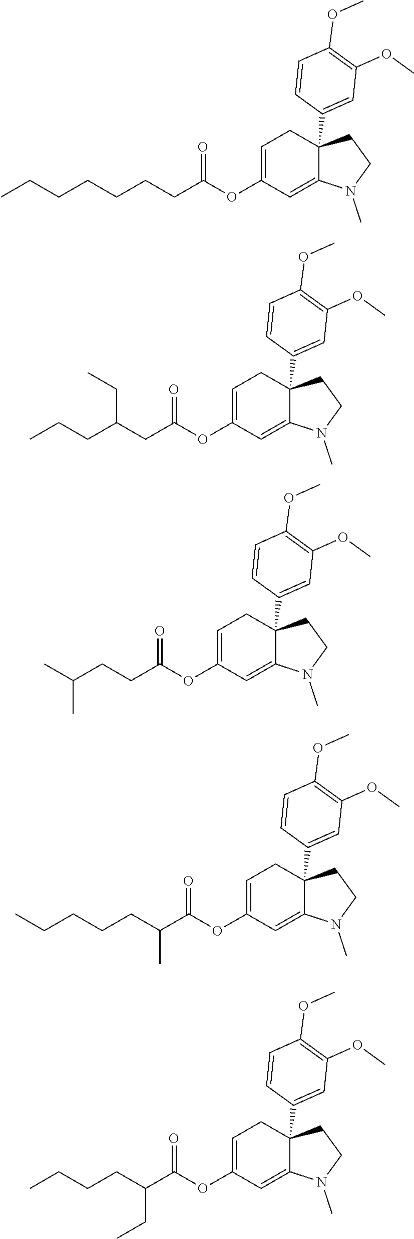

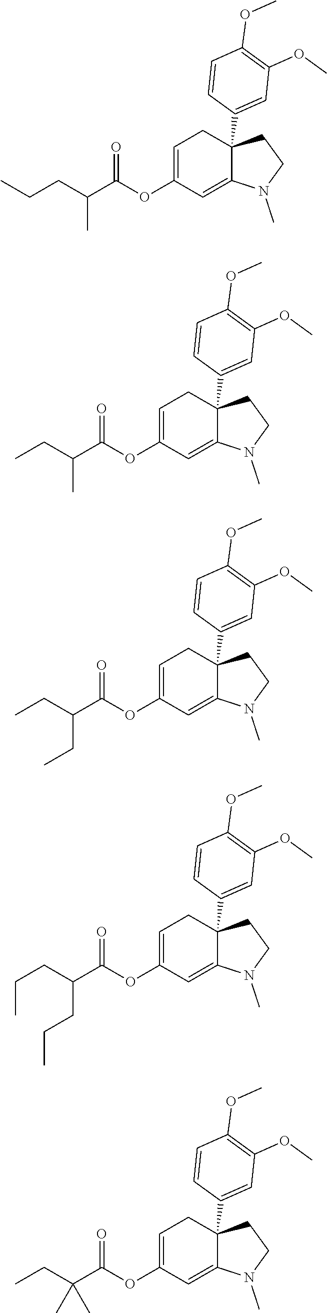

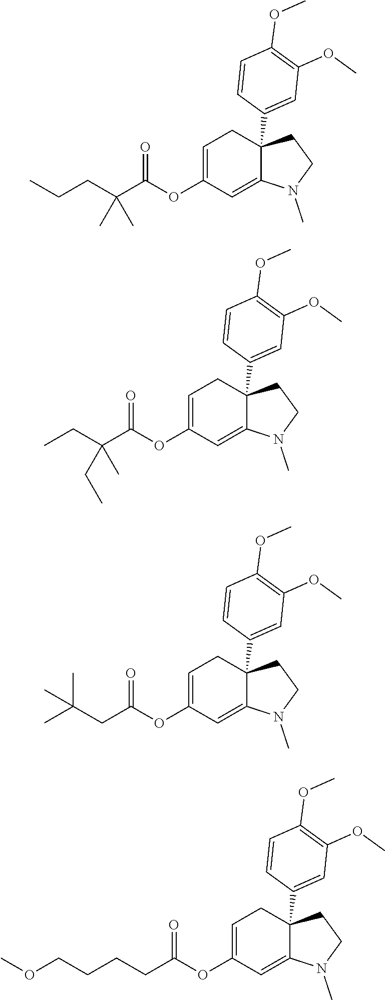

or a pharmaceutically acceptable salt thereof; wherein the compound has the absolute stereochemistry shown.

59. A compound of Formula (IA):

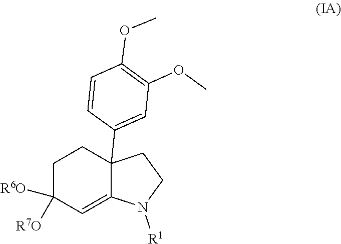

wherein R¹, R⁶, and R⁷ are as defined in embodiment 1,
or a pharmaceutically acceptable salt thereof.

60. A compound of formula (I)

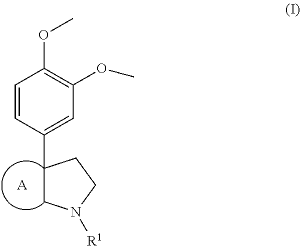

or a pharmaceutically acceptable salt thereof, wherein
R¹ is methyl; and
ring A is

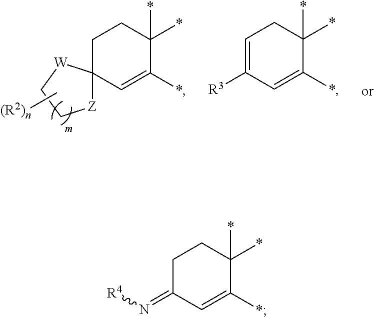

wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, C₃-C₁₀ cycloalkyl, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl or phenyl, wherein each hydrogen atom in 5- to 7-membered heteroaryl or phenyl is optionally substituted by —OH, C₁-C₃ alkyl, or C₁-C₃ alkanol;
m is 1 or 2;
n is 0, 1, or 2;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₃ alkyl, C₁-C₃ alkoxy, nitro, —N(C₁-C₃ alkyl)₂, C₁-C₃ haloalkyl, cyano, phenyl, or phenoxy;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, —C(O)-5- to 7-membered heteroaryl, —C(O)—C₁-C₈ alkyl, —C(O)—C₃-C₁₀ cycloalkyl, —C(O)— phenyl, —C(O)N(H)-phenyl, C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl.

61. A compound of formula (IIa):

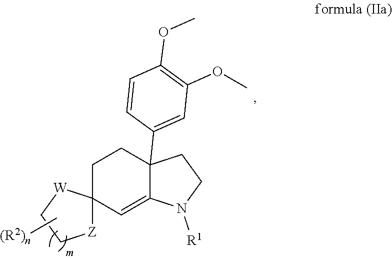

or a pharmaceutically acceptable salt thereof, wherein:
R¹ is methyl;
each of W and Z is independently O, NH, or S;
each R² is independently C₁-C₃ alkyl, —COOH, —CONH₂, phenyl, 5- to 7-membered heterocyclyl, or 5- to 7-membered heteroaryl, wherein each hydrogen atom in phenyl, 5- to 7-membered heterocyclyl and 5- to 7-membered heteroaryl is optionally substituted by —OH, C₁-C₃ alkyl, C₁-C₃ alkoxy, C₁-C₃ alkanol, or —NO₂, or two R⁴s on a single carbon atom combine to form ═O; or two adjacent R²s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol;
m is 1 or 2; and
n is 0, 1, or 2.

62. A compound of formula (IIa):

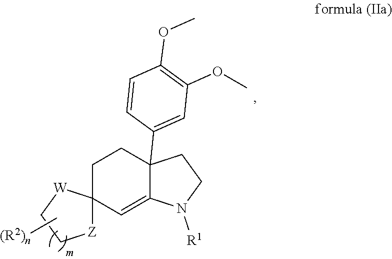

or a pharmaceutically acceptable salt thereof, wherein:
R¹ is methyl;
each of W and Z is independently O, NH, or S;
each R² is independently C₁-C₃ alkyl, C₂-C₆ alkenyl, —COOH, —CONH₂, C₃-C₁₀ cycloalkyl, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl or phenyl, wherein each hydrogen atom in 5- to 7-membered heteroaryl or phenyl is optionally substituted by —OH, C₁-C₃ alkyl, or C₁-C₃ alkanol;
m is 1 or 2; and
n is 0, 1, or 2.

63. A compound of formula (IIIa):

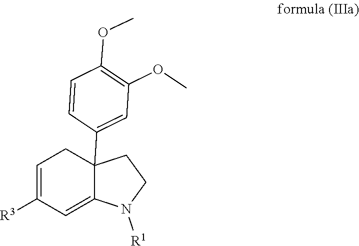

or a pharmaceutically acceptable salt thereof, wherein:
R¹ is methyl; and
R³ is —OSi(C₁-C₆ alkyl)₃ or —OC(O)C₁-C₆ alkyl.

64. A compound of formula (IIIa):

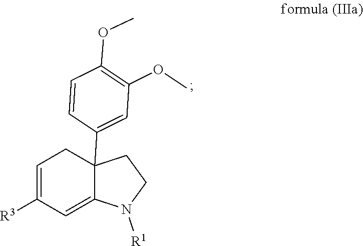

or a pharmaceutically acceptable salt thereof, wherein:
R¹ is methyl; and
R³ is-OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₃ alkyl, C₁-C₃ alkoxy, nitro, —N(C₁-C₃ alkyl)₂, C₁-C₃ haloalkyl, cyano, phenyl, or phenoxy.

65. A compound of formula (IVa):

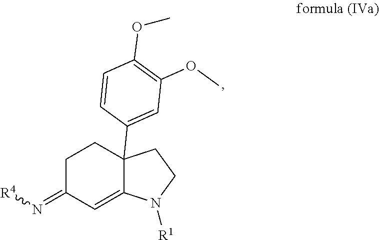

or a pharmaceutically acceptable salt thereof, wherein:
R¹ is methyl; and
R⁴ is OH, C₁-C₆ alkoxy, or —NHC(O)-5- to 7-membered heteroaryl, wherein each hydrogen atom in 5- to 7-membered heteroaryl is optionally substituted by —OH, C₁-C₃ alkyl, or C₁-C₃ alkanol.

66. A compound of formula (IVa):

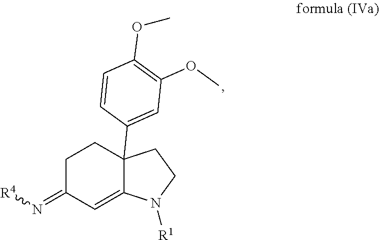

or a pharmaceutically acceptable salt thereof, wherein:
R¹ is methyl;
R⁴ is —OR⁵ or —N(R⁵)₂; and
each R⁵ is H, C₁-C₆ alkyl, C₃-C₆ heterocycloalkyl, C₃-C₁₀ cycloalkyl, C₁-C₆ alkoxy, phenyl, —C(O)-5- to 7-membered heteroaryl, —C(O)—C₁-C₈ alkyl, —C(O)—C₃-C₁₀ cycloalkyl, —C(O)— phenyl, —C(O)N(H)-phenyl, C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl.

67. A compound of formula (IIa-1):

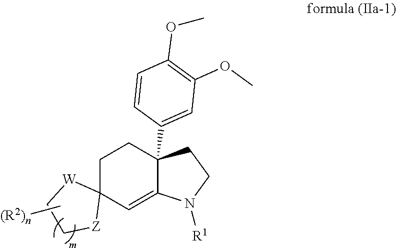

or a pharmaceutically acceptable salt thereof, wherein:
R¹ is methyl;
each of W and Z is independently O, NH, or S;
each R² is independently C₁-C₃ alkyl, —COOH, —CONH₂, phenyl, 5- to 7-membered heterocyclyl, or 5- to 7-membered heteroaryl, wherein each hydrogen atom in phenyl, 5- to 7-membered heterocyclyl and 5- to 7-membered heteroaryl is optionally substituted by —OH, C₁-C₃ alkyl, C₁-C₃ alkoxy, C₁-C₃ alkanol, or —NO₂, or two R⁴s on a single carbon atom combine to form ═O; or two adjacent R⁴s together with the carbon atoms to which they are attached combine to form 5- to 7-membered heteroaryl, wherein each hydrogen atom in 5- to 7-membered heteroaryl is optionally substituted by —OH, C₁-C₃ alkyl, or C₁-C₃ alkanol;
m is 1 or 2;
n is 0, 1, or 2; and
the compounds of formula (IIa-1) have the absolute stereochemistry shown.

68. A compound of formula (IIa-1):

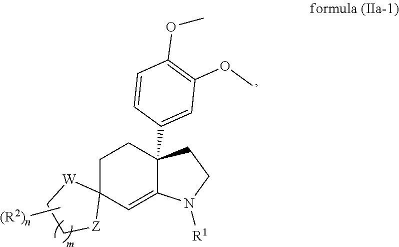

or a pharmaceutically acceptable salt thereof, wherein:
R¹ is methyl;
each of W and Z is independently O, NH, or S;
each R² is independently C₁-C₃ alkyl, C₂-C₆ alkenyl, —COOH, —CONH₂, C₃-C₁₀ cycloalkyl, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl or phenyl, wherein each hydrogen atom in 5- to 7-membered heteroaryl or phenyl is optionally substituted by —OH, C₁-C₃ alkyl, or C₁-C₃ alkanol;
m is 1 or 2;
n is 0, 1, or 2; and
the compounds of formula (IIa-1) have the absolute stereochemistry shown.

69. A compound of formula (IIIa-1):

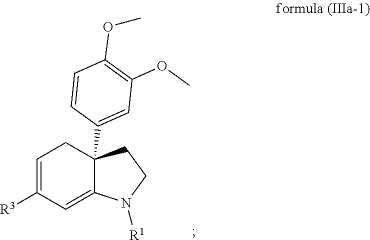

or a pharmaceutically acceptable salt thereof, wherein:
R¹ is methyl;
R³ is —OSi(C₁-C₆ alkyl)₃ or —OC(O)C₁-C₆ alkyl; and
the compounds of formula (IIIa-1) have the absolute stereochemistry shown.

70. A compound of formula (IIIa-1):

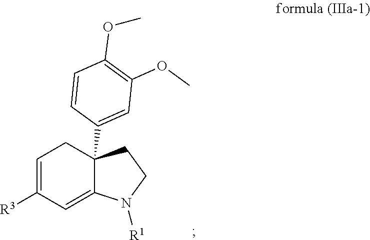

or a pharmaceutically acceptable salt thereof, wherein:
R¹ is methyl;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, —OC(O)-5- to 7-membered heteroaryl, wherein each hydrogen atom in C₁-C₆ alkyl, C₂-C₆ alkenyl, C₃-C₁₀ cycloalkyl, phenyl, and 5- to 7-membered heteroaryl is optionally substituted by halogen, C₁-C₃ alkyl, C₁-C₃ alkoxy, nitro, —N(C₁-C₃ alkyl)₂, C₁-C₃ haloalkyl, cyano, phenyl, or phenoxy; and
the compounds of formula (IIIa-1) have the absolute stereochemistry shown.

71. A compound of formula (IVa-1):

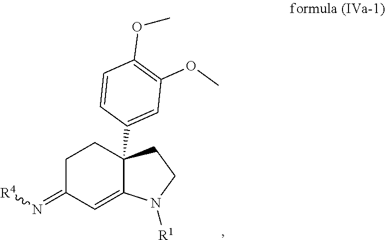

or a pharmaceutically acceptable salt thereof, wherein:
R¹ is methyl;
R⁴ is OH, C₁-C₆ alkoxy, or —NHC(O)-5- to 7-membered heteroaryl, wherein each hydrogen atom in 5- to 7-membered heteroaryl is optionally substituted by —OH, C₁-C₃ alkyl, or C₁-C₃ alkanol; and
the compounds of formula (IVa-1) have the absolute stereochemistry shown.

72. A compound of formula (IVa-1):

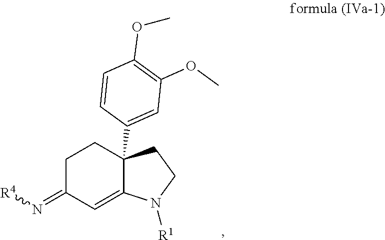

or a pharmaceutically acceptable salt thereof, wherein:
R¹ is methyl;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, —C(O)-5- to 7-membered heteroaryl, —C(O)—C₁-C₈ alkyl, —C(O)—C₃-C₁₀ cycloalkyl, —C(O)— phenyl, —C(O)N(H)-phenyl, C(O)N(H)-5- to 7-membered heteroaryl, —C(O)N(H)—C₁-C₆ alkyl, —C(O)C(O)OH, or —CONH₂, wherein each hydrogen atom in C₁-C₆ alkyl, C₃-C₆ heterocycloalkyl, C₃-C₁₀ cycloalkyl, C₁-C₆ alkoxy, phenyl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and
the compounds of formula (IVa-1) has the absolute stereochemistry shown.

73. A pharmaceutical composition, comprising a compound of any one of embodiments 1-72; and a pharmaceutically acceptable excipient.

74. A method of treating a mental health disorder, comprising administering to a mammal in need thereof an effective amount of a compound according to any one of embodiments 1-72.

75. The method of embodiment 74, wherein the mental health disorder is anxiety, stress, or depression.

76. The method of embodiment 74, wherein the mental health disorder is anxiety. 77. The method of embodiment 74, wherein the mental health disorder is stress. 78. The method of embodiment 74, wherein the mental health disorder is depression. 79. The method of any one of embodiments 74-78, wherein the mammal is a human.

### EXAMPLES

### Example 1: Synthesis of Δ⁷-Mesembrenone

Δ7-Mesembrenone can be prepared according to a process known in the literature (Takano et al. Tet. Lett. 1981, 22, 4479-4482), incorporated herein by reference in its entirety.

A total synthesis of Δ7-Mesembrenone is provided below, from Gallagher-Duval et al., “Functionalized Polyhydroquinolines from Amino Acids Using a Key One-Pot Cyclization Cascade and Application to the Synthesis of (i)-Δ7-Mesembrenone,” Org. Lett. 2021, 23, 21, 8606-8611 (Oct. 25, 2021), incorporated herein by reference in its entirety, including the chemical synthesis below.

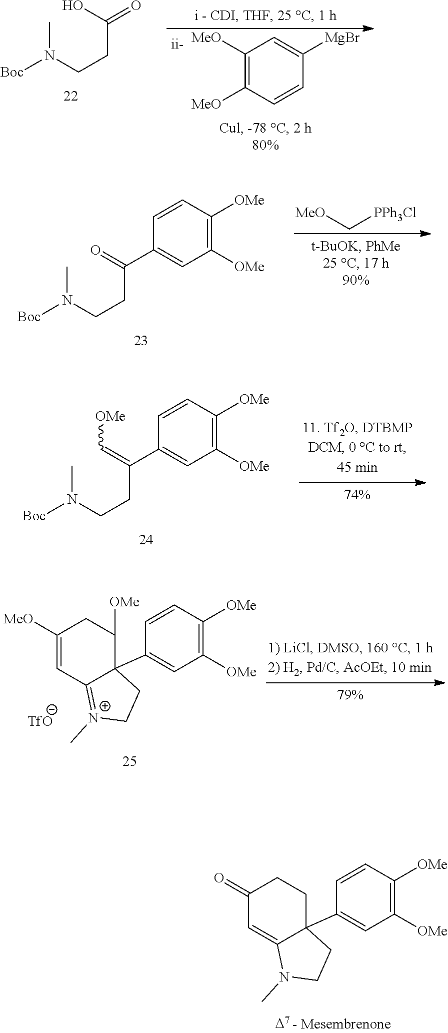

tert-Butyl N-(4-hydroxy-5-methylhexyl)-N-methylcarbamate (43). Following General Procedure A, a solution of 40 (2.03 g, 10.0 mmol) in THF (60 mL) was added to a suspension of LiAlH₄ (948 mg, 25.0 mmol) in THF (60 mL) followed by water (120 mL), Boc₂O (2.53 mL, 11.0 mmol) and saturated aqueous Rochelle salts (120 mL). The usual workup (Et₂O) afforded crude 41 (1.37 g, 67%) as a light yellow oil. Following General Procedure C, a solution of 41 (1.37 g, 6.73 mmol) in DCM (15 mL) was added to a solution oxalyl chloride (645 μL, 7.41 mmol) and DMSO (770 μL, 13.5 mmol) in DCM (25 mL), followed by triethylamine (4.6 mL, 34 mmol). Saturated aqueous NaHCO₃ (40 mL) was added. The usual workup (Et₂O) afforded crude 42 (1.26 g) as a yellow oil. A small portion was purified (20 to 40% AcOEt in hexanes) for characterization. 42: ¹H NMR (300 MHz, CDCl₃) δ (ppm) 9.79 (t, J=1.2 Hz, 1H), 3.25 (t, J=7.0 Hz, 2H), 2.84 (s, 3H), 2.46 (td, J=7.2, 1.2 Hz, 2H), 1.84 (qu, J=7.2 Hz, 2H), 1.45 (s, 9H). ¹³C NMR (75.5 MHz, CDCl₃) δ (ppm) 201.7 (d), 156.0 (s), 79.7 (s), 47.9 (t), 41.1 (t), 34.3 (q), 28.6 (q), 20.4 (t). IR (neat) ν (cm⁻¹) 2946, 1686, 1395, 1144. HRMS (ESI-TOF) m/z: [M+Na]⁺ Calcd for C₁₀H₁₉NO₃Na 224.1257; Found 224.1251. Isopropylmagnesium chloride (1.95 M in THF) was added to a solution of 42 (215 mg, 107 mmol) in THF (5 mL) at −78° C. The reaction mixture was stirred at −30° C. during 2 h then water was added. The usual workup (EtOAc) and purification (20 to 40% AcOEt in hexanes) afforded 43 (181 mg, 69% over 3 steps) as a colorless oil. 1H NMR (300 MHz, CDCl₃) δ (ppm) 3.30 (ddd, J=8.7, 5.0, 3.7 Hz, 1H), 3.28-3.01 (m, 2H), 2.77 (s, 3H), 2.24 (br s, 1H), 1.74-1.17 (m, 5H), 1.39 (s, 9H), 0.84 (d, J=6.8 H, 6H). ¹³C NMR (75.5 MHz, CDCl₃) as a mixture of rotamers δ (ppm) 156.0 (s), 79.2 (s), 76.3 (d), 49.0 (t) and 48.3 (t), 34.1 (q), 33.8 (d), 31.0 (t) and 30.8 (t), 28.5 (q), 24.4 (t), 18.8 (q) and 18.3 (q). IR (neat) ν (cm⁻¹) 2932, 1672, 1461, 1398, 1365, 1149. HRMS (ESI-TOF) m/z: [M+Na]⁺ Calcd for C₁₃H₂₇NO₃Na 268.1883; Found 268.1888.

tert-Butyl N-methyl-N-(5-methyl-4-oxohexyl)carbamate (44). Following General Procedure C, a solution of 43 (181 mg, 0.74 mmol) in DCM (1 mL) was added to a solution oxalyl chloride (70 μL, 0.81 mmol) and DMSO (159 μL, 1.48 mmol) in DCM (3 mL), followed by triethylamine (513 μL, 3.70 mmol). Saturated aqueous NaHCO₃ (5 mL) was added. The usual workup (DCM) and purification (20 to 25% AcOEt in hexanes) afforded 44 (154 mg, 86%) as a yellow oil. 1H NMR (300 MHz, CDCl₃) δ (ppm) 3.15 (t, J=7.0 Hz, 2H), 2.77 (s, 3H), 2.53 (sept, J=6.9 Hz, 1H), 2.39 (t, J=7.2 Hz, 2H), 1.72 (qu, J=7.1 Hz, 2H), 1.39 (s, 9H), 1.03 (d, J=6.9 Hz, 6H). ¹³C NMR (75.5 MHz, CDCl₃) as a mixture of rotamers δ (ppm) 214.0 (s), 155.9 (s), 79.3 (s), 48.2 (t) and 47.8 (t), 40.9 (d), 37.1 (t), 34.2 (q), 28.5 (q), 21.9 (t) and 21.7 (t), 18.2 (q). IR (neat) ν (cm⁻¹) 2967, 1690, 1391, 164, 1165, 1145. HRMS (ESI-TOF) m/z: [M+Na]⁺ Calcd for C₁₃H₂₅NO₃Na 266.1727; Found 266.1725.

tert-Butyl N-(4-(methoxymethylene)-5-methylhexyl)-N-methylcarbamate (8f). Following General Procedure B, a solution of 44 (154 mg, 0.63 mmol) in THF (2 mL) was added to a solution of n-butyllithium (2.5 M in hexanes, 557 μL, 1.39 mmol) and MeOCH₂PPh₃Cl (454 mg, 1.32 mmol) in THF (5 mL). The usual workup (Et₂O) and purification (5 to 20% Et₂O in hexanes) afforded 8f (79 mg, 46%, 69% based on recovered starting material) as a yellow oil and a 1:1 mixture of E/Z isomers. ¹H NMR (300 MHz, CDCl₃) as a mixture of isomers δ (ppm) 5.75 (s) and 5.64 (s) (1H), 3.51 (s, 3H), 3.24-3.11 (m, 2H), 2.83 (s, 3H), 2.79 (sept, J=7.0 Hz) and 2.17 (sept, J=6.8 Hz) (1H), 2.02-1.92 (m) and 1.85-1.76 (m) (2H), 1.68-1.50 (m, 2H), 1.45 (s) and 1.44 (s) (9H), 0.99 (d, J=7.0 Hz) and 0.97 (d, J=7.0 Hz) (6H). ¹³C NMR (75.5 MHz, CDCl₃) as a mixture of isomers δ (ppm) 156.0 (s), 141.8 (d) and 141.7 (d), 123.2 (s), 79.3 (s) and 79.1 (s), 59.5 (q) and 59.3 (q), 49.1 (t) and 48.7 (t), 34.3 (q), 30.7 (q) and 27.1 (q), 28.6 (q) and 27.1 (q), 27.8 (t) and 27.4 (t), 26.2 (t) and 23.6 (t), 22.2 (q) and 21.2. IR (neat) ν (cm⁻¹) 2966, 2930, 1696, 1395, 1170. HRMS (ESI-TOF) m/z: [M+Na]⁺ Calcd for C₁₅H₂₉NO₃Na 294.2040; Found 294.2042.

### Example 2: Synthesis of Compounds of Formula IIa

Compounds of Formula (IIa) could be prepared by treatment of a ketone, such as Δ7-mesembrenone, with an appropriate glycol derivative, and an acid catalyst, such as p-toluenesulfonic acid or methanesulfonic acid or boron trifloride etherate, in a solvent, such as toluene, at an elevated temperature.

TABLE 11
Exemplary Compounds of Formula IIa

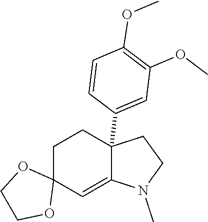 [SMILES: COc1ccc([C@]23CCN(C)C2=CC2(CC3)OCCO2)cc1OC]

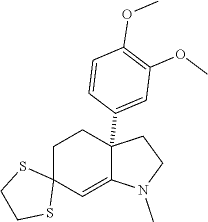 [SMILES: COc1ccc([C@]23CCN(C)C2=CC2(CC3)SCCS2)cc1OC]

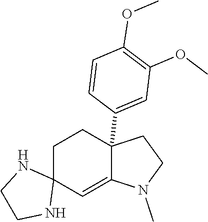 [SMILES: COc1ccc([C@]23CCN(C)C2=CC2(CC3)NCCN2)cc1OC]

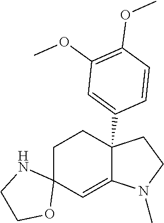 [SMILES: COc1ccc([C@]23CCN(C)C2=CC2(CC3)NCCO2)cc1OC]

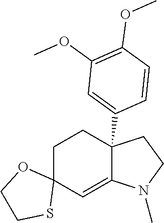 [SMILES: COc1ccc([C@]23CCN(C)C2=CC2(CC3)OCCS2)cc1OC]

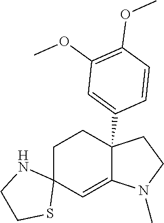 [SMILES: COc1ccc([C@]23CCN(C)C2=CC2(CC3)NCCS2)cc1OC]

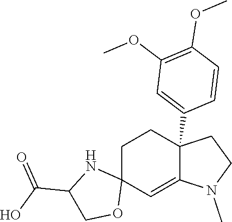 [SMILES: COc1ccc([C@]23CCN(C)C2=CC2(CC3)NC(C(=O)O)CO2)cc1OC]

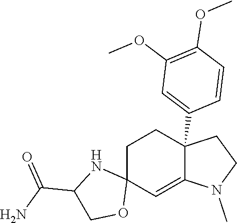 [SMILES: COc1ccc([C@]23CCN(C)C2=CC2(CC3)NC(C(N)=O)CO2)cc1OC]

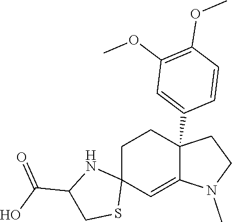 [SMILES: COc1ccc([C@]23CCN(C)C2=CC2(CC3)NC(C(=O)O)CS2)cc1OC]

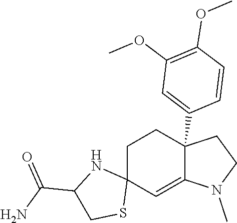 [SMILES: COc1ccc([C@]23CCN(C)C2=CC2(CC3)NC(C(N)=O)CS2)cc1OC]

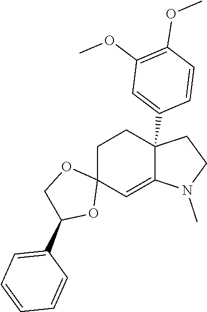 [SMILES: COc1ccc([C@]23CCN(C)C2=CC2(CC3)OC[C@H](c3ccccc3)O2)cc1OC]

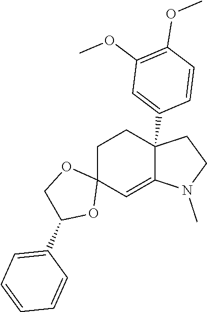 [SMILES: COc1ccc([C@]23CCN(C)C2=CC2(CC3)OC[C@@H](c3ccccc3)O2)cc1OC]

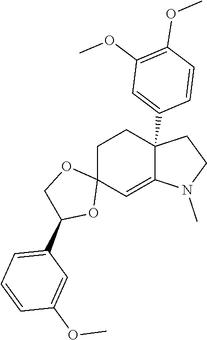 [SMILES: COc1cccc([C@H]2COC3(C=C4N(C)CC[C@]4(c4ccc(OC)c(OC)c4)CC3)O2)c1]

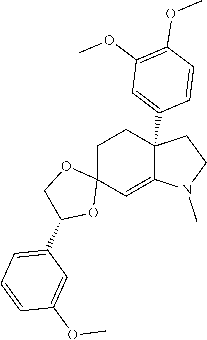 [SMILES: COc1cccc([C@@H]2COC3(C=C4N(C)CC[C@]4(c4ccc(OC)c(OC)c4)CC3)O2)c1]

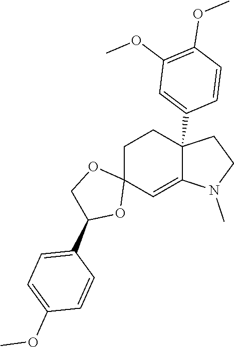 [SMILES: COc1ccc([C@H]2COC3(C=C4N(C)CC[C@]4(c4ccc(OC)c(OC)c4)CC3)O2)cc1]

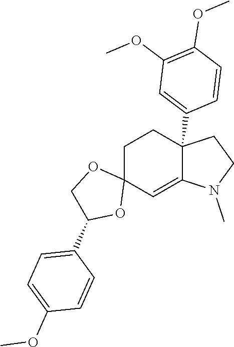 [SMILES: COc1ccc([C@@H]2COC3(C=C4N(C)CC[C@]4(c4ccc(OC)c(OC)c4)CC3)O2)cc1]

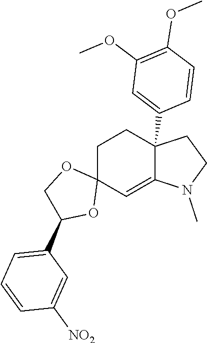 [SMILES: COc1ccc([C@]23CCN(C)C2=CC2(CC3)OC[C@H](c3cccc([N+](=O)[O-])c3)O2)cc1OC]

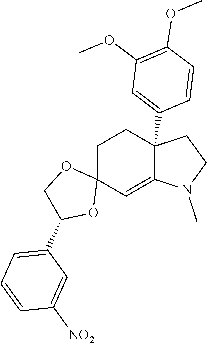 [SMILES: COc1ccc([C@]23CCN(C)C2=CC2(CC3)OC[C@@H](c3cccc([N+](=O)[O-])c3)O2)cc1OC]

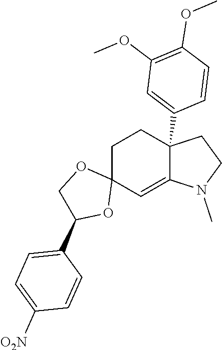 [SMILES: COc1ccc([C@]23CCN(C)C2=CC2(CC3)OC[C@H](c3ccc([N+](=O)[O-])cc3)O2)cc1OC]

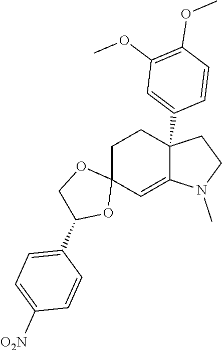 [SMILES: COc1ccc([C@]23CCN(C)C2=CC2(CC3)OC[C@@H](c3ccc([N+](=O)[O-])cc3)O2)cc1OC]

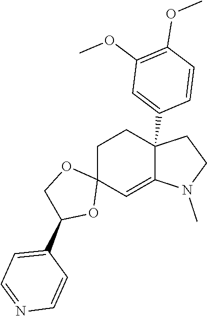 [SMILES: COc1ccc([C@]23CCN(C)C2=CC2(CC3)OC[C@H](c3ccncc3)O2)cc1OC]

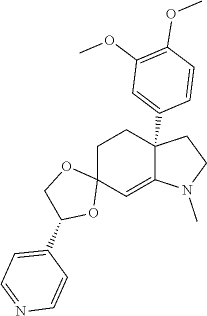 [SMILES: COc1ccc([C@]23CCN(C)C2=CC2(CC3)OC[C@@H](c3ccncc3)O2)cc1OC]

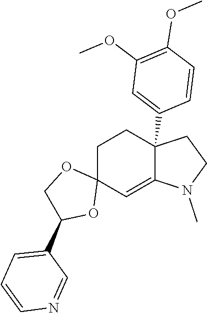 [SMILES: COc1ccc([C@]23CCN(C)C2=CC2(CC3)OC[C@H](c3cccnc3)O2)cc1OC]

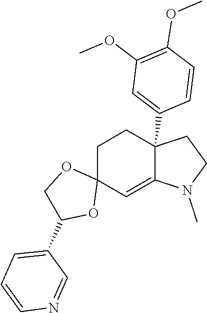

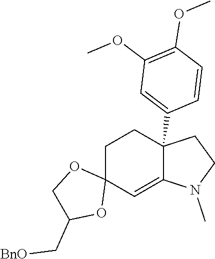 [SMILES: COc1ccc([C@]23CCN(C)C2=CC2(CC3)OCC(COCc3ccccc3)O2)cc1OC]

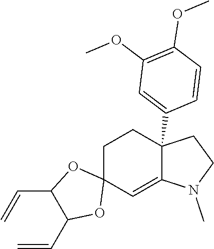 [SMILES: C=CC1OC2(C=C3N(C)CC[C@]3(c3ccc(OC)c(OC)c3)CC2)OC1C=C]

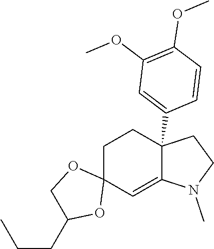 [SMILES: CCCC1COC2(C=C3N(C)CC[C@]3(c3ccc(OC)c(OC)c3)CC2)O1]

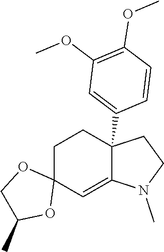 [SMILES: COc1ccc([C@]23CCN(C)C2=CC2(CC3)OC[C@H](C)O2)cc1OC]

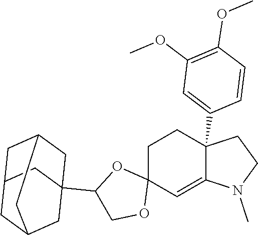 [SMILES: COc1ccc([C@]23CCN(C)C2=CC2(CC3)OCC(C34CC5CC(CC(C5)C3)C4)O2)cc1OC]

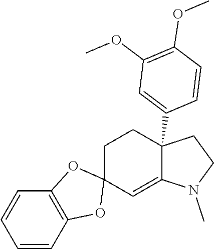 [SMILES: COc1ccc([C@]23CCN(C)C2=CC2(CC3)Oc3ccccc3O2)cc1OC]

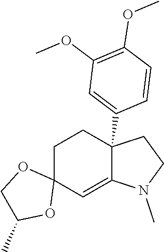 [SMILES: COc1ccc([C@]23CCN(C)C2=CC2(CC3)OC[C@@H](C)O2)cc1OC]

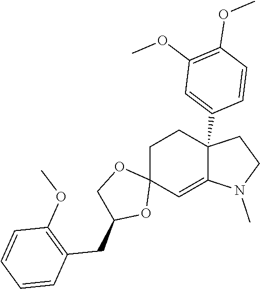 [SMILES: COc1ccccc1C[C@H]1COC2(C=C3N(C)CC[C@]3(c3ccc(OC)c(OC)c3)CC2)O1]

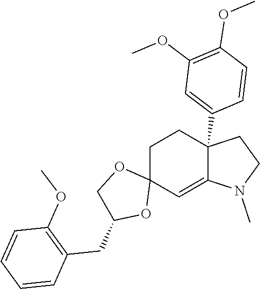 [SMILES: COc1ccccc1C[C@@H]1COC2(C=C3N(C)CC[C@]3(c3ccc(OC)c(OC)c3)CC2)O1]

### Example 3: Synthesis of Compounds of Formula IX

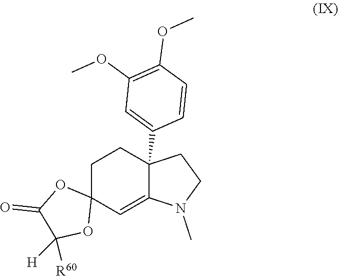

Compounds of Formula (IX) could be prepared by treatment of a ketone, such as Δ⁷-mesembrenone, with an appropriate alpha-hydroxy carboxylic acid, with a Lewis acid, such as boron trifluoride etherate, in a solvent, such as dichloromethane. For example, a compound of formula (IX) where R₆₀ is methyl could be obtained by reacting mesembrine with lactic acid (e.g., with TsOH, toluene). In some embodiments, the compound can be a lactate or glycolate derivative of Δ⁷-mesembrenone.

Table 4 provides non-limiting examples of certain compounds of Formula (IX).

TABLE 4
Exemplary Compounds of Formula IX

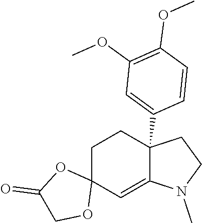 [SMILES: COc1ccc([C@]23CCN(C)C2=CC2(CC3)OCC(=O)O2)cc1OC]

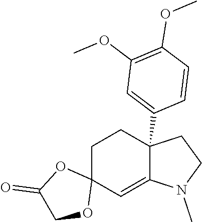

### Example 4: Synthesis of Compounds of Formula X

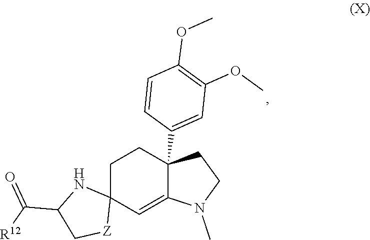

wherein Z is S or O; and R₁₂ is hydroxyl or amino.

Compounds of Formula (X) could be prepared by treatment of a ketone, such as Δ⁷-mesembrenone, with an appropriate alpha-amino carboxylic acid or alpha-amino amide, in a solvent, such as ethanol, at an elevated temperature.

Table 5 provides non-limiting examples of certain compounds of Formula (X).

TABLE 5
Exemplary Compounds of Formula X

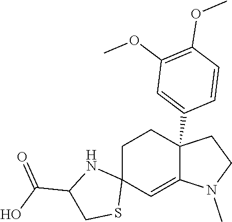 [SMILES: COc1ccc([C@]23CCN(C)C2=CC2(CC3)NC(C(=O)O)CS2)cc1OC]

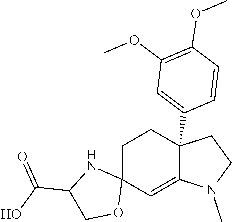 [SMILES: COc1ccc([C@]23CCN(C)C2=CC2(CC3)NC(C(=O)O)CO2)cc1OC]

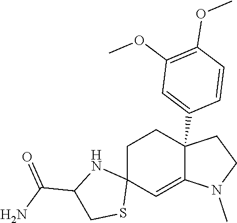 [SMILES: COc1ccc([C@]23CCN(C)C2=CC2(CC3)NC(C(N)=O)CS2)cc1OC]

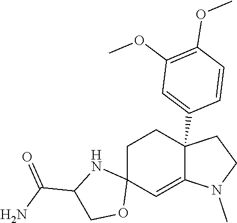 [SMILES: COc1ccc([C@]23CCN(C)C2=CC2(CC3)NC(C(N)=O)CO2)cc1OC]

### Example 5: Synthesis of Compounds of Formula X

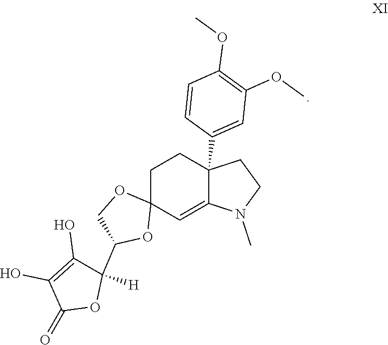

Compounds of Formula (XI) could be prepared by treatment of a ketone such as Δ⁷-mesembrenone, with a naturally occurring diol, such as ascorbic acid, with an acid catalyst, such as TsOH) in a solvent, such as toluene or acetone.

Table 6 provides non-limiting examples of certain compounds of Formula (XI).

TABLE 6
Exemplary Compound of Formula XI

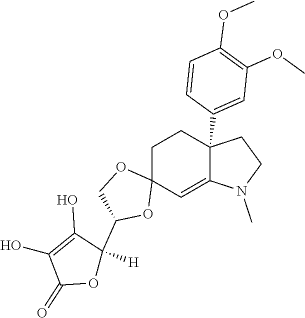

### Example 6: Synthesis of Compounds of Formula XII

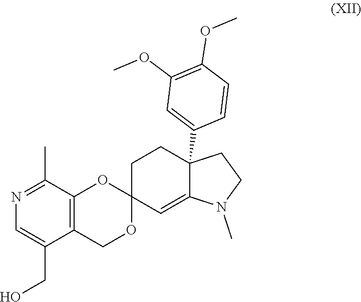 [SMILES: COc1ccc([C@]23CCN(C)C2=CC2(CC3)OCc3c(CO)cnc(C)c3O2)cc1OC]

The compound of formula (XII) could be prepared by treatment of a ketone such as Δ⁷-mesembrenone, with pyridoxine with an acid catalyst in a solvent.

Table 7 provides non-limiting examples of the compound of Formula (XII).

TABLE 7
Exemplary Compound of Formula XII

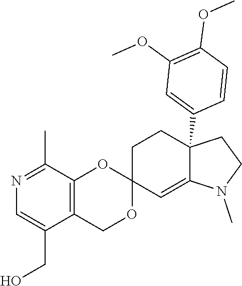 [SMILES: COc1ccc([C@]23CCN(C)C2=CC2(CC3)OCc3c(CO)cnc(C)c3O2)cc1OC]

### Example 7: Synthesis of Compounds of Formula IVa-1

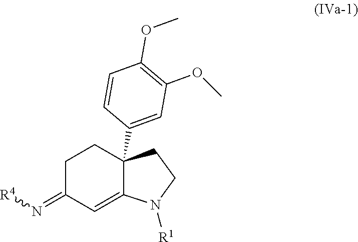

Compounds of Formula (IVa-1) could be prepared by treatment of a ketone such as Δ⁷-mesembrenone, with an appropriately substituted amine or hydrazine in the presence of an acid catalyst, such as acetic acid, in a solvent, such as ethanol, at an appropriate temperature.

Table 12 provides non-limiting examples of certain compounds of Formula (IVa-1).

TABLE 12
Exemplary Compounds of Formula (IVa-1)

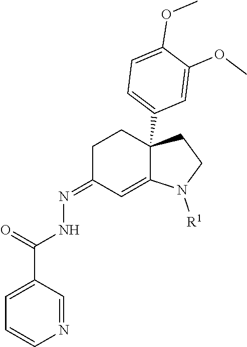

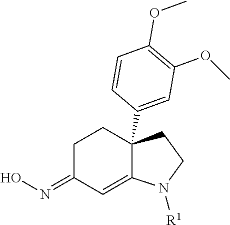

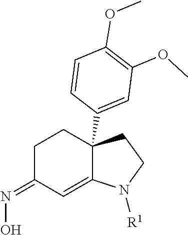

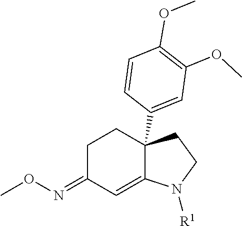

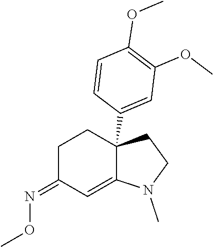

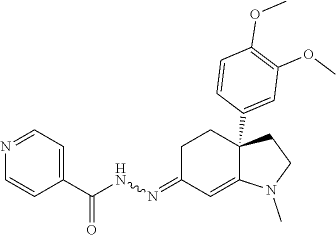 [SMILES: COc1ccc([C@@]23CCC(=NNC(=O)c4ccncc4)C=C2N(C)CC3)cc1OC]

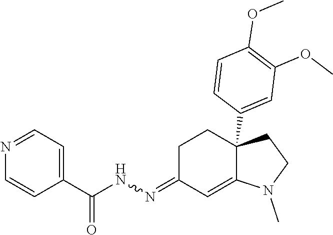 [SMILES: COc1ccc([C@@]23CCC(=NNC(=O)c4ccncc4)C=C2N(C)CC3)cc1OC]

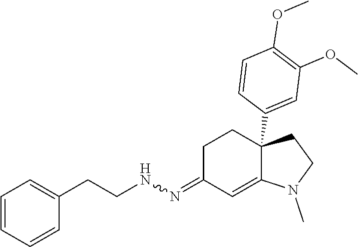 [SMILES: COc1ccc([C@@]23CCC(=NNCCc4ccccc4)C=C2N(C)CC3)cc1OC]

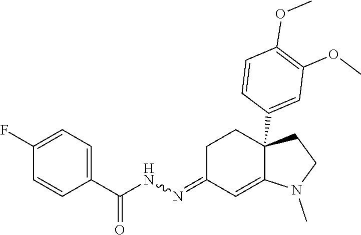 [SMILES: COc1ccc([C@@]23CCC(=NNC(=O)c4ccc(F)cc4)C=C2N(C)CC3)cc1OC]

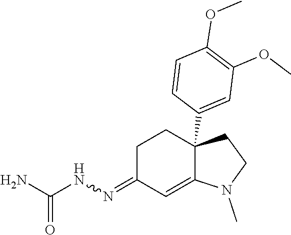 [SMILES: COc1ccc([C@@]23CCC(=NNC(N)=O)C=C2N(C)CC3)cc1OC]

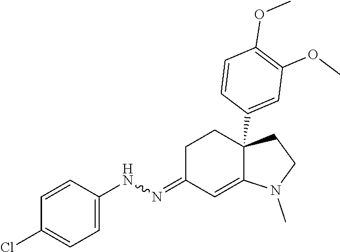 [SMILES: COc1ccc([C@@]23CCC(=NNc4ccc(Cl)cc4)C=C2N(C)CC3)cc1OC]

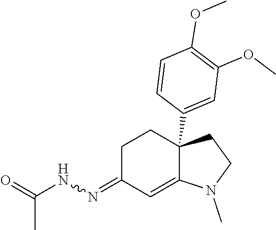 [SMILES: COc1ccc([C@@]23CCC(=NNC(C)=O)C=C2N(C)CC3)cc1OC]

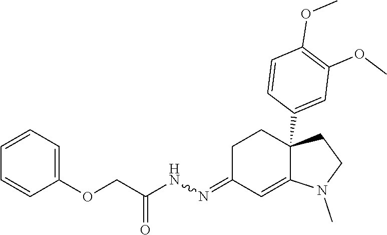 [SMILES: COc1ccc([C@@]23CCC(=NNC(=O)COc4ccccc4)C=C2N(C)CC3)cc1OC]

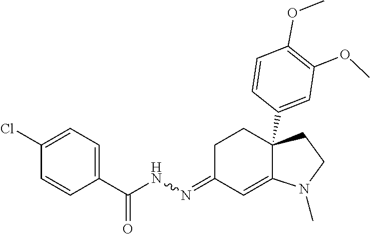 [SMILES: COc1ccc([C@@]23CCC(=NNC(=O)c4ccc(Cl)cc4)C=C2N(C)CC3)cc1OC]

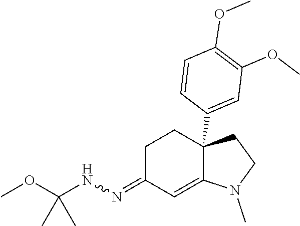

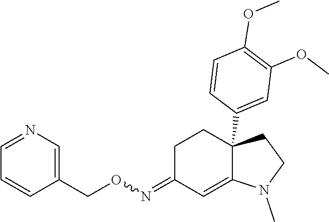 [SMILES: COc1ccc([C@@]23CCC(=NOCc4cccnc4)C=C2N(C)CC3)cc1OC]

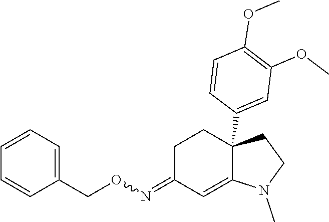 [SMILES: COc1ccc([C@@]23CCC(=NOCc4ccccc4)C=C2N(C)CC3)cc1OC]

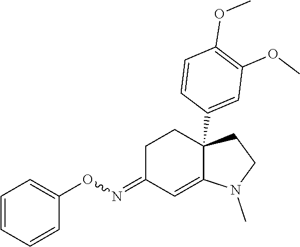 [SMILES: COc1ccc([C@@]23CCC(=NOc4ccccc4)C=C2N(C)CC3)cc1OC]

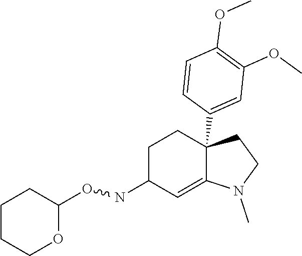 [SMILES: COc1ccc([C@@]23CCC(NOC4CCCCO4)C=C2N(C)CC3)cc1OC]

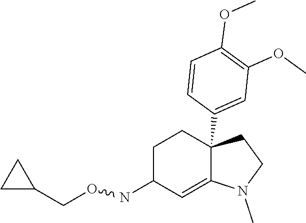 [SMILES: COc1ccc([C@@]23CCC(NOCC4CC4)C=C2N(C)CC3)cc1OC]

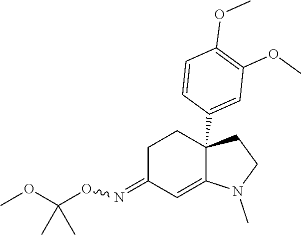

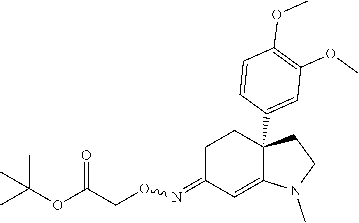 [SMILES: COc1ccc([C@@]23CCC(=NOCC(=O)OC(C)(C)C)C=C2N(C)CC3)cc1OC]

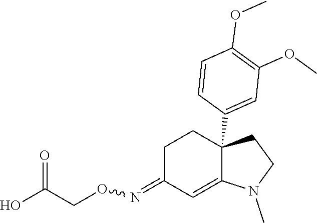 [SMILES: COc1ccc([C@@]23CCC(=NOCC(=O)O)C=C2N(C)CC3)cc1OC]

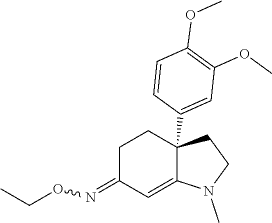 [SMILES: CCON=C1C=C2N(C)CC[C@]2(c2ccc(OC)c(OC)c2)CC1]

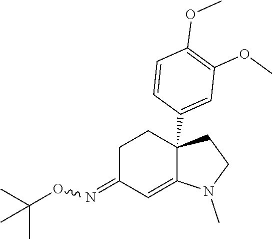 [SMILES: COc1ccc([C@@]23CCC(=NOC(C)(C)C)C=C2N(C)CC3)cc1OC]

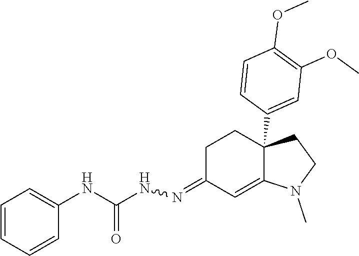 [SMILES: COc1ccc([C@@]23CCC(=NNC(=O)Nc4ccccc4)C=C2N(C)CC3)cc1OC]

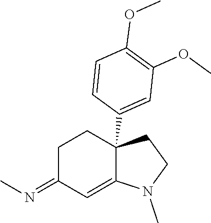

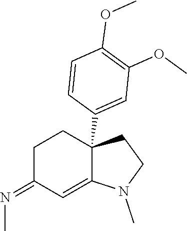 [SMILES: C/N=C1\C=C2N(C)CC[C@]2(c2ccc(OC)c(OC)c2)CC1]

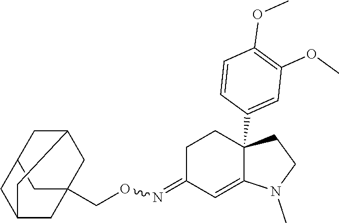 [SMILES: COc1ccc([C@@]23CCC(=NOCC45CC6CC(CC(C6)C4)C5)C=C2N(C)CC3)cc1OC]

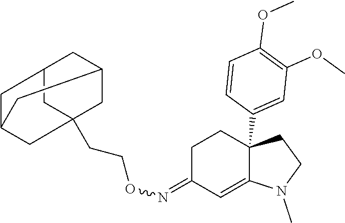 [SMILES: COc1ccc([C@@]23CCC(=NOCCC45CC6CC(CC(C6)C4)C5)C=C2N(C)CC3)cc1OC]

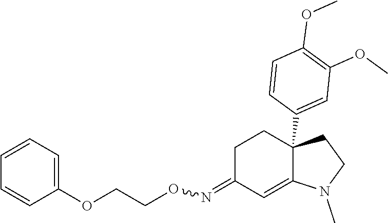 [SMILES: COc1ccc([C@@]23CCC(=NOCCOc4ccccc4)C=C2N(C)CC3)cc1OC]

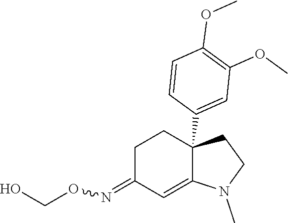 [SMILES: COc1ccc([C@@]23CCC(=NOCO)C=C2N(C)CC3)cc1OC]

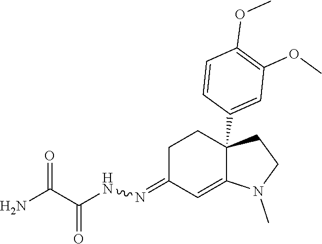 [SMILES: COc1ccc([C@@]23CCC(=NNC(=O)C(N)=O)C=C2N(C)CC3)cc1OC]

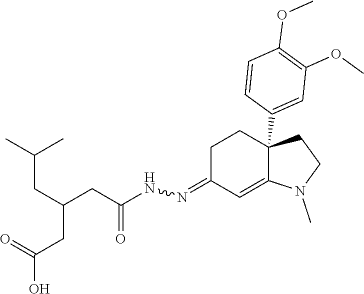 [SMILES: COc1ccc([C@@]23CCC(=NNC(=O)CC(CC(=O)O)CC(C)C)C=C2N(C)CC3)cc1OC]

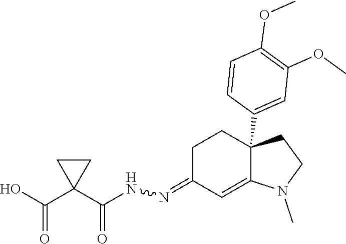 [SMILES: COc1ccc([C@@]23CCC(=NNC(=O)C4(C(=O)O)CC4)C=C2N(C)CC3)cc1OC]

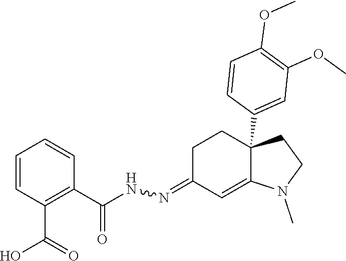 [SMILES: COc1ccc([C@@]23CCC(=NNC(=O)c4ccccc4C(=O)O)C=C2N(C)CC3)cc1OC]

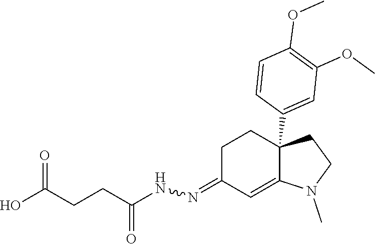 [SMILES: COc1ccc([C@@]23CCC(=NNC(=O)CCC(=O)O)C=C2N(C)CC3)cc1OC]

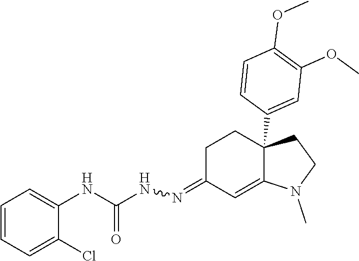 [SMILES: COc1ccc([C@@]23CCC(=NNC(=O)Nc4ccccc4Cl)C=C2N(C)CC3)cc1OC]

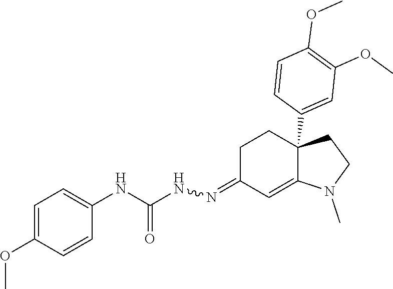 [SMILES: COc1ccc(NC(=O)NN=C2C=C3N(C)CC[C@]3(c3ccc(OC)c(OC)c3)CC2)cc1]

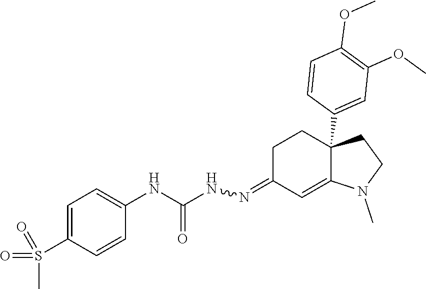 [SMILES: COc1ccc([C@@]23CCC(=NNC(=O)Nc4ccc(S(C)(=O)=O)cc4)C=C2N(C)CC3)cc1OC]

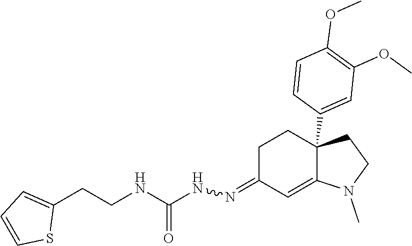 [SMILES: COc1ccc([C@@]23CCC(=NNC(=O)NCCc4cccs4)C=C2N(C)CC3)cc1OC]

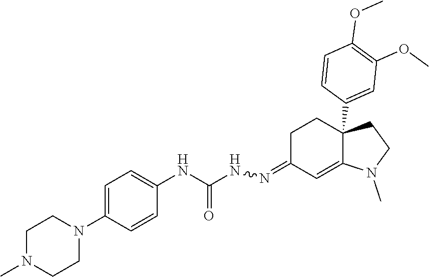

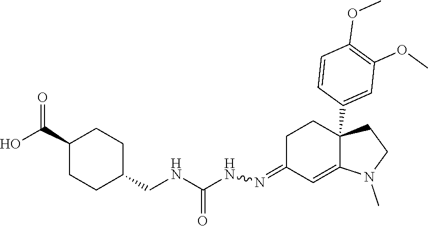 [SMILES: COc1ccc([C@@]23CCC(=NNC(=O)NC[C@H]4CC[C@H](C(=O)O)CC4)C=C2N(C)CC3)cc1OC]

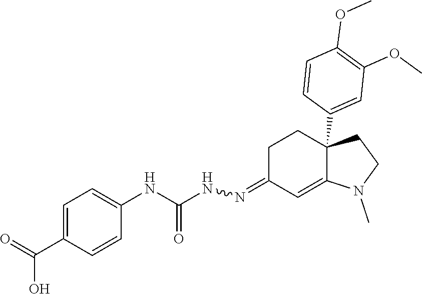 [SMILES: COc1ccc([C@@]23CCC(=NNC(=O)Nc4ccc(C(=O)O)cc4)C=C2N(C)CC3)cc1OC]

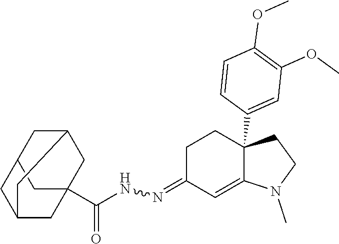 [SMILES: COc1ccc([C@@]23CCC(=NNC(=O)C45CC6CC(CC(C6)C4)C5)C=C2N(C)CC3)cc1OC]

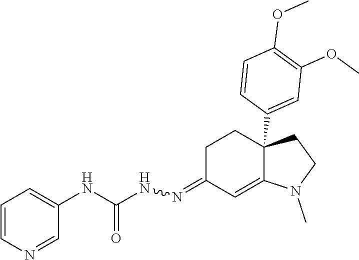 [SMILES: COc1ccc([C@@]23CCC(=NNC(=O)Nc4cccnc4)C=C2N(C)CC3)cc1OC]

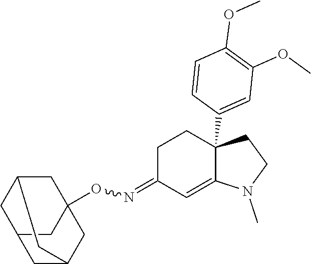 [SMILES: COc1ccc([C@@]23CCC(=NOC45CC6CC(CC(C6)C4)C5)C=C2N(C)CC3)cc1OC]

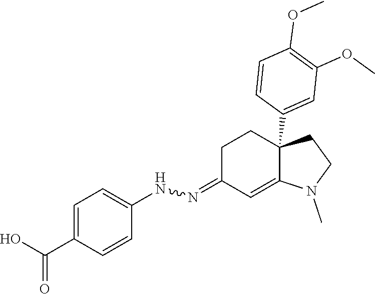 [SMILES: COc1ccc([C@@]23CCC(=NNc4ccc(C(=O)O)cc4)C=C2N(C)CC3)cc1OC]

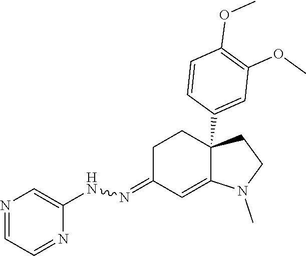 [SMILES: COc1ccc([C@@]23CCC(=NNc4cnccn4)C=C2N(C)CC3)cc1OC]

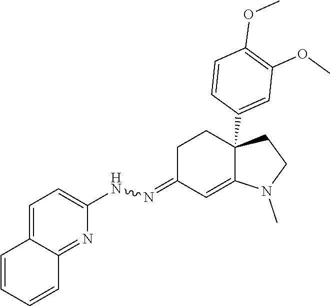 [SMILES: COc1ccc([C@@]23CCC(=NNc4ccc5ccccc5n4)C=C2N(C)CC3)cc1OC]

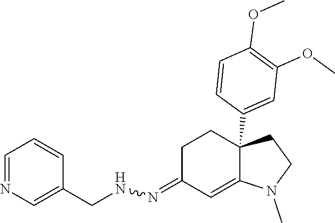 [SMILES: COc1ccc([C@@]23CCC(=NNCc4cccnc4)C=C2N(C)CC3)cc1OC]

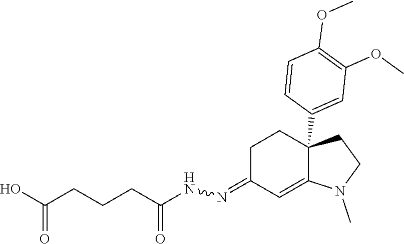 [SMILES: COc1ccc([C@@]23CCC(=NNC(=O)CCCC(=O)O)C=C2N(C)CC3)cc1OC]

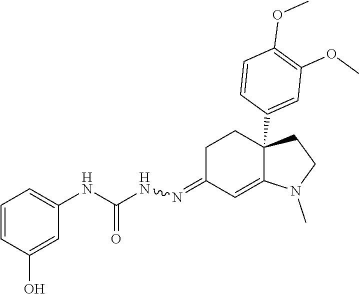 [SMILES: COc1ccc([C@@]23CCC(=NNC(=O)Nc4cccc(O)c4)C=C2N(C)CC3)cc1OC]

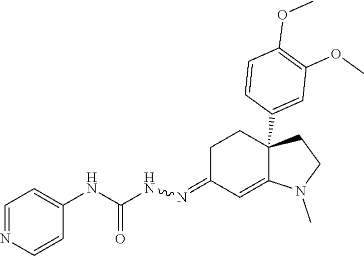 [SMILES: COc1ccc([C@@]23CCC(=NNC(=O)Nc4ccncc4)C=C2N(C)CC3)cc1OC]

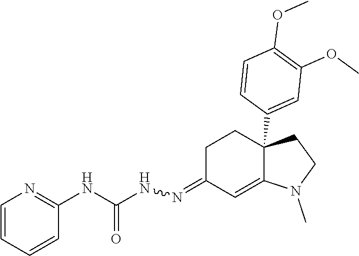 [SMILES: COc1ccc([C@@]23CCC(=NNC(=O)Nc4ccccn4)C=C2N(C)CC3)cc1OC]

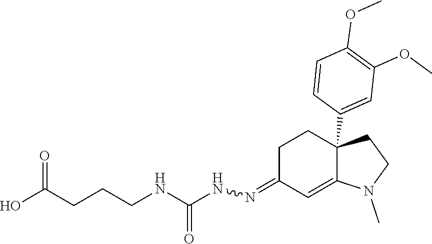 [SMILES: COc1ccc([C@@]23CCC(=NNC(=O)NCCCC(=O)O)C=C2N(C)CC3)cc1OC]

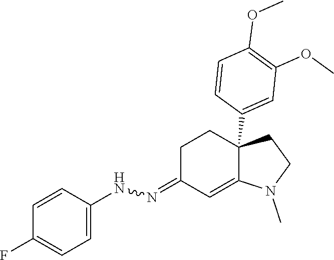 [SMILES: COc1ccc([C@@]23CCC(=NNc4ccc(F)cc4)C=C2N(C)CC3)cc1OC]

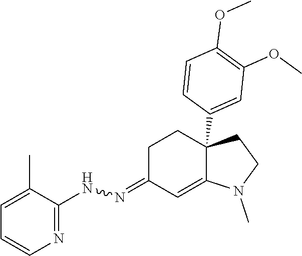 [SMILES: COc1ccc([C@@]23CCC(=NNc4ncccc4C)C=C2N(C)CC3)cc1OC]

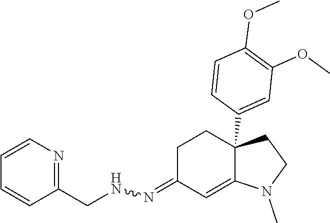 [SMILES: COc1ccc([C@@]23CCC(=NNCc4ccccn4)C=C2N(C)CC3)cc1OC]

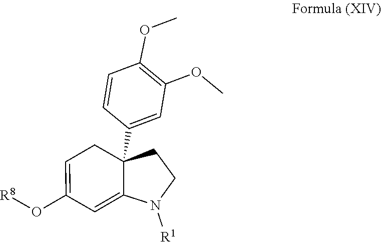

Compounds of Formula (XIV) including compounds of Formula (XIV-1) could be prepared by treating a ketone, such as Δ⁷-mesembrenone, with an appropriate base, such as potassium tert-butoxide, and an acid anhydride, acyl chloride, or cholorsilane, in a solvent, such as THF.

TABLE 13
Exemplary Compounds of Formula (XIV)

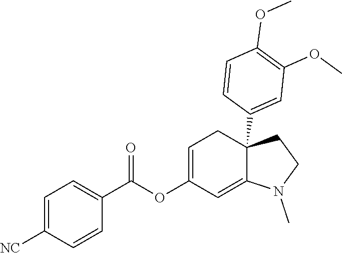 [SMILES: COc1ccc([C@@]23CC=C(OC(=O)c4ccc(C#N)cc4)C=C2N(C)CC3)cc1OC]

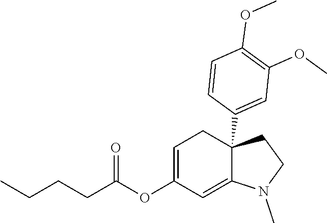 [SMILES: CCCCC(=O)OC1=CC[C@@]2(c3ccc(OC)c(OC)c3)CCN(C)C2=C1]

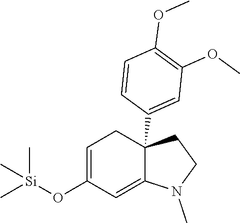

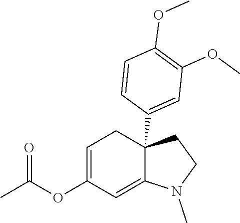 [SMILES: COc1ccc([C@@]23CC=C(OC(C)=O)C=C2N(C)CC3)cc1OC]

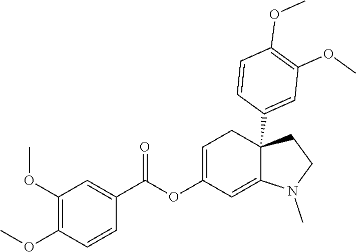

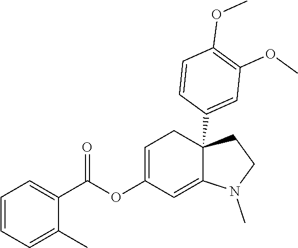 [SMILES: COc1ccc([C@@]23CC=C(OC(=O)c4ccccc4C)C=C2N(C)CC3)cc1OC]

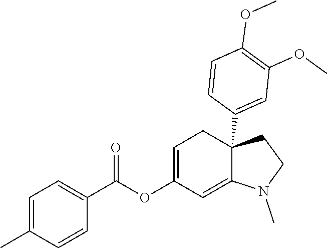

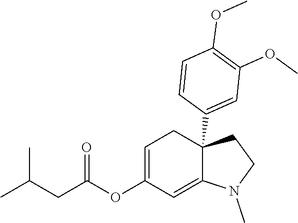 [SMILES: COc1ccc([C@@]23CC=C(OC(=O)CC(C)C)C=C2N(C)CC3)cc1OC]

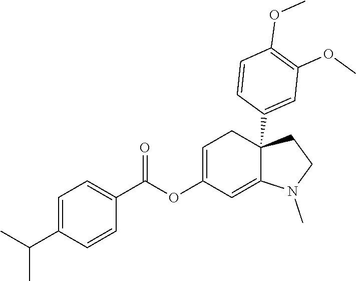 [SMILES: COc1ccc([C@@]23CC=C(OC(=O)c4ccc(C(C)C)cc4)C=C2N(C)CC3)cc1OC]

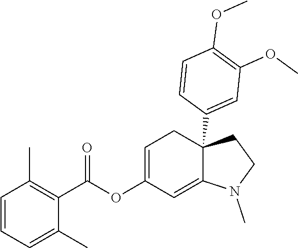 [SMILES: COc1ccc([C@@]23CC=C(OC(=O)c4c(C)cccc4C)C=C2N(C)CC3)cc1OC]

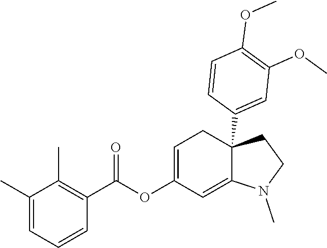

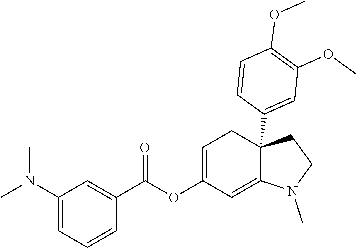

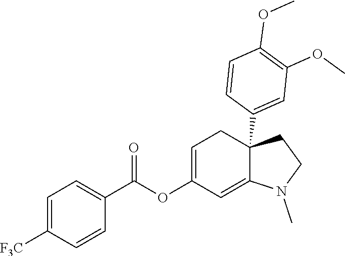 [SMILES: COc1ccc([C@@]23CC=C(OC(=O)c4ccc(C(F)(F)F)cc4)C=C2N(C)CC3)cc1OC]

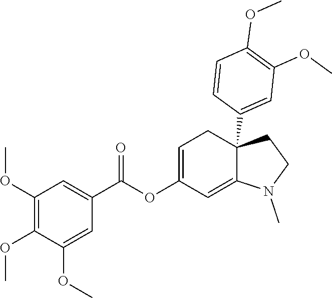 [SMILES: COc1ccc([C@@]23CC=C(OC(=O)c4cc(OC)c(OC)c(OC)c4)C=C2N(C)CC3)cc1OC]

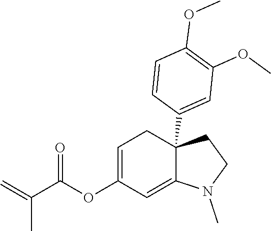 [SMILES: C=C(C)C(=O)OC1=CC[C@@]2(c3ccc(OC)c(OC)c3)CCN(C)C2=C1]

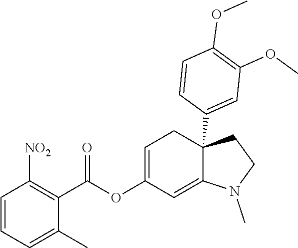 [SMILES: COc1ccc([C@@]23CC=C(OC(=O)c4c(C)cccc4[N+](=O)[O-])C=C2N(C)CC3)cc1OC]

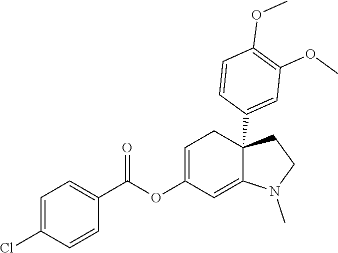 [SMILES: COc1ccc([C@@]23CC=C(OC(=O)c4ccc(Cl)cc4)C=C2N(C)CC3)cc1OC]

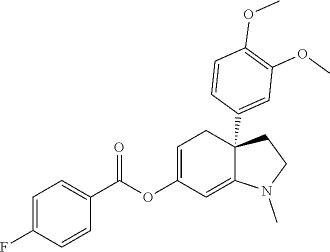 [SMILES: COc1ccc([C@@]23CC=C(OC(=O)c4ccc(F)cc4)C=C2N(C)CC3)cc1OC]

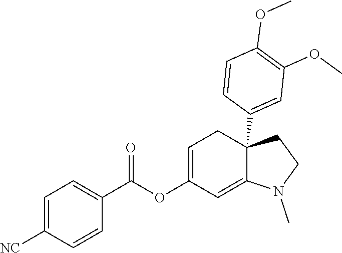 [SMILES: COc1ccc([C@@]23CC=C(OC(=O)c4ccc(C#N)cc4)C=C2N(C)CC3)cc1OC]

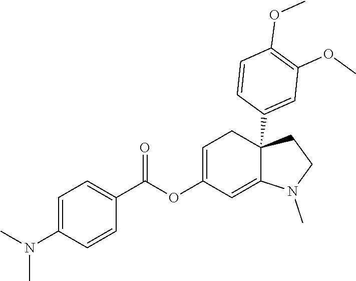

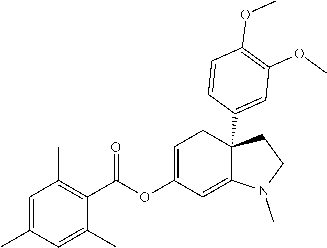

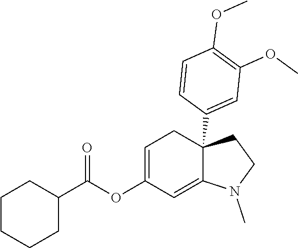 [SMILES: COc1ccc([C@@]23CC=C(OC(=O)C4CCCCC4)C=C2N(C)CC3)cc1OC]

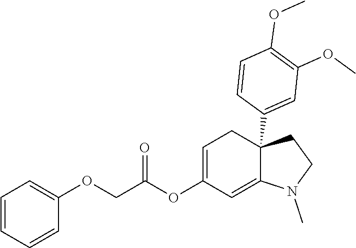 [SMILES: COc1ccc([C@@]23CC=C(OC(=O)COc4ccccc4)C=C2N(C)CC3)cc1OC]

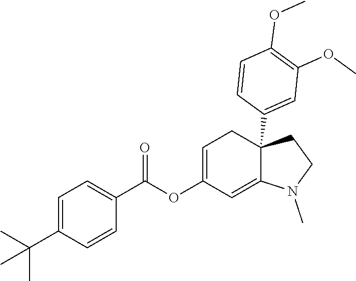 [SMILES: COc1ccc([C@@]23CC=C(OC(=O)c4ccc(C(C)(C)C)cc4)C=C2N(C)CC3)cc1OC]

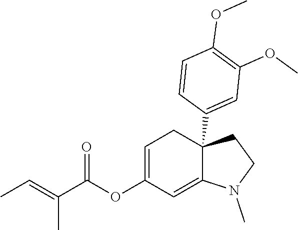 [SMILES: C/C=C(\C)C(=O)OC1=CC[C@@]2(c3ccc(OC)c(OC)c3)CCN(C)C2=C1]

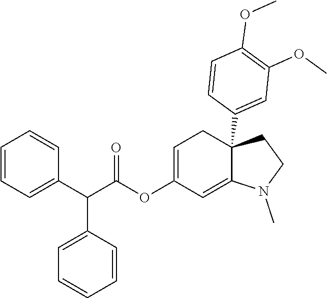 [SMILES: COc1ccc([C@@]23CC=C(OC(=O)C(c4ccccc4)c4ccccc4)C=C2N(C)CC3)cc1OC]

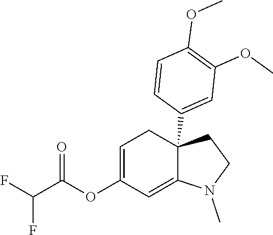 [SMILES: COc1ccc([C@@]23CC=C(OC(=O)C(F)F)C=C2N(C)CC3)cc1OC]

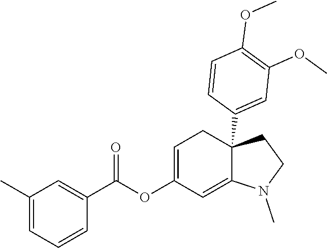

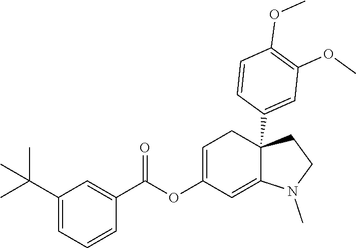 [SMILES: COc1ccc([C@@]23CC=C(OC(=O)c4cccc(C(C)(C)C)c4)C=C2N(C)CC3)cc1OC]

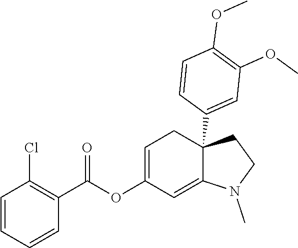 [SMILES: COc1ccc([C@@]23CC=C(OC(=O)c4ccccc4Cl)C=C2N(C)CC3)cc1OC]

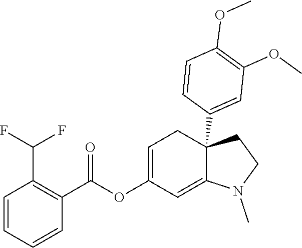 [SMILES: COc1ccc([C@@]23CC=C(OC(=O)c4ccccc4C(F)F)C=C2N(C)CC3)cc1OC]

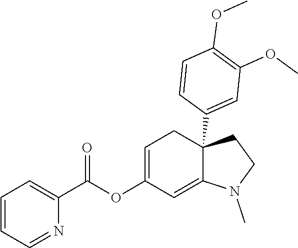 [SMILES: COc1ccc([C@@]23CC=C(OC(=O)c4ccccn4)C=C2N(C)CC3)cc1OC]

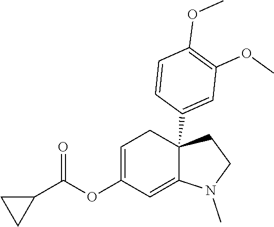 [SMILES: COc1ccc([C@@]23CC=C(OC(=O)C4CC4)C=C2N(C)CC3)cc1OC]

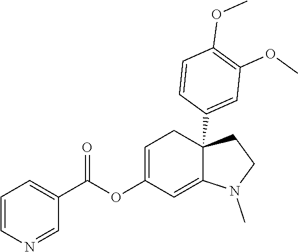 [SMILES: COc1ccc([C@@]23CC=C(OC(=O)c4cccnc4)C=C2N(C)CC3)cc1OC]

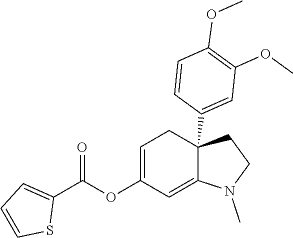 [SMILES: COc1ccc([C@@]23CC=C(OC(=O)c4cccs4)C=C2N(C)CC3)cc1OC]

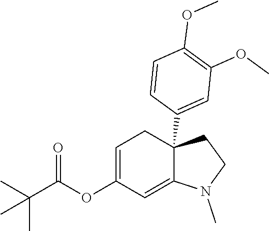 [SMILES: COc1ccc([C@@]23CC=C(OC(=O)C(C)(C)C)C=C2N(C)CC3)cc1OC]

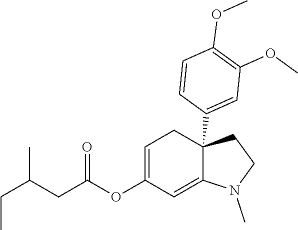 [SMILES: CCC(C)CC(=O)OC1=CC[C@@]2(c3ccc(OC)c(OC)c3)CCN(C)C2=C1]

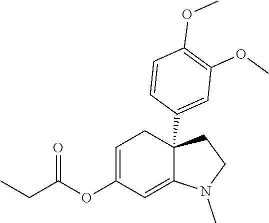 [SMILES: CCC(=O)OC1=CC[C@@]2(c3ccc(OC)c(OC)c3)CCN(C)C2=C1]

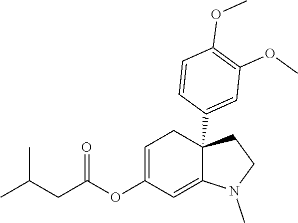 [SMILES: COc1ccc([C@@]23CC=C(OC(=O)CC(C)C)C=C2N(C)CC3)cc1OC]

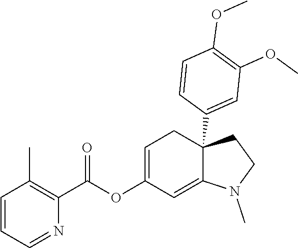 [SMILES: COc1ccc([C@@]23CC=C(OC(=O)c4ncccc4C)C=C2N(C)CC3)cc1OC]

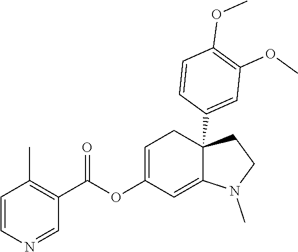 [SMILES: COc1ccc([C@@]23CC=C(OC(=O)c4cnccc4C)C=C2N(C)CC3)cc1OC]

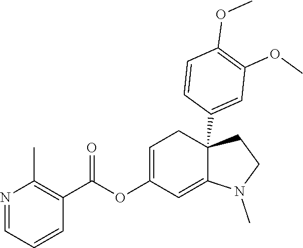 [SMILES: COc1ccc([C@@]23CC=C(OC(=O)c4cccnc4C)C=C2N(C)CC3)cc1OC]

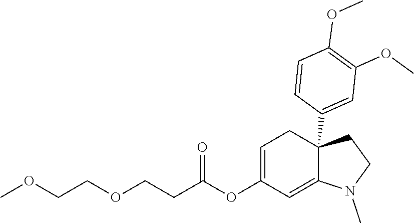

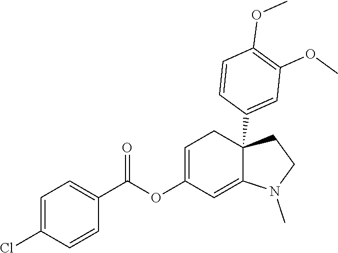 [SMILES: COc1ccc([C@@]23CC=C(OC(=O)c4ccc(Cl)cc4)C=C2N(C)CC3)cc1OC]

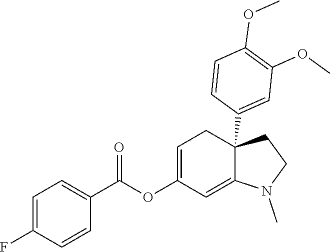 [SMILES: COc1ccc([C@@]23CC=C(OC(=O)c4ccc(F)cc4)C=C2N(C)CC3)cc1OC]

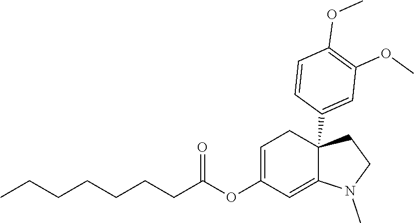 [SMILES: CCCCCCCC(=O)OC1=CC[C@@]2(c3ccc(OC)c(OC)c3)CCN(C)C2=C1]

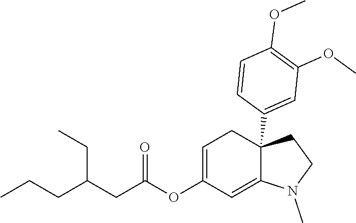 [SMILES: CCCC(CC)CC(=O)OC1=CC[C@@]2(c3ccc(OC)c(OC)c3)CCN(C)C2=C1]

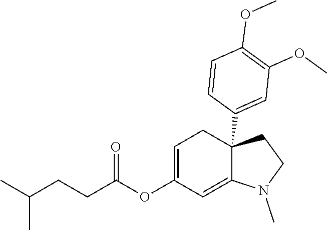 [SMILES: COc1ccc([C@@]23CC=C(OC(=O)CCC(C)C)C=C2N(C)CC3)cc1OC]

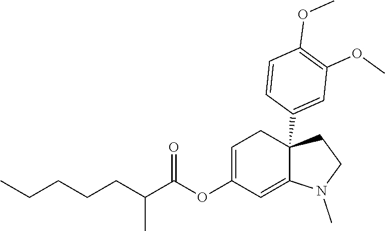 [SMILES: CCCCCC(C)C(=O)OC1=CC[C@@]2(c3ccc(OC)c(OC)c3)CCN(C)C2=C1]

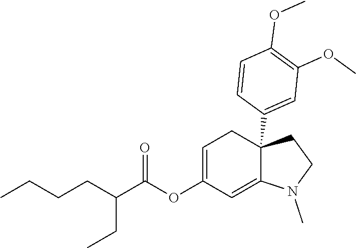 [SMILES: CCCCC(CC)C(=O)OC1=CC[C@@]2(c3ccc(OC)c(OC)c3)CCN(C)C2=C1]

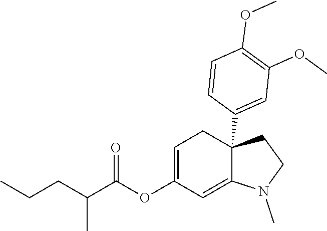 [SMILES: CCCC(C)C(=O)OC1=CC[C@@]2(c3ccc(OC)c(OC)c3)CCN(C)C2=C1]

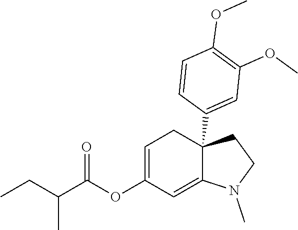 [SMILES: CCC(C)C(=O)OC1=CC[C@@]2(c3ccc(OC)c(OC)c3)CCN(C)C2=C1]

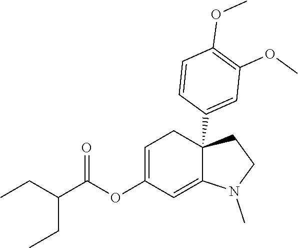 [SMILES: CCC(CC)C(=O)OC1=CC[C@@]2(c3ccc(OC)c(OC)c3)CCN(C)C2=C1]

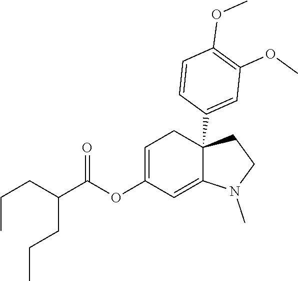 [SMILES: CCCC(CCC)C(=O)OC1=CC[C@@]2(c3ccc(OC)c(OC)c3)CCN(C)C2=C1]

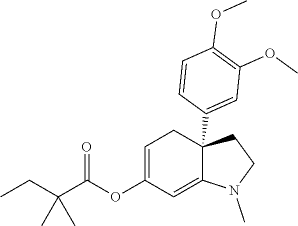 [SMILES: CCC(C)(C)C(=O)OC1=CC[C@@]2(c3ccc(OC)c(OC)c3)CCN(C)C2=C1]

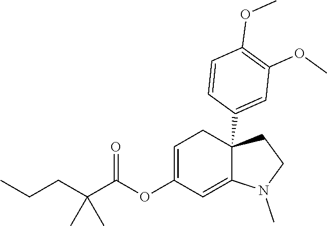 [SMILES: CCCC(C)(C)C(=O)OC1=CC[C@@]2(c3ccc(OC)c(OC)c3)CCN(C)C2=C1]

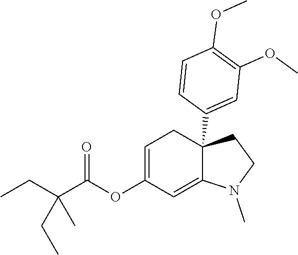 [SMILES: CCC(C)(CC)C(=O)OC1=CC[C@@]2(c3ccc(OC)c(OC)c3)CCN(C)C2=C1]

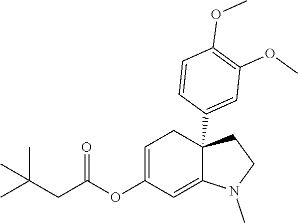 [SMILES: COc1ccc([C@@]23CC=C(OC(=O)CC(C)(C)C)C=C2N(C)CC3)cc1OC]

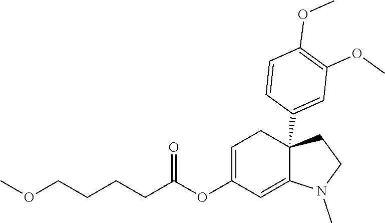

### Example A1: Hydrolysis Assay

Dilute 50 uL of compound (10 mM) in DMSO into 950 μL of HCl aqueous solution (0.01 M) to a final concentration of 0.5 mM. Measure hydrolysis kinetics under a certain temperature by LCMS at a certain time interval. Record the corresponding LCMS chromatograms and calculate the conversions according to the equation:

$$conversion ( \% ) = peak area 1 / ( peak area 1 + peak area 2 ) * 100 \%$$

where peak area 1 is the integration of hydrolyzed product and peak area 2 is the integration of starting material.

### Example A2: Hydrolysis Assay

### Condition 1 (SGF)

Dilute 50 μL of compound (10 mM) in DMSO into 950 μL of Simulated Gastric Fluid (SGF) with pepsin (pH 1.5) to a final concentration of 0.5 mM. Conduct hydrolysis kinetics at 37° C. Record the corresponding LCMS chromatograms and calculate the conversions according to the equation:

$$conversion ( \% ) = peak area 1 / ( peak area 1 + peak area 2 ) * 100 \%$$

where peak area 1 is the integration of hydrolyzed product and peak area 2 is the integration of starting material.

### Condition 2 (DI Water)

Dilute 50 uL of compound (10 mM) in DMSO into 950 uL of DI water to a final concentration of 0.5 mM. Conduct hydrolysis kinetics at 25° C. Record the corresponding LCMS chromatograms and calculate the conversions according to the equation:

$$conversion ( \% ) = peak area 1 / ( peak area 1 + peak area 2 ) * 100 \%$$

where peak area 1 is the integration of hydrolyzed product and peak area 2 is the integration of starting material.

### Example A3: Plasma Stability Assay

Measure compound stability in mouse and human plasma by incubating compound with mouse or human plasma at 37° C. in a microwell plate for sequential time points, monitoring for compound disappearance. Prepare a 20 mM stock solution of test compound by dissolving an appropriate amount of compound in DMSO. Dilute with additional DMSO to prepare a stock of 1 mM. Further dilute a 1 mM stock 200-fold in mice or human plasma (pre-warmed at 37° C. for 15 minutes) to attain a final concentration of 5 μM (0.5% DMSO). Spike a positive control and a test compound into a 400 μL aliquot (n=2) of mice or human plasma and incubate in a CO₂ incubator at 37° C. Withdraw 50 μL aliquots at 0, 0.25, 0.5, 1, 2, 4 hrs and immediately quench with 400 μL of ice-cold acetonitrile containing internal standard. Store at minus 2-8° C. Thaw to room temperature and then vortex for 5 min. Centriguge at 4000 RPM for 15 min at 4° C. Transfer an aliquot of 100 μL to a 96-well deep plate and analyze by LC-MS/MS. Quantify the concentrations of test compound and positive control in the samples using LC-MS/MS. Report results as % of compound remaining at time point.

### Example A4: Plasma Stability Assay

Measure compound stability in rat and human plasma by incubating compound with rat or human plasma at 37° C. in a microwell plate for sequential time points, monitoring for compound disappearance. Prepare a 1 mM stock solution of test compound by dissolving appropriate amount of compound in DMSO. Dilute the 1 mM stock 200-fold in rat or human plasma to attain a final concentration of 10 μM (0.5% DMSO). Add 50 μL aliquots of positive control and test compound spiked into rat or human plasma (n=2) to a pre-warmed plate (37° C.) and shake at 50 rpm. At each time point, stop the reaction by adding 500 μL of acetonitrile containing internal standards (100 nM aprozolam, 200 nM caffeine, 100 nM tolbutamide). Vortex the sample for 10 minutes, then centrifuge at 3,220 g for 30 minutes to precipitate proteins. Transfer 100 μL of the supernatant to a new plate. Dilute the supernatant with ultrapure water according to the LC-MS signal response and peak shape. Quantify the concentrations of test compounds and positive control in the test samples using LC-MS/MS. Report results as % of compound remaining at each time point.

### Example BI: SERT Inhibition Assay

Measure SERT inhibition using a Neruotransmitter Transportation Fluorescence assay. Prepare stable 5HTT cells in a 384 microwell plate. Prepare compounds in assay buffer (20 mM HEPES in HBSS, 0.1% BSA). Add the compounds to the plated cells and incubate for 30 minutes at 37° C. Add 25 μL of dye solution (Molecular Devices Neurotransmitter Transporter Uptake Assay Kit) per well and incubate for 30 minutes at 37° C. Read the plates on a plate reader.

We claim:

1.-19. (canceled)

20. A compound of formula (I): 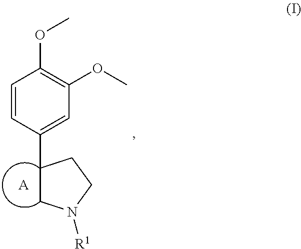
or a pharmaceutically acceptable salt thereof, wherein
R¹ is C₁-C₇ alkyl or H; and
ring A is 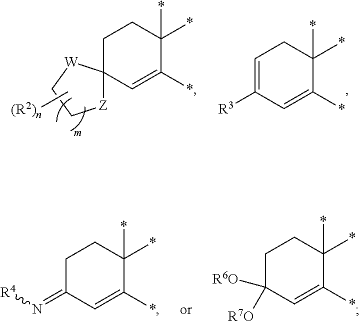
wherein * denotes the attachment points of ring A to the compound of formula (I), and wherein
each of W and Z is independently O, NH, or S;
each R² is independently C₁-C₃ alkyl, C₂-C₆ alkenyl, —COOH, —CONH₂, C₃-C₁₀ cycloalkyl, phenyl, 5- to 7-membered heterocyclyl, or 5- to 7-membered heteroaryl, wherein each hydrogen atom in C₁-C₃ alkyl, C₂-C₆ alkenyl, phenyl, 5- to 7-membered heterocyclyl and 5- to 7-membered heteroaryl is optionally substituted by —OH, C₁-C₃ alkyl, C₁-C₃ alkoxy, aryloxy, C₁-C₃ alkanol, or —NO₂, wherein each hydrogen atom in C₁-C₃ alkoxy and aryloxy is optionally substituted by C₁-C₃ alkoxy or phenyl; or two R²s on a single carbon atom combine to form ═O; or two adjacent R²s together with the carbon atoms to which they are attached combine to form 5- to 7-membered heteroaryl or phenyl, wherein each hydrogen atom in 5- to 7-membered heteroaryl or phenyl is optionally substituted by —OH, C₁-C₃ alkyl, or C₁-C₃ alkanol;
m is 1 or 2;
n is 0, 1, 2, or 3;
R³ is —OSi(C₁-C₆ alkyl)₃, —OC(O)C₁-C₆ alkyl, —OC(O)C₂-C₆ alkenyl, —OC(O)C₃-C₁₀ cycloalkyl, —OC(O)phenyl, or —OC(O)-5- to 7-membered heteroaryl; wherein each hydrogen atom in —OC(O)C₁-C₆ alkyl, —OC(O)C₂-C₆ alkenyl, —OC(O)C₃-C₁₀ cycloalkyl, —OC(O)phenyl, and —OC(O)₅- to 7-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, phenyl, phenoxy or —O(CH₂)ₚOCH₃;
p is 2, 3, or 4;
R⁴ is —OR⁵ or —N(R⁵)₂;
each R⁵ is H, C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₅ alkyl, —C(O)—C₃-C₁₀ cycloalkyl, —C(O)-phenyl, —C(O)N(H)-phenyl, —C(O)N(H)-5- to 7-membered heteroaryl, —C(O)N(H)—C₁-C₆ alkyl, —C(O)C(O)OH, or —CONH₂; wherein each hydrogen atom in C₁-C₆ alkyl, C₃-C₆ heterocycloalkyl, C₃-C₁₀ cycloalkyl, C₁-C₆ alkoxy, phenyl, 5- to 10-membered heteroaryl, —C(O)-5- to 7-membered heteroaryl, —C(O)—C₁-C₈ alkyl, —C(O)—C₃-C₁₀ cycloalkyl, —C(O)-phenyl, —C(O)N(H)-phenyl, —C(O)N(H)-5- to 7-membered heteroaryl, and —C(O)N(H)—C₁-C₆ alkyl is optionally substituted by halogen, OH, C₁-C₃ alkyl, C₃-C₁₀ cycloalkyl optionally substituted by —COOH, phenyl, 5- to 7-membered heteroaryl, aryloxy, C₁-C₆ alkoxy, C₁-C₃ alkanol, C₃-C₆ heterocycloalkyl optionally substituted with methyl, —COOH, —S(O)₂CH₃, —C(O)C₁-C₆ alkyl, or —C(O)OC₁-C₆ alkyl; and
each of R⁶ and R⁷ is independently C₁-C₃ alkyl.

21. The compound of claim 20, wherein R¹ is methyl.

22. The compound of claim 21, wherein the compound is a compound is of formula (IIa-1): 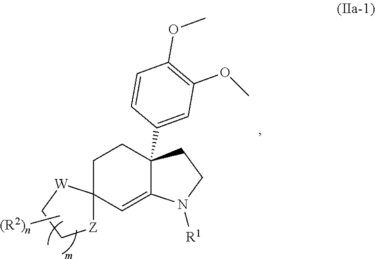
or a pharmaceutically acceptable salt thereof.

23. The compound of claim 22, or a pharmaceutically acceptable salt thereof, wherein n=0; and m=1.

24. The compound of claim 22, or a pharmaceutically acceptable salt thereof, wherein m=2.

25. The compound of claim 22, or a pharmaceutically acceptable salt thereof, wherein each R² is independently C₁-C₃ alkyl, —COOH, —CONH₂, C₃-C₁₀ cycloalkyl, C₂-C₆ alkenyl, phenyl, 5- to 7-membered heterocyclyl, or 5- to 6-membered heteroaryl; wherein each hydrogen atom in C₁-C₃alkyl, C₂-C₆ alkenyl, phenyl, 5- to 7-membered heterocyclyl and 5- to 6-membered heteroaryl is optionally substituted by —OH, C₁-C₃ alkyl, C₁-C₃ alkoxy, aryloxy, C₁-C₃ alkanol, or —NO₂; wherein each hydrogen atom in C₁-C₃ alkoxy and aryloxy is optionally substituted by C₁-C₃ alkoxy or phenyl.

26. The compound of claim 22, or a pharmaceutically acceptable salt thereof, wherein
n is 2 or 3; and
two R²s on a single carbon atom combine to form ═O; or
two adjacent R²s together with the carbon atoms to which they are attached combine to form 5- to 7-membered heteroaryl or phenyl; wherein each hydrogen atom in 5- to 7-membered heteroaryl or phenyl is optionally substituted by —OH, C₁-C₃ alkyl, or C₁-C₃ alkanol.

27. The compound of claim 20, wherein the compound is a compound of formula (IIIa-1): 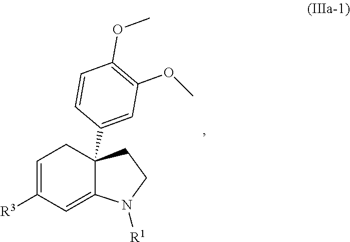
or a pharmaceutically acceptable salt thereof.

28. The compound of claim 27, wherein R³ is —OC(O)C₁-C₆ alkyl, —OC(O)C₂-C₆ alkenyl, —OC(O)C₃-C₆ cycloalkyl, —OC(O)phenyl, or —OC(O)-6-membered heteroaryl;
wherein each hydrogen atom in the —OC(O)C₁-C₆ alkyl or —OC(O)C₂-C₆ alkenyl is optionally substituted by halogen, C₁-C₆ alkyl, C₁-C₃ alkoxy, nitro, —N(C₁-C₃ alkyl)₂, C₁-C₃ haloalkyl, cyano, phenyl, phenoxy or —O(CH₂)ₚOCH₃; and
wherein each hydrogen atom in the —OC(O)C₃-C₆ cycloalkyl, —OC(O)phenyl, and —OC(O)₆-membered heteroaryl is optionally substituted by halogen, C₁-C₆ alkyl, C₁-C₃ alkoxy, nitro, —N(C₁-C₃ alkyl)₂, C₁-C₃ haloalkyl, cyano, or —O(CH₂)ₚOCH₃.

29. The compound of claim 28, wherein R³ is —OC(O)(phenyl), —OC(O)(C₁-C₆ alkyl) or —OC(O)(C₃-C₆ cycloalkyl).

30. The compound of claim 29, wherein R³ is

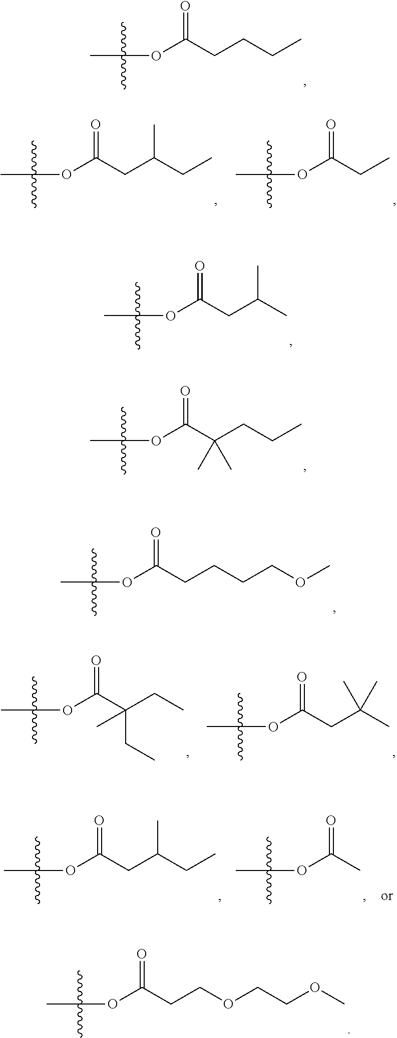

31. The compound of claim 21, wherein the compound is a compound of formula (Iva-1): 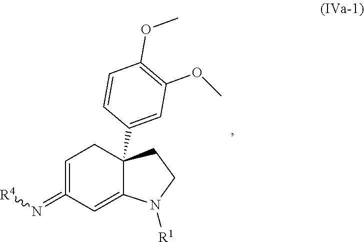
or a pharmaceutically acceptable salt thereof.

32. The compound of claim 31, wherein R⁴ is —OR⁵; and R⁵ is H, C₁-C₆ alkyl, C₃-C₁₀ cycloalkyl, C₃-C₆ heterocycloalkyl or phenyl; wherein each hydrogen atom in C₁-C₆ alkyl, C₃-C₆ heterocycloalkyl and phenyl is optionally substituted with —OH, C₁-C₆ alkoxy, 5- to 7-membered heteroaryl, phenyl, phenoxy, C₃-C₆ cycloalkyl, —C(O)OC₁-C₆ alkyl, C₃-C₁₀ cycloalkyl, or —COOH.

33. The compound of claim 31, wherein R⁴ is OH, C₁-C₆ alkoxy, or —NHC(O)-5- to 6-membered heteroaryl; wherein each hydrogen atom in —NHC(O)-5- to 6-membered heteroaryl is optionally substituted by —OH, C₁-C₃ alkyl, or C₁-C₃ alkanol.

34. The compound of claim 31, wherein R⁴ is —N(R⁵)₂; and each R⁵ is independently H, —C₁-C₆ alkyl, 5- to 10-membered heteroaryl, —C(O)-5- to 7-membered heteroaryl, —C(O)-phenyl, phenyl, —C(O)—C₁-C₃ alkyl, —C(O)—C₃-C₁₀ cycloalkyl, —C(O)N(H)-phenyl, —C(O)N(H)-pyridyl, —C(O)C(O)OH, or —CONH₂; wherein each hydrogen atom in —C₁-C₆ alkyl, 5- to 10-membered heteroaryl, —C(O)-5- to 7-membered heteroaryl, —C(O)-phenyl, phenyl, —C(O)—C₁-C₃ alkyl, and —C(O)N(H)-phenyl is optionally substituted by halogen, aryloxy, —COOH, phenyl, —S(O)₂CH₃, C₁-C₃ alkoxy, C₃-C₆ cycloalkyl optionally substituted by —COOH, 5- to 7-membered heteroaryl, or C₃-C₆ heterocycloalkyl optionally substituted by methyl.

35. The compound of claim 21, wherein the compound is a compound of formula (IA): 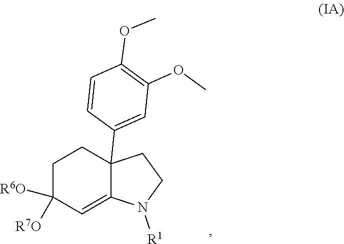
or a pharmaceutically acceptable salt thereof,

36. The compound of claim 21, wherein the compound is a compound of Formula (IX), 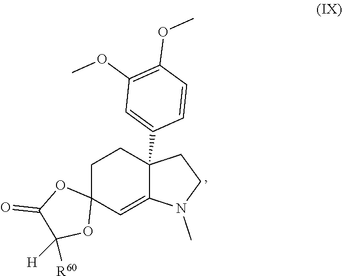
or a pharmaceutically acceptable salt thereof; wherein R₆₀ is hydrogen or methyl.

37. The compound of claim 21, wherein the compound is a compound of Formula (X), 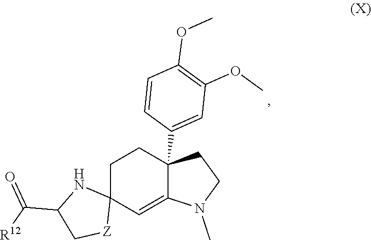
or a pharmaceutically acceptable salt thereof; wherein Z is S or O; and R₁₂ is hydroxyl or amino.

38. A compound of Formula (XV) 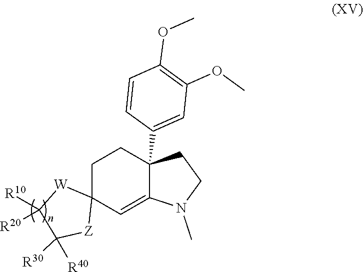
or a pharmaceutically acceptable salt thereof, wherein
n is 0 or 1;
W and Z are each independently CH₂, O, S or NH, provided that at least one of W or Z is O, S or NH; and
R₁₀, R₂₀, R₃₀ and R₄₀ are each hydrogen.

39. A compound selected from the group consisting of:
a. a compound of Formula (XI) 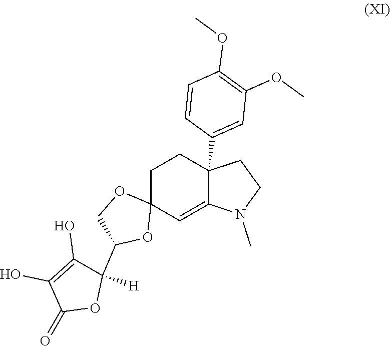
or a pharmaceutically acceptable salt thereof;
b. a compound of Formula (XII) 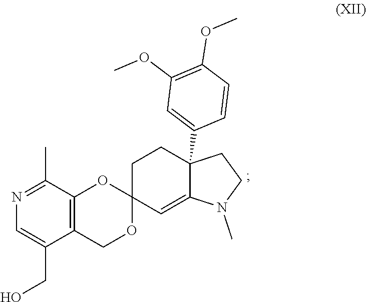 [SMILES: COc1ccc([C@]23CCN(C)C2=CC2(CC3)OCc3c(CO)cnc(C)c3O2)cc1OC]
or a pharmaceutically acceptable salt thereof;
c. a compound selected from the group consisting of: 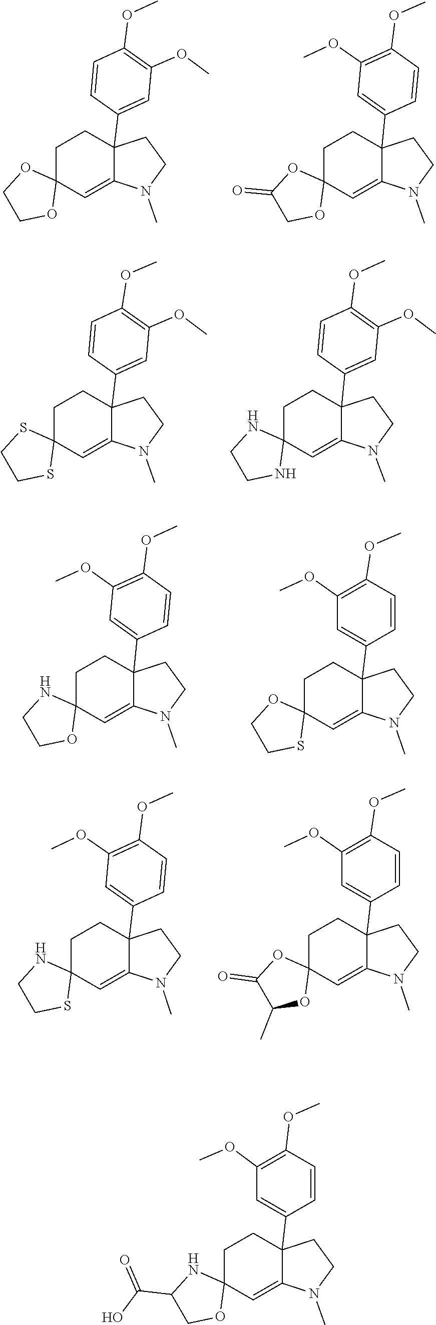 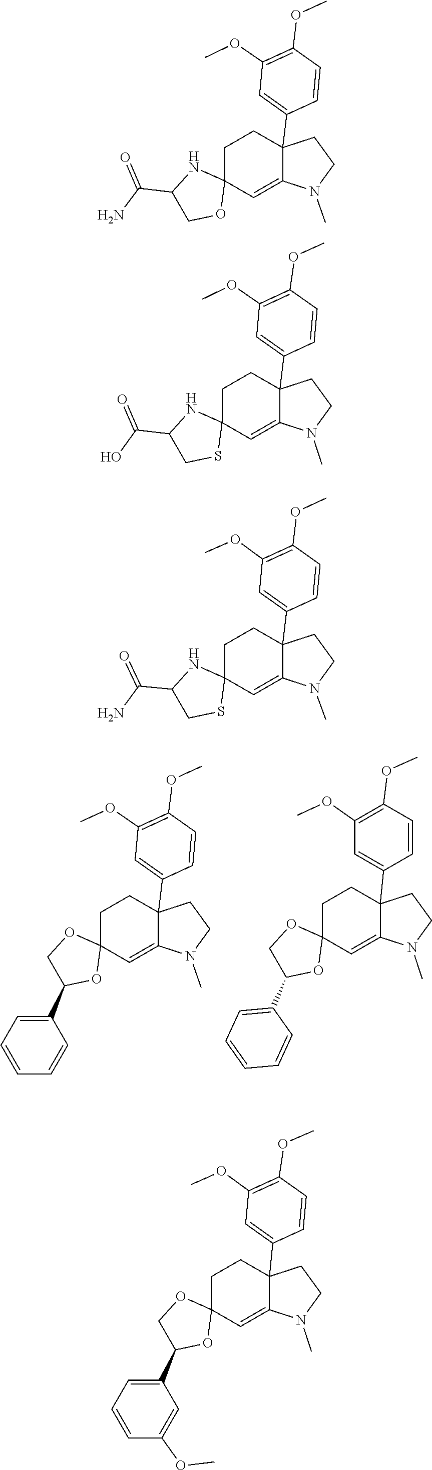 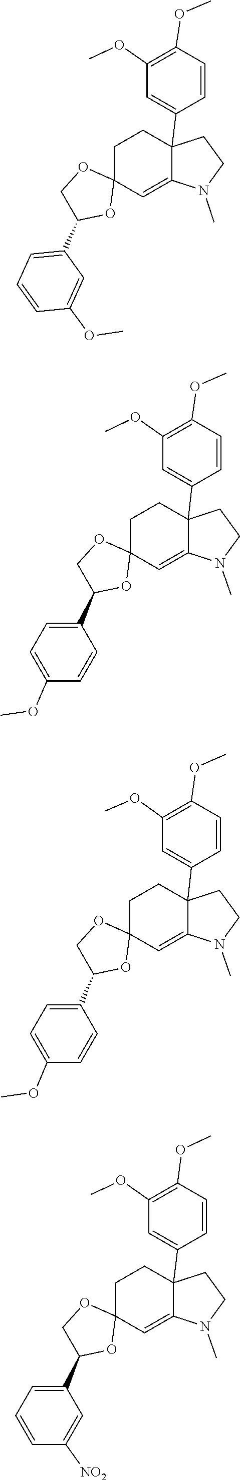 [SMILES: COc1cccc([C@@H]2COC3(C=C4N(C)CCC4(c4ccc(OC)c(OC)c4)CC3)O2)c1 ; COc1ccc([C@H]2COC3(C=C4N(C)CCC4(c4ccc(OC)c(OC)c4)CC3)O2)cc1 ; COc1ccc([C@@H]2COC3(C=C4N(C)CCC4(c4ccc(OC)c(OC)c4)CC3)O2)cc1 ; COc1ccc(C23CCN(C)C2=CC2(CC3)OC[C@H](c3cccc([N+](=O)[O-])c3)O2)cc1OC] 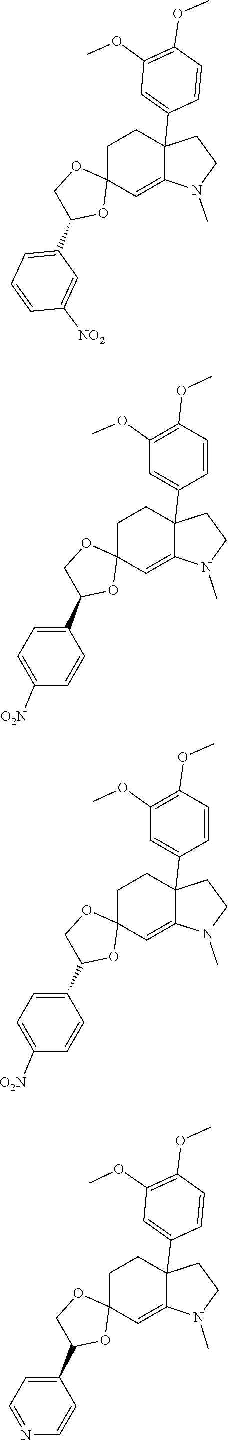 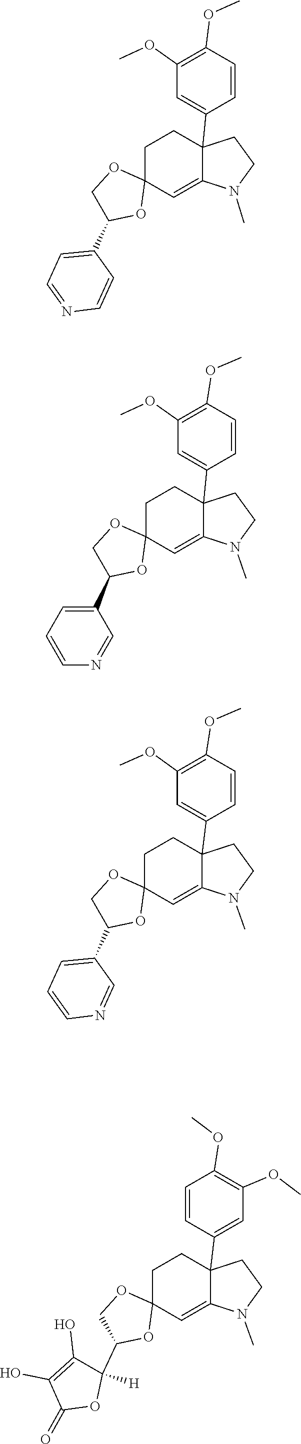 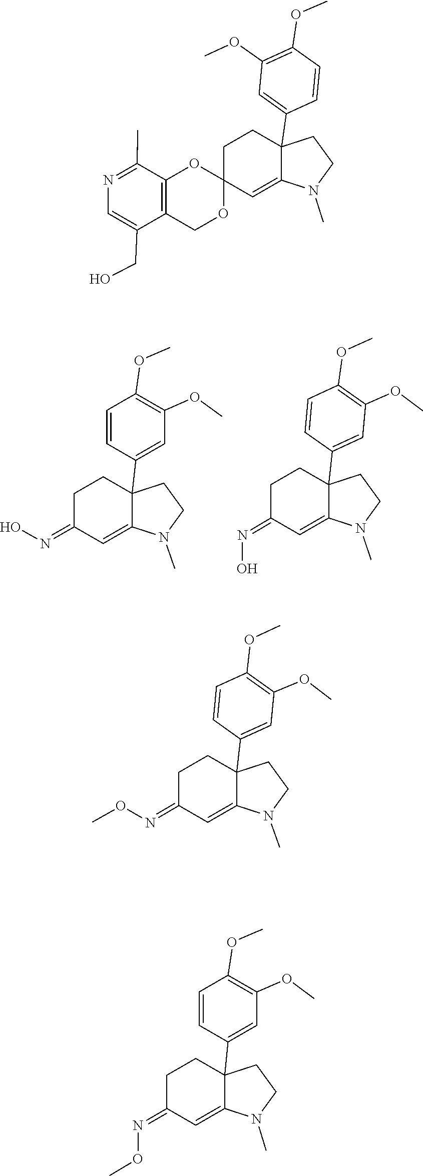 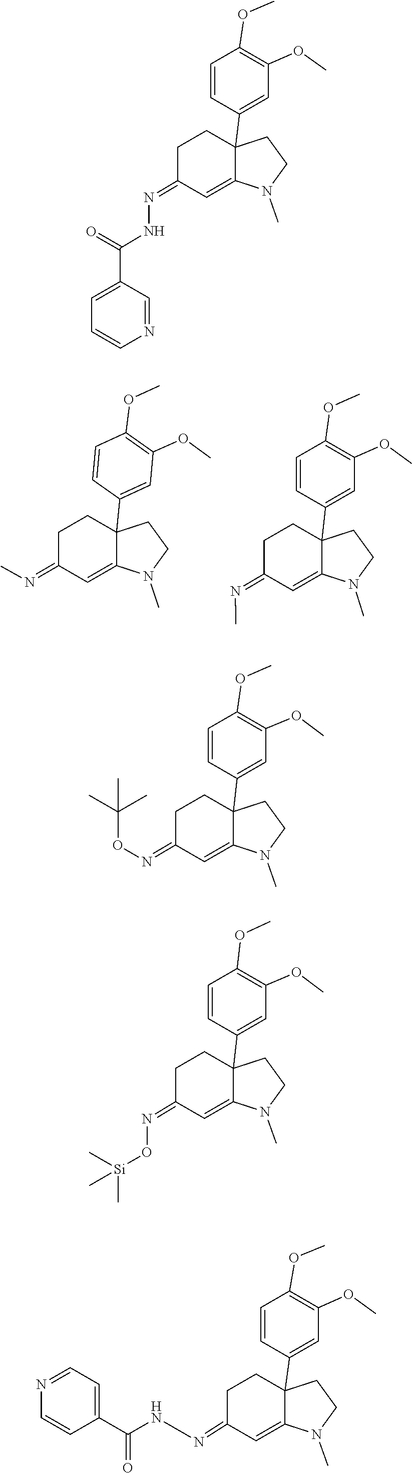 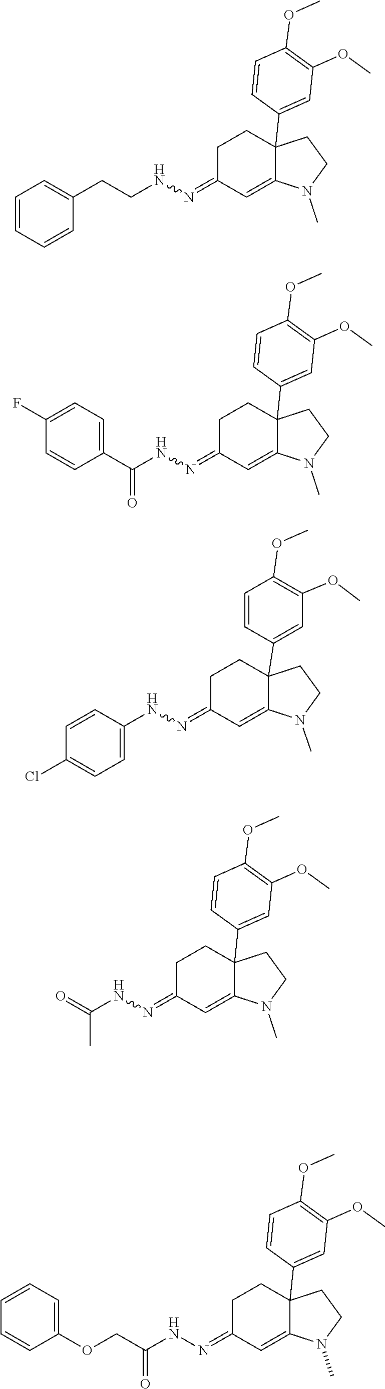 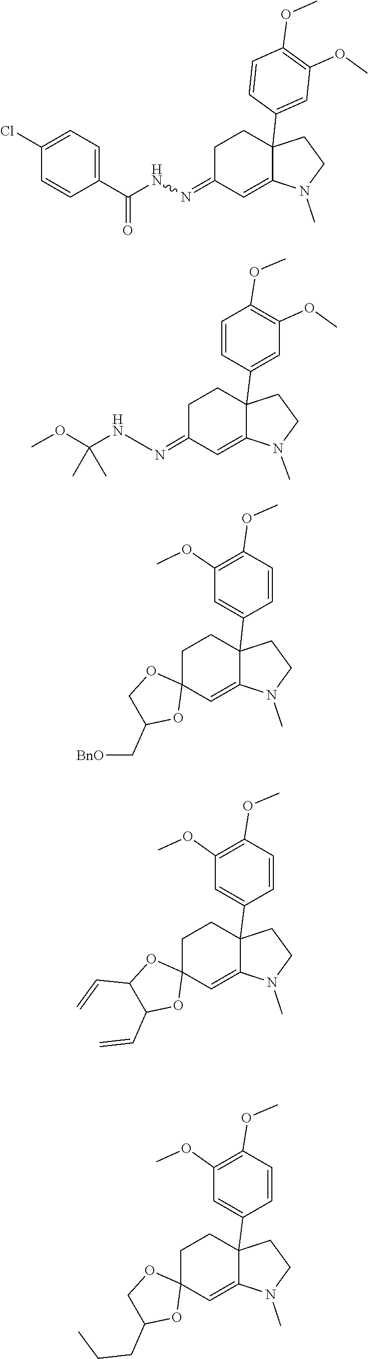 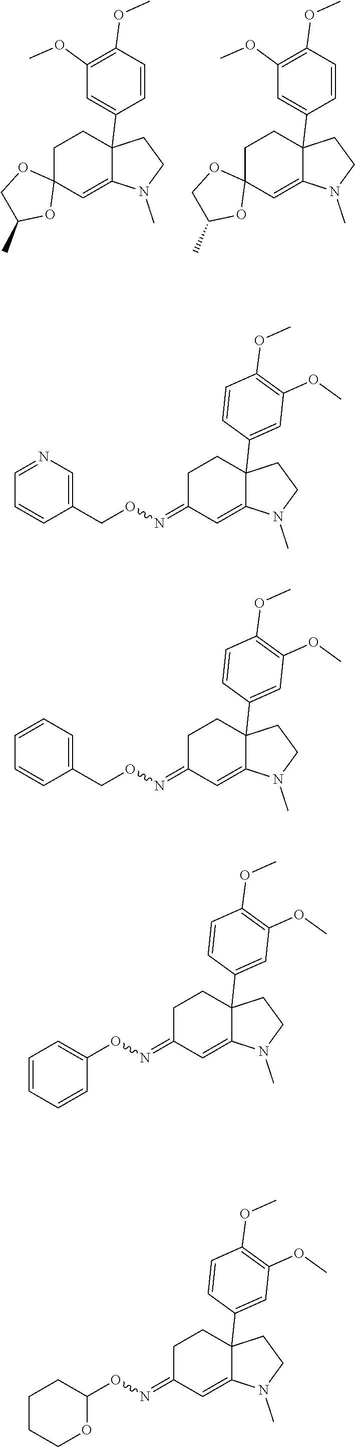 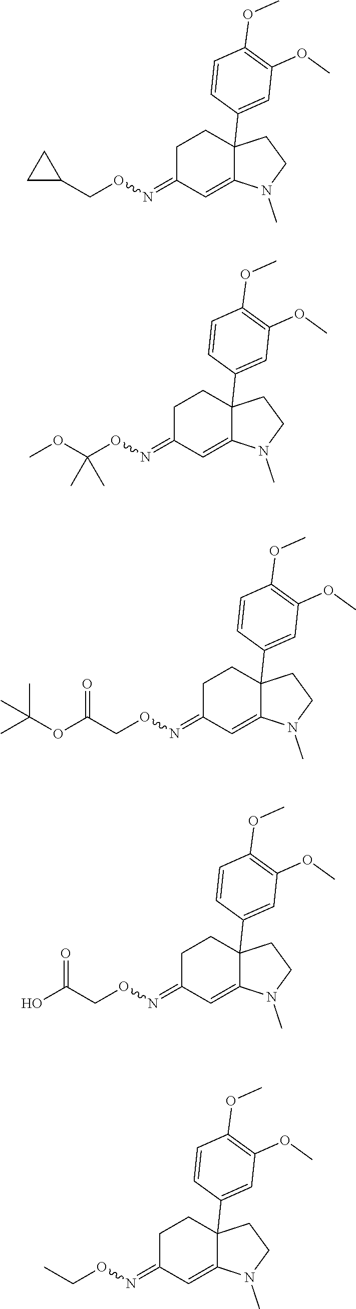 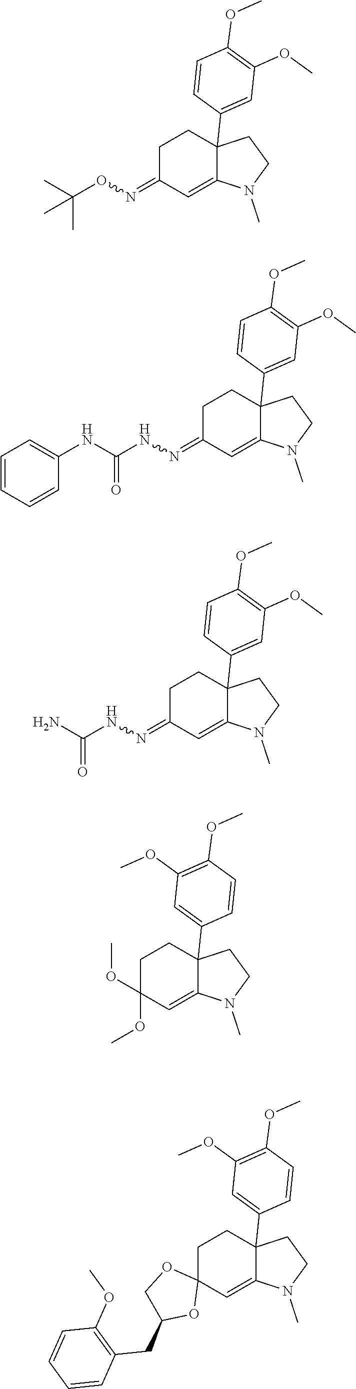 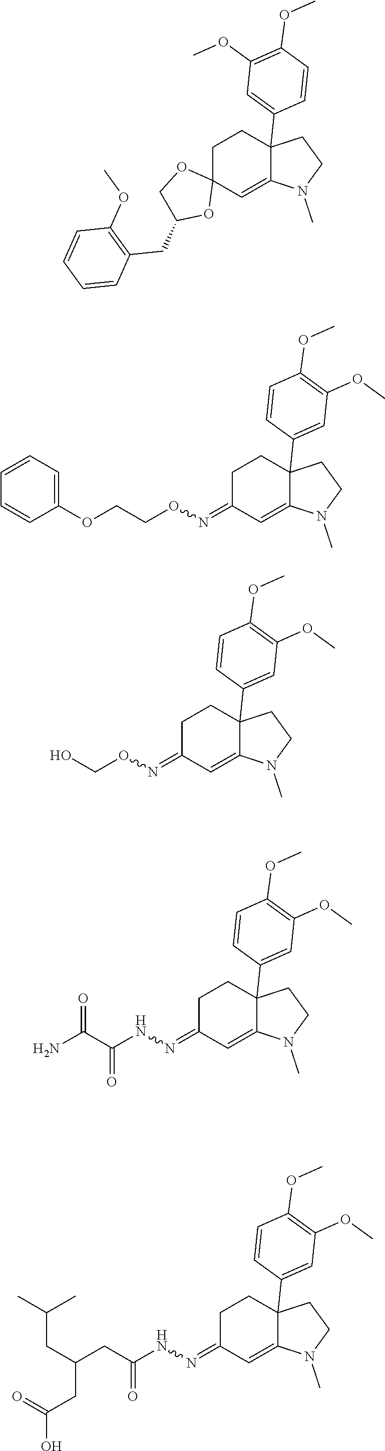 [SMILES: COc1ccccc1C[C@@H]1COC2(C=C3N(C)CCC3(c3ccc(OC)c(OC)c3)CC2)O1 ; COc1ccc(C23CCC(=NOCCOc4ccccc4)C=C2N(C)CC3)cc1OC ; COc1ccc(C23CCC(=NOCO)C=C2N(C)CC3)cc1OC ; COc1ccc(C23CCC(=NNC(=O)C(N)=O)C=C2N(C)CC3)cc1OC ; COc1ccc(C23CCC(=NNC(=O)CC(CC(=O)O)CC(C)C)C=C2N(C)CC3)cc1OC] 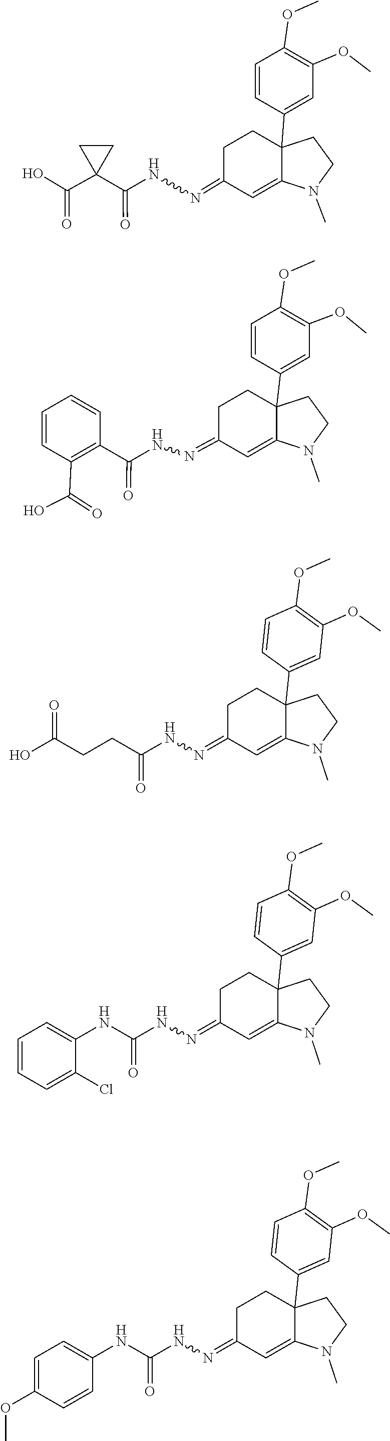 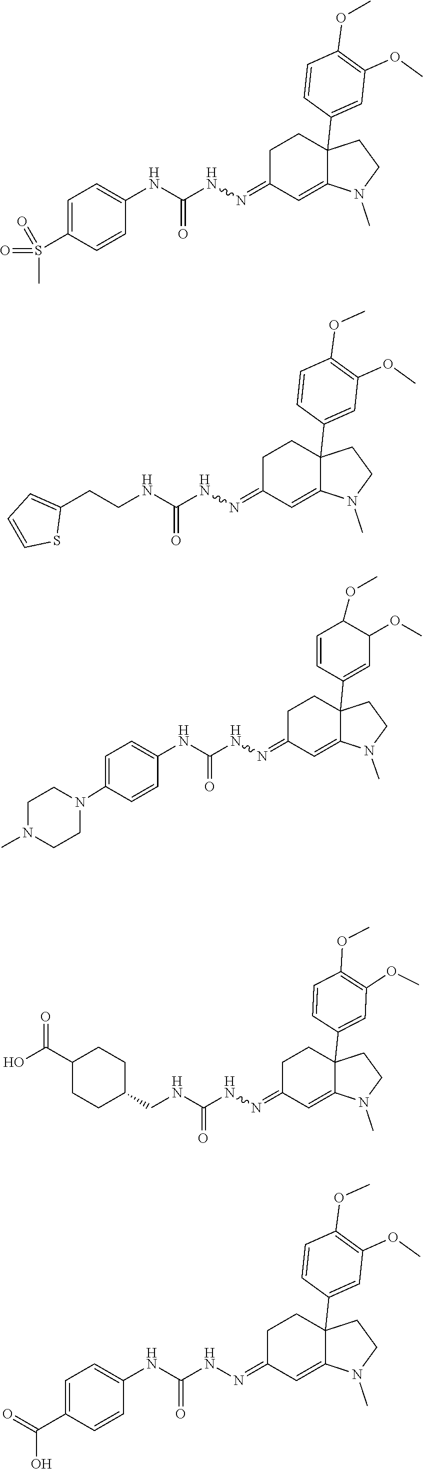 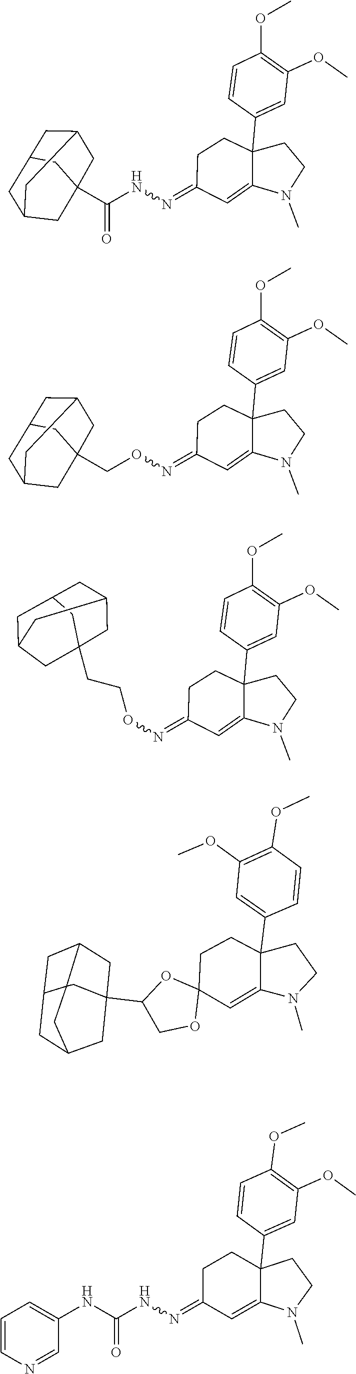 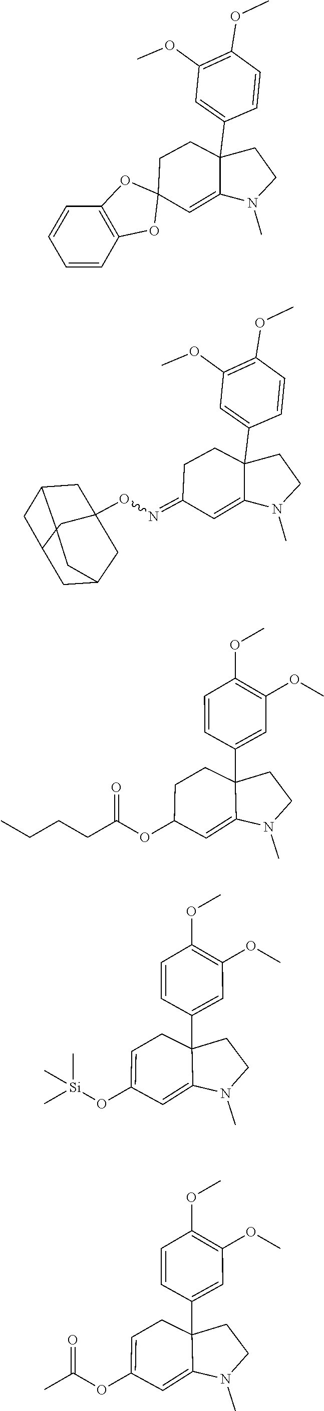 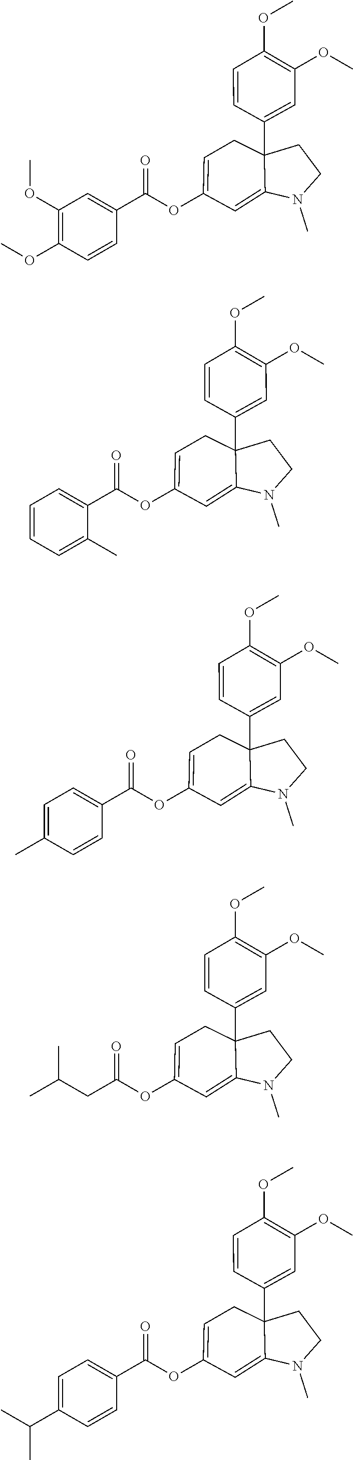 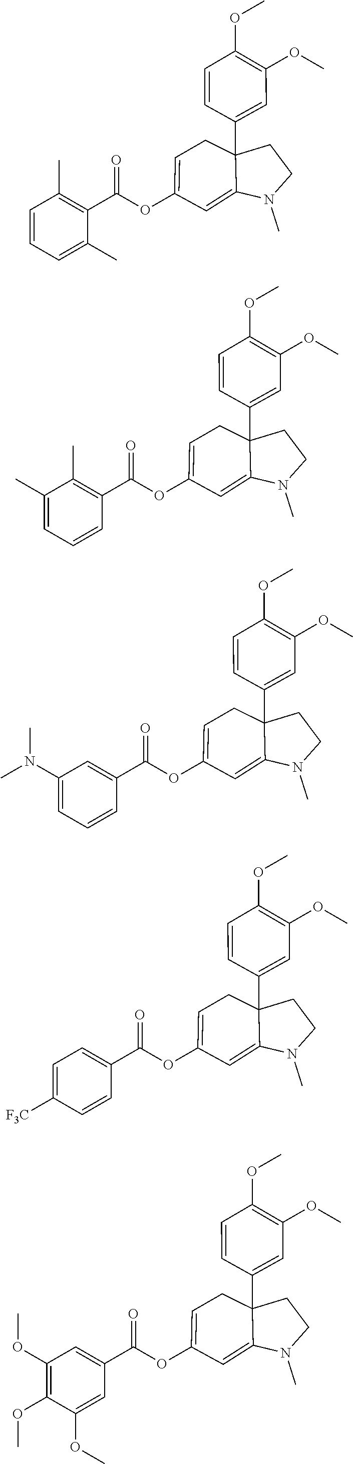 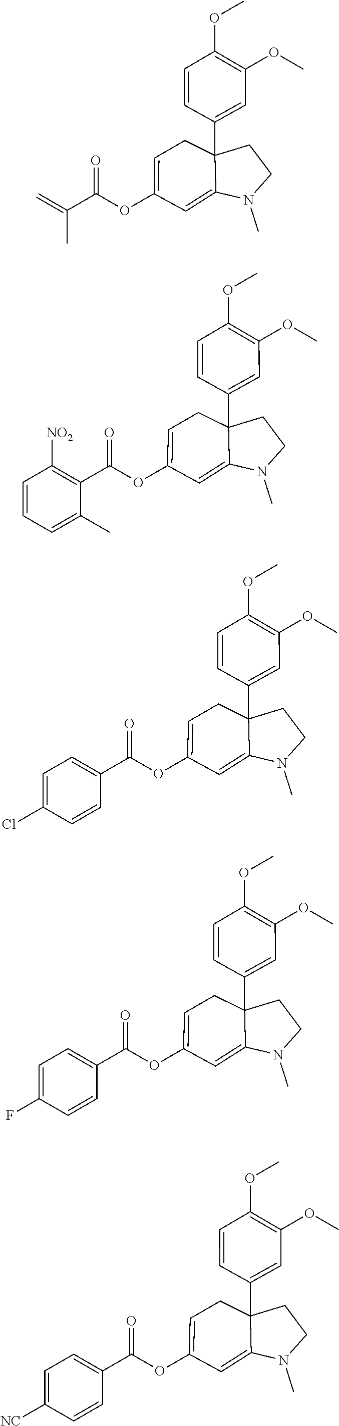 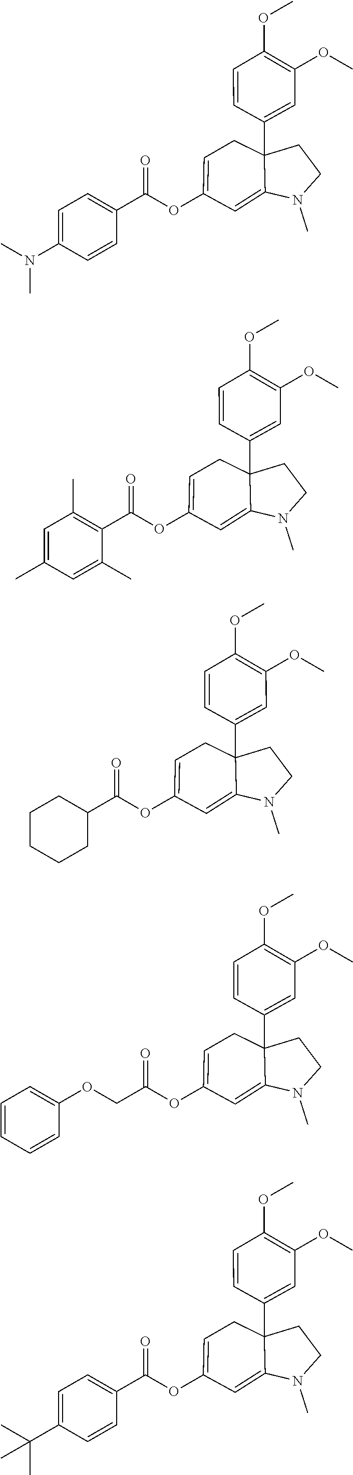 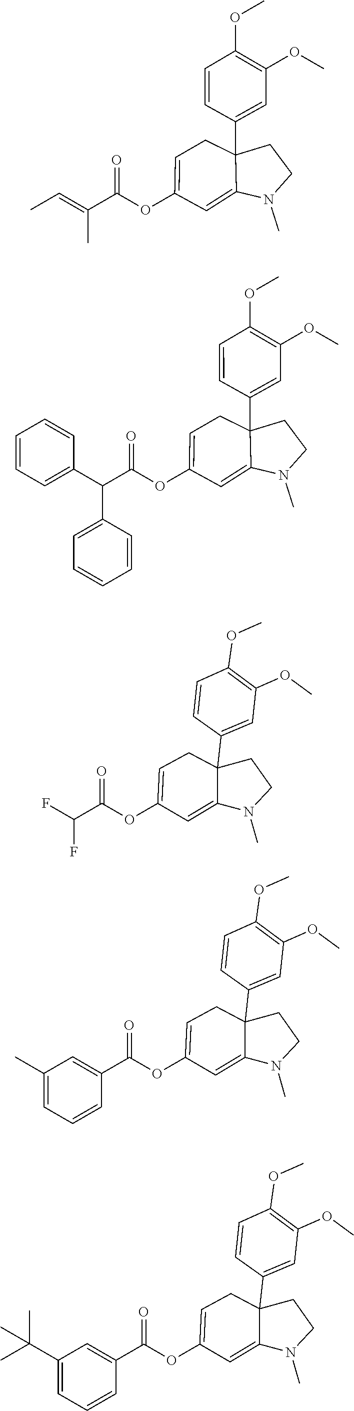 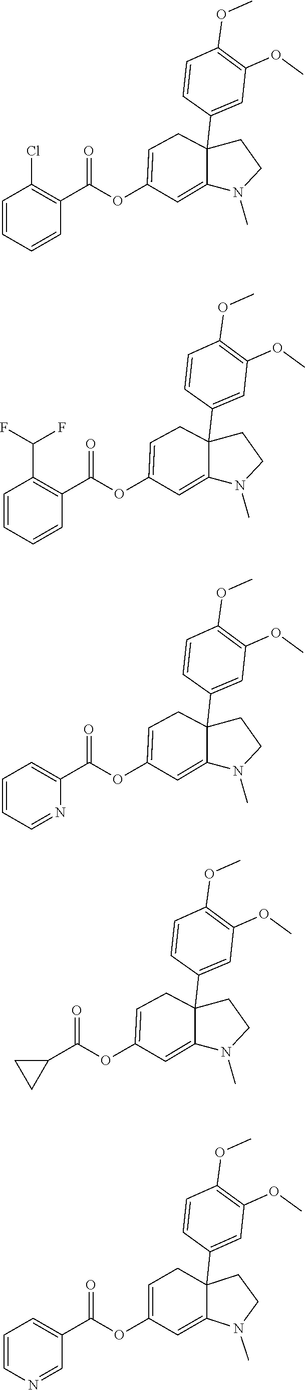 [SMILES: COc1ccc(C23CC=C(OC(=O)c4ccccc4Cl)C=C2N(C)CC3)cc1OC ; COc1ccc(C23CC=C(OC(=O)c4ccccc4C(F)F)C=C2N(C)CC3)cc1OC ; COc1ccc(C23CC=C(OC(=O)c4ccccn4)C=C2N(C)CC3)cc1OC ; COc1ccc(C23CC=C(OC(=O)C4CC4)C=C2N(C)CC3)cc1OC ; COc1ccc(C23CC=C(OC(=O)c4cccnc4)C=C2N(C)CC3)cc1OC] 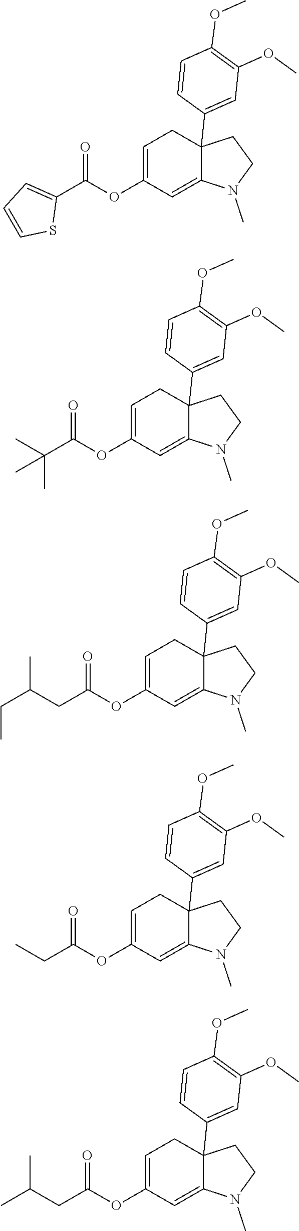 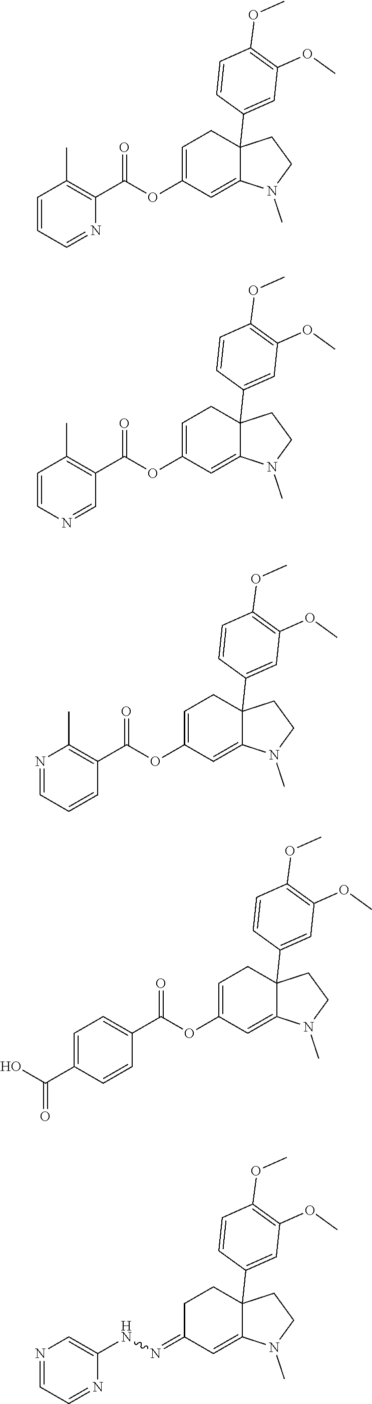 [SMILES: COc1ccc(C23CC=C(OC(=O)c4ncccc4C)C=C2N(C)CC3)cc1OC ; COc1ccc(C23CC=C(OC(=O)c4cnccc4C)C=C2N(C)CC3)cc1OC ; COc1ccc(C23CC=C(OC(=O)c4cccnc4C)C=C2N(C)CC3)cc1OC ; COc1ccc(C23CC=C(OC(=O)c4ccc(C(=O)O)cc4)C=C2N(C)CC3)cc1OC ; COc1ccc(C23CCC(=NNc4cnccn4)C=C2N(C)CC3)cc1OC] 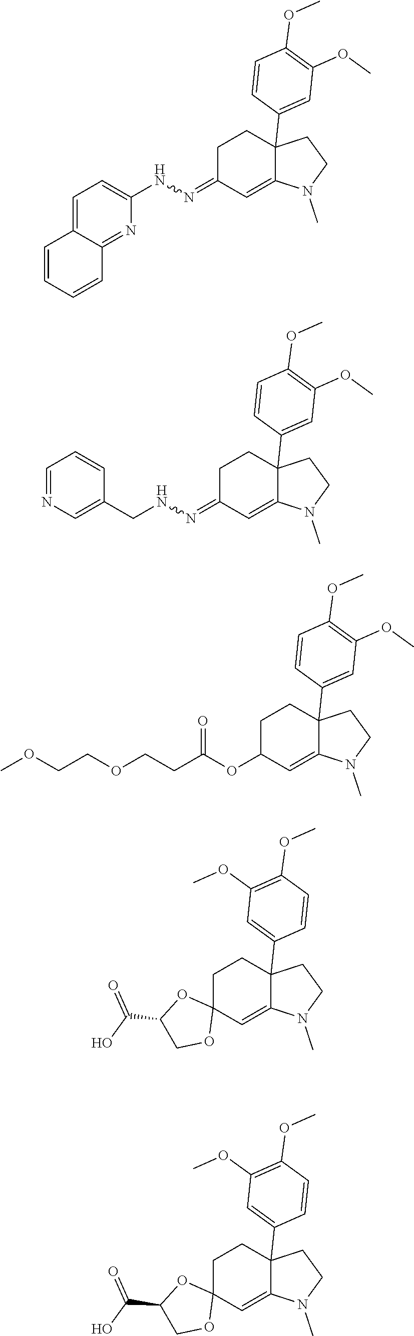 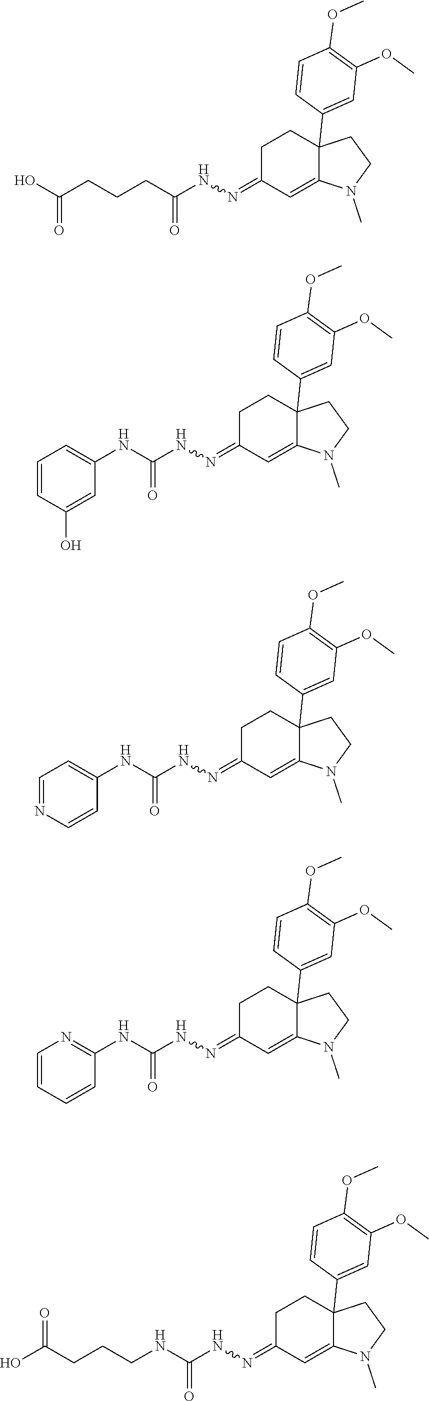 [SMILES: COc1ccc(C23CCC(=NNC(=O)CCCC(=O)O)C=C2N(C)CC3)cc1OC ; COc1ccc(C23CCC(=NNC(=O)Nc4cccc(O)c4)C=C2N(C)CC3)cc1OC ; COc1ccc(C23CCC(=NNC(=O)Nc4ccncc4)C=C2N(C)CC3)cc1OC ; COc1ccc(C23CCC(=NNC(=O)Nc4ccccn4)C=C2N(C)CC3)cc1OC ; COc1ccc(C23CCC(=NNC(=O)NCCCC(=O)O)C=C2N(C)CC3)cc1OC] 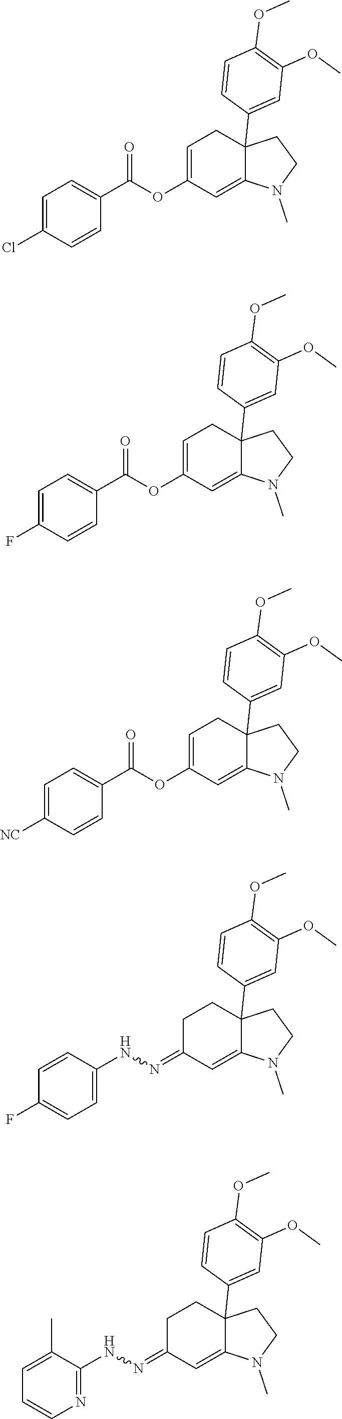 [SMILES: COc1ccc(C23CC=C(OC(=O)c4ccc(Cl)cc4)C=C2N(C)CC3)cc1OC ; COc1ccc(C23CC=C(OC(=O)c4ccc(F)cc4)C=C2N(C)CC3)cc1OC ; COc1ccc(C23CC=C(OC(=O)c4ccc(C#N)cc4)C=C2N(C)CC3)cc1OC ; COc1ccc(C23CCC(=NNc4ccc(F)cc4)C=C2N(C)CC3)cc1OC ; COc1ccc(C23CCC(=NNc4ncccc4C)C=C2N(C)CC3)cc1OC] 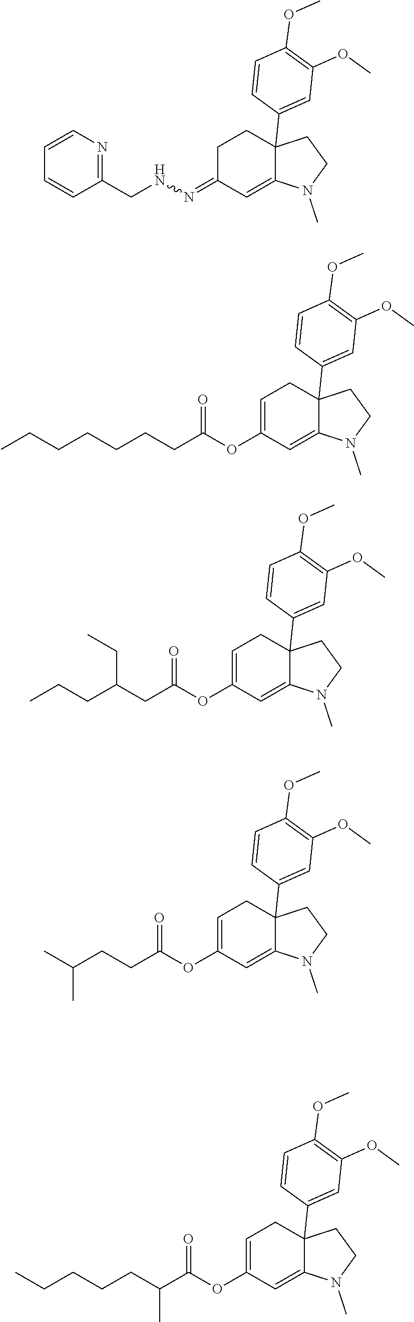 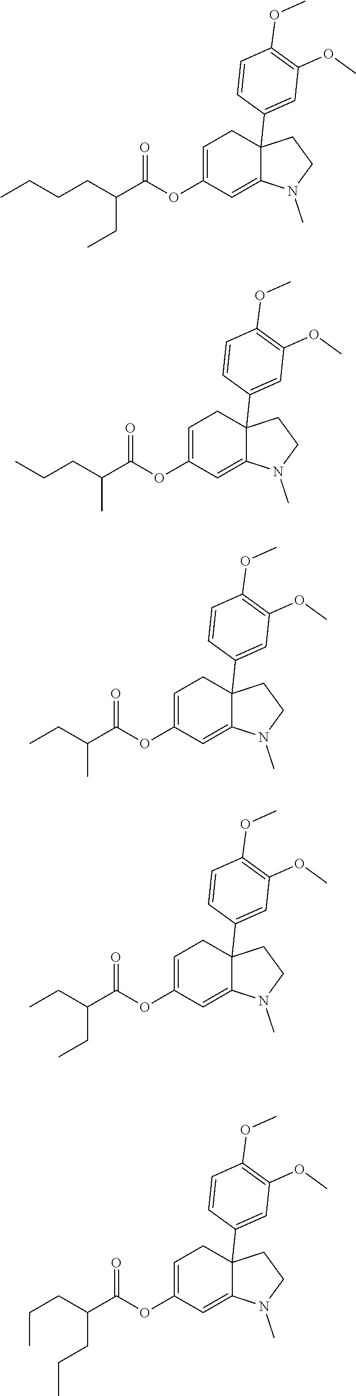 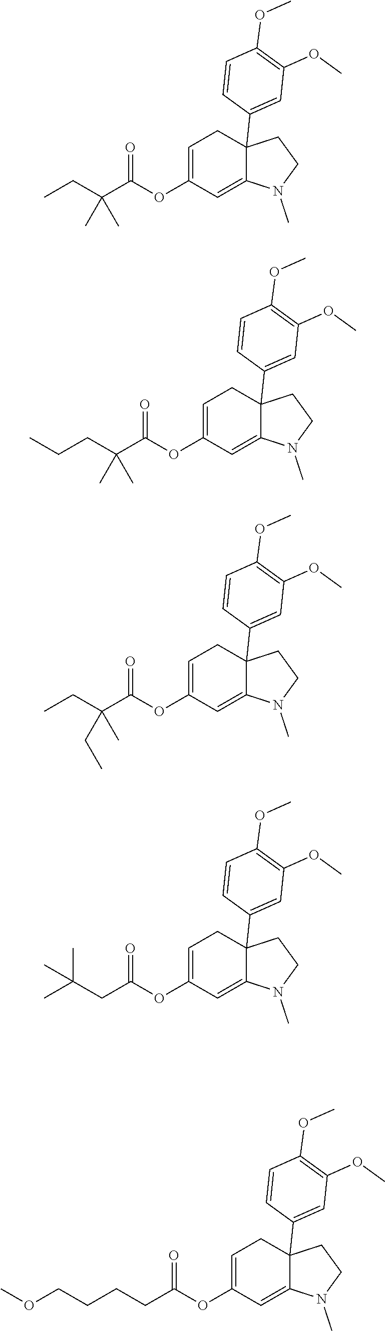
or a pharmaceutically acceptable salt thereof;
d. a compound selected from the group consisting of: 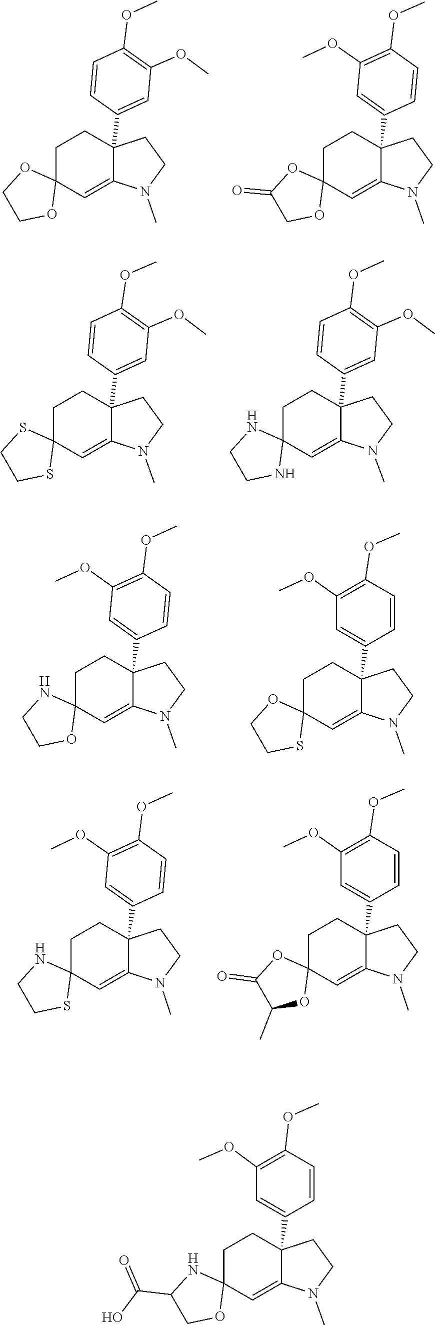 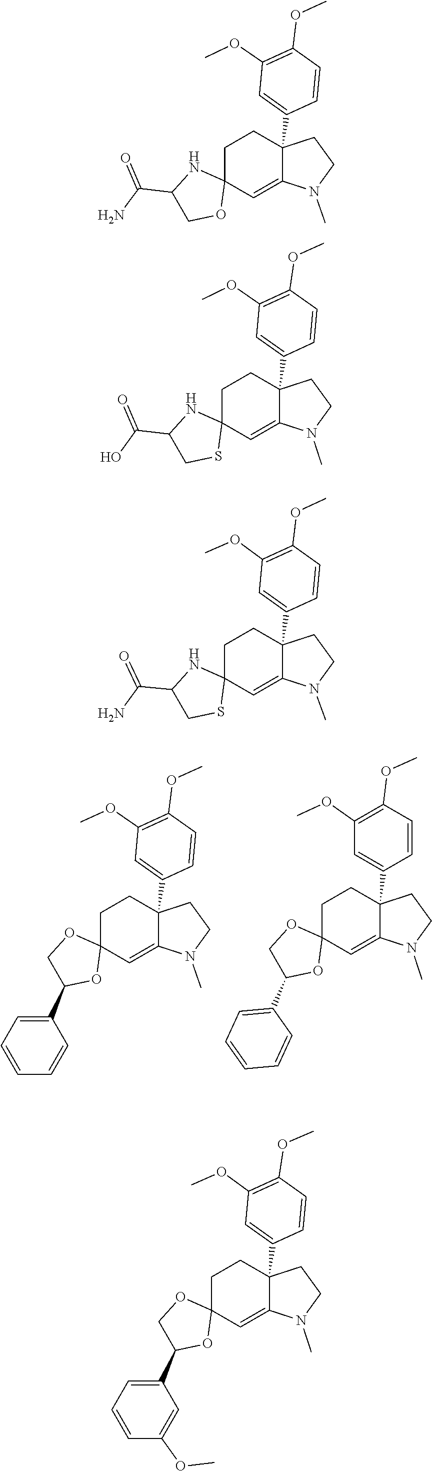 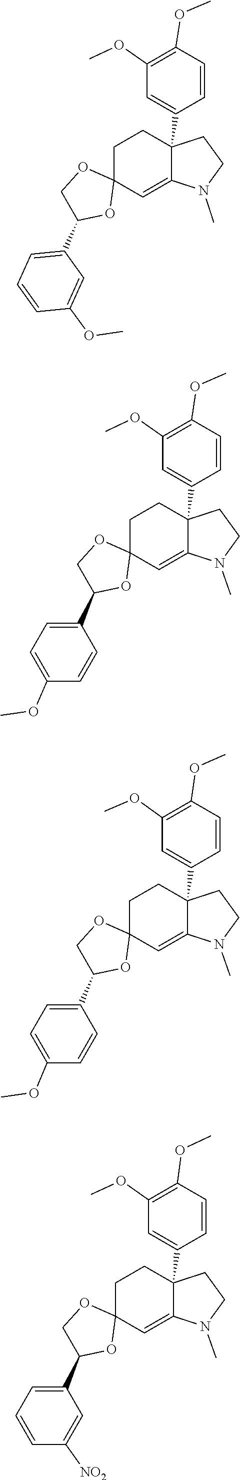 [SMILES: COc1cccc([C@@H]2COC3(C=C4N(C)CC[C@]4(c4ccc(OC)c(OC)c4)CC3)O2)c1 ; COc1ccc([C@H]2COC3(C=C4N(C)CC[C@]4(c4ccc(OC)c(OC)c4)CC3)O2)cc1 ; COc1ccc([C@@H]2COC3(C=C4N(C)CC[C@]4(c4ccc(OC)c(OC)c4)CC3)O2)cc1 ; COc1ccc([C@]23CCN(C)C2=CC2(CC3)OC[C@H](c3cccc([N+](=O)[O-])c3)O2)cc1OC] 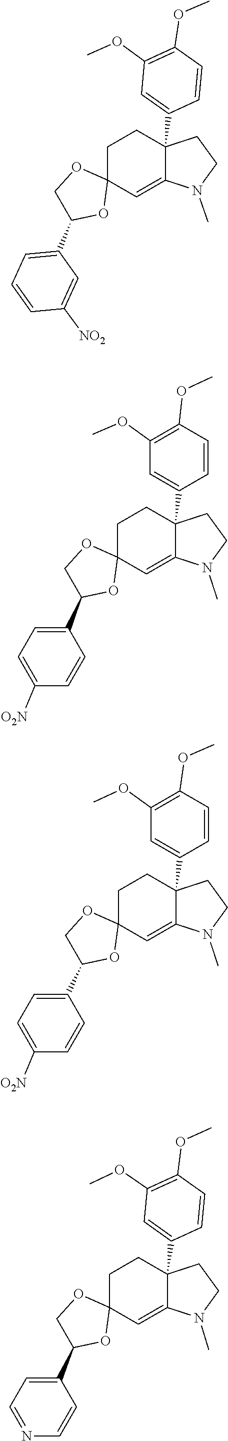 [SMILES: COc1ccc([C@]23CCN(C)C2=CC2(CC3)OC[C@@H](c3cccc([N+](=O)[O-])c3)O2)cc1OC ; COc1ccc([C@]23CCN(C)C2=CC2(CC3)OC[C@H](c3ccc([N+](=O)[O-])cc3)O2)cc1OC ; COc1ccc([C@]23CCN(C)C2=CC2(CC3)OC[C@@H](c3ccc([N+](=O)[O-])cc3)O2)cc1OC ; COc1ccc([C@]23CCN(C)C2=CC2(CC3)OC[C@H](c3ccncc3)O2)cc1OC] 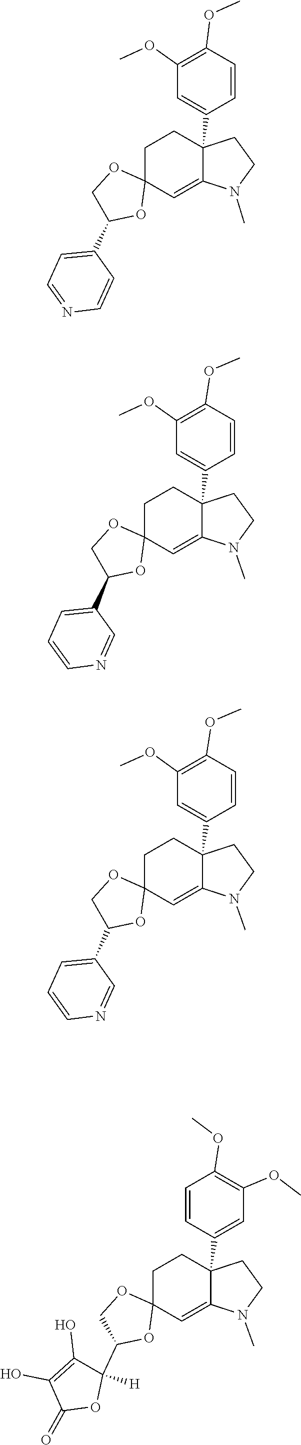 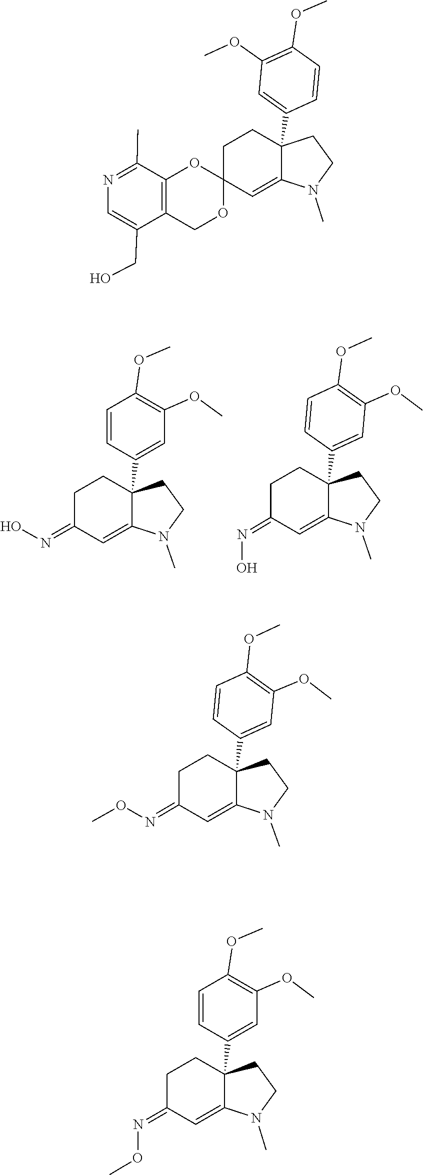 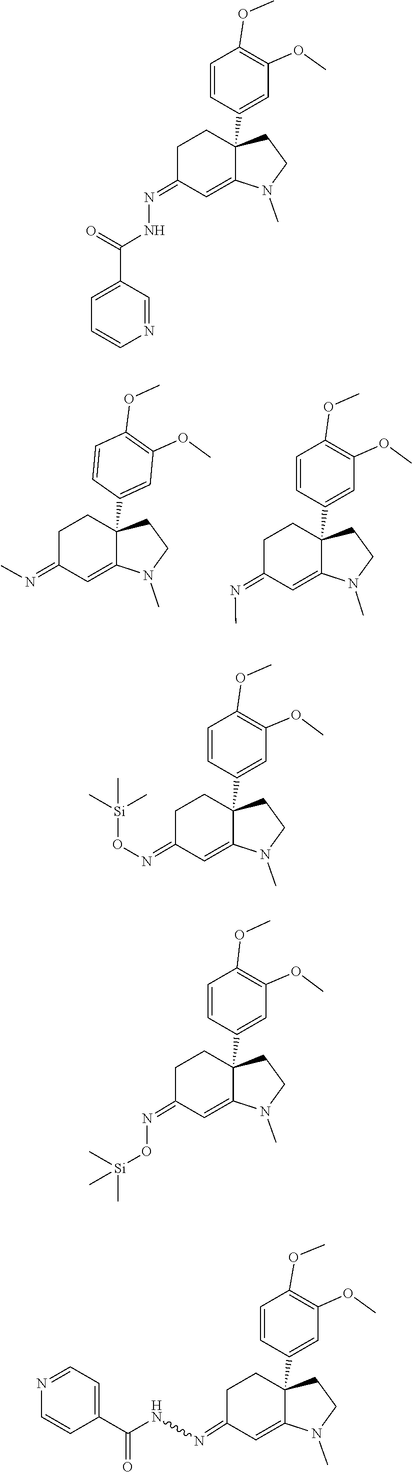 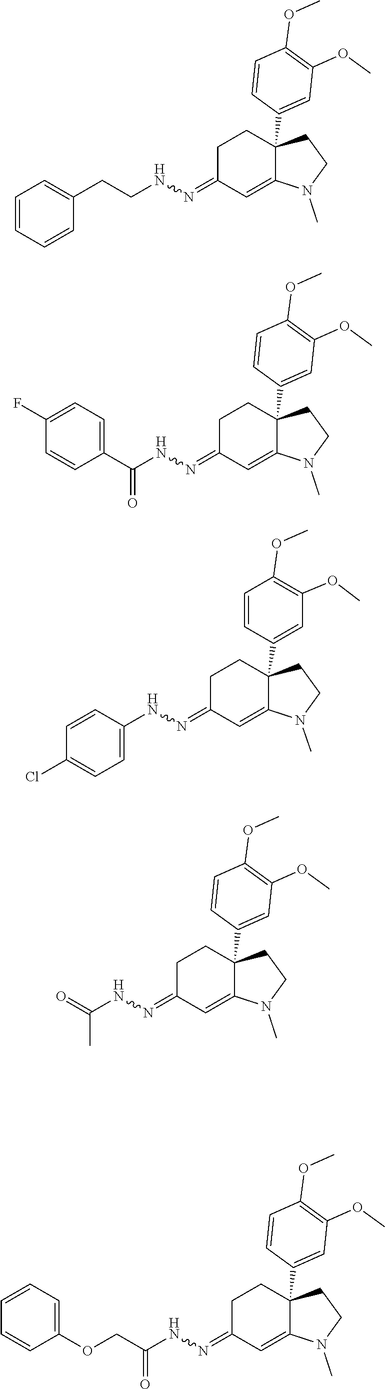 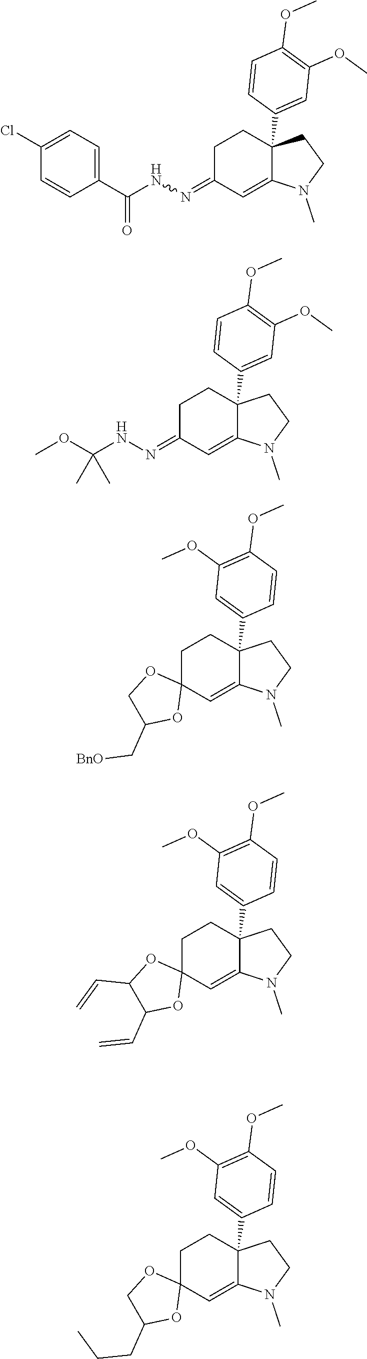 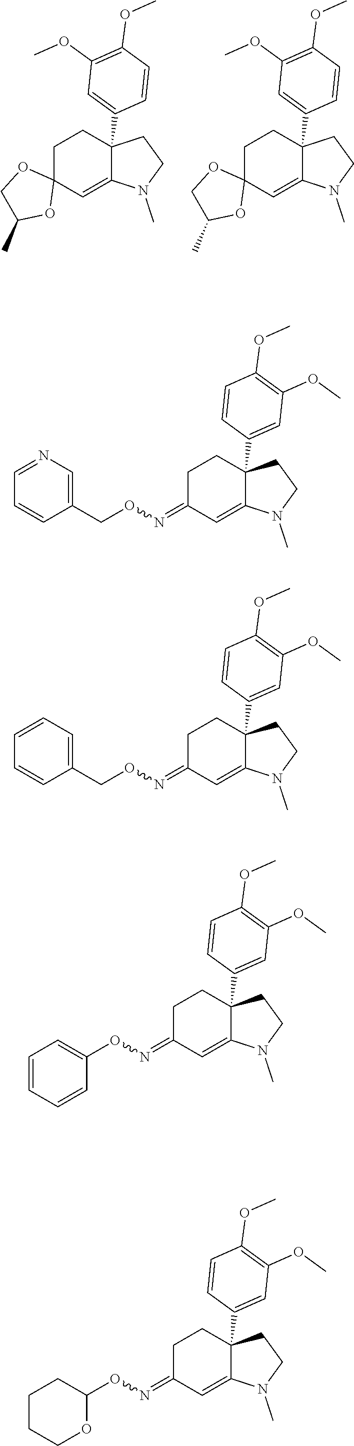 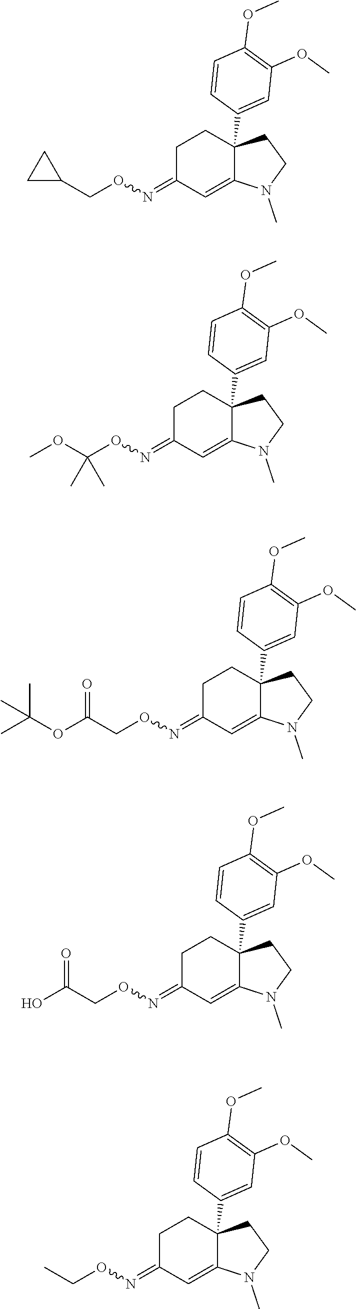 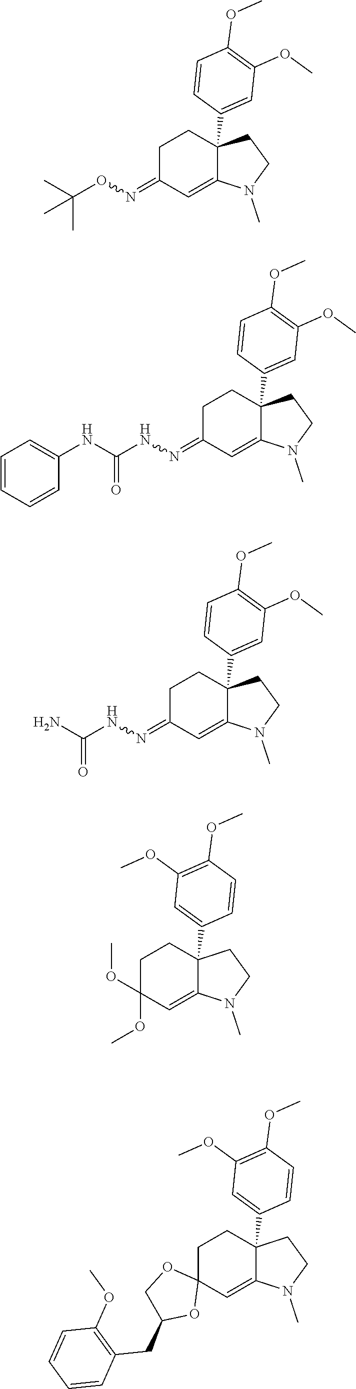 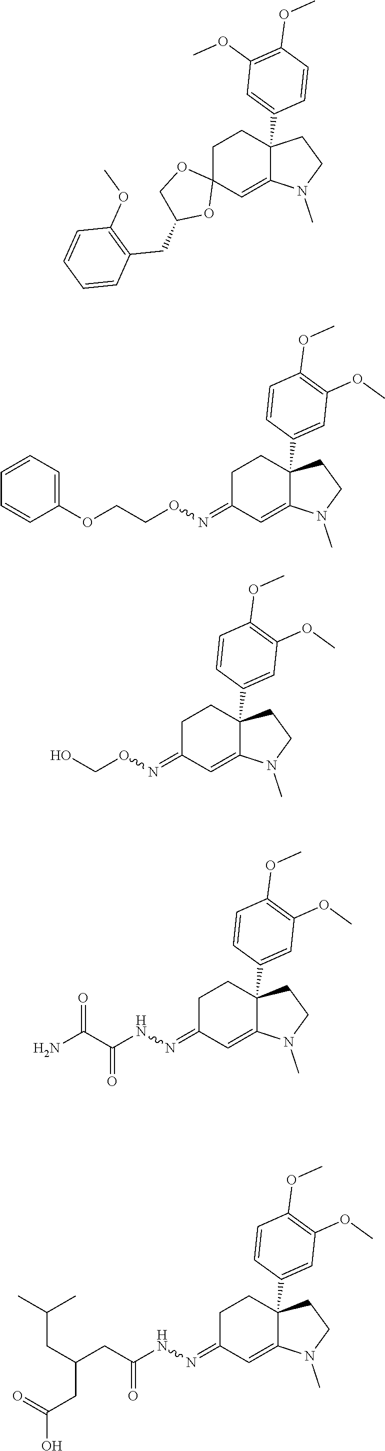 [SMILES: COc1ccccc1C[C@@H]1COC2(C=C3N(C)CC[C@]3(c3ccc(OC)c(OC)c3)CC2)O1 ; COc1ccc([C@@]23CCC(=NOCCOc4ccccc4)C=C2N(C)CC3)cc1OC ; COc1ccc([C@@]23CCC(=NOCO)C=C2N(C)CC3)cc1OC ; COc1ccc([C@@]23CCC(=NNC(=O)C(N)=O)C=C2N(C)CC3)cc1OC ; COc1ccc([C@@]23CCC(=NNC(=O)CC(CC(=O)O)CC(C)C)C=C2N(C)CC3)cc1OC] 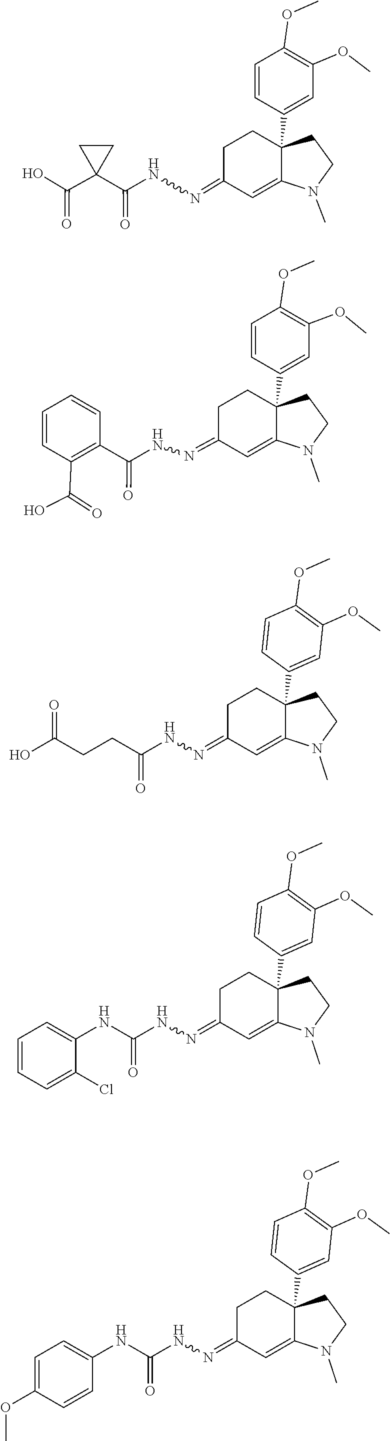 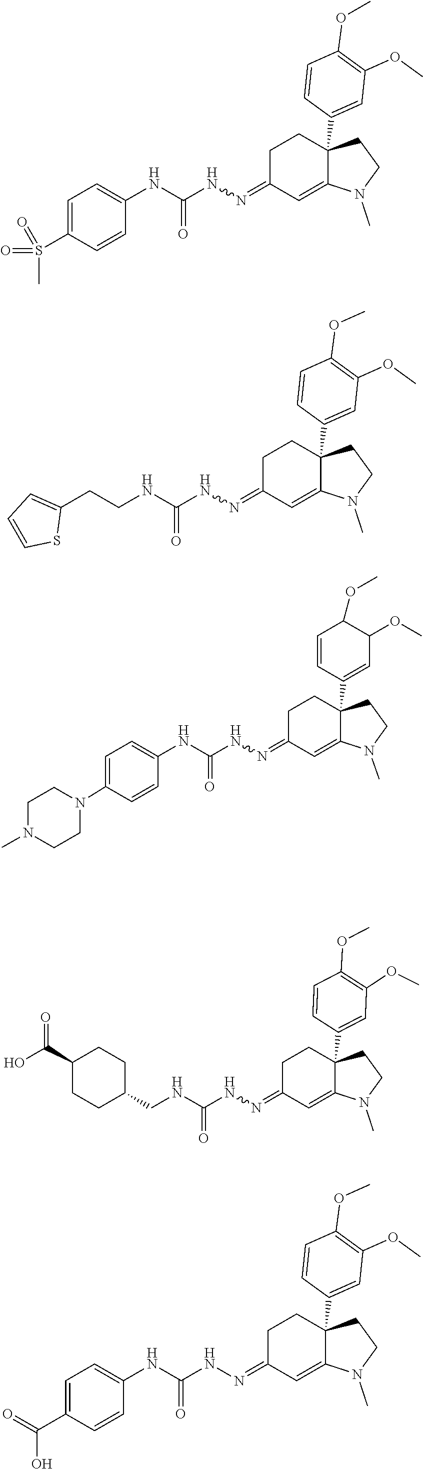 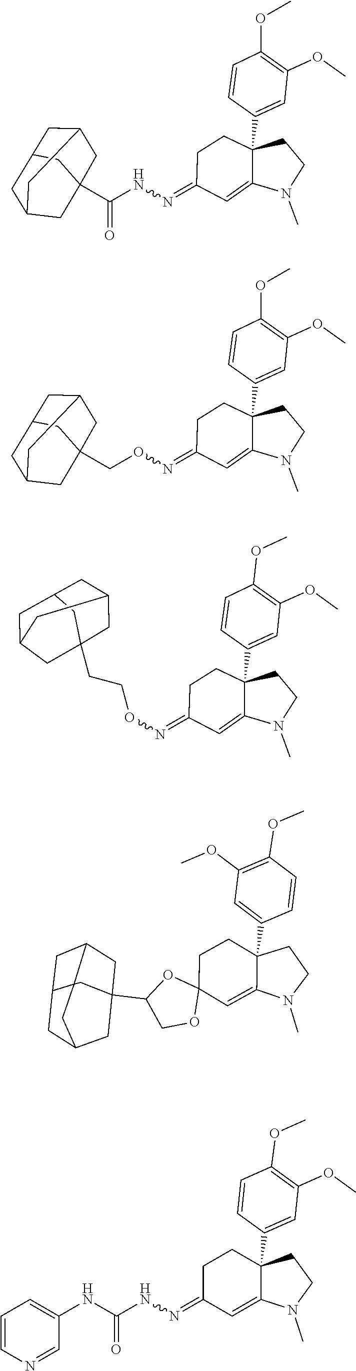 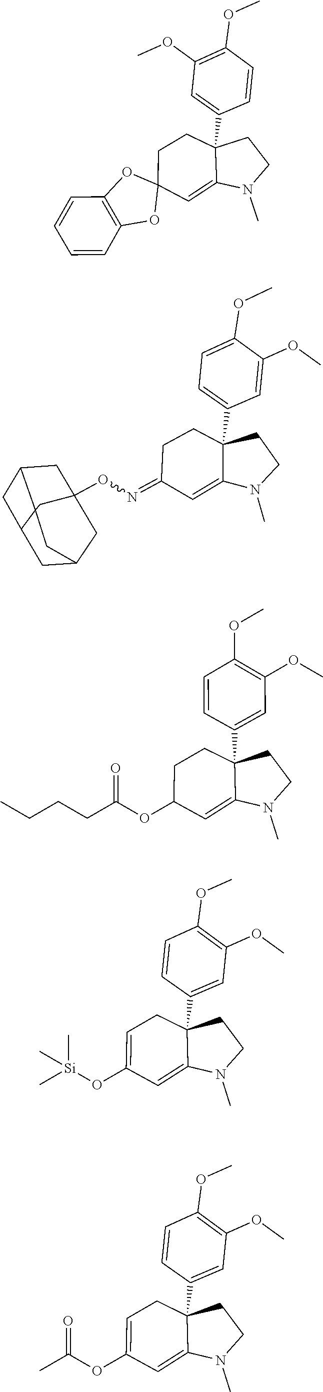 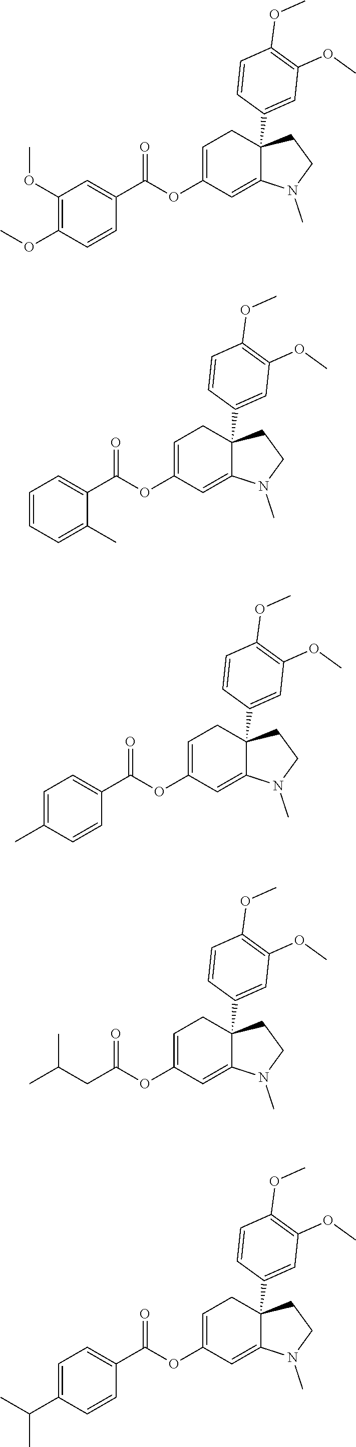 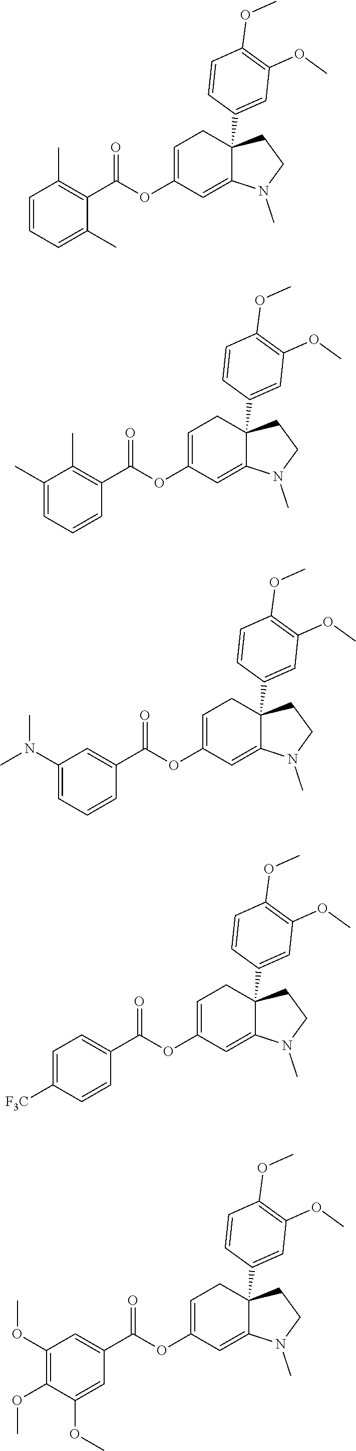 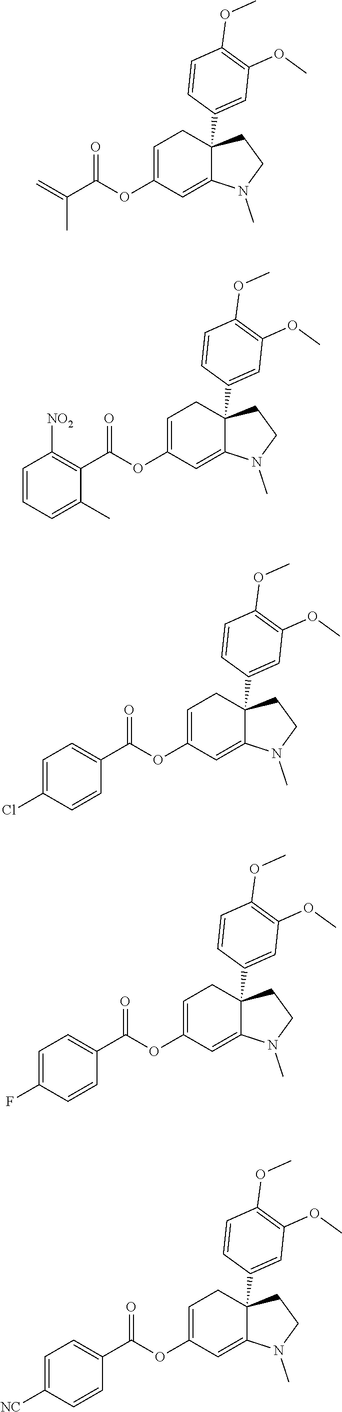 [SMILES: C=C(C)C(=O)OC1=CC[C@@]2(c3ccc(OC)c(OC)c3)CCN(C)C2=C1 ; COc1ccc([C@@]23CC=C(OC(=O)c4c(C)cccc4[N+](=O)[O-])C=C2N(C)CC3)cc1OC ; COc1ccc([C@@]23CC=C(OC(=O)c4ccc(Cl)cc4)C=C2N(C)CC3)cc1OC ; COc1ccc([C@@]23CC=C(OC(=O)c4ccc(F)cc4)C=C2N(C)CC3)cc1OC ; COc1ccc([C@@]23CC=C(OC(=O)c4ccc(C#N)cc4)C=C2N(C)CC3)cc1OC] 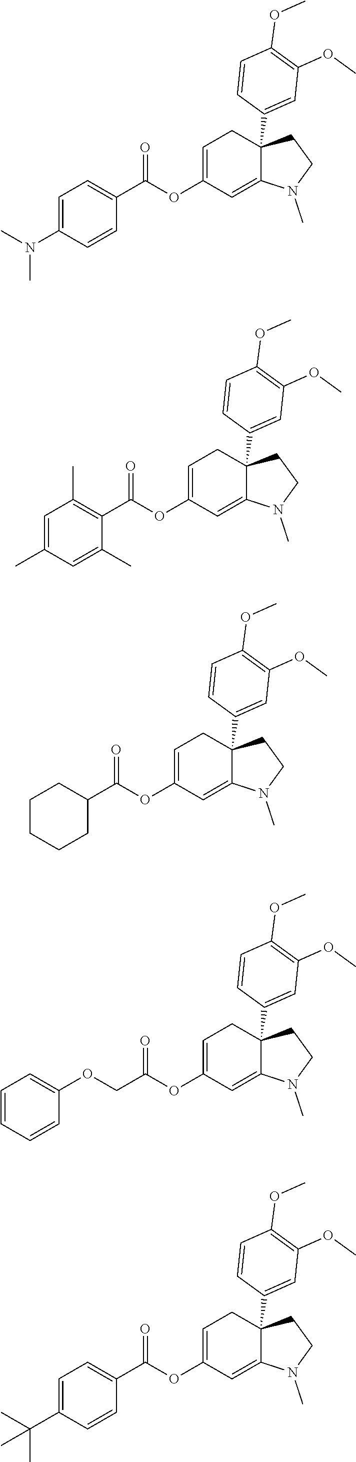 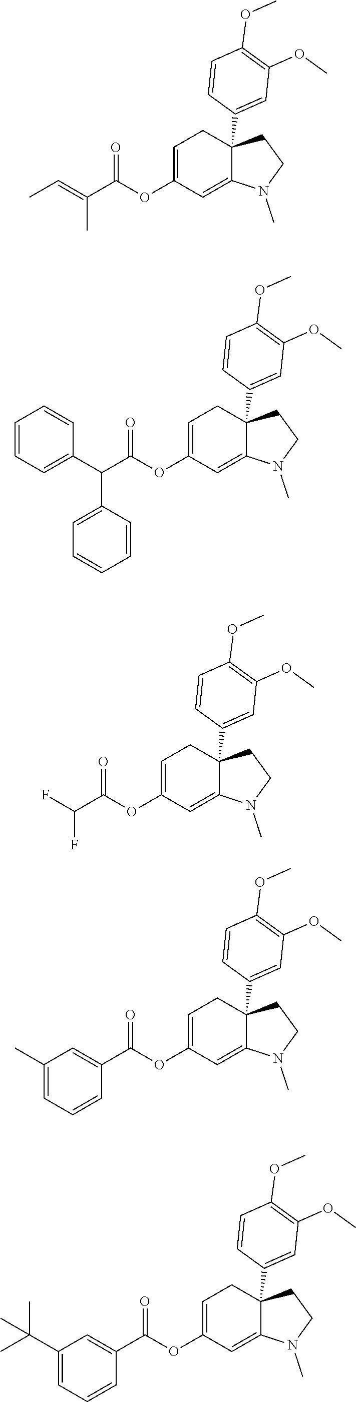 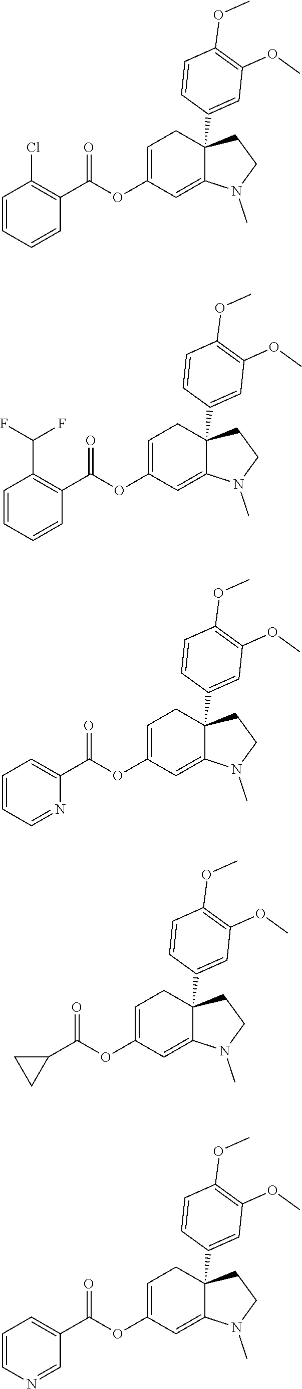 [SMILES: COc1ccc([C@@]23CC=C(OC(=O)c4ccccc4Cl)C=C2N(C)CC3)cc1OC ; COc1ccc([C@@]23CC=C(OC(=O)c4ccccc4C(F)F)C=C2N(C)CC3)cc1OC ; COc1ccc([C@@]23CC=C(OC(=O)c4ccccn4)C=C2N(C)CC3)cc1OC ; COc1ccc([C@@]23CC=C(OC(=O)C4CC4)C=C2N(C)CC3)cc1OC ; COc1ccc([C@@]23CC=C(OC(=O)c4cccnc4)C=C2N(C)CC3)cc1OC] 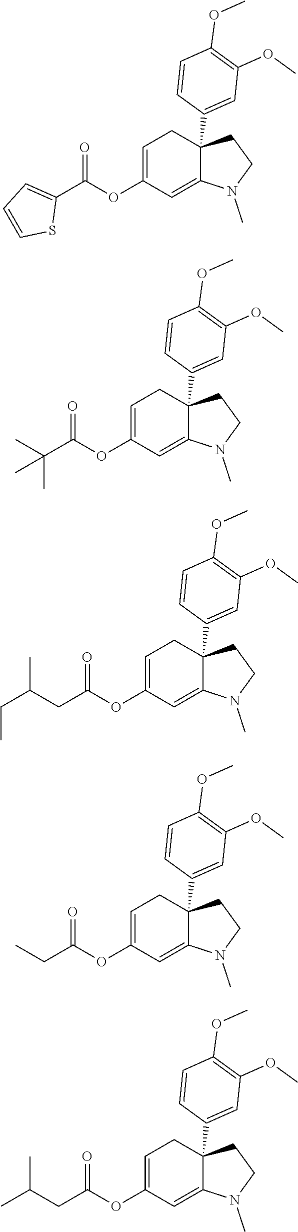 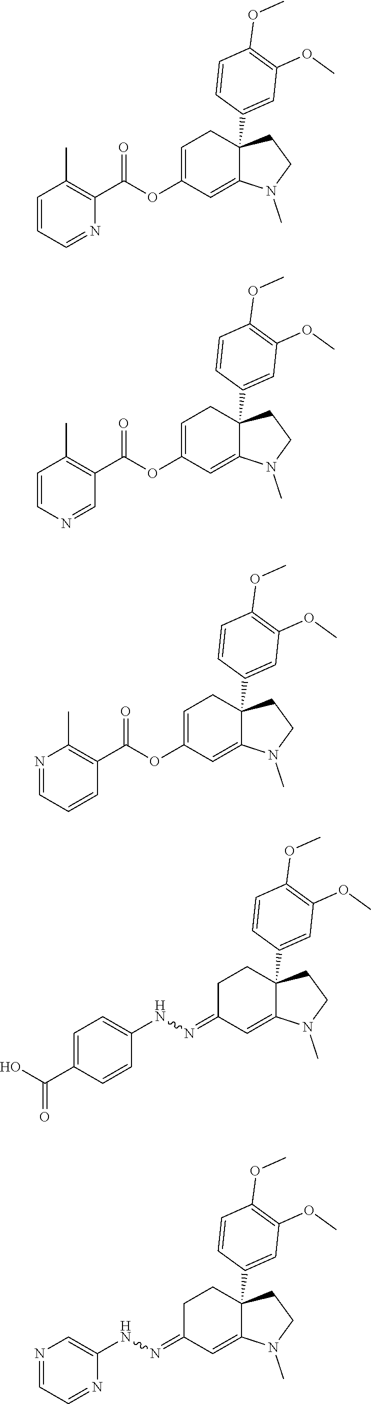 [SMILES: COc1ccc([C@@]23CC=C(OC(=O)c4ncccc4C)C=C2N(C)CC3)cc1OC ; COc1ccc([C@@]23CC=C(OC(=O)c4cnccc4C)C=C2N(C)CC3)cc1OC ; COc1ccc([C@@]23CC=C(OC(=O)c4cccnc4C)C=C2N(C)CC3)cc1OC ; COc1ccc([C@@]23CCC(=NNc4ccc(C(=O)O)cc4)C=C2N(C)CC3)cc1OC ; COc1ccc([C@@]23CCC(=NNc4cnccn4)C=C2N(C)CC3)cc1OC] 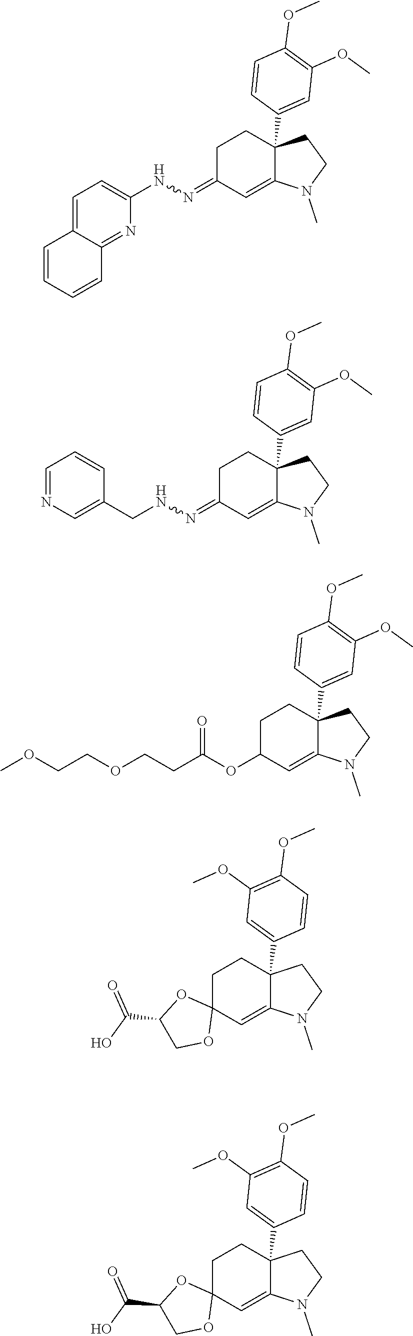 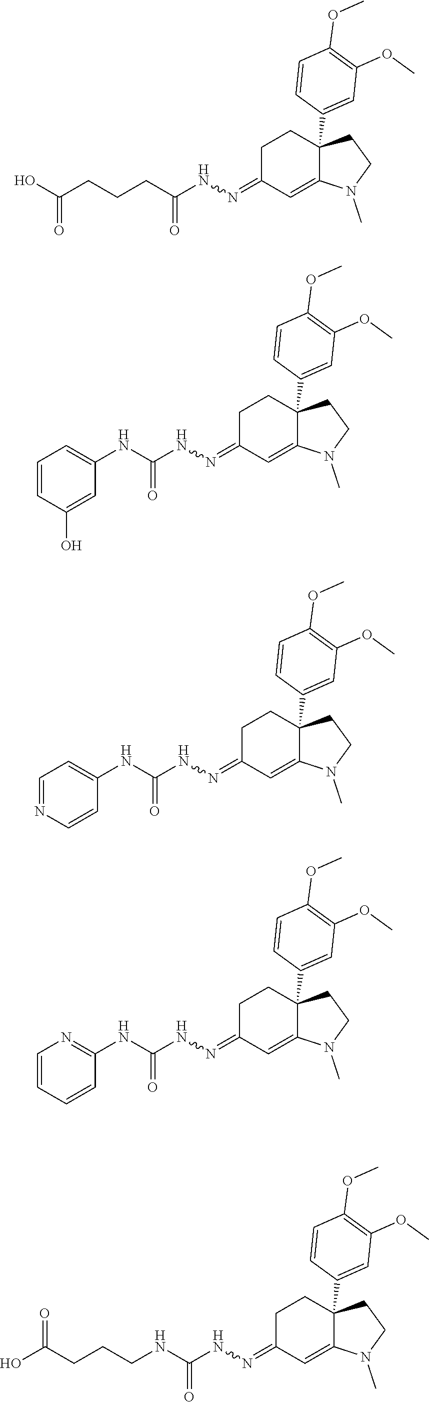 [SMILES: COc1ccc([C@@]23CCC(=NNC(=O)CCCC(=O)O)C=C2N(C)CC3)cc1OC ; COc1ccc([C@@]23CCC(=NNC(=O)Nc4cccc(O)c4)C=C2N(C)CC3)cc1OC ; COc1ccc([C@@]23CCC(=NNC(=O)Nc4ccncc4)C=C2N(C)CC3)cc1OC ; COc1ccc([C@@]23CCC(=NNC(=O)Nc4ccccn4)C=C2N(C)CC3)cc1OC ; COc1ccc([C@@]23CCC(=NNC(=O)NCCCC(=O)O)C=C2N(C)CC3)cc1OC] 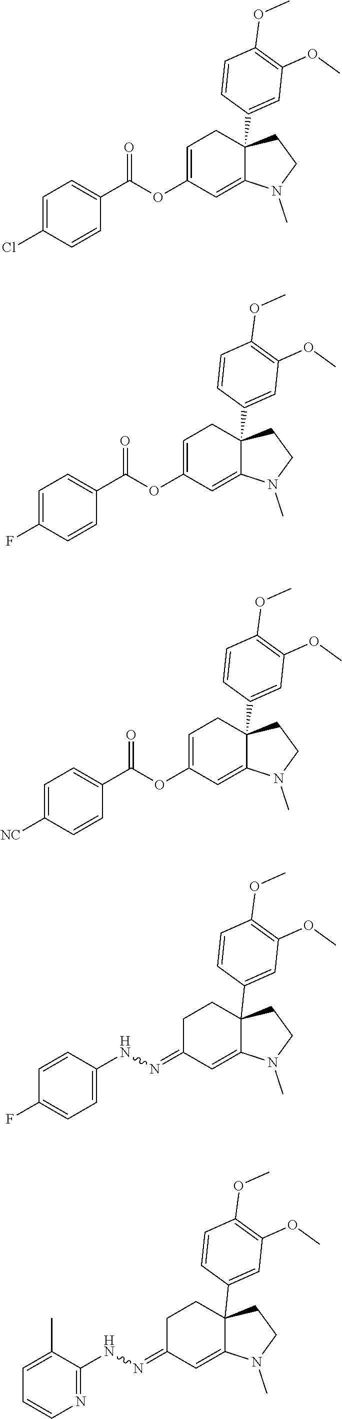 [SMILES: COc1ccc([C@@]23CC=C(OC(=O)c4ccc(Cl)cc4)C=C2N(C)CC3)cc1OC ; COc1ccc([C@@]23CC=C(OC(=O)c4ccc(F)cc4)C=C2N(C)CC3)cc1OC ; COc1ccc([C@@]23CC=C(OC(=O)c4ccc(C#N)cc4)C=C2N(C)CC3)cc1OC ; COc1ccc([C@@]23CCC(=NNc4ccc(F)cc4)C=C2N(C)CC3)cc1OC ; COc1ccc([C@@]23CCC(=NNc4ncccc4C)C=C2N(C)CC3)cc1OC] 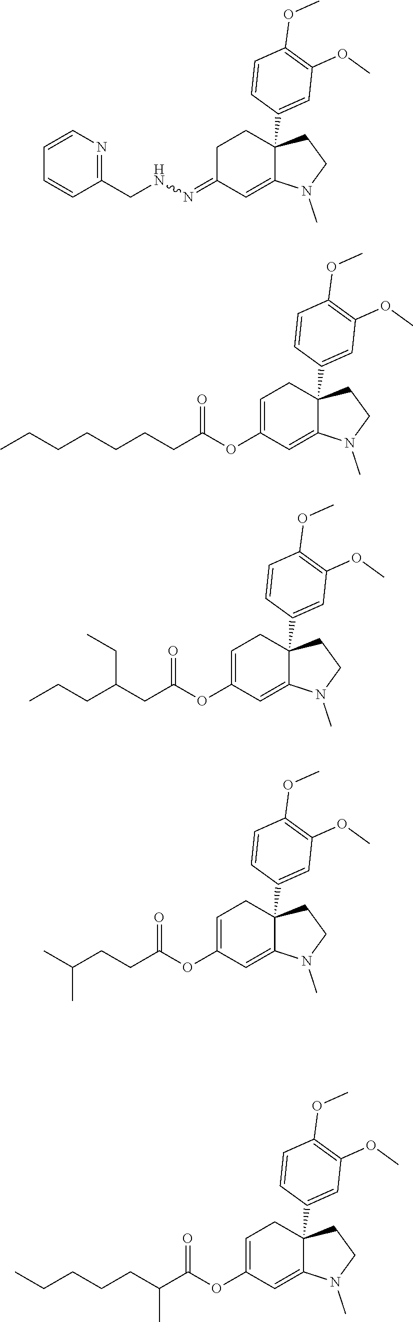 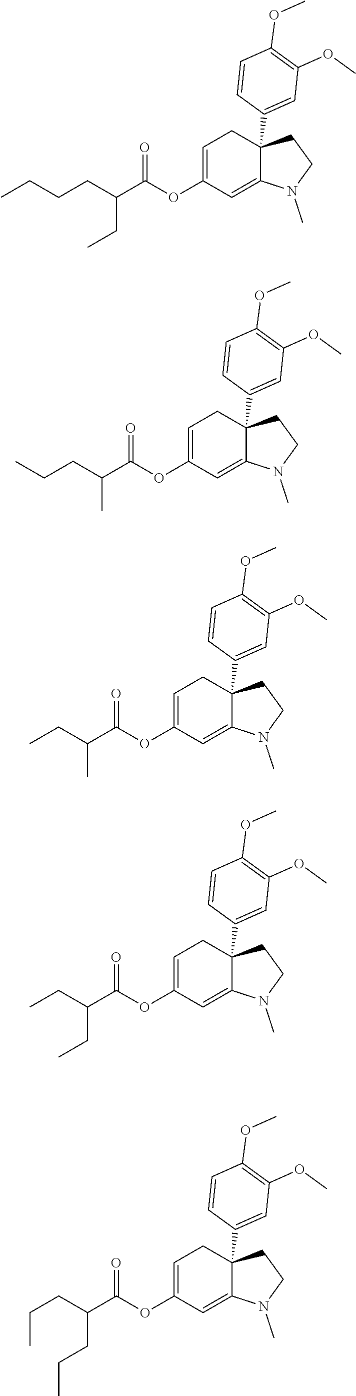 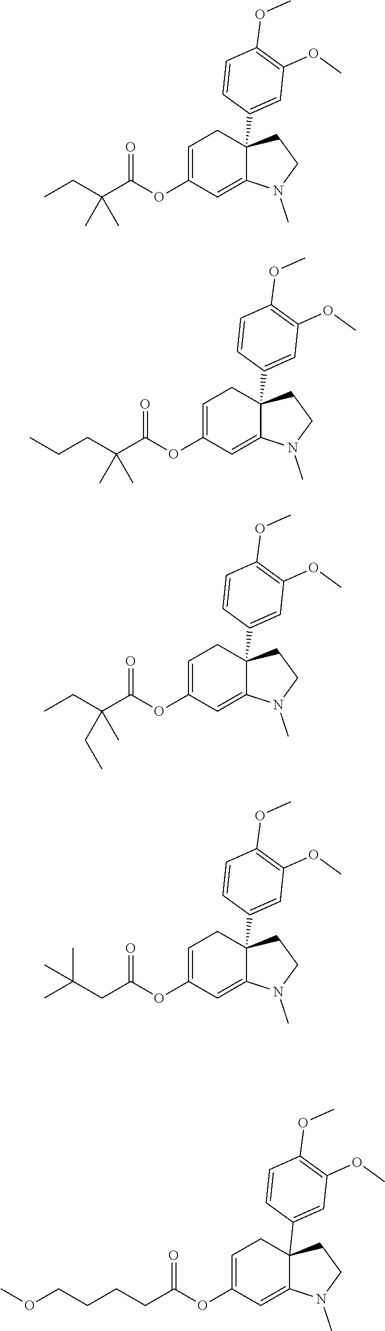
or a pharmaceutically acceptable salt thereof; and
e. a compound selected from the group consisting of: 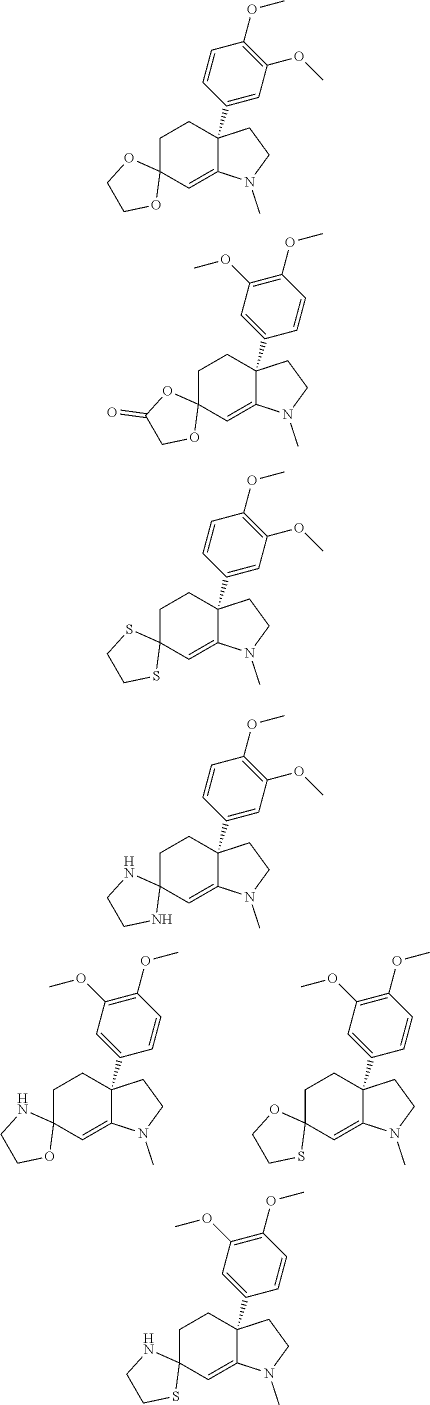 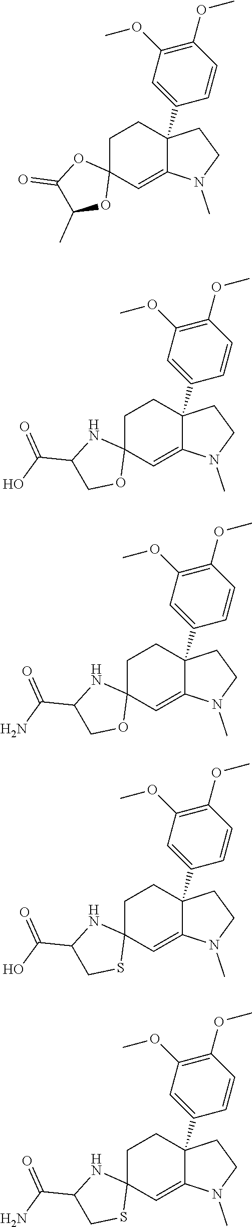 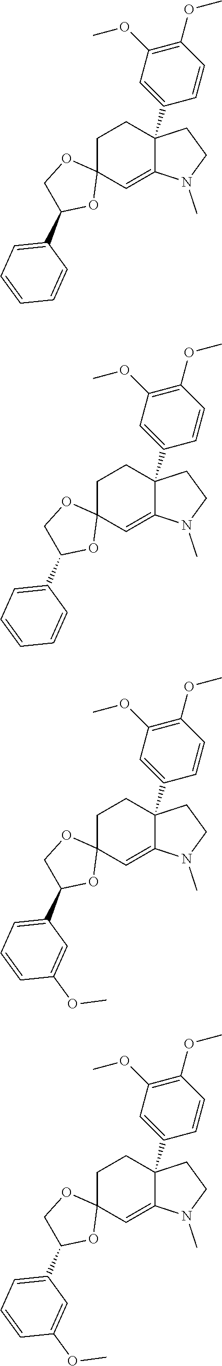 [SMILES: COc1ccc([C@]23CCN(C)C2=CC2(CC3)OC[C@H](c3ccccc3)O2)cc1OC ; COc1ccc([C@]23CCN(C)C2=CC2(CC3)OC[C@@H](c3ccccc3)O2)cc1OC ; COc1cccc([C@H]2COC3(C=C4N(C)CC[C@]4(c4ccc(OC)c(OC)c4)CC3)O2)c1 ; COc1cccc([C@@H]2COC3(C=C4N(C)CC[C@]4(c4ccc(OC)c(OC)c4)CC3)O2)c1] 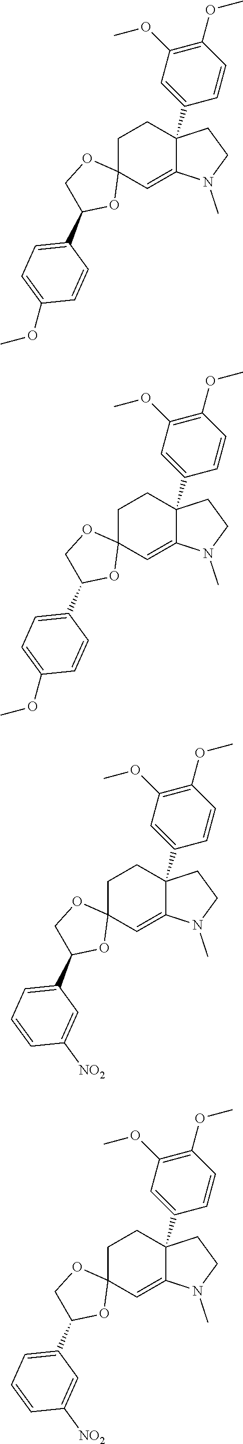 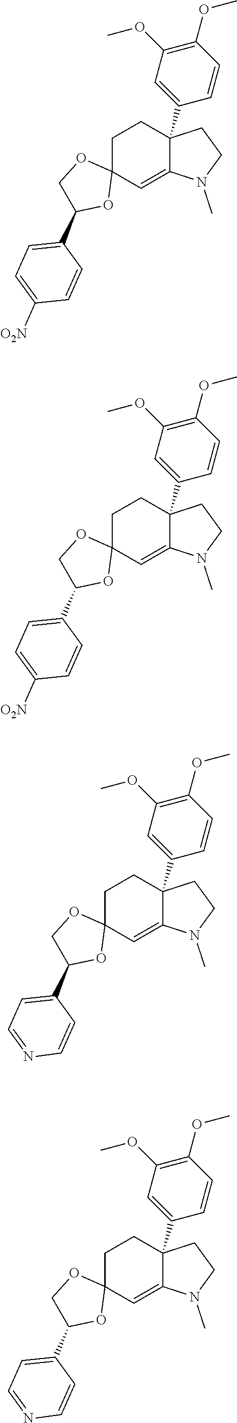 [SMILES: COc1ccc([C@]23CCN(C)C2=CC2(CC3)OC[C@H](c3ccc([N+](=O)[O-])cc3)O2)cc1OC ; COc1ccc([C@]23CCN(C)C2=CC2(CC3)OC[C@@H](c3ccc([N+](=O)[O-])cc3)O2)cc1OC ; COc1ccc([C@]23CCN(C)C2=CC2(CC3)OC[C@H](c3ccncc3)O2)cc1OC ; COc1ccc([C@]23CCN(C)C2=CC2(CC3)OC[C@@H](c3ccncc3)O2)cc1OC] 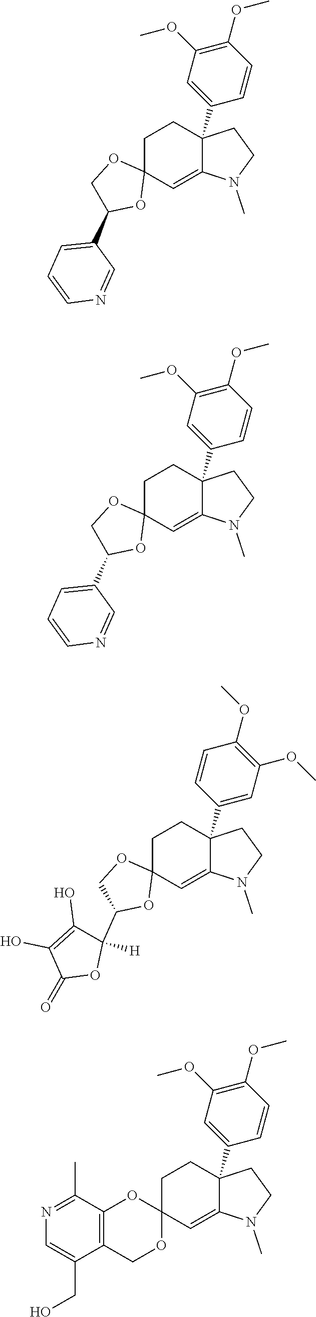 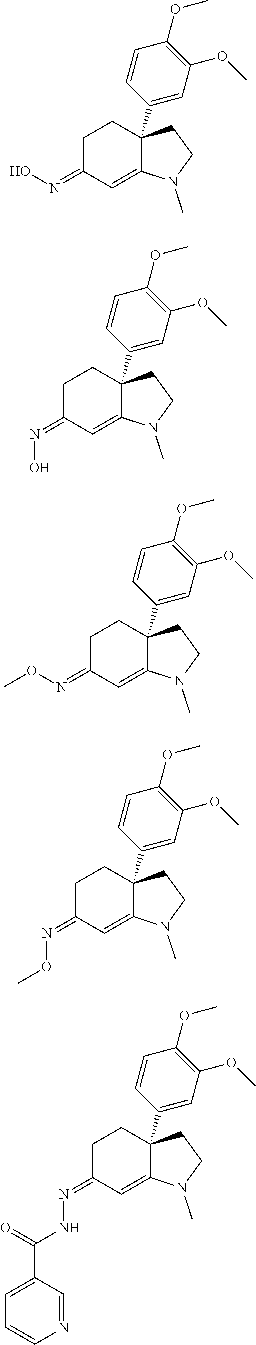 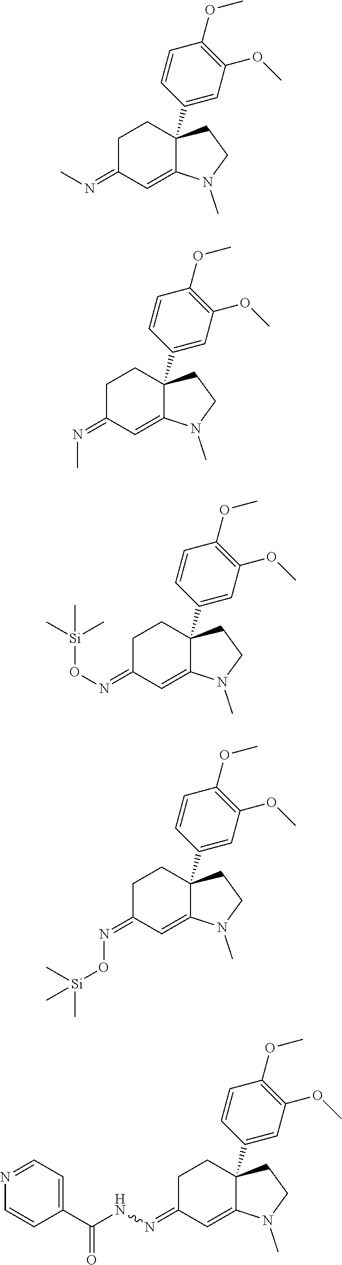 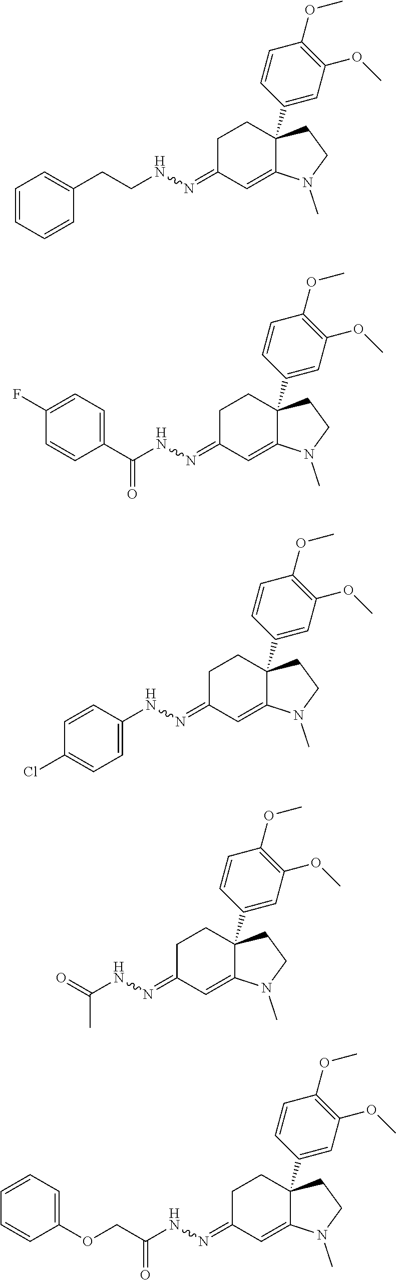 [SMILES: COc1ccc([C@@]23CCC(=NNCCc4ccccc4)C=C2N(C)CC3)cc1OC ; COc1ccc([C@@]23CCC(=NNC(=O)c4ccc(F)cc4)C=C2N(C)CC3)cc1OC ; COc1ccc([C@@]23CCC(=NNc4ccc(Cl)cc4)C=C2N(C)CC3)cc1OC ; COc1ccc([C@@]23CCC(=NNC(C)=O)C=C2N(C)CC3)cc1OC ; COc1ccc([C@@]23CCC(=NNC(=O)COc4ccccc4)C=C2N(C)CC3)cc1OC] 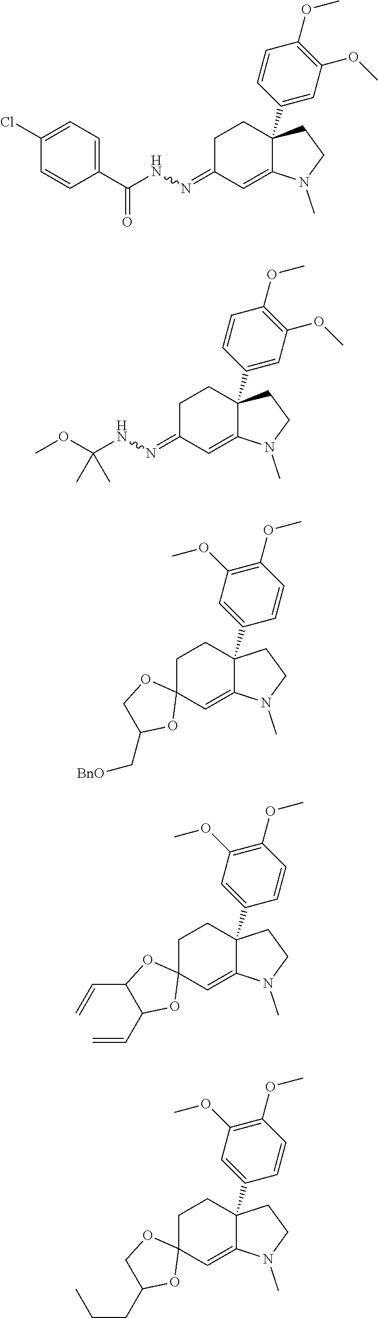 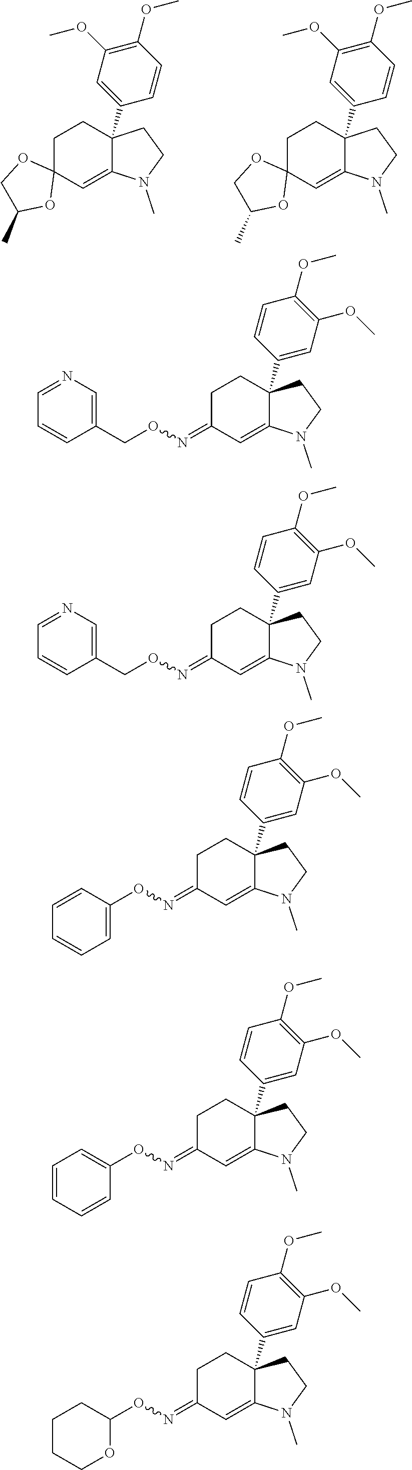 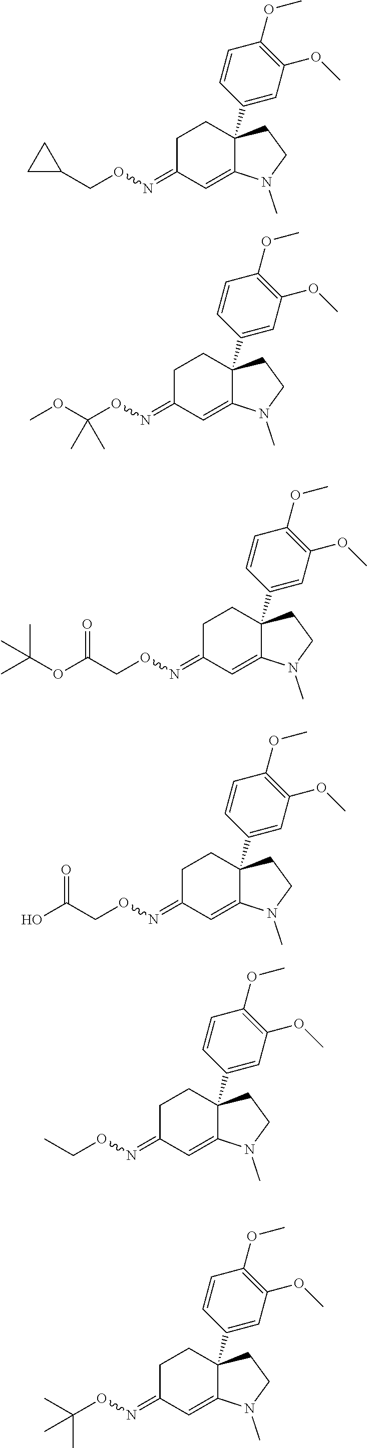 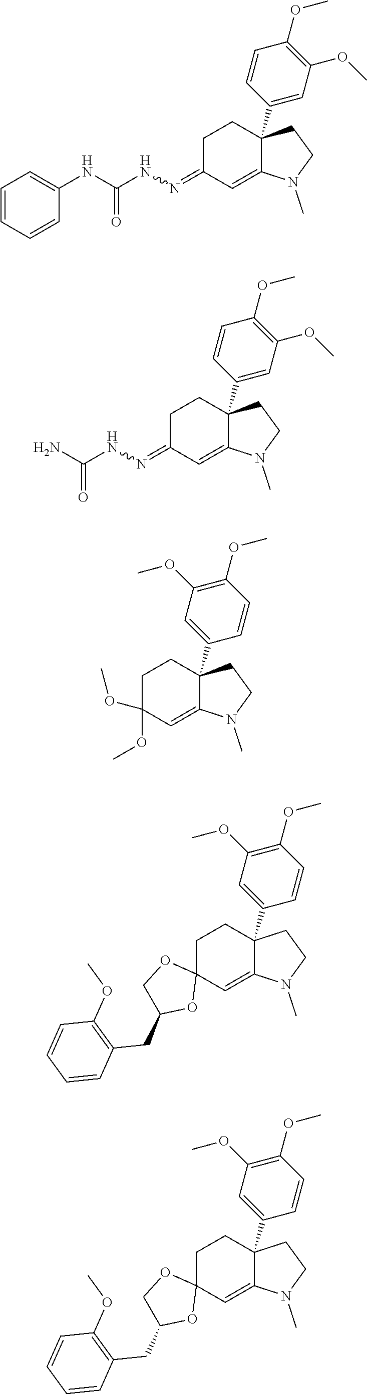 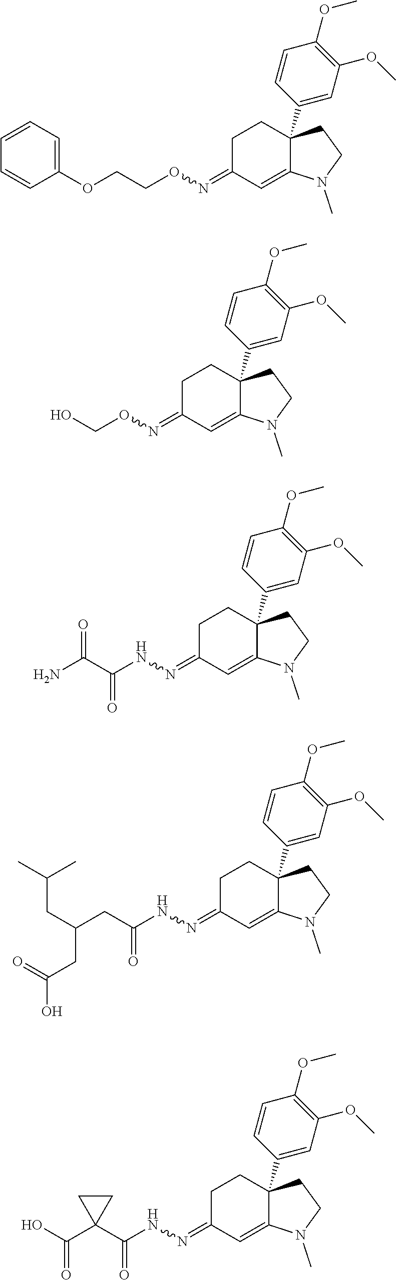 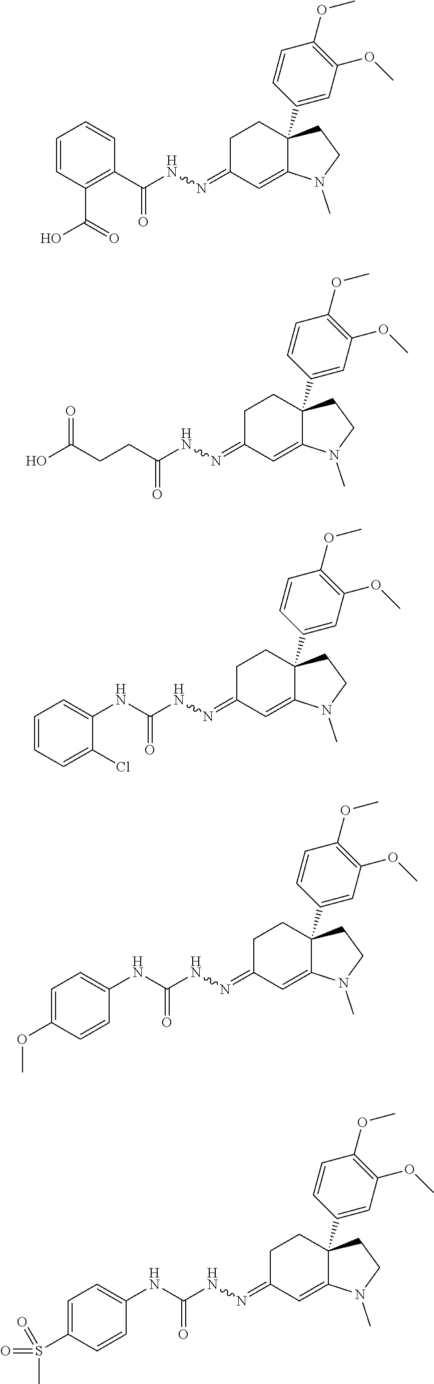 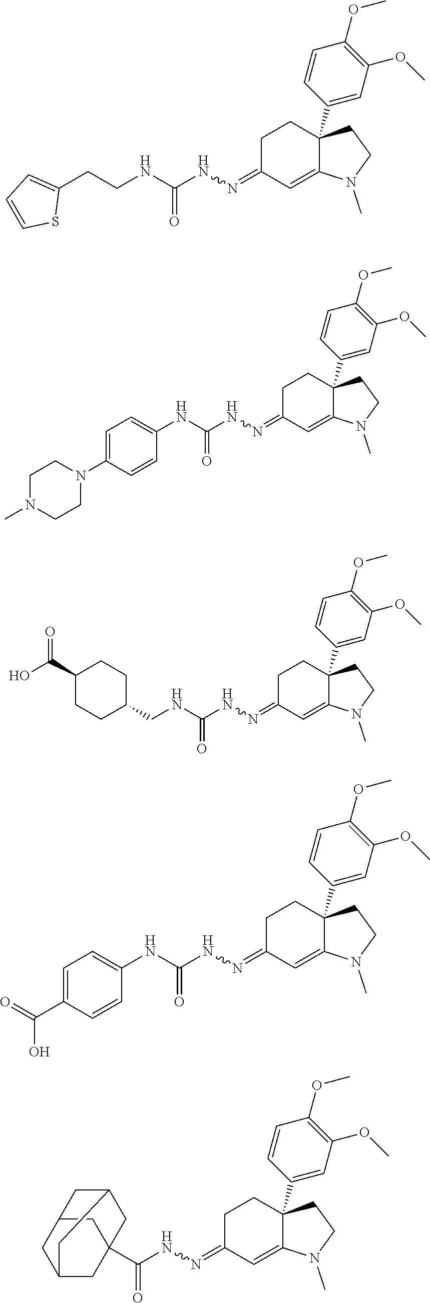 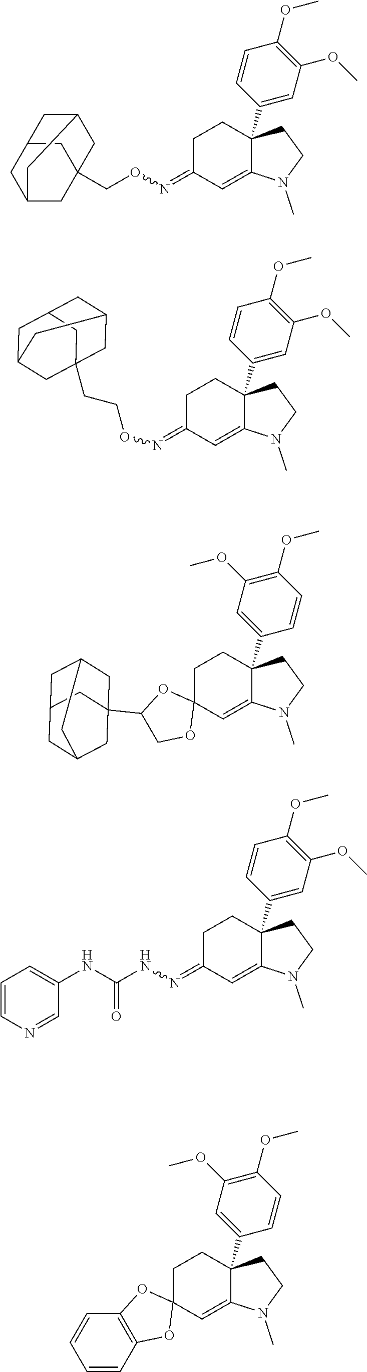 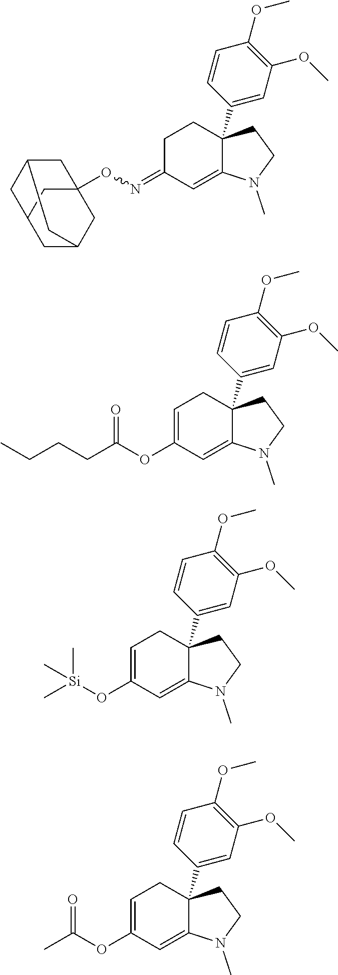 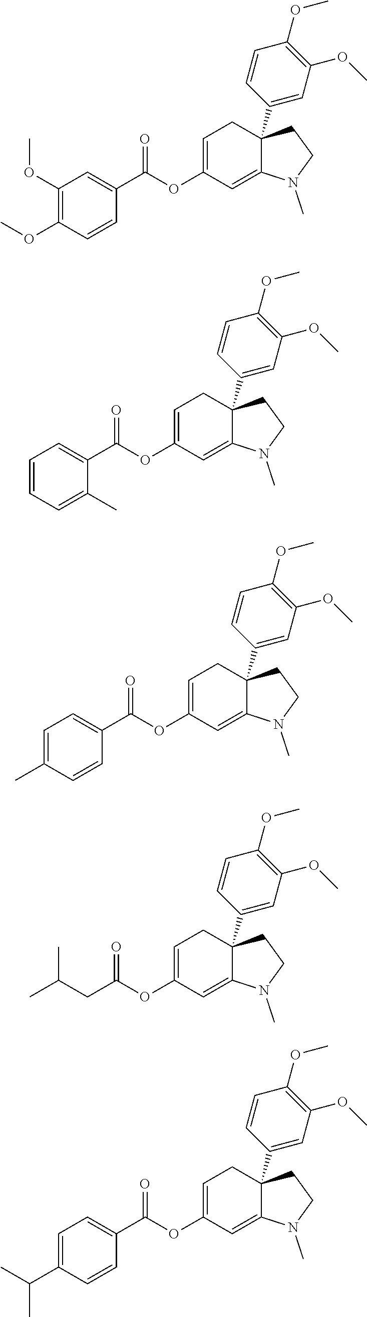 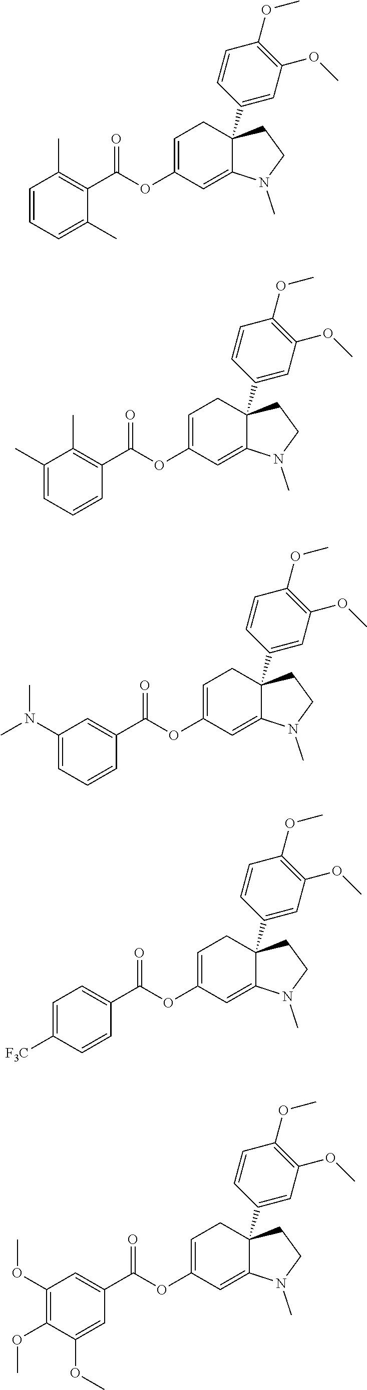 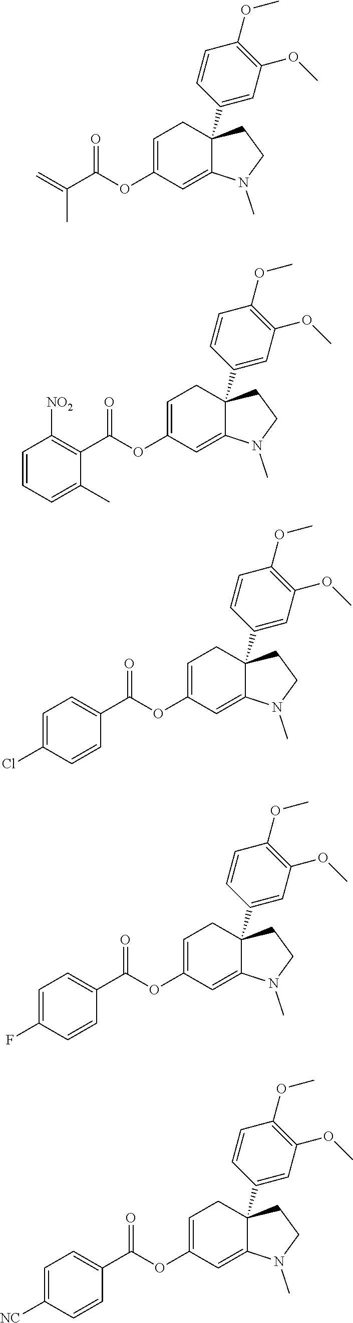 [SMILES: C=C(C)C(=O)OC1=CC[C@@]2(c3ccc(OC)c(OC)c3)CCN(C)C2=C1 ; COc1ccc([C@@]23CC=C(OC(=O)c4c(C)cccc4[N+](=O)[O-])C=C2N(C)CC3)cc1OC ; COc1ccc([C@@]23CC=C(OC(=O)c4ccc(Cl)cc4)C=C2N(C)CC3)cc1OC ; COc1ccc([C@@]23CC=C(OC(=O)c4ccc(F)cc4)C=C2N(C)CC3)cc1OC ; COc1ccc([C@@]23CC=C(OC(=O)c4ccc(C#N)cc4)C=C2N(C)CC3)cc1OC] 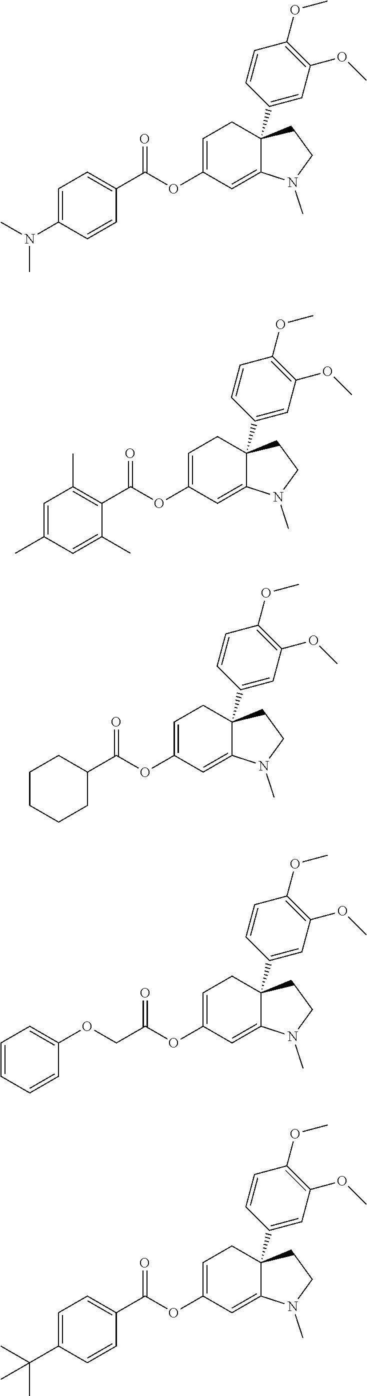 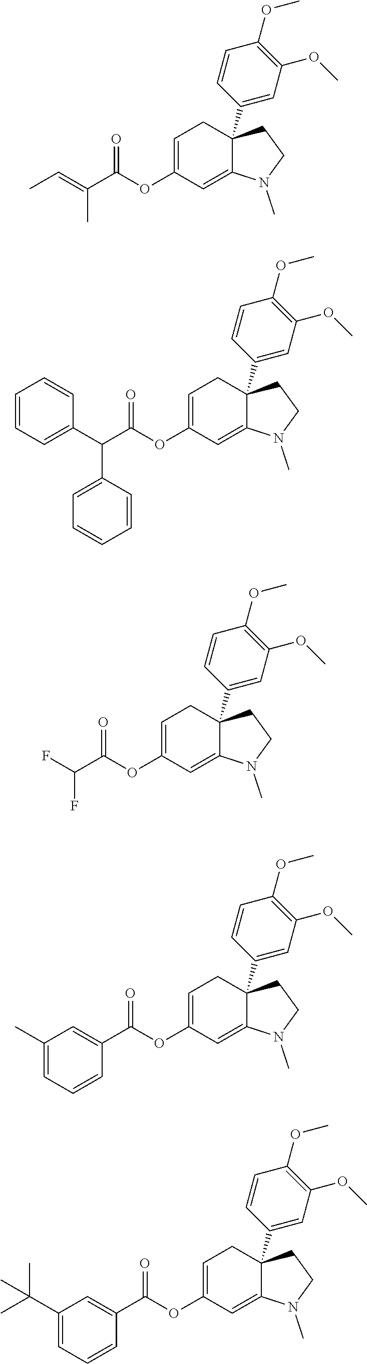 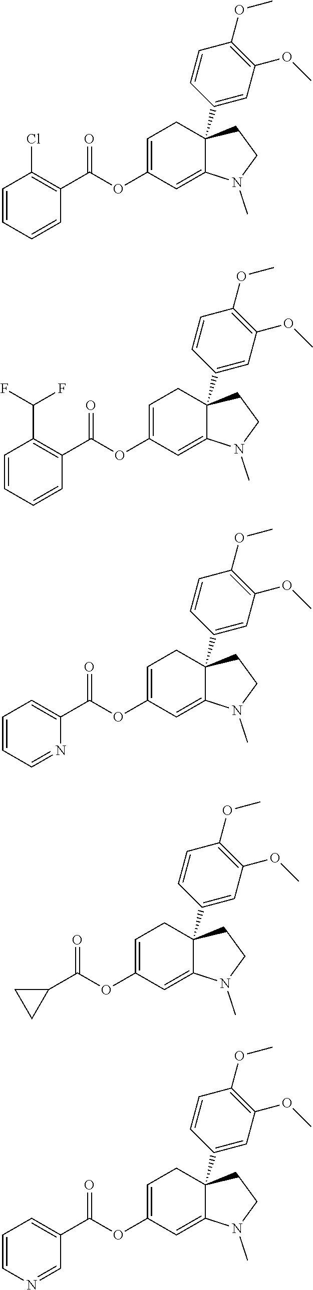 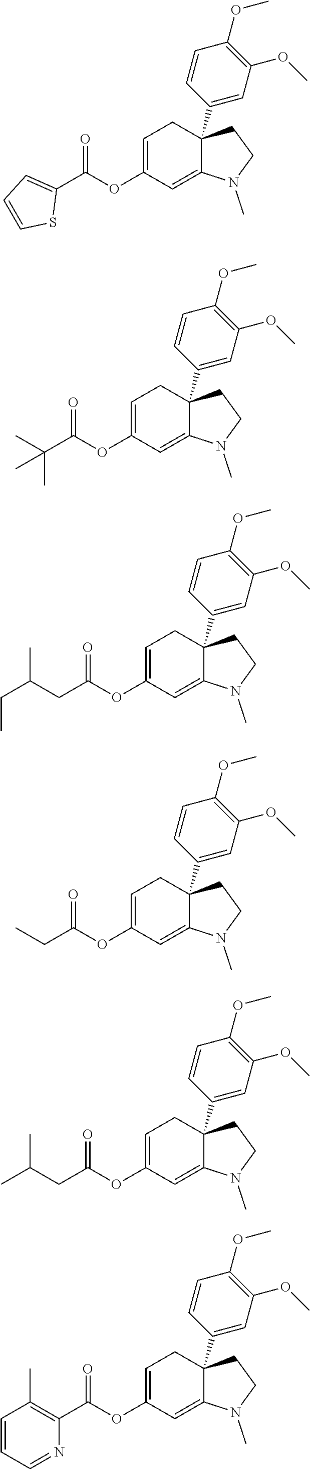 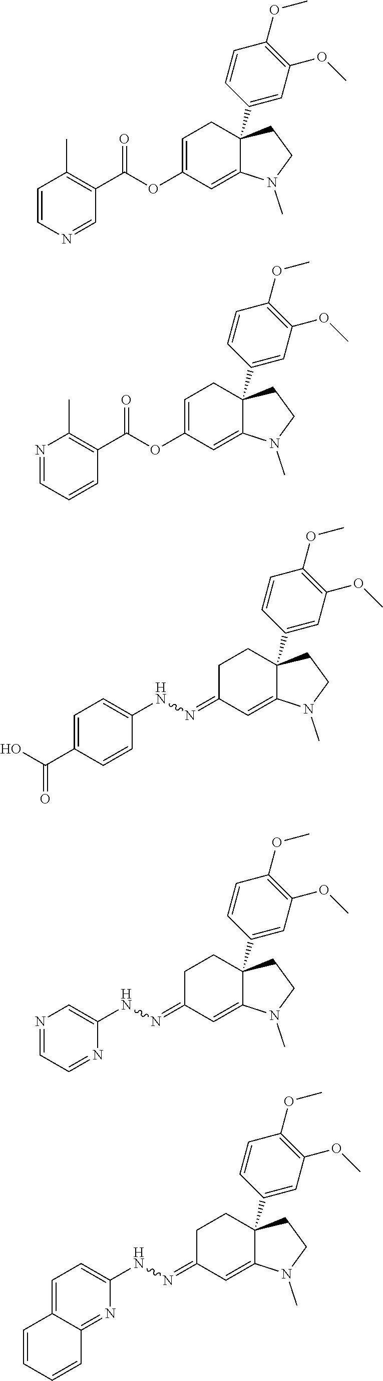 [SMILES: COc1ccc([C@@]23CC=C(OC(=O)c4cnccc4C)C=C2N(C)CC3)cc1OC ; COc1ccc([C@@]23CC=C(OC(=O)c4cccnc4C)C=C2N(C)CC3)cc1OC ; COc1ccc([C@@]23CCC(=NNc4ccc(C(=O)O)cc4)C=C2N(C)CC3)cc1OC ; COc1ccc([C@@]23CCC(=NNc4cnccn4)C=C2N(C)CC3)cc1OC ; COc1ccc([C@@]23CCC(=NNc4ccc5ccccc5n4)C=C2N(C)CC3)cc1OC] 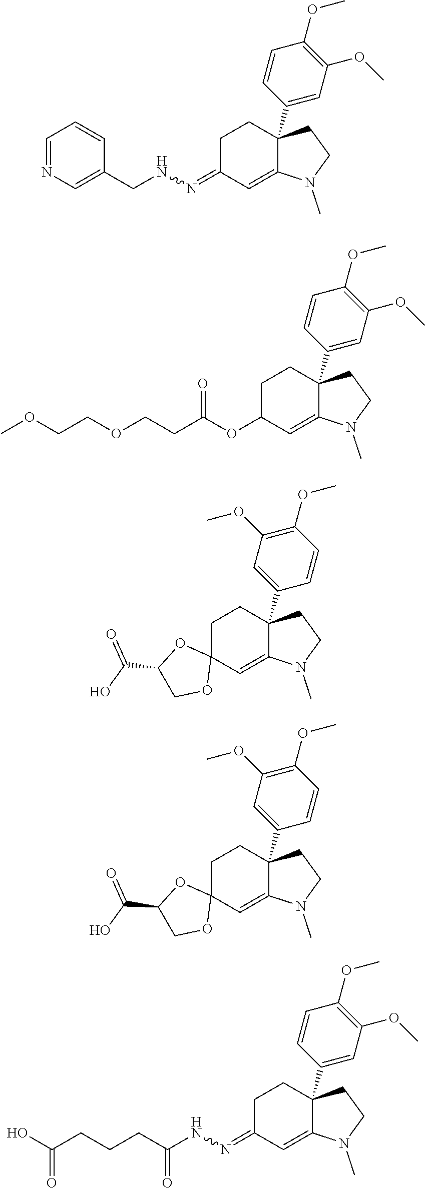 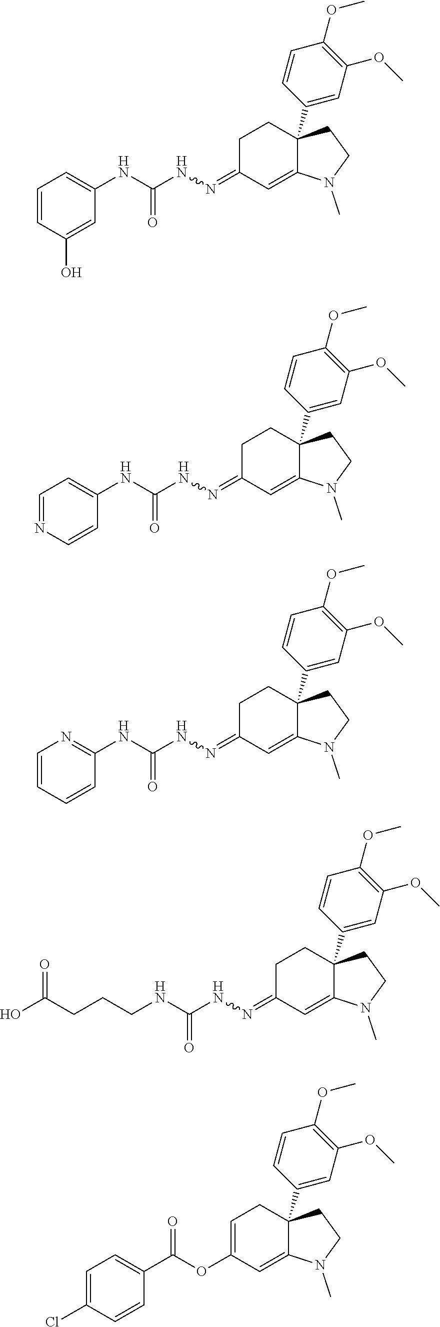 [SMILES: COc1ccc([C@@]23CCC(=NNC(=O)Nc4cccc(O)c4)C=C2N(C)CC3)cc1OC ; COc1ccc([C@@]23CCC(=NNC(=O)Nc4ccncc4)C=C2N(C)CC3)cc1OC ; COc1ccc([C@@]23CCC(=NNC(=O)Nc4ccccn4)C=C2N(C)CC3)cc1OC ; COc1ccc([C@@]23CCC(=NNC(=O)NCCCC(=O)O)C=C2N(C)CC3)cc1OC ; COc1ccc([C@@]23CC=C(OC(=O)c4ccc(Cl)cc4)C=C2N(C)CC3)cc1OC] 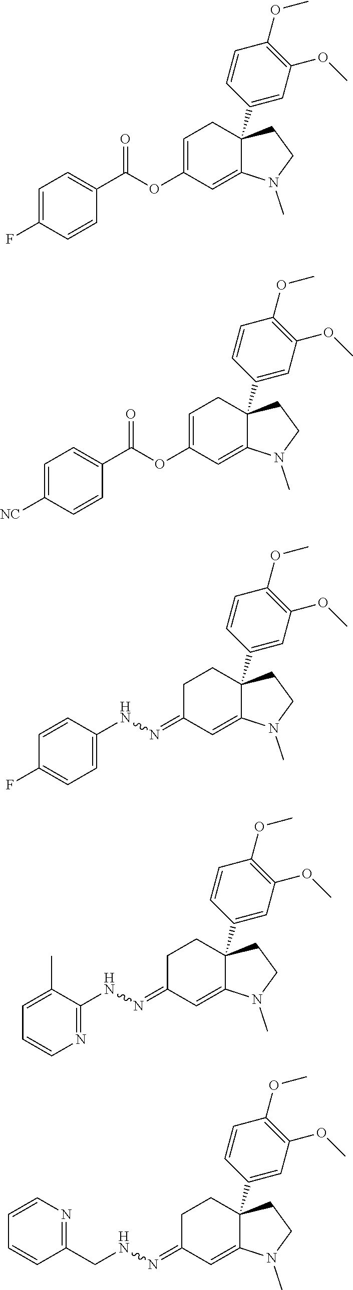 [SMILES: COc1ccc([C@@]23CC=C(OC(=O)c4ccc(F)cc4)C=C2N(C)CC3)cc1OC ; COc1ccc([C@@]23CC=C(OC(=O)c4ccc(C#N)cc4)C=C2N(C)CC3)cc1OC ; COc1ccc([C@@]23CCC(=NNc4ccc(F)cc4)C=C2N(C)CC3)cc1OC ; COc1ccc([C@@]23CCC(=NNc4ncccc4C)C=C2N(C)CC3)cc1OC ; COc1ccc([C@@]23CCC(=NNCc4ccccn4)C=C2N(C)CC3)cc1OC] 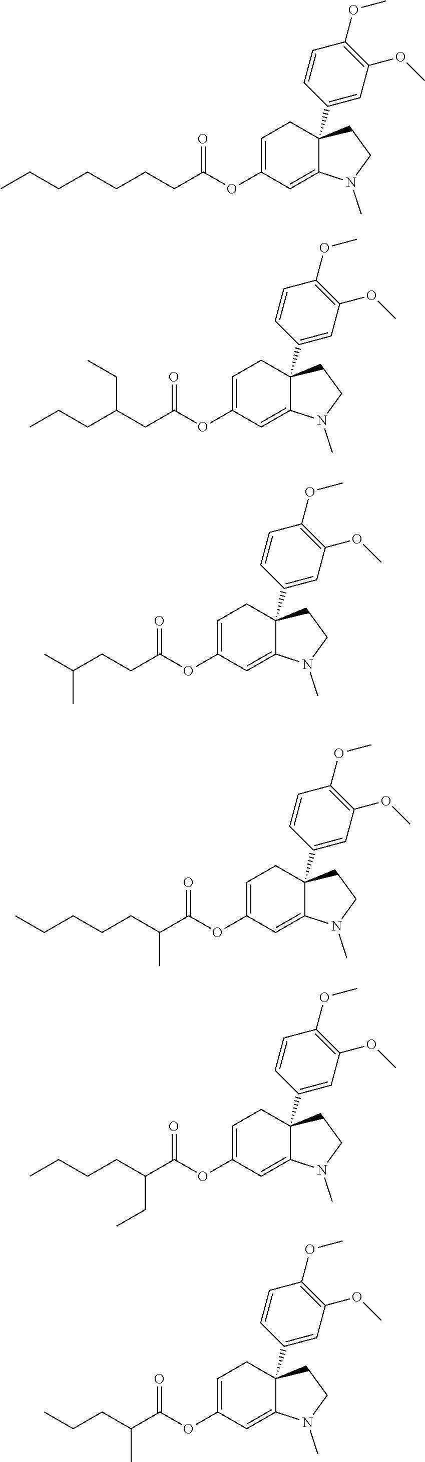 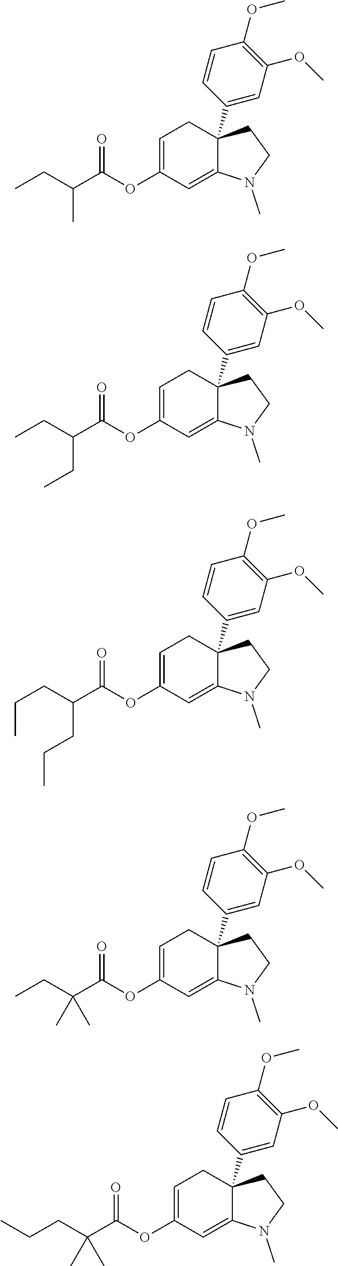 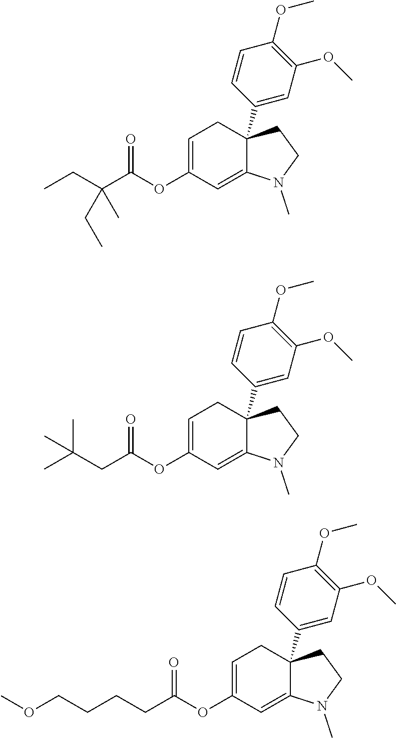
or a pharmaceutically acceptable salt thereof;
wherein the compound has the absolute stereochemistry shown.
